(19) United States
(12) Patent Application Publication
(10) Pub. No.: US 2009/0203694 A1
(43) Pub. Date: Aug. 13, 2009

(54) INHIBITORS OF UNDECAPRENYL PYROPHOSPHATE SYNTHASE

(76) Inventors: Timothy Brian Hurley, Boston, MA (US); Stefan Peukert, Arlington, MA (US); Sompong Wattanasin, Waltham, MA (US)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/375,129

(22) PCT Filed: Jul. 25, 2007
(86) PCT No.: PCT/US07/74303

§ 371 Date: Jan. 26, 2009

Related U.S. Application Data
(60) Provisional application No. 60/820,368, filed on Jul. 26, 2006.

(51) Int. Cl. A61K 31/5377; C07D 401/12; A61K 31/454; C07D 413/12; C07D 471/10; A61K 31/438; A61K 31/4439; A61P 31/04

(52) U.S. Cl. 514/235.5; 546/208; 514/326; 544/141; 546/16; 514/278; 514/341; 546/272.7

(57) ABSTRACT

The present invention relates to compounds that are selective and/or potent inhibitors of UPPS. In addition to compounds which inhibit UPPS, the invention also provides pharmaceutical compositions comprising these compounds and methods of using these compounds for treating bacterial disease, such as bacterial infection.

### RELATED APPLICATIONS

This application claims priority from U.S. Provisional patent application 60/820,368, filed July 2006, which application is hereby expressly incorporated herein in its entirety, including formulae and exemplification. This application is related to U.S. Provisional Application 60/820,367, filed on Jul. 26, 2006, which is hereby expressly incorporated by reference herein in its entirety, including formulae and exemplification.

### BACKGROUND OF THE INVENTION

Prenyltransferases are enzymes important in lipid, peptidoglycan, and glycoprotein biosynthesis. These enzymes act on molecules having a five-carbon isoprenoid substrate. Prenyltransferases are classified into two major subgroups according to whether they catalyze the cis- or trans-prenylation of products in the prenyl chain elongation. E-type prenyltransferases catalyze trans-prenylation and z-type prenyltransferases catalyze cis-prenylation.

Bacterial undecaprenyl pyrophosphate synthase (UPPS), also known as undecaprenyl diphosphate synthase, is a z-type prenyltransferase that catalyzes the sequential condensation of eight molecules of isoprenyl pyrophosphate (IPP) with trans, trans-farnesyl pyrophosphate (FPP) to produce the 55-carbon molecule termed undecaprenyl pyrophosphate. Undecaprenyl pyrophosphate is released from the synthase and dephosphorylated to form undecaprenyl phosphate that serves as the essential carbohydrate and lipid carrier in bacterial cell wall and lipopolysaccharide biosynthesis.

Emerging resistance to currently used antibacterial agents has generated an urgent need for antibiotics acting by different mechanisms. Undecaprenyl pyrophosphate synthase exists ubiquitously in bacteria and plays an essential and critical roll in the cell wall biosynthesis pathway. Thus, undecaprenyl pyrophosphate synthase is essential for cell viability and provides a valid and unexploited molecular target for antibacterial drug discovery.

### SUMMARY OF THE INVENTION

The present invention relates to compounds which inhibit the activity of UPPS, the use of these compounds for treating bacterial disease, pharmaceutical compositions comprising these compounds, as well as methods of identifying these compounds.

Accordingly, in one aspect, the invention pertains, at least in part, to a compound of Formula VII:

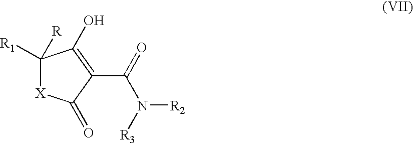

wherein

X is selected from the group consisting of NRₓCRₓRₓ and O;

R is selected from the group consisting of H, an aliphatic group (e.g., alkyl, alkenyl, alkynyl etc.), a carbocyclic group (e.g., saturated or unsaturated), a heterocyclic group (e.g., saturated or unsaturated), halogen, CN, CO₂Rₐ, —C(O)Rₐ, —CORₐ, C(O)NRₐRₐ, which may be optionally substituted, wherein each Rₐ is independently selected from the group consisting of H, an aliphatic group, a carbocyclic group, and a heterocyclic group; or R and R₁, taken together, may form a substituted or unsubstituted spiro heterocyclic or carbocyclic ring, which may optionally be substituted (e.g., by an aliphatic group, a carbocyclic group, or a heterocyclic group);

R₁ and Rₓ are independently selected from the group consisting of H, -M₁, -M₁-M₂, -Z-M₂, and -M₁-Z-M₂; or R and R₁, taken together, may form a substituted or unsubstituted spiro heterocyclic or carbocyclic ring, which may optionally be substituted;

M₁ and M₂ are independently selected from the group consisting of H, an aliphatic group, a carbocyclic group, a heterocyclic group, which may be optionally substituted;

Z is selected from the group consisting of —O—, —NH—, —CR_{z}R_{z}—, —S—, —S(O)—, —C(O)—, —NHC(O)—, —C(O)NH—, —NHC(O)CH₂O—, —S(O)₂—, —CH(OH)—, —CH(OR_{z}), —C(O)CH₂—, —CH₂C(O)—, —CH₂CH(OH)—, —CH₂CH(OR_{z})—, —CH(OH)CH₂—, —CH(OR_{z})CH₂—, and any combination thereof, wherein each R_{z} is independently selected from the group consisting of H, an aliphatic group, a carbocyclic group, a heterocyclic group, hydroxy, and alkoxy;

R₂ is selected from the group consisting of H, an aliphatic group, a carbocyclic group, and a heterocyclic group (e.g., selected from the group consisting of phenyl, benzyl, isobutyl, cyclohexyl, cyclohexylmethyl, m-methoxy phenyl, alkyl, aryl, and heterocycle);

R₃ is selected from the group consisting of -G₁, -G₁-G₂, —Y-G₂, and -G₁-Y-G₂;

G₁ and G₂ are independently selected from H, an aliphatic group, a carbocyclic group, and a heterocyclic group, which may be optionally substituted with one or more of substituents; and

Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂—, —CH(OH)—, —CH(OR_{y}), —C(O)CH₂—, —CH₂C(O)—, —CH₂CH(OH)—, —CH₂CH(OR_{y})—, —CH(OH)CH₂—, —CH(OR_{y})CH₂—, and any combination thereof, wherein each R_{y} is independently selected from the group consisting of H, an aliphatic group, a carbocyclic group, a heterocyclic group, hydroxy, and alkoxy.

It will be noted that the structure of some of the compounds of this invention includes asymmetric carbon atoms. It is to be understood accordingly that the isomers arising from such asymmetry (e.g., all enantiomers and diastereomers) are included within the scope of this invention unless indicated otherwise. Such isomers can be obtained in substantially pure form by classical separation techniques and by stereochemically controlled synthesis. That is, unless otherwise stipulated, any chiral carbon center may be of either (R)- or (S)-stereochemistry. Furthermore, alkenes can include either the E- or Z-geometry, where appropriate. Additionally, one skilled in the art will appreciate that the chemical structures as drawn may represent a number of possible tautomers, and the present invention also includes those tautomers.

Accordingly, another embodiment of the invention is a substantially pure single stereoisomer or a mixture of stereoisomers, e.g., pre-determined to be within specific amounts.

Moreover, it should be understood that the compounds of the present invention, comprise compounds that satisfy valency requirements known to the ordinarily skilled artisan. Additionally, compounds of the present invention comprise stable compounds as well as though compounds that may be modified, e.g., chemically or through appropriate formulation, to become stable. In certain embodiments, such stability is guided by time periods that are sufficient to allow administration to and/or treatment of a subject.

In addition, compounds of the invention further include derivatives of the compounds depicted below modified to adjust at least one chemical or physical property of a depicted compound. In certain embodiments, the modification comprises substitution of a carbon atom with a heteroatom or addition of a heteroatom-containing substituent (e.g., substituted by a substituent selected from the group consisting of hydroxy, alkoxy, heterocycle and an acyl group), such that one or more of the chemical or physical properties of the depicted compound have been enhanced, e.g., with respect to potency or selectivity. For example, particular embodiments of substituted alkyl moieties may be —CH₂OH or —CH₂OCH₃.

In another aspect, the invention is directed to a compound of Formula VIII:

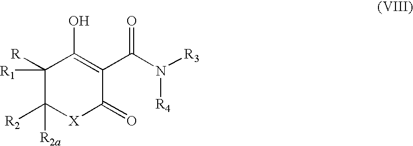

wherein

X is selected from the group consisting of NRₓ and O;

R is absent or selected from the group consisting of H, an aliphatic group (e.g., alkyl, alkenyl, alkynyl etc.), a carbocyclic group (e.g., saturated or unsaturated), a heterocyclic group (e.g., saturated or unsaturated), halogen, NO₂, CN, ORₐ, NRₐRₐ, CO₂Rₐ, —C(O)Rₐ, —CORₐ, NRₐC(O)Rₐ, NRₐC(O)NRₐRₐ, NRₐRₐC(O)O—, C(O)NRₐRₐ, which may be optionally substituted, wherein each Rₐ is independently selected from the group consisting of H, an aliphatic group, a carbocyclic group, and a heterocyclic group; or R and R₁; taken together, may form a substituted or unsubstituted spiro heterocyclic or carbocyclic ring, which may optionally be substituted (e.g., by an aliphatic group, a carbocyclic group, or a heterocyclic group); or R₂ and R₂ₐ, taken together, may form a substituted or unsubstituted spiro heterocyclic or carbocyclic ring, which may optionally be substituted (e.g., by an aliphatic group, a carbocyclic group, or a heterocyclic group);

R₂ₐ is absent or selected from the group consisting of H, an aliphatic group (e.g., alkyl, alkenyl, alkynyl etc.), a carbocyclic group (e.g., saturated or unsaturated), a heterocyclic group (e.g., saturated or unsaturated), halogen, CN, CO₂Rₐ, —C(O)Rₐ, —CORₐ, C(O)NRₐRₐ, which may be optionally substituted, wherein each Rₐ is independently selected from the group consisting of H, an aliphatic group, a carbocyclic group, and a heterocyclic group; or R and R₁, taken together, may form a substituted or unsubstituted spiro heterocyclic or carbocyclic ring, which may optionally be substituted (e.g., by an aliphatic group, a carbocyclic group, or a heterocyclic group); or R₂ and R₂ₐ, taken together, may form a substituted or unsubstituted spiro heterocyclic or carbocyclic ring, which may optionally be substituted (e.g., by an aliphatic group, a carbocyclic group, or a heterocyclic group);

R₁, R₂, and Rₓ are independently selected from the group consisting of H, -M₁, -M₁-M₂, -Z-M₂, and -M₁-Z-M₂; or R and R₁, taken together, may form a substituted or unsubstituted spiro heterocyclic or carbocyclic ring, which may optionally be substituted; or R₂ and R₂ₐ, taken together, may form a substituted or unsubstituted spiro heterocyclic or carbocyclic ring, which may optionally be substituted;

M₁ and M₂ are independently selected from the group consisting of H, an aliphatic group, a carbocyclic group, a heterocyclic group, which may be optionally substituted;

Z is selected from the group consisting of —O—, —NH—, —CR_{z}R_{z}—, —S—, —S(O)—, —C(O)—, —NHC(O)—, —C(O)NH—, —NHC(O)CH₂O—, —S(O)₂—, —CH(OH)—, —CH(OR_{z}), —C(O)CH₂—, —CH₂C(O)—, —CH₂CH(OH)—, —CH₂CH(OR_{z})—, —CH(OH)CH₂—, —CH(OR_{z})CH₂—, and any combination thereof, wherein each R_{z} is independently selected from the group consisting of H, an aliphatic group, a carbocyclic group, a heterocyclic group, hydroxy, and alkoxy;

R₃ is selected from the group consisting of -G₁, -G₁-G₂, —Y-G₂, and -G₁-Y-G₂;

G₁ and G₂ are independently selected from H, an aliphatic group, a carbocyclic group, and a heterocyclic group, which may be optionally substituted with one or more of substituents;

Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂—, —CH(OH)—, —CH(OR_{y}), —C(O)CH₂—, —CH₂C(O)—, —CH₂CH(OH)—, —CH₂CH(OR_{y})—, —CH(OH)CH₂—, —CH(OR_{y})CH₂—, and any combination thereof, wherein each R_{y} is independently selected from the group consisting of H, an aliphatic group, a carbocyclic group, a heterocyclic group, hydroxy, and alkoxy; and

R₄ is selected from the group consisting of H, an aliphatic group, a carbocyclic group, and a heterocyclic group (e.g., selected from the group consisting of phenyl, benzyl, isobutyl, cyclohexyl, cyclohexylmethyl, m-methoxy phenyl, alkyl, aryl, and heterocycle).

In another aspect, the invention is directed to a compound of Formula IX:

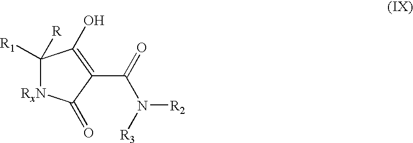

wherein

R is selected from the group consisting of H, benzyl, pyridinyl, tetrahydro-pyranyl, methyl-1H-imidazolyl, cyclohexylmethyl, phenethyl, p-chlorobenzyl, carboxylic acid benzyl ester, propionic acid tert-butyl ester, tert-butyl ester, ethanone, hydroxy, methoxy, ethoxy, propoxy, butoxy, t-butoxy, phenyl, isobutyl, methyl, ethyl, propyl, butyl, cyclohexyl, cyclohexylmethyl, m-methoxy phenyl, carboxylic acid 2-methoxy-ethyl ester, 3,3-dimethyl-butan-1-one, 2,2-dimethyl-propan-1-one, carboxylic acid methyl ester, alkyl, halogen, CN, CO₂Rₐ, —C(O)Rₐ, —CORₐ, C(O)NRₐRₐ, aryl, and heterocycle, which may be optionally substituted with methoxy or 2-methoxy-ethoxy, wherein each Rₐ is independently selected from the group consisting of H, alkyl, aryl, and heterocycle; or R and R₁, taken together, may form a substituted or unsubstituted spiro heterocyclic or carbocyclic ring, which may optionally be substituted with a benzyl group;

R₁ and Rₓ are independently selected from the group consisting of H, benzyl, pyridinyl, tetrahydro-pyranyl, methyl-1H-imidazolyl, cyclohexylmethyl, phenethyl, p-chlorobenzyl, carboxylic acid benzyl ester, propionic acid tert-butyl ester, tert-butyl ester, ethanone, propoxy, t-butoxy, phenyl, isobutyl, methyl, ethyl, propyl, butyl, cyclohexyl, cyclohexylmethyl, m-methoxy phenyl, carboxylic acid 2-methoxy-ethyl ester, 3,3-dimethyl-butan-1-one, 2,2-dimethyl-propan-1-one, carboxylic acid methyl ester, alkyl, halogen, CN, CO₂R_{b}, —C(O)R_{b}, —COR_{b}, C(O)NR_{b}R_{b}, aryl, and heterocycle, which may be optionally substituted with methoxy or 2-methoxy-ethoxy, wherein each R_{b} is independently selected from the group consisting of H, alkyl, aryl, and heterocycle; or R and R₁, taken together, may form a substituted or unsubstituted spiro heterocyclic or carbocyclic ring, which may optionally be substituted with a benzyl group;

R₂ is selected from the group consisting of H, phenyl, benzyl, isobutyl, cyclohexyl, cyclohexylmethyl, m-methoxy phenyl, alkyl, aryl, and heterocycle;

R₃ is selected from the group consisting of -G₁, -G₁-G₂, —Y-G₂, and -G₁-Y-G₂;

G₁ and G₂ are independently selected from the group consisting of phenyl, cyclohexyl, cyclopentyl, 4-indanyl, pyrimidinyl, N-morpholino, furanyl, thiophenyl, pyrrolyl, N-1H-pyridin-2-onyl, bicyclo[4.2.0]octa-1,3,5-trien-3-yl, 1-indanyl, naphthalenyl, tetrahydro-naphthalenyl, pyrazine, [1,2,3]thiadiazolyl, 3-isoxazolyl, 5-indolyl, 2,3-dihydro-indol-6-yl, indazol-5-yl, benzo[2,1,3]thiadiazol-5-yl, cycloheptyl, isopropyl-[1,3,4]thiadiazolyl, benzothiazolyl, 3-methyl-butyl, 1H-pyrazolyl, oxazolyl, piperidinyl, 1H-imidazolyl, pyrrolidinyl, piperazinyl, 1H-[1,2,4]triazolyl, and pyridinyl, which may be optionally substituted with one or more of substituent moieties selected from the group consisting of CF₃, OCF₃, iodo, chloro, bromo, —C(O)NH₂, —O(CH₂)₅CH₃, carboxylic acid methyl ester, phenyl, p-methoxy phenyl, —NHC(O)NH₂, —C(O)O(CH₂)₂N(CH₂CH₃)₂, t-butyl, fluoro, methoxy, hydroxy, isopropyl, cyano, isopropenyl tetrahydropyran, benzyl, amino, —NHC(O)OC(CH₃)₃, —C(O)OH, —C(O)CH₃, —CH₂CO₂H, methyl, and —(CH₂)₂—OH; and

Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂—, and any combination thereof, wherein each R_{y} is independently selected from the group consisting of H, alkyl, aryl, heterocycle, hydroxy, or alkoxy.

An addition embodiment of the invention relates to a compound of Formula X:

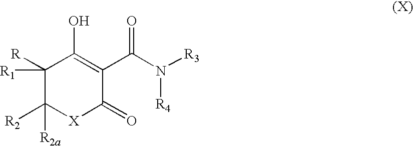

wherein

X is selected from the group consisting of NRₓCRₓRₓ and O;

R₂ and R₂ₐ are absent or independently selected from the group consisting of H, benzyl, pyridinyl, tetrahydro-pyranyl, methyl-1H-imidazolyl, cyclohexylmethyl, phenethyl, p-chlorobenzyl, carboxylic acid benzyl ester, propionic acid tert-butyl ester, tert-butyl ester, ethanone, hydroxy, methoxy, ethoxy, propoxy, butoxy, t-butoxy, phenyl, isobutyl, methyl, ethyl, propyl, butyl, cyclohexyl, cyclohexylmethyl, m-methoxy phenyl, carboxylic acid 2-methoxy-ethyl ester, 3,3-dimethyl-butan-1-one, 2,2-dimethyl-propan-1-one, carboxylic acid methyl ester, alkyl, halogen, CN, CO₂Rₐ, —C(O)Rₐ, —CORₐ, C(O)NRₐRₐ, aryl, and heterocycle, which may be optionally substituted with methoxy or 2-methoxy-ethoxy, wherein each Rₐ is independently selected from the group consisting of H, alkyl, aryl, and heterocycle; or R₂ and R₂ₐ, taken together, may form a substituted or unsubstituted spiro heterocyclic or carbocyclic ring, which may optionally be substituted; or R and R₂ are absent;

R₁, R, and each Rₓ are independently selected from the group consisting of H, benzyl, pyridinyl, tetrahydro-pyranyl, methyl-1H-imidazolyl, cyclohexylmethyl, phenethyl, p-chlorobenzyl, carboxylic acid benzyl ester, propionic acid tert-butyl ester, tert-butyl ester, ethanone, hydroxy, methoxy, ethoxy, propoxy, butoxy, t-butoxy, phenyl, isobutyl, methyl, ethyl, propyl, butyl, cyclohexyl, cyclohexylmethyl, m-methoxy phenyl, carboxylic acid 2-methoxy-ethyl ester, 3,3-dimethyl-butan-1-one, 2,2-dimethyl-propan-1-one, carboxylic acid methyl ester, alkyl, halogen, NO₂, CN, OR_{b}, NR_{b}R_{b}, CO₂R_{b}, —C(O)R_{b}, —COR_{b}, NR_{b}C(O)R_{b}, NR_{b}C(O)NR_{b}R_{b}, NR_{b}R_{b}C(O)O—, C(O)NR_{b}R_{b}, aryl, and heterocycle, which may be optionally substituted with methoxy or 2-methoxy-ethoxy, wherein each R_{b} is independently selected from the group consisting of H, alkyl, aryl, and heterocycle; or R and R₁, taken together, may form a substituted or unsubstituted spiro heterocyclic or carbocyclic ring, which may optionally be substituted; or R and R₂ are absent;

R₃ is selected from the group consisting of -G₁, -G₁-G₂, —Y-G₂, and -G₁-Y-G₂;

G₁ and G₂ are independently selected from the group consisting of phenyl, cyclohexyl, cyclopentyl, 4-indanyl, pyrimidinyl, N-morpholino, furanyl, thiophenyl, pyrrolyl, N-1H-pyridin-2-onyl, bicyclo[4.2.0]octa-1,3,5-trien-3-yl, 1-indanyl, naphthalenyl, tetrahydro-naphthalenyl, pyrazine, [1,2,3]thiadiazolyl, 3-isoxazolyl, 5-indolyl, 2,3-dihydro-indol-6-yl, indazol-5-yl, benzo[2,1,3]thiadiazol-5-yl, cycloheptyl, isopropyl-[1,3,4]thiadiazolyl, benzothiazolyl, 3-methyl-butyl, 1H-pyrazolyl, oxazolyl, piperidinyl, 1H-imidazolyl, pyrrolidinyl, piperazinyl, 1H-[1,2,4]triazolyl, and pyridinyl, which may be optionally substituted with one or more of substituent moieties selected from the group consisting of CF₃, OCF₃, iodo, chloro, bromo, —C(O)NH₂, —O(CH₂)₅CH₃, carboxylic acid methyl ester, phenyl, p-methoxy phenyl, —NHC(O)NH₂, —C(O)O(CH₂)₂N(CH₂CH₃)₂, t-butyl, fluoro, methoxy, hydroxy, isopropyl, cyano, isopropenyl tetrahydropyran, benzyl, amino, —NHC(O)OC(CH₃)₃, —C(O)OH, —C(O)CH₃, —CH₂CO₂H, methyl, and —(CH₂)₂—OH;

Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂—, and any combination thereof, wherein each R_{y} is independently selected from the group consisting of H, alkyl, aryl, heterocycle, hydroxy, or alkoxy; and

R₄ is selected from the group consisting of H, phenyl, benzyl, isobutyl, cyclohexyl, cyclohexylmethyl, m-methoxy phenyl, alkyl, aryl, and heterocycle.

Another aspect of the invention pertains to a compound of Formula XI:

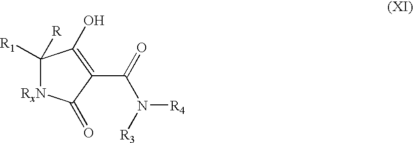

wherein

R₁, R, and Rₓ are independently selected from the group consisting of H, benzyl, pyridinyl, tetrahydro-pyranyl, methyl-1H-imidazolyl, cyclohexylmethyl, phenethyl, p-chlorobenzyl, carboxylic acid benzyl ester, propionic acid tert-butyl ester, tert-butyl ester, ethanone, hydroxy, methoxy, ethoxy, propoxy, butoxy, t-butoxy, phenyl, isobutyl, methyl, ethyl, propyl, butyl, cyclohexyl, cyclohexylmethyl, m-methoxy phenyl, carboxylic acid 2-methoxy-ethyl ester, 3,3-dimethyl-butan-1-one, 2,2-dimethyl-propan-1-one, carboxylic acid methyl ester, alkyl, halogen, CN, CO₂Rₐ, —C(O)Rₐ, —CORₐ, C(O)NRₐRₐ, aryl, and heterocycle, which may be optionally substituted with methoxy or 2-methoxy-ethoxy, wherein each Rₐ is independently selected from the group consisting of H, alkyl, aryl, and heterocycle;

R₃ is selected from the group consisting of -G₁, -G₁-G₂, —Y-G₂, and -G₁-Y-G₂;

G₁ and G₂ are independently selected from the group consisting of phenyl, cyclohexyl, cyclopentyl, 4-indanyl, pyrimidinyl, N-morpholino, furanyl, thiophenyl, pyrrolyl, N-1H-pyridin-2-onyl, bicyclo[4.2.0]octa-1,3,5-trien-3-yl, 1-indanyl, naphthalenyl, tetrahydro-naphthalenyl, pyrazine, [1,2,3]thiadiazolyl, 3-isoxazolyl, 5-indolyl, 2,3-dihydro-indol-6-yl, indazol-5-yl, benzo[2,1,3]thiadiazol-5-yl, cycloheptyl, isopropyl-[1,3,4]thiadiazolyl, benzothiazolyl, 3-methyl-butyl, 1H-pyrazolyl, oxazolyl, piperidinyl, 1H-imidazolyl, pyrrolidinyl, piperazinyl, 1H-[1,2,4]triazolyl, and pyridinyl, which may be optionally substituted with one or more of substituent moieties selected from the group consisting of CF₃, OCF₃, iodo, chloro, bromo, —C(O)NH₂, —O(CH₂)₅CH₃, carboxylic acid methyl ester, phenyl, p-methoxy phenyl, —NHC(O)NH₂, —C(O)O(CH₂)₂N(CH₂CH₃)₂, t-butyl, fluoro, methoxy, hydroxy, isopropyl, cyano, isopropenyl tetrahydropyran, benzyl, amino, —NHC(O)OC(CH₃)₃, —NHC(O)OC(CH₃)₃, —C(O)CH₃, —CH₂CO₂H, methyl, and —(CH₂)₂—OH;

Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂—, and any combination thereof, wherein each R_{y} is independently selected from the group consisting of H, alkyl, aryl, heterocycle, hydroxy, or alkoxy; and

R₄ is selected from the group consisting of H, phenyl, benzyl, isobutyl, cyclohexyl, cyclohexylmethyl, m-methoxy phenyl, alkyl, aryl, and heterocycle.

In yet another aspect, the invention is directed to a compound of Formula XII:

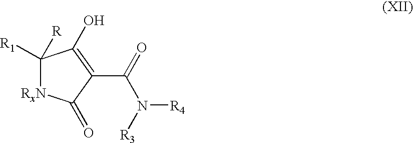

wherein

R is selected from the group consisting of H, alkyl, halogen, CN, CO₂Rₐ, and CONRₐRₐ, wherein each Rₐ is independently selected from the group consisting of H, alkyl, aryl, and heterocycle; or R and R₁, taken together, may form a substituted or unsubstituted spiro heterocyclic or carbocyclic ring, which may optionally be substituted with a benzyl group;

R₁ is selected from the group consisting of H, phenyl, benzyl, ethyl, methyl, isobutyl, pyridinyl, tetrahydro-pyranyl, methyl-1H-imidazolyl, cyclohexylmethyl, phenethyl, p-chlorobenzyl, carboxylic acid benzyl ester, propionic acid tert-butyl ester; or R and R₁, taken together, may form a substituted or unsubstituted spiro heterocyclic or carbocyclic ring, which may optionally be substituted with a benzyl group;

R₂ is selected from the group consisting of H, phenyl, benzyl, isobutyl, cyclohexyl, cyclohexylmethyl, m-methoxy phenyl;

R₃ is selected from the group consisting of -G₁, -G₁-G₂, —Y-G₂, and -G₁-Y-G₂;

G₁ and G₂ are independently selected from the group consisting of 4-indanyl, cyclohexyl, furanyl, pyrrolyl, N-1H-pyridin-2-onyl, and benzothiazolyl, thiophenyl, oxazolyl, pyridinyl, piperidinyl, piperazinyl, N-morpholino, 1H-Pyrazolyl, phenyl, 1H-[1,2,4]triazolyl, 1H-imidazolyl, and pyrimidinyl, which may be optionally substituted with one or more of substituent moieties selected from the group consisting of methoxy, ethyl, methyl, CF₃, cyano, benzyl, phenyl, p-methoxy phenyl, fluoro, tert-butyl, chloro, —(CH₂)₅CH₃, isopropyl, isopropenyl, carboxylic acid methyl ester, methyl-dimethyl-amine, —SCH₃, —C(O)NH, —NHC(O)OC(CH₃)₃, —(CH₂)₂—OH, and —S(O)₂CH₃;

Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂—, and any combination thereof, wherein each R_{y} is independently selected from the group consisting of H, alkyl, aryl, heterocycle, hydroxy, or alkoxy; and

Rₓ is selected from the group consisting of H, phenyl, benzyl, isobutyl, cyclohexyl, cyclohexylmethyl, m-methoxy phenyl, alkyl, aryl, and heterocycle.

An additional aspect of the invention is a method for treating bacterial disease comprising administering to a subject a compound of the following formula

R-Q₁-T

wherein

R is a functionalizing moiety;

Q₁ is a monocyclic hydroxydicarbonyl moiety; and

T is a tail moiety,

such that a bacterial disease is treated in the subject. Exemplary compounds, include, but are not limited to compounds of Formulae I-XII.

In another embodiment, the present invention is a method for treating bacterial disease comprising administering a potent and selective undecaprenyl pyrophosphate synthase (UPPS) inhibitor to a subject, such that a bacterial disease is treated in the subject.

Another embodiment of the invention pertains to a method for treating bacterial disease comprising administering a selective UPPS inhibitor to a subject, such that a bacterial disease is treated in the subject.

In yet another embodiment of the invention pertains to a method for treating bacterial disease comprising administering a potent UPPS inhibitor to a subject, such that a bacterial disease is treated in the subject.

Another embodiment of the invention is a method for inhibiting undecaprenyl pyrophosphate synthase (UPPS) comprising administering to a bacterium compromised subject an activity-enhanced UPPS inhibitor, such that UPPS is inhibited in the subject.

An additional embodiment of the invention relates to a method for selectively inhibiting undecaprenyl pyrophosphate synthase (UPPS) comprising the step of administering to a bacterium compromised subject an activity-enhanced UPPS inhibitor wherein the UPPS/FPPS specificity ratio is less than or equal to about 0.02, e.g., less than or equal to about 0.01, e.g., less than or equal to about 0.002, e.g., less than or equal to about 0.001, e.g., less than or equal to about 0.0002, e.g., less than or equal to about 0.0001, such that UPPS is selectively inhibited in the subject.

In another embodiment, the invention is directed to a method for treating a bacterium compromised subject comprising the step of administering to a bacterium compromised subject an activity-enhanced UPPS inhibitor effective to treat a disease or disorder associated with a UPPS enabled bacterium, such that the bacterium compromised subject is treated.

An additional embodiment of the invention is directed to a method for inhibiting undecaprenyl pyrophosphate synthase (UPPS) comprising the step of contacting UPPS with an activity-enhanced UPPS inhibitor, such that UPPS is inhibited.

In another aspect, the invention pertains to a pharmaceutical composition comprising a therapeutically effective amount of a compound of the invention, and a pharmaceutically acceptable carrier.

In yet another aspect, the invention is directed to a packaged pharmaceutical composition comprising a container holding a therapeutically effective amount of a compound of the invention, e.g., a potent and/or selective UPPS inhibitor; and instructions for using the compound to treat a bacterial disease.

Another aspect of the invention pertains to a method for identifying an activity-enhanced UPPS inhibitor comprising

screening drug candidates for threshold activity;

confirming that the molecular structure of a selected drug candidate contains a hydroxydicarbonyl moiety;

analyzing said selected drug candidate to ensure enhanced selectivity or potency;

determining that said selected drug candidate possesses a UPPS/FPPS specificity ratio is less than or equal to about 0.02, e.g., less than or equal to about 0.01, e.g., less than or equal to about 0.002, e.g., less than or equal to about 0.001, e.g., less than or equal to about 0.0002, e.g., less than or equal to about 0.0001, or the selected IC₅₀ of the drug candidate against UPPS is less than or equal to about 2.0 μM, e.g., less than or equal to about 1.0 μM, e.g., less than or equal to about 0.5 μM, e.g., less than or equal to about 0.1 μM, e.g., less than or equal to about 0.05 μM, e.g., less than or equal to about 0.01 μM, e.g., less than or equal to about 0.005 μM; and

identifying said selected drug candidate as an activity-enhanced UPPS inhibitor.

### DETAILED DESCRIPTION OF THE INVENTION

The compounds provided by the present invention are inhibitors of UPPS. In particular embodiments, the compounds of the invention are selective and/or potent inhibitors of UPPS. In addition, the invention also provides pharmaceutical compositions comprising these compounds and methods of using these compounds for treating bacterial disease, such as bacterial infection.

### DEFINITIONS

For convenience, the definitions of several terms that will be used throughout the specification have been assembled below:

The term “aliphatic group” includes organic moieties characterized by straight or branched-chains, typically having between 1 and 22 carbon atoms, e.g., between 1 and 8 carbon atoms, e.g., between 1 and 6 carbon atoms. In complex structures, the chains may be branched, bridged, or cross-linked. Aliphatic groups include alkyl groups, alkenyl groups, alkynyl groups, and any combination thereof.

As used herein, “alkyl” groups include saturated hydrocarbons having one or more carbon atoms, e.g., between 1 and 22 carbon atoms, e.g., between 1 and 8 carbon atoms, e.g., between 1 and 6 carbon atoms, including straight-chain alkyl groups (e.g., methyl, ethyl, propyl, butyl, pentyl, hexyl, heptyl, octyl, nonyl, decyl, etc.), cyclic alkyl groups (or “cycloalkyl” or “alicyclic”) (e.g., cyclopropyl, cyclopentyl, cyclohexyl, cycloheptyl, cyclooctyl, etc.), branched-chain alkyl groups (isopropyl, tert-butyl, sec-butyl, isobutyl, etc.), and alkyl-substituted alkyl groups (e.g., alkyl-substituted cycloalkyl groups and cycloalkyl-substituted alkyl groups).

In certain embodiments, a straight-chain or branched-chain alkyl group may have 30 or fewer carbon atoms in its backbone, e.g., C₁-C₃₀ for straight-chain or C₃-C₃₀ for branched-chain. In certain embodiments, a straight-chain or branched-chain alkyl group may have 20 or fewer carbon atoms in its backbone, e.g., C₁-C₂₀ for straight-chain or C₃-C₂₀ for branched-chain, and in more particular embodiments 18 or fewer. Likewise, in certain embodiments cycloalkyl groups have from 3-10 carbon atoms in their ring structure, and in more particular embodiments have 3-7 carbon atoms in the ring structure. The term “lower alkyl” refers to alkyl groups having from 1 to 6 carbons in the chain, and to cycloalkyl groups having from 3 to 6 carbons in the ring structure.

In certain embodiments, the alkyl group (e.g., straight, branched, cyclic, and lower alkyl group) is substituted. In particular embodiments, the alkyl group is substituted with one or more halogens, e.g., F. In a specific embodiment, the alkyl group is perfluorinated, e.g., CF₃. Moreover, the alkyl group, in combination with halogen substitution(s) would be understood to be a haloalkyl moiety. Accordingly, and for convenience herein, reference to an alkyl moiety may also incorporate haloalkyl moieties, regardless of whether specific embodiments recited herein are differentiated by explicitly making reference to haloalkly moieties.

Unless the number of carbons is otherwise specified, “lower” as in “lower aliphatic,” “lower alkyl,” “lower alkenyl,” etc. as used herein means that the moiety has at least one and less than about 8 carbon atoms. In certain embodiments, a straight-chain or branched-chain lower alkyl group has 6 or fewer carbon atoms in its backbone (e.g., C₁-C₆ for straight-chain, C₃-C₆ for branched-chain), and in particular embodiments, 4 or fewer. Likewise, in certain embodiments cycloalkyl groups have from 3-8 carbon atoms in their ring structure, and in more particular embodiments have 5 or 6 carbons in the ring structure. The term “C₁-C₆” as in “C₁-C₆ alkyl” means alkyl groups containing 1 to 6 carbon atoms.

Moreover, unless otherwise specified the term alkyl includes both “unsubstituted alkyls” and “substituted alkyls,” the latter of which refers to alkyl groups having substituents replacing one or more hydrogens on one or more carbons of the hydrocarbon backbone. Such substituents may include, for example, alkenyl, alkynyl, halogen, hydroxyl, alkylcarbonyloxy, arylcarbonyloxy, alkoxycarbonyloxy, aryloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclic, alkylaryl, or aromatic (including heteroaromatic) groups.

An “arylalkyl” group is an alkyl group substituted with an aryl group (e.g., phenylmethyl (i.e., benzyl)). An “alkylaryl” moiety is an aryl group substituted with an alkyl group (e.g., p-methylphenyl (i.e., p-tolyl)). The term “n-alkyl” means a straight-chain (i.e., unbranched) unsubstituted alkyl group. An “alkylene” group is a divalent analog of the corresponding alkyl group. Examples of alkylene groups include ethylene (—CH₂CH₂—), propylene (—CH₂CH₂CH₂—), butylene (—CH₂CH₂CH₂CH₂—) and 1-methyethylene (—CH(CH₃)CH₂—). The terms “alkenyl”, “alkynyl” and “alkenylene” refer to unsaturated aliphatic groups analogous to alkyls, but which contain at least one double or triple carbon-carbon bond respectively. Examples of alkenylene groups include ethenylene (—CH═CH—), propenylene (—CH═CHCH₂—), 2-butenylene (—CH₂CH═CHCH₂—) and 1-methyethenylene (—C(CH₃)CH—). Suitable alkenyl and alkynyl groups include groups having 2 to about 12 carbon atoms, preferably from 2 to about 6 carbon atoms.

The term “haloalkyl” describes alkyl moieties that contain one or more of the same or different halogen substituents, e.g., F or Cl. In particular, the term “haloalkyl” includes alkyl moieties comprising one halogen group, alkyl moieties that are perfluorinated, as well as any level of halogenation in between the two extremes. Examplary haloalkyl moieties include, but are not limited to —CF₃, —CH₂F, —CHF₂, —CF₂CF₃, —CF₂CF₃, —CHFCF₃, —CF₂CF₃, —CF₂CF₂H, and —CF₂CHF₂. In addition, haloalkyl groups may be straight chain or branched and may be optionally substituted with additional substituents (i.e., other than the halogen substituents). In particular embodiments, the haloalkyl is —CF₃.

The term “aromatic or aromatic group” and “aryl or aryl group” includes unsaturated and aromatic cyclic hydrocarbons (e.g., benzyl or phenyl) as well as unsaturated and aromatic heterocycles containing one or more rings. Aryl groups may also be fused or bridged with a bond (e.g., biphenyl), alicyclic or heterocyclic rings that are not aromatic so as to form a polycycle (e.g., tetralin). An “arylene” group is a divalent analog of an aryl group.

The term “carbocycle or carbocyclic group” includes any possible saturated or unsaturated closed ring alkyl groups (or “cycloalkyl” or “alicyclic” or “carbocyclic” groups) (e.g., cyclopropyl, cyclopentyl, cyclohexyl, cycloheptyl, cyclooctyl, etc.), any possible C₃-C₁₂ saturated or unsaturated halogenated closed ring alkyl groups, and substituted or unsubstituted aromatic groups, e.g., phenyl. In certain embodiments, the carbocyclic group is a substituted or unsubstituted C₃-C₁₀ carbocyclic ring.

The term “heterocyclic group” includes closed ring structures analogous to carbocyclic groups in which one or more of the carbon atoms in the ring is an element other than carbon, for example, nitrogen, sulfur, or oxygen (e.g. cyclic ethers, lactones, lactams, azitidines). Heterocyclic groups may be saturated or unsaturated. Heterocyclic groups may be halogenated. Additionally, heterocyclic groups (such as pyrrolyl, pyridyl, isoquinolyl, quinolyl, purinyl, and furyl) may have aromatic character, in which case they may be referred to as “heteroaryl” or “heteroaromatic” groups. In certain embodiments, the heterocyclic group is a substituted or unsubstituted C₃-C₁₀ heterocyclic rings.

Unless otherwise stipulated, carbocyclic and heterocyclic (including heteroaryl) groups may also be substituted at one or more constituent atoms. Examples of heteroaromatic and heteroalicyclic groups may have 1 to 3 separate or fused rings with 3 to about 8 members per ring and one or more N, O, or S heteroatoms. In general, the term “heteroatom” includes atoms of any element other than carbon or hydrogen, preferred examples of which include nitrogen, oxygen, sulfur, and phosphorus. Heterocyclic groups may be saturated or unsaturated or aromatic.

Examples of heterocycles include, but are not limited to, acridinyl; azocinyl; benzimidazolyl; benzofuranyl; benzothiofuranyl; benzothiophenyl; benzoxazolyl; benzthiazolyl; benztriazolyl; benztetrazolyl; benzisoxazolyl; benzisothiazolyl; benzimidazolinyl; carbazolyl; 4aH-carbazolyl; carbolinyl; chromanyl; chromenyl; cinnolinyl; decahydroquinolinyl; 2H,6H-1,5,2-dithiazinyl; dihydrofuro[2,3-b]tetrahydrofuran; furanyl; furazanyl; imidazolidinyl; imidazolinyl; imidazolyl; 1H-indazolyl; indolenyl; indolinyl; indolizinyl; indolyl; 3H-indolyl; isobenzofuranyl; isochromanyl; isoindazolyl; isoindolinyl; isoindolyl; isoquinolinyl; isothiazolyl; isoxazolyl; methylenedioxyphenyl; morpholinyl; naphthyridinyl; octahydroisoquinolinyl; oxadiazolyl; 1,2,3-oxadiazolyl; 1,2,4-oxadiazolyl; 1,2,5-oxadiazolyl; 1,3,4-oxadiazolyl; oxazolidinyl; oxazolyl; oxazolidinyl; pyrimidinyl; phenanthridinyl; phenanthrolinyl; phenazinyl; phenothiazinyl; phenoxathiinyl; phenoxazinyl; phthalazinyl; piperazinyl; piperidinyl; piperidonyl; 4-piperidonyl; piperonyl; pteridinyl; purinyl; pyranyl; pyrazinyl; pyrazolidinyl; pyrazolinyl; pyrazolyl; pyridazinyl; pyridooxazole; pyridoimidazole; pyridothiazole; pyridinyl; pyridyl; pyrimidinyl; pyrrolidinyl; pyrrolinyl; 2H-pyrrolyl; pyrrolyl; quinazolinyl; quinolinyl; 4H-quinolizinyl; quinoxalinyl; quinuclidinyl; tetrahydrofuranyl; tetrahydroisoquinolinyl; tetrahydroquinolinyl; tetrazolyl; 6H-1,2,5-thiadiazinyl; 1,2,3-thiadiazolyl; 1,2,4-thiadiazolyl; 1,2,5-thiadiazolyl; 1,3,4-thiadiazolyl; thianthrenyl; thiazolyl; thienyl; thienothiazolyl; thienooxazolyl; thienoimidazolyl; thiophenyl; triazinyl; 1,2,3-triazolyl; 1,2,4-triazolyl; 1,2,5-triazolyl; 1,3,4-triazolyl; and xanthenyl. Preferred heterocycles include, but are not limited to, pyridinyl; furanyl; thienyl; pyrrolyl; pyrazolyl; pyrrolidinyl; imidazolyl; indolyl; benzimidazolyl; 1H-indazolyl; oxazolidinyl; benzotriazolyl; benzisoxazolyl; oxindolyl; benzoxazolinyl; and isatinoyl groups. Also included are fused ring and spiro compounds containing, for example, the above heterocycles.

A common hydrocarbon aryl group is a phenyl group having one ring. Two-ring hydrocarbon aryl groups include naphthyl, indenyl, benzocyclooctenyl, benzocycloheptenyl, pentalenyl, and azulenyl groups, as well as the partially hydrogenated analogs thereof such as indanyl and tetrahydronaphthyl. Exemplary three-ring hydrocarbon aryl groups include acephthylenyl, fluorenyl, phenalenyl, phenanthrenyl, and anthracenyl groups.

Aryl groups also include heteromonocyclic aryl groups, i.e., single-ring heteroaryl groups, such as thienyl, furyl, pyranyl, pyrrolyl, imidazolyl, pyrazolyl, pyridinyl, pyrazinyl, pyrimidinyl, and pyridazinyl groups; and oxidized analogs thereof such as pyridonyl, oxazolonyl, pyrazolonyl, isoxazolonyl, and thiazolonyl groups. The corresponding hydrogenated (i.e., non-aromatic) heteromonocyclic groups include pyrrolidinyl, pyrrolinyl, imidazolidinyl, imidazolinyl, pyrazolidinyl, pyrazolinyl, piperidyl and piperidino, piperazinyl, and morpholino and morpholinyl groups.

Aryl groups also include fused two-ring heteroaryls such as indolyl, isoindolyl, indolizinyl, indazolyl, quinolinyl, isoquinolinyl, phthalazinyl, quinoxalinyl, quinazolinyl, cinnolinyl, chromenyl, isochromenyl, benzothienyl, benzimidazolyl, benzothiazolyl, purinyl, quinolizinyl, isoquinolonyl, quinolonyl, naphthyridinyl, and pteridinyl groups, as well as the partially hydrogenated analogs such as chromanyl, isochromanyl, indolinyl, isoindolinyl, and tetrahydroindolyl groups. Aryl groups also include fused three-ring groups such as phenoxathiinyl, carbazolyl, phenanthridinyl, acridinyl, perimidinyl, phenanthrolinyl, phenazinyl, phenothiazinyl, phenoxazinyl, and dibenzofuranyl groups.

Some typical aryl groups include substituted or unsubstituted 5- and 6-membered single-ring groups. In another aspect, each Ar group may be selected from the group consisting of substituted or unsubstituted phenyl, pyrrolyl, furyl, thienyl, thiazolyl, isothiaozolyl, imidazolyl, triazolyl, tetrazolyl, pyrazolyl, oxazolyl, isooxazolyl, pyridinyl, pyrazinyl, pyridazinyl, and pyrimidinyl groups. Further examples include substituted or unsubstituted phenyl, 1-naphthyl, 2-naphthyl, biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl groups.

The term “amine” or “amino,” as used herein, refers to an unsubstituted or substituted moiety of the formula —NR^{a}R^{b}, in which each R^{a} and R^{b} are each independently hydrogen, alkyl, aryl, or heterocyclyl, or each R^{a} and R^{b}, taken together with the nitrogen atom to which they are attached, form a cyclic moiety having from 3 to 8 atoms in the ring. Thus, the term amino includes cyclic amino moieties such as piperidinyl or pyrrolidinyl groups, unless otherwise stated. Thus, the term “alkylamino” as used herein means an alkyl group having an amino group attached thereto. Suitable alkylamino groups include groups having 1 to about 12 carbon atoms, e.g., from 1 to about 6 carbon atoms. The term amino includes compounds or moieties in which a nitrogen atom is covalently bonded to at least one carbon or heteroatom. The term “dialkylamino” includes groups wherein the nitrogen atom is bound to at least two alkyl groups. The term “arylamino” and “diarylamino” include groups wherein the nitrogen is bound to at least one or two aryl groups, respectively. The term “alkylarylamino” refers to an amino group which is bound to at least one alkyl group and at least one aryl group. The term “alkaminoalkyl” refers to an alkyl, alkenyl, or alkynyl group substituted with an alkylamino group. The term “amide” or “aminocarbonyl” includes compounds or moieties which contain a nitrogen atom which is bound to the carbon of a carbonyl or a thiocarbonyl group. The term “azaalkyl” refers to an alkyl group in which one or more —CH₂— units have been replaced by an —N(R)— group, where R is hydrogen or C₁-C₄-alkyl. If an azaalkyl group includes two or more N(R) groups, any two N(R) groups are separated by one or more carbon atoms.

The terms “alkylthio” or “thiaalkoxy” refers to an alkyl group, having a sulfhydryl group attached thereto. Suitable alkylthio groups include groups having 1 to about 12 carbon atoms, e.g., from 1 to about 6 carbon atoms. The term “thiaalkyl” refers to an alkyl group in which one or more —CH₂— units have been replaced by a sulfur atom. If a thiaalkyl group includes two or more sulfur atoms, any two sulfur atoms are separated by one or more carbon atoms.

The term “alkylcarboxyl” as used herein means an alkyl group having a carboxyl group attached thereto.

The term “alkoxy” as used herein means an alkyl group having an oxygen atom attached thereto. Representative alkoxy groups include groups having 1 to about 12 carbon atoms, e.g., between 1 and 8 carbon atoms, e.g., between 1 and 6 carbon atoms, e.g., methoxy, ethoxy, propoxy, tert-butoxy and the like. Examples of alkoxy groups include methoxy, ethoxy, isopropyloxy, propoxy, butoxy, and pentoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc., as well as perhalogenated alkyloxy groups. The term “oxaalkyl” refers to an alkyl group in which one or more—CH₂— units have been replaced by an oxygen atom. If an oxaalkyl group includes two or more oxygen atoms, any two oxygen atoms are separated by one or more carbon atoms.

The term “acylamino” includes moieties wherein an amino moiety is bonded to an acyl group. For example, the acylamino group includes alkylcarbonylamino, arylcarbonylamino, carbamoyl and ureido groups.

The terms “alkoxyalkyl”, “alkylaminoalkyl” and “thioalkoxyalkyl” include alkyl groups, as described above, which further include oxygen, nitrogen or sulfur atoms replacing one or more carbons of the hydrocarbon backbone.

The term “carbonyl” or “carboxy” includes compounds and moieties which contain a carbon connected with a double bond to an oxygen atom. Examples of moieties which contain a carbonyl include aldehydes, ketones, carboxylic acids, amides, esters, anhydrides, etc.

The term “ether” or “ethereal” includes compounds or moieties which contain an oxygen atom bonded to two carbon atoms. For example, an ether or ethereal group includes “alkoxyalkyl” which refers to an alkyl, alkenyl, or alkynyl group substituted with an alkoxy group.

The term “nitro” means —NO₂; the term “halogen” or “halogen” or “halo” designates —F, —Cl, —Br or —I; the term “thiol,” “thio,” or “mercapto” means SH; and the term “hydroxyl” or “hydroxy” means —OH.

The term “acyl” refers to a carbonyl group that is attached through its carbon atom to a hydrogen (i.e., a formyl), an aliphatic group (e.g., acetyl), an aromatic group (e.g., benzoyl), and the like. The term “substituted acyl” includes acyl groups where one or more of the hydrogen atoms on one or more carbon atoms are replaced by, for example, an alkyl group, alkynyl group, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless otherwise specified, the chemical moieties of the compounds of the invention, including those groups discussed above, may be “substituted or unsubstituted.” In some embodiments, the term “substituted” means that the moiety has substituents placed on the moiety other than hydrogen (i.e., in most cases, replacing a hydrogen), which allow the molecule to perform its intended function. In certain embodiments, examples of substituents include moieties selected from substituted or unsubstituted aliphatic moieties. In particular embodiments, the exemplary substituents include, but are not limited to, straight or branched alkyl (e.g., C₁-C₅), cycloalkyl (e.g., C₃-C₈), alkoxy (e.g., C₁-C₆), thioalkyl (e.g., C₁-C₆), alkenyl (e.g., C₂-C₆), alkynyl (e.g., C₂-C₆), heterocyclic, carbocyclic, aryl (e.g., phenyl), aryloxy (e.g., phenoxy), arylkyl (e.g., benzyl), aryloxyalkyl (e.g., phenyloxyalkyl), arylacetamidoyl, alkylaryl, heteroaralkyl, alkylcarbonyl and arylcarbonyl or other such acyl group, heteroarylcarbonyl, and heteroaryl groups, as well as (CR′R″)₀₋₃NR′R″ (e.g., —NH₂), (CR′R″)₀₋₃CN (e.g., —CN), —NO₂, halogen (e.g., —F, —Cl, —Br, or —I), (CR′R″)₀₋₃C(halogen)₃ (e.g., —CF₃), (CR′R″)₀₋₃CH(halogen)₂, (CR′R″)₀₋₃CH₂(halogen), (CR′R″)₀₋₃CONR′R″, (CR′R″)₀₋₃(CNH)NR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₃R′ (e.g., —SO₃H), (CR′R″)₀₋₃O(CR′R″)₀₋₃H (e.g., —CH₂OCH₃ and —OCH₃), (CR′R″)₀₋₃S(CR′R″)₀₋₃H (e.g., —SH and —SCH₃), (CR′R″)₀₋₃OH (e.g., —OH), (CR′R″)₀₋₃COR′, (CR′R″)₀₋₃ (substituted or unsubstituted phenyl), (CR′R″)₀₋₃(C₃-C₈ cycloalkyl), (CR′R″)₀₋₃CO₂R′ (e.g., —CO₂H), and (CR′R″)₀₋₃OR′ groups, wherein R′ and R″ are each independently hydrogen, a C₁-C₅ alkyl, C₂-C₅ alkenyl, C₂-C₅ alkynyl, or aryl group; or the side chain of any naturally occurring amino acid.

In another embodiment, a substituent may be selected from straight or branched alkyl (e.g., C₁-C₅), cycloalkyl (e.g., C₃-C₈), alkoxy (e.g., C₁-C₆), thioalkyl (e.g., C₁-C₆), alkenyl (e.g., C₂-C₆), alkynyl (e.g., C₂-C₆), heterocyclic, carbocyclic, aryl (e.g., phenyl), aryloxy (e.g., phenoxy), aralkyl (e.g., benzyl), aryloxyalkyl (e.g., phenyloxyalkyl), arylacetamidoyl, alkylaryl, heteroaralkyl, alkylcarbonyl and arylcarbonyl or other such acyl group, heteroarylcarbonyl, or heteroaryl group, (CR′R″)₀₋₁₀NR′R″ (e.g., —NH₂), (CR′R″)₀₋₁₀CN (e.g., —CN), NO₂, halogen (e.g., F, Cl, Br, or I), (CR′R″)₀₋₁₀C(halogen)₃ (e.g., —CF₃), (CR′R″)₀₋₁₀CH(halogen)₂, (CR′R″)₀₋₁₀CH₂(halogen), (CR′R″)₀₋₁₀CONR′R″, (CR′R″)₀₋₁₀(CNH)NR′R″, (CR′R″)₀₋₁₀S(O)₁₋₂NR′R″, (CR′R″)₀₋₁₀CHO, (CR′R″)₀₋₁₀(CR′R″)₀₋₁₀H, (CR′R″)₀₋₁₀S(O)₀₋₃R′ (e.g., —SO₃H), (CR′R″)₀₋₁₀O(CR′R″)₀₋₁₀H (e.g., —CH₂OCH₃ and —OCH₃), (CR′R″)₀₋₁₀S(CR′R″)₀₋₃H (e.g., —SH and —SCH₃), (CR′R″)₀₋₁₀OH (e.g., —OH), (CR′R″)₀₋₁₀COR′, (CR′R″)₀₋₁₀ (substituted or unsubstituted phenyl), (CR′R″)₀₋₁₀(C₃-C₈ cycloalkyl), (CR′R″)₀₋₁₀CO₂R′ (e.g., —CO₂H), or (CR′R″)₀₋₁₀OR′ group, or the side chain of any naturally occurring amino acid; wherein R′ and R″ are each independently hydrogen, a C₁-C₅ alkyl, C₂-C₅ alkenyl, C₂-C₅ alkynyl, or aryl group, or R′ and R″ taken together are a benzylidene group or a —(CH₂)₂O(CH₂)₂— group.

It will be understood that “substitution” or “substituted with” includes the implicit proviso that such substitution is in accordance with the permitted valence of the substituted atom and the substituent, and that the substitution results in a stable compound, e.g., which does not spontaneously undergo transformation such as by rearrangement, cyclization, elimination, etc. As used herein, the term “substituted” is meant to include all permissible substituents of organic compounds. In a broad aspect, the permissible substituents include acyclic and cyclic, branched and unbranched, carbocyclic and heterocyclic, aromatic and nonaromatic substituents of organic compounds. The permissible substituents can be one or more. It should further be understood that the substituents described herein may be attached to the moiety that is substituted in any orientation (regardless of whether such attachment orientation is indicated herein by the manner of description, e.g., by a dash)

In certain embodiments, a “substituent” may be selected from the group consisting of, for example, CF₃, OCF₃, iodo, chloro, bromo, —C(O)NH₂, —O(CH₂)₅CH₃, carboxylic acid methyl ester, phenyl, p-methoxy phenyl, —NHC(O)NH₂, —C(O)O(CH₂)₂N(CH₂CH₃)₂, t-butyl, fluoro, methoxy, hydroxy, isopropyl, cyano, isopropenyl tetrahydropyran, benzyl, amino, —C(O)OH, —C(O)CH₃, —CH₂CO₂H, methyl, —(CH₂)₂—OH, methoxy, 2-methoxy-ethoxy, pyrrolidinyl, 4-methylpiperazinyl, piperazinyl, H, alkyl, halogen, NO₂, CN, OR_{b}, NR_{b}R_{b}, CO₂R_{b}, —C(O)R_{b}, —COR_{b}, NR_{b}C(O)R_{b}, NR_{b}C(O)NR_{b}R_{b}, NR_{b}R_{b}C(O)O—, C(O)NR_{b}R_{b}, aryl, and heterocycle, which may be optionally substituted with methoxy or 2-methoxy-ethoxy, wherein each R_{b} is independently selected from the group consisting of H, alkyl, aryl, and heterocycle, tert-butyl ester, ethanone, methyl, ethyl, carboxylic acid 2-methoxy-ethyl ester, 3,3-dimethyl-butan-1-one, 2,2-dimethyl-propan-1-one, hydroxy, methoxy, ethoxy, propoxy, butoxy, and t-butoxy.

In certain embodiments, the substituent may be selected from the group consisting of H, an aliphatic group (e.g., alkyl, alkenyl, alkynyl etc.), a carbocyclic group (e.g., saturated or unsaturated), a heterocyclic group (e.g., saturated or unsaturated), halogen, NO₂, CN, ORₐ, NRₐRₐ, CO₂Rₐ, —C(O)Rₐ, —CORₐ, NRₐC(O)Rₐ, NRₐC(O)NRₐRₐ, NRₐRₐC(O)O—, C(O)NRₐRₐ, which may be optionally substituted, wherein each Rₐ is independently selected from the group consisting of H, an aliphatic group, a carbocyclic group, and a heterocyclic group.

### Compounds of the Invention

The compounds of the invention, e.g., Formulae I-XII, particular compounds thereof (and substituted derivatives as described herein) are intended to be within the scope of the invention, i.e., regardless of their activity. Accordingly, the compounds of the invention include, but are not limited to compounds of the following formula:

R-Q₁-T

wherein R is a functionalizing moiety; Q is a hydroxydicarbonyl moiety; and T is a tail moiety.

The language “hydroxydicarbonyl moiety” describes a core moiety of certain compounds of the invention, i.e., Q, which comprise the following moiety:

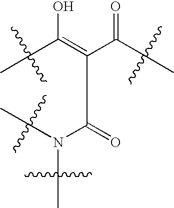

The skilled artisan would understand that such moieties may comprise a substructure of a ring system by cyclization of the left side of the depicted structure, for example, including but are not limited to monocyclic rings multi-cyclic, e.g., bicyclic (such as fused bicyclic), rings containing this hydroxydicarbonyl moiety. In particular embodiments, the hydroxydicarbonyl moiety is five or six membered monocyclic ring containing this hydroxydicarbonyl moiety. In another particular embodiment, the hydroxydicarbonyl moiety is nine-, ten-, or eleven-membered bicyclic ring containing this hydroxydicarbonyl moiety. It should be understood that, in certain embodiments of the invention, the hydroxydicarbonyl moiety is useful as a phosphate mimic.

The language “Functionalizing Moiety” describes a moiety of certain compounds of the invention that may be used to functionalize the hydroxydicarbonyl moiety, i.e., the Q moiety, which comprises a substituent (e.g., including spiro type substituents) that allows the compound of the invention to perform its intended function. For example, in certain embodiments of the invention, the functionalizing moiety is -M₁, -M₁-M₂, -Z-M₂, and -M₁-Z-M₂, wherein M₁ and M₂ are independently selected from the group consisting of H, an aliphatic group, a carbocyclic group, a heterocyclic group, which may be optionally substituted; and Z is a linking moiety.

In certain embodiments, the functionalizing moiety may be selected from the group consisting of H, an aliphatic group (e.g., alkyl, alkenyl, alkynyl etc.), a carbocyclic group (e.g., saturated or unsaturated), a heterocyclic group (e.g., saturated or unsaturated), halogen, NO₂, CN, ORₐ, NRₐRₐ, CO₂Rₐ, —C(O)Rₐ, —CORₐ, NRₐC(O)Rₐ, NRₐC(O)NRₐRₐ, NRₐRₐC(O)O, C(O)NRₐRₐ, which may be optionally substituted, wherein each Rₐ is independently selected from the group consisting of H, an aliphatic group, a carbocyclic group, and a heterocyclic group.

The language “tail moiety” describes a moiety of certain compounds of the invention that is linked to the hydroxydicarbonyl moiety and may be used to occupy the hydrophobic cleft of the UPP synthase enzyme, and include moieties that allow the compound of the invention to perform its intended function. Exemplary Tail Moieties include, but are not limited to moieties such as -G₁, -G₁-G₂, —Y-G₂, and -G₁-Y-G₂, wherein G₁ and G₂ are independently selected from the group consisting of H, an aliphatic group, a carbocyclic group, and a heterocyclic group, which may be optionally substituted with one or more of substituents; and Y is a linking moiety.

It should be noted that the functionalizing moiety and the tail moiety may be modified to adjust at least one chemical or physical property of the compounds of the invention. In certain embodiments, the modification comprises substitution of a carbon atom with a heteroatom or addition of a heteroatom-containing substituent (e.g., substituted by a substituent selected from the group consisting of hydroxy, alkoxy, heterocycle and an acyl group), such that one or more of the chemical or physical properties of the depicted compound have been enhanced, e.g., with respect to potency or selectivity. In certain embodiments, the modification is made to adjust one or more of the following attributes: acidity, lypohilicity, solubility. Moreover, such adjustment may result from the substitution itself, i.e., a direct effect, or the adjustment may indirectly result from the affect on the compound as a whole, e.g., by conformation changes. In certain embodiments, the modification comprises substitution of a carbon atom with a heteroatom or addition of a heteroatom-containing substituent, such that one or more of the chemical or physical properties of R-Q₁-T have been enhanced. In particular embodiments, R or T is substituted by a substituent selected from the group consisting of hydroxy, alkoxy, heterocycle and an acyl group.

The “linking moiety,” may contain 1-8 atoms or may be a bond, and serves as the connection point through which tail moiety or functionalizing moiety is linked to the hydroxydicarbonyl moiety of the compounds of the invention, wherein 3 atoms directly connect the tail moiety to the hydroxydicarbonyl moiety. In certain embodiments, the linking moiety may comprise, but is not limited to, substituted or unsubstituted alkyl (e.g., methylene chains), amide groups, acyl groups, heteroatoms, or a combination thereof. In specific embodiments, the linking moiety may be of —O—, —NH—, —CR_{y}R_{y}—, —S—, —S(O)—, —C(O)—, —NHC(O)—, —C(O)NH—, —NHC(O)CH₂O—, —S(O)₂—, —CH(OH)—, —CH(OR_{y}), —C(O)CH₂—, —CH₂C(O)—, —CH₂CH(OH)—, —CH₂CH(OR_{y})—, —CH(OH)CH₂—, —CH(OR_{y})CH₂—, and any combination thereof, wherein each R_{y} is independently selected from the group consisting of H, an aliphatic group, a carbocyclic group, a heterocyclic group, hydroxy, and alkoxy.

In one embodiment, a compound of the invention is represented by the following formula:

R-Q₁-T

wherein R is a functionalizing moiety; Q₁ is a monocyclic hydroxydicarbonyl moiety; and T is a tail moiety. In particular embodiments, T is selected from the group consisting of -G₁, -G₁-G₂, —Y-G₂, and -G₁-Y-G₂, and wherein G₁ and G₂ are independently selected from the group consisting of substituted or unsubstituted saturated or unsaturated heterocyclic or carbocyclic rings; and Y is a linking moiety.

In another embodiment, R-Q-T is represented by one of the following formulae

Rₘ-Q₁-T;

R-Q₁-Tₘ;

Rₘ-Q₁-Tₘ;

wherein Rₘ is a functionalizing moiety modified to adjust at least one chemical or physical property of R-Q₁-T; Tₘ is a tail moiety modified to adjust at least one chemical or physical property of R-Q₁-T; and Q₁ is defined as noted hereinabove. In certain embodiments, the modification comprises substitution of a carbon atom with a heteroatom or addition of a heteroatom-containing substituent, e.g., wherein R or T is substituted by a substituent selected from the group consisting of hydroxy, alkoxy, heterocycle and an acyl group, such that one or more of the chemical or physical properties of R-Q₁-T have been enhanced.

### A. Compounds of Formula I

Another embodiment of the invention pertains to a compound of Formula I:

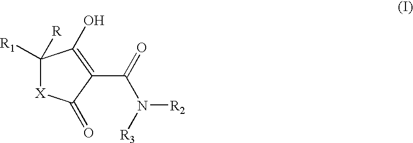

wherein

X is selected from the group consisting of NRₓCRₓRₓ and O;

R is selected from the group consisting of H, an aliphatic group (e.g., alkyl, alkenyl, alkynyl etc.), a carbocyclic group (e.g., saturated or unsaturated), a heterocyclic group (e.g., saturated or unsaturated), halogen, NO₂, CN, ORₐ, NRₐRₐ, CO₂Rₐ, —C(O)Rₐ, —CORₐ, NRₐC(O)Rₐ, NRₐC(O)NRₐRₐ, NRₐRₐC(O)O—, C(O)NRₐRₐ, which may be optionally substituted, wherein each Rₐ is independently selected from the group consisting of H, an aliphatic group, a carbocyclic group, and a heterocyclic group; or R and R₁, taken together, may form a substituted or unsubstituted spiro heterocyclic or carbocyclic ring, which may optionally be substituted (e.g., by an aliphatic group, a carbocyclic group, or a heterocyclic group);

R₁ and Rₓ are independently selected from the group consisting of H, -M₁, -M₁-M₂, -Z-M₂, and -M₁-Z-M₂; or R and R₁; taken together, may form a substituted or unsubstituted spiro heterocyclic or carbocyclic ring, which may optionally be substituted;

M₁ and M₂ are independently selected from the group consisting of H, an aliphatic group, a carbocyclic group, a heterocyclic group, which may be optionally substituted;

Z is selected from the group consisting of —O—, —NH—, —CR_{z}R_{z}—, —S—, —S(O)—, —C(O)—, —NHC(O)—, —C(O)NH—, —NHC(O)CH₂O—, —S(O)₂—, —CH(OH)—, —CH(OR_{z}), —C(O)CH₂—, —CH₂C(O)—, —CH₂CH(OH)—, —CH₂CH(OR_{z})—, —CH(OH)CH₂—, —CH(OR_{z})CH₂—, and any combination thereof, wherein each R_{z} is independently selected from the group consisting of H, an aliphatic group, a carbocyclic group, a heterocyclic group, hydroxy, and alkoxy;

R₂ is selected from the group consisting of H, an aliphatic group, a carbocyclic group, and a heterocyclic group (e.g., selected from the group consisting of phenyl, benzyl, isobutyl, cyclohexyl, cyclohexylmethyl, m-methoxy phenyl, alkyl, aryl, and heterocycle);

R₃ is selected from the group consisting of -G₁, -G₁-G₂, —Y-G₂, and -G₁-Y-G₂;

G₁ and G₂ are independently selected from H, an aliphatic group, a carbocyclic group, and a heterocyclic group, which may be optionally substituted with one or more of substituents; and

Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂—, —CH(OH)—, —CH(OR_{y}), —C(O)CH₂—, —CH₂C(O)—, —CH₂CH(OH)—, —CH₂CH(OR_{y})—, —CH(OH)CH₂—, —CH(OR_{y})CH₂—, and any combination thereof, wherein each R_{y} is independently selected from the group consisting of H, an aliphatic group, a carbocyclic group, a heterocyclic group, hydroxy, and alkoxy.

In certain embodiments, G₁ is a mono or bicyclic aromatic or heteroaromatic group which may be optionally substituted with one or more substituents selected from the group consisting of H, an aliphatic group, a carbocyclic group, a heterocyclic group, an acyl group, an aliphatic group, halogen, —NO₂, trifluoromethyl, difluoromethyoxy, trifluoromethyoxy, azido, —CN, —OR_{g}, —SR_{g}—NR_{g}R_{g}, —CO₂R_{g}, —C(O)R_{g}, —NR_{g}C(O)R_{g}, —NR_{g}C(O)NR_{g}R_{g}, —C(O)NR_{g}R_{g}, NR_{g}SO₂R_{g}, —SO₂NR_{g}R_{g}, —C(O)OR_{g}, —OC(O)R_{g}, —NR_{g}C(O)OR_{g}, C(O)NR_{g}R_{g}, —SO₂R_{g}, —(CH₂)₂—OR_{g} and —CH₂NR_{g}R_{g}, wherein R_{g} is selected from H, aliphatic, carbocyclic, heterocyclic and heteroaromatic groups.

In certain embodiments, G₂ is an aliphatic group, or a mono or bicyclic carbocyclic or heterocyclic group (e.g., aromatic or heteroaromatic group) which may be optionally substituted with one or more substituents selected from the group consisting of H, an aliphatic group, a carbocyclic group, a heterocyclic group, an acyl group, halogen, —NO₂, trifluoromethyl, difluoromethyoxy, trifluoromethyoxy, azido, —CN, —OR_{g}, —SR_{g}—NR_{g}R_{g}, —CO₂R_{g}, —C(O)R_{g}, —NR_{g}C(O)R_{g}, —NR_{g}C(O)NR_{g}R_{g}, —C(O)NR_{g}R_{g}, NR_{g}SO₂R_{g}, —SO₂NR_{g}R_{g}, —C(O)OR_{g}, —OC(O)R_{g}, —NR_{g}C(O)OR_{g}, C(O)NR_{g}R_{g}, —SO₂R_{g}, —(CH₂)₂—OR_{g} and —CH₂NR_{g}R_{g}, wherein R_{g} is selected from H, aliphatic, carbocyclic, heterocyclic and heteroaromatic groups.

### B. Compounds of Formula II

In another embodiment, the invention is directed to a compound of Formula II:

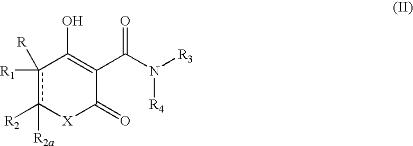

wherein

[symbol] represents a single or a double bond;

X is selected from the group consisting of NRₓCRₓRₓ and O;

R and R₂ₐ are absent or independently selected from the group consisting of H, an aliphatic group (e.g., alkyl, alkenyl, alkynyl etc.), a carbocyclic group (e.g., saturated or unsaturated), a heterocyclic group (e.g., saturated or unsaturated), halogen, NO₂, CN, ORₐ, NRₐRₐ, CO₂Rₐ, —C(O)Rₐ, —CORₐ, NRₐC(O)Rₐ, NRₐC(O)NRₐRₐ, NRₐRₐC(O)O—, C(O)NRₐRₐ, which may be optionally substituted, wherein each Rₐ is independently selected from the group consisting of H, an aliphatic group, a carbocyclic group, and a heterocyclic group; or R and R₁, taken together, may form a substituted or unsubstituted spiro heterocyclic or carbocyclic ring, which may optionally be substituted (e.g., by an aliphatic group, a carbocyclic group, or a heterocyclic group); or R₂ and R₂ₐ, taken together, may form a substituted or unsubstituted spiro heterocyclic or carbocyclic ring, which may optionally be substituted (e.g., by an aliphatic group, a carbocyclic group, or a heterocyclic group);

R₁, R₂, and Rₓ are independently selected from the group consisting of H, -M₁, -M₁-M₂, -Z-M₂, and -M₁-Z-M₂; or R and R₁, taken together, may form a substituted or unsubstituted spiro heterocyclic or carbocyclic ring, which may optionally be substituted; or R₂ and R₂ₐ, taken together, may form a substituted or unsubstituted spiro heterocyclic or carbocyclic ring, which may optionally be substituted;

M₁ and M₂ are independently selected from the group consisting of H, an aliphatic group, a carbocyclic group, a heterocyclic group, which may be optionally substituted;

Z is selected from the group consisting of —O—, —NH—, —CR_{z}R_{z}—, —S—, —S(O)—, —C(O)—, —NHC(O)—, —C(O)NH—, —NHC(O)CH₂O—, —S(O)₂—, —CH(OH)—, —CH(OR_{z}), —C(O)CH₂—, —CH₂C(O)—, —CH₂CH(OH)—, —CH₂CH(OR_{z})—, —CH(OH)CH₂—, —CH(OR_{z})CH₂—, and any combination thereof, wherein each R_{z} is independently selected from the group consisting of H, an aliphatic group, a carbocyclic group, a heterocyclic group, hydroxy, and alkoxy;

R₃ is selected from the group consisting of -G₁, -G₁-G₂, —Y-G₂, and -G₁-Y-G₂;

G₁ and G₂ are independently selected from H, an aliphatic group, a carbocyclic group, and a heterocyclic group, which may be optionally substituted with one or more of substituents;

Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂—, —CH(OH)—, —CH(OR_{y}), —C(O)CH₂—, —CH₂C(O)—, —CH₂CH(OH)—, —CH₂CH(OR_{y})—, —CH(OH)CH₂—, —CH(OR_{y})CH₂—, and any combination thereof, wherein each R_{y} is independently selected from the group consisting of H, an aliphatic group, a carbocyclic group, a heterocyclic group, hydroxy, and alkoxy; and

R₄ is selected from the group consisting of H, an aliphatic group, a carbocyclic group, and a heterocyclic group (e.g., selected from the group consisting of phenyl, benzyl, isobutyl, cyclohexyl, cyclohexylmethyl, m-methoxy phenyl, alkyl, aryl, and heterocycle).

In certain embodiments, G₁ is a mono or bicyclic aromatic or heteroaromatic group which may be optionally substituted with one or more substituents selected from the group consisting of H, an aliphatic group, a carbocyclic group, a heterocyclic group, an acyl group, an aliphatic group, halogen, —NO₂, trifluoromethyl, difluoromethyoxy, trifluoromethyoxy, azido, —CN, —OR_{g}, —SR_{g}—NR_{g}R_{g}, —CO₂R_{g}, —C(O)R_{g}, —NR_{g}C(O)R_{g}, —NR_{g}C(O)NR_{g}R_{g}, —C(O)NR_{g}R_{g}, NR_{g}SO₂R_{g}, —SO₂NR_{g}R_{g}, —C(O)OR_{g}, —OC(O)R_{g}, —NR_{g}C(O)OR_{g}, C(O)NR_{g}R_{g}, —SO₂R_{g}, —(CH₂)₂—OR_{g} and —CH₂NR_{g}R_{g}, wherein R_{g} is selected from H, aliphatic, carbocyclic, heterocyclic and heteroaromatic groups.

In certain embodiments, G₂ is an aliphatic group, or a mono or bicyclic carbocyclic or heterocyclic group (e.g., aromatic or heteroaromatic group) which may be optionally substituted with one or more substituents selected from the group consisting of H, an aliphatic group, a carbocyclic group, a heterocyclic group, an acyl group, halogen, —NO₂, trifluoromethyl, difluoromethyoxy, trifluoromethyoxy, azido, —CN, —OR_{g}, —SR_{g}—NR_{g}R_{g}, —CO₂R_{g}, —C(O)R_{g}, —NR_{g}C(O)R_{g}, —NR_{g}C(O)NR_{g}R_{g}, —C(O)NR_{g}R_{g}, NR_{g}SO₂R_{g}, —SO₂NR_{g}R_{g}, —C(O)OR_{g}, —OC(O)R_{g}, —NR_{g}C(O)OR_{g}, C(O)NR_{g}R_{g}, —SO₂R_{g}, —(CH₂)₂—OR_{g} and —CH₂NR_{g}R_{g}, wherein R_{g} is selected from H, aliphatic, carbocyclic, heterocyclic and heteroaromatic groups.

### C. Compounds of Formula III

In another embodiment, the compound of the invention is represented by Formula III:

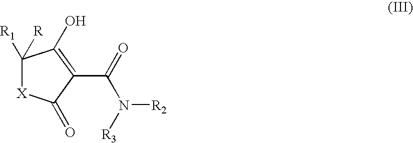

wherein

X is selected from the group consisting of NRₓCRₓRₓ and O;

R is selected from the group consisting of H, benzyl, pyridinyl, tetrahydro-pyranyl, methyl-1H-imidazolyl, cyclohexylmethyl, phenethyl, p-chlorobenzyl, carboxylic acid benzyl ester, propionic acid tert-butyl ester, tert-butyl ester, ethanone, hydroxy, methoxy, ethoxy, propoxy, butoxy, t-butoxy, phenyl, isobutyl, methyl, ethyl, propyl, butyl, cyclohexyl, cyclohexylmethyl, m-methoxy phenyl, carboxylic acid 2-methoxy-ethyl ester, 3,3-dimethyl-butan-1-one, 2,2-dimethyl-propan-1-one, carboxylic acid methyl ester, alkyl, halogen, NO₂, CN, ORₐ, NRₐRₐ, CO₂Rₐ, —C(O)Rₐ, —CORₐ, NRₐC(O)Rₐ, NRₐC(O)NRₐRₐ, NRₐRₐC(O)O—, C(O)NRₐRₐ, aryl, and heterocycle, which may be optionally substituted with methoxy or 2-methoxy-ethoxy, wherein each Rₐ is independently selected from the group consisting of H, alkyl, aryl, and heterocycle; or R and R₁, taken together, may form a substituted or unsubstituted spiro heterocyclic or carbocyclic ring, which may optionally be substituted with a benzyl group;

R₁ and Rₓ are independently selected from the group consisting of H, benzyl, pyridinyl, tetrahydro-pyranyl, methyl-1H-imidazolyl, cyclohexylmethyl, phenethyl, p-chlorobenzyl, carboxylic acid benzyl ester, propionic acid tert-butyl ester, tert-butyl ester, ethanone, hydroxy, methoxy, ethoxy, propoxy, butoxy, t-butoxy, phenyl, isobutyl, methyl, ethyl, propyl, butyl, cyclohexyl, cyclohexylmethyl, m-methoxy phenyl, carboxylic acid 2-methoxy-ethyl ester, 3,3-dimethyl-butan-1-one, 2,2-dimethyl-propan-1-one, carboxylic acid methyl ester, alkyl, halogen, NO₂, CN, OR_{b}, NR_{b}R_{b}, CO₂R_{b}, —C(O)R_{b}, —COR_{b}, NR_{b}C(O)R_{b}, NR_{b}C(O)NR_{b}R_{b}, NR_{b}R_{b}C(O)O—, C(O)NR_{b}R_{b}, aryl, and heterocycle, which may be optionally substituted with methoxy or 2-methoxy-ethoxy, wherein each R_{b} is independently selected from the group consisting of H, alkyl, aryl, and heterocycle; or R and R₁, taken together, may form a substituted or unsubstituted spiro heterocyclic or carbocyclic ring, which may optionally be substituted with a benzyl group;

R₂ is selected from the group consisting of H, phenyl, benzyl, isobutyl, cyclohexyl, cyclohexylmethyl, m-methoxy phenyl, alkyl, aryl, and heterocycle;

R₃ is selected from the group consisting of -G₁, -G₁-G₂, —Y-G₂, and -G₁-Y-G₂;

G₁ and G₂ are independently selected from the group consisting of phenyl, cyclohexyl, cyclopentyl, 4-indanyl, pyrimidinyl, N-morpholino, furanyl, thiophenyl, pyrrolyl, N-1H-pyridin-2-onyl, bicyclo[4.2.0]octa-1,3,5-trien-3-yl, 1-indanyl, naphthalenyl, tetrahydro-naphthalenyl, pyrazine, [1,2,3]thiadiazolyl, 3-isoxazolyl, 5-indolyl, 2,3-dihydro-indol-6-yl, indazol-5-yl, benzo[2,1,3]thiadiazol-5-yl, cycloheptyl, isopropyl-[1,3,4]thiadiazolyl, benzothiazolyl, 3-methyl-butyl, 1H-pyrazolyl, oxazolyl, piperidinyl, 1H-imidazolyl, pyrrolidinyl, piperazinyl, 1H-[1,2,4]triazolyl, and pyridinyl, which may be optionally substituted with one or more of substituent moieties selected from the group consisting of CF₃, OCF₃, iodo, chloro, bromo, —C(O)NH₂, —O(CH₂)₅CH₃, carboxylic acid methyl ester, phenyl, p-methoxy phenyl, —NHC(O)NH₂, —C(O)O(CH₂)₂N(CH₂CH₃)₂, t-butyl, fluoro, methoxy, hydroxy, isopropyl, cyano, isopropenyl tetrahydropyran, benzyl, amino, —NHC(O)OC(CH₃)₃, —C(O)OH, —C(O)CH₃, —CH₂CO₂H, methyl, and —(CH₂)₂—OH; and

Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂—, and any combination thereof, wherein each R_{y} is independently selected from the group consisting of H, alkyl, aryl, heterocycle, hydroxy, or alkoxy.

In certain embodiments G₁ is selected from the group consisting of phenyl, 4-indanyl, pyrimidinyl, cyclohexyl, cyclopentyl, cycloheptyl, isopropyl-[1,3,4]thiadiazolyl, benzothiazolyl, 3-methyl-butyl, 1H-Pyrazolyl, and 1H-[1,2,4]triazolyl, pyridinyl, which may be optionally substituted with one or more of substituent moieties selected from the group consisting of CF₃, OCF₃, iodo, —C(O)NH₂, —O(CH₂)₅CH₃, carboxylic acid methyl ester, phenyl, p-methoxy phenyl, —NHC(O)NH₂, —C(O)O(CH₂)₂N(CH₂CH₃)₂, t-butyl, methyl-dimethyl-amine, cyano, ethyl, benzyl, methyl, fluoro, chloro, —SCH₃, —S(O)₂CH₃, methoxy, and —(CH₂)₂—OH.

In certain additional embodiments, G₂ is selected from the group consisting of phenyl, N-morpholino, furanyl, thiophenyl, pyrrolyl, N-1H-pyridin-2-onyl, and benzothiazolyl, cyclohexyl, oxazolyl, piperidinyl, 1H-pyrazolyl, 1H-imidazolyl, pyrrolidinyl, and piperazinyl, which may be optionally substituted with one or more of substituent moieties selected from the group consisting of methyl, ethyl, benzyl, cyano, CF₃, carboxylic acid methyl ester, methyl-dimethyl-amine, —SCH₃, —C(O)NH₂, —(CH₂)₂—OH, —S(O)₂CH₃, chloro and bromo.

### D. Compounds of Formula IV

In another embodiment, the compound of the invention is represented by Formula IV:

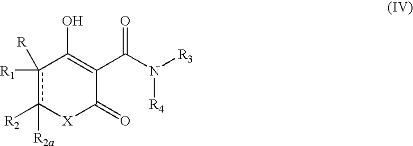

wherein

[symbol] represents a single or a double bond;

X is selected from the group consisting of NRₓCRₓRₓ and O;

R and R₂ₐ are absent or independently selected from the group consisting of H, benzyl, pyridinyl, tetrahydro-pyranyl, methyl-1H-imidazolyl, cyclohexylmethyl, phenethyl, p-chlorobenzyl, carboxylic acid benzyl ester, propionic acid tert-butyl ester, tert-butyl ester, ethanone, hydroxy, methoxy, ethoxy, propoxy, butoxy, t-butoxy, phenyl, isobutyl, methyl, ethyl, propyl, butyl, cyclohexyl, cyclohexylmethyl, m-methoxy phenyl, carboxylic acid 2-methoxy-ethyl ester, 3,3-dimethyl-butan-1-one, 2,2-dimethyl-propan-1-one, carboxylic acid methyl ester, alkyl, halogen, NO₂, CN, ORₐ, NRₐRₐ, CO₂Rₐ, —C(O)Rₐ, —CORₐ, NRₐC(O)Rₐ, NRₐC(O)NRₐRₐ, NRₐRₐC(O)O—, C(O)NRₐRₐ, aryl, and heterocycle, which may be optionally substituted with methoxy or 2-methoxy-ethoxy, wherein each Rₐ is independently selected from the group consisting of H, alkyl, aryl, and heterocycle;

R₁, R₂, each Rₓ are independently selected from the group consisting of H, benzyl, pyridinyl, tetrahydro-pyranyl, methyl-1H-imidazolyl, cyclohexylmethyl, phenethyl, p-chlorobenzyl, carboxylic acid benzyl ester, propionic acid tert-butyl ester, tert-butyl ester, ethanone, hydroxy, methoxy, ethoxy, propoxy, butoxy, t-butoxy, phenyl, isobutyl, methyl, ethyl, propyl, butyl, cyclohexyl, cyclohexylmethyl, m-methoxy phenyl, carboxylic acid 2-methoxy-ethyl ester, 3,3-dimethyl-butan-1-one, 2,2-dimethyl-propan-1-one, carboxylic acid methyl ester, alkyl, halogen, NO₂, CN, OR_{b}, NR_{b}R_{b}, CO₂R_{b}, —C(O)R_{b}, —COR_{b}, NR_{b}C(O)R_{b}, NR_{b}C(O)NR_{b}R_{b}, NR_{b}R_{b}C(O)O—, C(O)NR_{b}R_{b}, aryl, and heterocycle, which may be optionally substituted with methoxy or 2-methoxy-ethoxy, wherein each R_{b} is independently selected from the group consisting of H, alkyl, aryl, and heterocycle;

R₃ is selected from the group consisting of -G₁, -G₁-G₂, —Y-G₂, and -G₁-Y-G₂;

G₁ and G₂ are independently selected from the group consisting of phenyl, cyclohexyl, cyclopentyl, 4-indanyl, pyrimidinyl, N-morpholino, furanyl, thiophenyl, pyrrolyl, N-1H-pyridin-2-onyl, bicyclo[4.2.0]octa-1,3,5-trien-3-yl, 1-indanyl, naphthalenyl, tetrahydro-naphthalenyl, pyrazine, [1,2,3]thiadiazolyl, 3-isoxazolyl, 5-indolyl, 2,3-dihydro-indol-6-yl, indazol-5-yl, benzo[2,1,3]thiadiazol-5-yl, cycloheptyl, isopropyl-[1,3,4]thiadiazolyl, benzothiazolyl, 3-methyl-butyl, 1H-pyrazolyl, oxazolyl, piperidinyl, 1H-imidazolyl, pyrrolidinyl, piperazinyl, 1H-[1,2,4]triazolyl, and pyridinyl, which may be optionally substituted with one or more of substituent moieties selected from the group consisting of CF₃, OCF₃, iodo, chloro, bromo, —C(O)NH₂, —O(CH₂)₅CH₃, carboxylic acid methyl ester, phenyl, p-methoxy phenyl, —NHC(O)NH₂, —C(O)O(CH₂)₂N(CH₂CH₃)₂, t-butyl, fluoro, methoxy, hydroxy, isopropyl, cyano, isopropenyl tetrahydropyran, benzyl, amino, —NHC(O)OC(CH₃)₃, —C(O)OH, —C(O)CH₃, —CH₂CO₂H, methyl, and —(CH₂)₂—OH;

Y is selected from the group consisting of —O—, —NH—, —CR_{y}R_{y}—, —S—, —S(O)—, —C(O)—, —NHC(O)—, —C(O)NH—, —NHC(O)CH₂O— —S(O)₂—, and any combination thereof, wherein each R_{y} is independently selected from the group consisting of H, alkyl, aryl, heterocycle, hydroxy, or alkoxy; and

R₄ is selected from the group consisting of H, phenyl, benzyl, isobutyl, cyclohexyl, cyclohexylmethyl, m-methoxy phenyl, alkyl, aryl, and heterocycle. In a particular embodiment, X is NRₓ, e.g., wherein R₄ is H.

### E. Compounds of Formula V

In an additional embodiment, the compound of the invention is represented by Formula V:

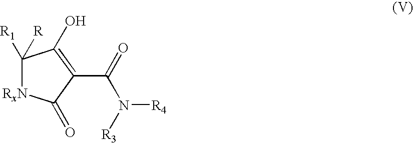

wherein

R₁, R, and Rₓ are independently selected from the group consisting of H, benzyl, pyridinyl, tetrahydro-pyranyl, methyl-1H-imidazolyl, cyclohexylmethyl, phenethyl, p-chlorobenzyl, carboxylic acid benzyl ester, propionic acid tert-butyl ester, tert-butyl ester, ethanone, hydroxy, methoxy, ethoxy, propoxy, butoxy, t-butoxy, phenyl, isobutyl, methyl, ethyl, propyl, butyl, cyclohexyl, cyclohexylmethyl, m-methoxy phenyl, carboxylic acid 2-methoxy-ethyl ester, 3,3-dimethyl-butan-1-one, 2,2-dimethyl-propan-1-one, carboxylic acid methyl ester, alkyl, halogen, NO₂, CN, ORₐ, NRₐRₐ, CO₂Rₐ, —C(O)Rₐ, —CORₐ, NRₐC(O)Rₐ, NRₐC(O)NRₐRₐ, NRₐRₐC(O)O—, C(O)NRₐRₐ, aryl, and heterocycle, which may be optionally substituted with methoxy or 2-methoxy-ethoxy, wherein each Rₐ is independently selected from the group consisting of H, alkyl, aryl, and heterocycle;

R₃ is selected from the group consisting of -G₁, -G₁-G₂, —Y-G₂, and -G₁-Y-G₂;

G₁ and G₂ are independently selected from the group consisting of phenyl, cyclohexyl, cyclopentyl, 4-indanyl, pyrimidinyl, N-morpholino, furanyl, thiophenyl, pyrrolyl, N-1H-pyridin-2-onyl, bicyclo[4.2.0]octa-1,3,5-trien-3-yl, 1-indanyl, naphthalenyl, tetrahydro-naphthalenyl, pyrazine, [1,2,3]thiadiazolyl, 3-isoxazolyl, 5-indolyl, 2,3-dihydro-indol-6-yl, indazol-5-yl, benzo[2,1,3]thiadiazol-5-yl, cycloheptyl, isopropyl-[1,3,4]thiadiazolyl, benzothiazolyl, 3-methyl-butyl, 1H-pyrazolyl, oxazolyl, piperidinyl, 1H-imidazolyl, pyrrolidinyl, piperazinyl, 1H-[1,2,4]triazolyl, and pyridinyl, which may be optionally substituted with one or more of substituent moieties selected from the group consisting of CF₃, OCF₃, iodo, chloro, bromo, —C(O)NH₂, —O(CH₂)₅CH₃, carboxylic acid methyl ester, phenyl, p-methoxy phenyl, —NHC(O)NH₂, —C(O)O(CH₂)₂N(CH₂CH₃)₂, t-butyl, fluoro, methoxy, hydroxy, isopropyl, cyano, isopropenyl tetrahydropyran, benzyl, amino, —NHC(O)OC(CH₃)₃, —NHC(O)OC(CH₃)₃, —C(O)OH, —C(O)CH₃, —CH₂CO₂H, methyl, and —(CH₂)₂—OH;

Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂—, and any combination thereof, wherein each R_{y} is independently selected from the group consisting of H, alkyl, aryl, heterocycle, hydroxy, or alkoxy; and

R₄ is selected from the group consisting of H, phenyl, benzyl, isobutyl, cyclohexyl, cyclohexylmethyl, m-methoxy phenyl, alkyl, aryl, and heterocycle.

### F. Compounds of Formula VI

In an additional embodiment, the compound of the invention is represented by Formula VI:

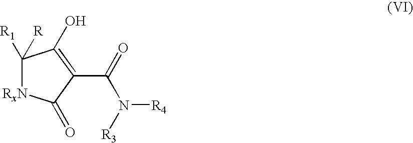

wherein

R is selected from the group consisting of H, alkyl, halogen, NO₂, CN, ORₐ, NRₐRₐ, CO₂Rₐ, and CONRₐRₐ, wherein each Rₐ is independently selected from the group consisting of H, alkyl, aryl, and heterocycle; or R and R₁, taken together, may form a substituted or unsubstituted spiro heterocyclic or carbocyclic ring, which may optionally be substituted with a benzyl group;

R₁ is selected from the group consisting of H, phenyl, benzyl, ethyl, methyl, isobutyl, pyridinyl, tetrahydro-pyranyl, methyl-1H-imidazolyl, cyclohexylmethyl, phenethyl, p-chlorobenzyl, carboxylic acid benzyl ester, propionic acid tert-butyl ester; or R and R₁, taken together, may form a substituted or unsubstituted spiro heterocyclic or carbocyclic ring, which may optionally be substituted with a benzyl group;

R₂ is selected from the group consisting of H, phenyl, benzyl, isobutyl, cyclohexyl, cyclohexylmethyl, m-methoxy phenyl;

R₃ is selected from the group consisting of -G₁, -G₁-G₂, —Y-G₂, and -G₁-Y-G₂; G₁ and G₂ are independently selected from the group consisting of 4-indanyl, cyclohexyl, furanyl, pyrrolyl, N-1H-pyridin-2-onyl, and benzothiazolyl, thiophenyl, oxazolyl, pyridinyl, piperidinyl, piperazinyl, N-morpholino, 1H-Pyrazolyl, phenyl, 1H-[1,2,4]triazolyl, 1H-imidazolyl, and pyrimidinyl, which may be optionally substituted with one or more of substituent moieties selected from the group consisting of methoxy, ethyl, methyl, CF₃, cyano, benzyl, phenyl, p-methoxy phenyl, fluoro, tert-butyl, chloro, —(CH₂)₅CH₃, isopropyl, isopropenyl, carboxylic acid methyl ester, methyl-dimethyl-amine, —SCH₃, —C(O)NH, —NHC(O)OC(CH₃)₃, —(CH₂)₂—OH, and —S(O)₂CH₃;

Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂—, and any combination thereof, wherein each R_{y} is independently selected from the group consisting of H, alkyl, aryl, heterocycle, hydroxy, or alkoxy; and

Rₓ is selected from the group consisting of H, phenyl, benzyl, isobutyl, cyclohexyl, cyclohexylmethyl, m-methoxy phenyl, alkyl, aryl, and heterocycle.

### G. Compounds of Formula VII

Another embodiment of the invention pertains to a compound of Formula VII:

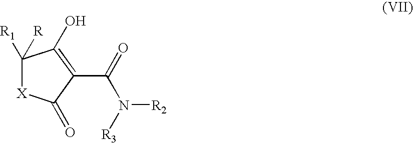

wherein

X is selected from the group consisting of NRₓCRₓRₓ and O;

R is selected from the group consisting of H, an aliphatic group (e.g., alkyl, alkenyl, alkynyl etc.), a carbocyclic group (e.g., saturated or unsaturated), a heterocyclic group (e.g., saturated or unsaturated), halogen, CN, CO₂Rₐ, —C(O)Rₐ, —CORₐ, C(O)NRₐRₐ, which may be optionally substituted, wherein each Rₐ is independently selected from the group consisting of H, an aliphatic group, a carbocyclic group, and a heterocyclic group; or R and R₁, taken together, may form a substituted or unsubstituted spiro heterocyclic or carbocyclic ring, which may optionally be substituted (e.g., by an aliphatic group, a carbocyclic group, or a heterocyclic group);

R₁ and Rₓ are independently selected from the group consisting of H, -M₁, -M₁-M₂, -Z-M₂, and -M₁-Z-M₂; or R and R₁, taken together, may form a substituted or unsubstituted spiro heterocyclic or carbocyclic ring, which may optionally be substituted;

M₁ and M₂ are independently selected from the group consisting of H, an aliphatic group, a carbocyclic group, a heterocyclic group, which may be optionally substituted;

Z is selected from the group consisting of —O—, —NH—, —CR_{z}R_{z}—, —S—, —S(O)—, —C(O)—, —NHC(O)—, —C(O)NH—, —NHC(O)CH₂O—, —S(O)₂—, —CH(OH)—, —CH(OR_{z}), —C(O)CH₂—, —CH₂C(O)—, —CH₂CH(OH)—, —CH₂CH(OR_{z})—, —CH(OH)CH₂—, —CH(OR_{z})CH₂—, and any combination thereof, wherein each R_{z} is independently selected from the group consisting of H, an aliphatic group, a carbocyclic group, a heterocyclic group, hydroxy, and alkoxy;

R₂ is selected from the group consisting of H, an aliphatic group, a carbocyclic group, and a heterocyclic group (e.g., selected from the group consisting of phenyl, benzyl, isobutyl, cyclohexyl, cyclohexylmethyl, m-methoxy phenyl, alkyl, aryl, and heterocycle);

R₃ is selected from the group consisting of -G₁, -G₁-G₂, —Y-G₂, and -G₁-Y-G₂;

G₁ and G₂ are independently selected from H, an aliphatic group, a carbocyclic group, and a heterocyclic group, which may be optionally substituted with one or more of substituents; and

Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂—, —CH(OH)—, —CH(OR_{y}), —C(O)CH₂—, —CH₂C(O)—, —CH₂CH(OH)—, —CH₂CH(OR_{y})—, —CH(OH)CH₂—, —CH(OR_{y})CH₂—, and any combination thereof, wherein each R_{y} is independently selected from the group consisting of H, an aliphatic group, a carbocyclic group, a heterocyclic group, hydroxy, and alkoxy.

In certain embodiments, G₁ is a mono or bicyclic aromatic or heteroaromatic group which may be optionally substituted with one or more substituents selected from the group consisting of H, an aliphatic group, a carbocyclic group, a heterocyclic group, an acyl group, an aliphatic group, halogen, —NO₂, trifluoromethyl, difluoromethyoxy, trifluoromethyoxy, azido, —CN, —OR_{g}, —SR_{g}—NR_{g}R_{g}, —CO₂R_{g}, —C(O)R_{g}, —NR_{g}C(O)R_{g}, —NR_{g}C(O)NR_{g}R_{g}, —C(O)NR_{g}R_{g}, NR_{g}SO₂R_{g}, —SO₂NR_{g}R_{g}, —C(O)OR_{g}, —OC(O)R_{g}, —NR_{g}C(O)OR_{g}, C(O)NR_{g}R_{g}, —SO₂R_{g}, —(CH₂)₂—OR_{g} and —CH₂NR_{g}R_{g}, wherein R_{g} is selected from H, aliphatic, carbocyclic, heterocyclic and heteroaromatic groups.

In certain embodiments, G₂ is an aliphatic group, or a mono or bicyclic carbocyclic or heterocyclic group (e.g., aromatic or heteroaromatic group) which may be optionally substituted with one or more substituents selected from the group consisting of H, an aliphatic group, a carbocyclic group, a heterocyclic group, an acyl group, halogen, —NO₂, trifluoromethyl, difluoromethyoxy, trifluoromethyoxy, azido, —CN, —OR_{g}, —SR_{g}—NR_{g}R_{g}, —CO₂R_{g}, —C(O)R_{g}, —NR_{g}C(O)R_{g}, —NR_{g}C(O)NR_{g}R_{g}, —C(O)NR_{g}R_{g}, NR_{g}SO₂R_{g}, —SO₂NR_{g}R_{g}, —C(O)OR_{g}, —OC(O)R_{g}, —NR_{g}C(O)OR_{g}, C(O)NR_{g}R_{g}, —SO₂R_{g}, —(CH₂)₂—OR_{g} and —CH₂NR_{g}R_{g}, wherein R_{g} is selected from H, aliphatic, carbocyclic, heterocyclic and heteroaromatic groups.

### H. Compounds of Formula VIII

In another embodiment, the invention is directed to a compound of Formula VIII:

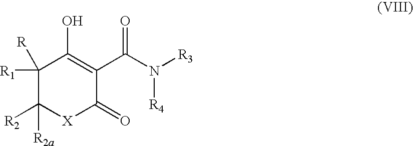

wherein

X is selected from the group consisting of NRₓ and O;

R is absent or selected from the group consisting of H, an aliphatic group (e.g., alkyl, alkenyl, alkynyl etc.), a carbocyclic group (e.g., saturated or unsaturated), a heterocyclic group (e.g., saturated or unsaturated), halogen, NO₂, CN, ORₐ, NRₐRₐ, CO₂Rₐ, —C(O)Rₐ, —CORₐ, NRₐC(O)Rₐ, NRₐC(O)NRₐRₐ, NRₐRₐC(O)O—, C(O)NRₐRₐ, which may be optionally substituted, wherein each Rₐ is independently selected from the group consisting of H, an aliphatic group, a carbocyclic group, and a heterocyclic group; or R and R₁, taken together, may form a substituted or unsubstituted spiro heterocyclic or carbocyclic ring, which may optionally be substituted (e.g., by an aliphatic group, a carbocyclic group, or a heterocyclic group); or R₂ and R₂ₐ, taken together, may form a substituted or unsubstituted spiro heterocyclic or carbocyclic ring, which may optionally be substituted (e.g., by an aliphatic group, a carbocyclic group, or a heterocyclic group);

R₂ₐ is absent or selected from the group consisting of H, an aliphatic group (e.g., alkyl, alkenyl, alkynyl etc.), a carbocyclic group (e.g., saturated or unsaturated), a heterocyclic group (e.g., saturated or unsaturated), halogen, CN, CO₂Rₐ, —C(O)Rₐ, —CORₐ, C(O)NRₐRₐ, which may be optionally substituted, wherein each Rₐ is independently selected from the group consisting of H, an aliphatic group, a carbocyclic group, and a heterocyclic group; or R and R₁, taken together, may form a substituted or unsubstituted spiro heterocyclic or carbocyclic ring, which may optionally be substituted (e.g., by an aliphatic group, a carbocyclic group, or a heterocyclic group); or R₂ and R₂ₐ, taken together, may form a substituted or unsubstituted spiro heterocyclic or carbocyclic ring, which may optionally be substituted (e.g., by an aliphatic group, a carbocyclic group, or a heterocyclic group);

R₁, R₂, and Rₓ are independently selected from the group consisting of H, -M₁, -M₁-M₂, -Z-M₂, and -M₁-Z-M₂; or R and R₁, taken together, may form a substituted or unsubstituted spiro heterocyclic or carbocyclic ring, which may optionally be substituted; or R₂ and R₂ₐ, taken together, may form a substituted or unsubstituted spiro heterocyclic or carbocyclic ring, which may optionally be substituted;

M₁ and M₂ are independently selected from the group consisting of H, an aliphatic group, a carbocyclic group, a heterocyclic group, which may be optionally substituted;

Z is selected from the group consisting of —O—, —NH—, —CR_{z}R_{z}—, —S—, —S(O)—, —C(O)—, —NHC(O)—, —C(O)NH—, —NHC(O)CH₂O—, —S(O)₂—, —CH(OH)—, —CH(OR_{z}), —C(O)CH₂—, —CH₂C(O)—, —CH₂CH(OH)—, —CH₂CH(OR_{z})—, —CH(OH)CH₂—, —CH(OR_{z})CH₂—, and any combination thereof, wherein each R_{z} is independently selected from the group consisting of H, an aliphatic group, a carbocyclic group, a heterocyclic group, hydroxy, and alkoxy;

R₃ is selected from the group consisting of -G₁, -G₁-G₂, —Y-G₂, and -G₁-Y-G₂;

G₁ and G₂ are independently selected from H, an aliphatic group, a carbocyclic group, and a heterocyclic group, which may be optionally substituted with one or more of substituents;

Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂—, —CH(OH)—, —CH(OR_{y}), —C(O)CH₂—, —CH₂C(O)—, —CH₂CH(OH)—, —CH₂CH(OR_{y})—, —CH(OH)CH₂—, —CH(OR_{y})CH₂—, and any combination thereof, wherein each R_{y} is independently selected from the group consisting of H, an aliphatic group, a carbocyclic group, a heterocyclic group, hydroxy, and alkoxy; and

R₄ is selected from the group consisting of H, an aliphatic group, a carbocyclic group, and a heterocyclic group (e.g., selected from the group consisting of phenyl, benzyl, isobutyl, cyclohexyl, cyclohexylmethyl, m-methoxy phenyl, alkyl, aryl, and heterocycle).

In certain embodiments, G₁ is a mono or bicyclic aromatic or heteroaromatic group which may be optionally substituted with one or more substituents selected from the group consisting of H, an aliphatic group, a carbocyclic group, a heterocyclic group, an acyl group, an aliphatic group, halogen, —NO₂, trifluoromethyl, difluoromethyoxy, trifluoromethyoxy, azido, —CN, —OR_{g}, —SR_{g}—NR_{g}R_{g}, —CO₂R_{g}, —C(O)R_{g}, —NR_{g}C(O)R_{g}, —NR_{g}C(O)NR_{g}R_{g}, —C(O)NR_{g}R_{g}, NR_{g}SO₂R_{g}, —SO₂NR_{g}R_{g}, —C(O)OR_{g}, —OC(O)R_{g}, —NR_{g}C(O)OR_{g}, C(O)NR_{g}R_{g}, —SO₂R_{g}, —(CH₂)₂—OR_{g} and —CH₂NR_{g}R_{g}, wherein R_{g} is selected from H, aliphatic, carbocyclic, heterocyclic and heteroaromatic groups.

In certain embodiments, G₂ is an aliphatic group, or a mono or bicyclic carbocyclic or heterocyclic group (e.g., aromatic or heteroaromatic group) which may be optionally substituted with one or more substituents selected from the group consisting of H, an aliphatic group, a carbocyclic group, a heterocyclic group, an acyl group, halogen, —NO₂, trifluoromethyl, difluoromethyoxy, trifluoromethyoxy, azido, —CN, —OR_{g}, —SR_{g}—NR_{g}R_{g}, —CO₂R_{g}, —C(O)R_{g}, —NR_{g}C(O)R_{g}, —NR_{g}C(O)NR_{g}R_{g}, —C(O)NR_{g}R_{g}, NR_{g}SO₂R_{g}, —SO₂NR_{g}R_{g}, —C(O)OR_{g}, —OC(O)R_{g}, —NR_{g}C(O)OR_{g}, C(O)NR_{g}R_{g}, —SO₂R_{g}, —(CH₂)₂—OR_{g} and —CH₂NR_{g}R_{g}, wherein R_{g} is selected from H, aliphatic, carbocyclic, heterocyclic and heteroaromatic groups.

### I. Compounds of Formula IX

In another embodiment, the compound of the invention is represented by Formula IX:

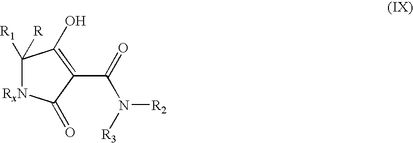

wherein

R is selected from the group consisting of H, benzyl, pyridinyl, tetrahydro-pyranyl, methyl-1H-imidazolyl, cyclohexylmethyl, phenethyl, p-chlorobenzyl, carboxylic acid benzyl ester, propionic acid tert-butyl ester, tert-butyl ester, ethanone, hydroxy, methoxy, ethoxy, propoxy, butoxy, t-butoxy, phenyl, isobutyl, methyl, ethyl, propyl, butyl, cyclohexyl, cyclohexylmethyl, m-methoxy phenyl, carboxylic acid 2-methoxy-ethyl ester, 3,3-dimethyl-butan-1-one, 2,2-dimethyl-propan-1-one, carboxylic acid methyl ester, alkyl, halogen, CN, CO₂Rₐ, —C(O)Rₐ, —CORₐ, C(O)NRₐRₐ, aryl, and heterocycle, which may be optionally substituted with methoxy or 2-methoxy-ethoxy, wherein each Rₐ is independently selected from the group consisting of H, alkyl, aryl, and heterocycle; or R and R₁, taken together, may form a substituted or unsubstituted spiro heterocyclic or carbocyclic ring, which may optionally be substituted with a benzyl group;

R₁ and Rₓ are independently selected from the group consisting of H, benzyl, pyridinyl, tetrahydro-pyranyl, methyl-1H-imidazolyl, cyclohexylmethyl, phenethyl, p-chlorobenzyl, carboxylic acid benzyl ester, propionic acid tert-butyl ester, tert-butyl ester, ethanone, propoxy, t-butoxy, phenyl, isobutyl, methyl, ethyl, propyl, butyl, cyclohexyl, cyclohexylmethyl, m-methoxy phenyl, carboxylic acid 2-methoxy-ethyl ester, 3,3-dimethyl-butan-1-one, 2,2-dimethyl-propan-1-one, carboxylic acid methyl ester, alkyl, halogen, CN, CO₂R_{b}, —C(O)R_{b}, —COR_{b}, C(O)NR_{b}R_{b}, aryl, and heterocycle, which may be optionally substituted with methoxy or 2-methoxy-ethoxy, wherein each R_{b} is independently selected from the group consisting of H, alkyl, aryl, and heterocycle; or R and R₁, taken together, may form a substituted or unsubstituted spiro heterocyclic or carbocyclic ring, which may optionally be substituted with a benzyl group;

R₂ is selected from the group consisting of H, phenyl, benzyl, isobutyl, cyclohexyl, cyclohexylmethyl, m-methoxy phenyl, alkyl, aryl, and heterocycle;

R₃ is selected from the group consisting of -G₁, -G₁-G₂, —Y-G₂, and -G₁-Y-G₂;

G₁ and G₂ are independently selected from the group consisting of phenyl, cyclohexyl, cyclopentyl, 4-indanyl, pyrimidinyl, N-morpholino, furanyl, thiophenyl, pyrrolyl, N-1H-pyridin-2-onyl, bicyclo[4.2.0]octa-1,3,5-trien-3-yl, 1-indanyl, naphthalenyl, tetrahydro-naphthalenyl, pyrazine, [1,2,3]thiadiazolyl, 3-isoxazolyl, 5-indolyl, 2,3-dihydro-indol-6-yl, indazol-5-yl, benzo[2,1,3]thiadiazol-5-yl, cycloheptyl, isopropyl-[1,3,4]thiadiazolyl, benzothiazolyl, 3-methyl-butyl, 1H-pyrazolyl, oxazolyl, piperidinyl, 1H-imidazolyl, pyrrolidinyl, piperazinyl, 1H-[1,2,4]triazolyl, and pyridinyl, which may be optionally substituted with one or more of substituent moieties selected from the group consisting of CF₃, OCF₃, iodo, chloro, bromo, —C(O)NH₂, —O(CH₂)₅CH₃, carboxylic acid methyl ester, phenyl, p-methoxy phenyl, —NHC(O)NH₂, —C(O)O(CH₂)₂N(CH₂CH₃)₂, t-butyl, fluoro, methoxy, hydroxy, isopropyl, cyano, isopropenyl tetrahydropyran, benzyl, amino, —NHC(O)OC(CH₃)₃, —C(O)OH, —C(O)CH₃, —CH₂CO₂H, methyl, and —(CH₂)₂—OH; and

Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂—, and any combination thereof, wherein each R_{y} is independently selected from the group consisting of H, alkyl, aryl, heterocycle, hydroxy, or alkoxy.

In certain embodiments G₁ is selected from the group consisting of phenyl, 4-indanyl, pyrimidinyl, cyclohexyl, cyclopentyl, cycloheptyl, isopropyl-[1,3,4]thiadiazolyl, benzothiazolyl, 3-methyl-butyl, 1H-Pyrazolyl, and 1H-[1,2,4]triazolyl, pyridinyl, which may be optionally substituted with one or more of substituent moieties selected from the group consisting of CF₃, OCF₃, iodo, —C(O)NH₂, —O(CH₂)₅CH₃, carboxylic acid methyl ester, phenyl, p-methoxy phenyl, —NHC(O)NH₂, —C(O)O(CH₂)₂N(CH₂CH₃)₂, t-butyl, methyl-dimethyl-amine, cyano, ethyl, benzyl, methyl, fluoro, chloro, —SCH₃, —S(O)₂CH₃, methoxy, and —(CH₂)₂—OH.

In certain additional embodiments, G₂ is selected from the group consisting of phenyl, N-morpholino, furanyl, thiophenyl, pyrrolyl, N-1H-pyridin-2-onyl, and benzothiazolyl, cyclohexyl, oxazolyl, piperidinyl, 1H-pyrazolyl, 1H-imidazolyl, pyrrolidinyl, and piperazinyl, which may be optionally substituted with one or more of substituent moieties selected from the group consisting of methyl, ethyl, benzyl, cyano, CF₃, carboxylic acid methyl ester, methyl-dimethyl-amine, —SCH₃, —C(O)NH₂, —(CH₂)₂—OH, —S(O)₂CH₃, chloro and bromo.

### J. Compounds of Formula X

In another embodiment, the compound of the invention is represented by Formula X:

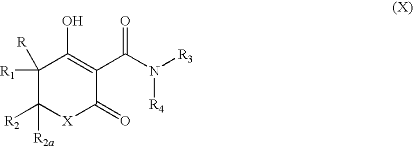

wherein

X is selected from the group consisting of NRₓCRₓRₓ and O;

R₂ and R₂ₐ are absent or independently selected from the group consisting of H, benzyl, pyridinyl, tetrahydro-pyranyl, methyl-1H-imidazolyl, cyclohexylmethyl, phenethyl, p-chlorobenzyl, carboxylic acid benzyl ester, propionic acid tert-butyl ester, tert-butyl ester, ethanone, hydroxy, methoxy, ethoxy, propoxy, butoxy, t-butoxy, phenyl, isobutyl, methyl, ethyl, propyl, butyl, cyclohexyl, cyclohexylmethyl, m-methoxy phenyl, carboxylic acid 2-methoxy-ethyl ester, 3,3-dimethyl-butan-1-one, 2,2-dimethyl-propan-1-one, carboxylic acid methyl ester, alkyl, halogen, CN, CO₂Rₐ, —C(O)Rₐ, —CORₐ, C(O)NRₐRₐ, aryl, and heterocycle, which may be optionally substituted with methoxy or 2-methoxy-ethoxy, wherein each Rₐ is independently selected from the group consisting of H, alkyl, aryl, and heterocycle; or R₂ and R₂ₐ, taken together, may form a substituted or unsubstituted spiro heterocyclic or carbocyclic ring, which may optionally be substituted; or R and R₂ are absent;

R₁, R, and each Rₓ are independently selected from the group consisting of H, benzyl, pyridinyl, tetrahydro-pyranyl, methyl-1H-imidazolyl, cyclohexylmethyl, phenethyl, p-chlorobenzyl, carboxylic acid benzyl ester, propionic acid tert-butyl ester, tert-butyl ester, ethanone, hydroxy, methoxy, ethoxy, propoxy, butoxy, t-butoxy, phenyl, isobutyl, methyl, ethyl, propyl, butyl, cyclohexyl, cyclohexylmethyl, m-methoxy phenyl, carboxylic acid 2-methoxy-ethyl ester, 3,3-dimethyl-butan-1-one, 2,2-dimethyl-propan-1-one, carboxylic acid methyl ester, alkyl, halogen, NO₂, CN, OR_{b}, NR_{b}R_{b}, CO₂R_{b}, —C(O)R_{b}, —COR_{b}, NR_{b}C(O)R_{b}, NR_{b}C(O)NR_{b}R_{b}, NR_{b}R_{b}C(O)O—, C(O)NR_{b}R_{b}, aryl, and heterocycle, which may be optionally substituted with methoxy or 2-methoxy-ethoxy, wherein each R_{b} is independently selected from the group consisting of H, alkyl, aryl, and heterocycle; or R and R₁, taken together, may form a substituted or unsubstituted spiro heterocyclic or carbocyclic ring, which may optionally be substituted; or R and R₂ are absent;

R₃ is selected from the group consisting of -G₁, -G₁-G₂, —Y-G₂, and -G₁-Y-G₂;

G₁ and G₂ are independently selected from the group consisting of phenyl, cyclohexyl, cyclopentyl, 4-indanyl, pyrimidinyl, N-morpholino, furanyl, thiophenyl, pyrrolyl, N-1H-pyridin-2-onyl, bicyclo[4.2.0]octa-1,3,5-trien-3-yl, 1-indanyl, naphthalenyl, tetrahydro-naphthalenyl, pyrazine, [1,2,3]thiadiazolyl, 3-isoxazolyl, 5-indolyl, 2,3-dihydro-indol-6-yl, indazol-5-yl, benzo[2,1,3]thiadiazol-5-yl, cycloheptyl, isopropyl-[1,3,4]thiadiazolyl, benzothiazolyl, 3-methyl-butyl, 1H-pyrazolyl, oxazolyl, piperidinyl, 1H-imidazolyl, pyrrolidinyl, piperazinyl, 1H-[1,2,4]triazolyl, and pyridinyl, which may be optionally substituted with one or more of substituent moieties selected from the group consisting of CF₃, OCF₃, iodo, chloro, bromo, —C(O)NH₂, —O(CH₂)₅CH₃, carboxylic acid methyl ester, phenyl, p-methoxy phenyl, —NHC(O)NH₂, —C(O)O(CH₂)₂N(CH₂CH₃)₂, t-butyl, fluoro, methoxy, hydroxy, isopropyl, cyano, isopropenyl tetrahydropyran, benzyl, amino, —NHC(O)OC(CH₃)₃, —C(O)OH, —C(O)CH₃, —CH₂CO₂H, methyl, and —(CH₂)₂—OH;

Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂—, and any combination thereof, wherein each R_{y} is independently selected from the group consisting of H, alkyl, aryl, heterocycle, hydroxy, or alkoxy; and

R₄ is selected from the group consisting of H, phenyl, benzyl, isobutyl, cyclohexyl, cyclohexylmethyl, m-methoxy phenyl, alkyl, aryl, and heterocycle. In a particular embodiment, X is NRₓ, e.g., wherein R₄ is H.

### K. Compounds of Formula XI

In an additional embodiment, the compound of the invention is represented by Formula XI:

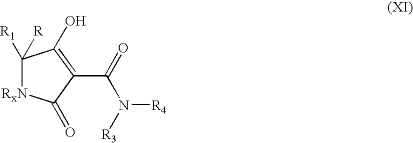

wherein

R₁, R, and Rₓ are independently selected from the group consisting of H, benzyl, pyridinyl, tetrahydro-pyranyl, methyl-1H-imidazolyl, cyclohexylmethyl, phenethyl, p-chlorobenzyl, carboxylic acid benzyl ester, propionic acid tert-butyl ester, tert-butyl ester, ethanone, hydroxy, methoxy, ethoxy, propoxy, butoxy, t-butoxy, phenyl, isobutyl, methyl, ethyl, propyl, butyl, cyclohexyl, cyclohexylmethyl, m-methoxy phenyl, carboxylic acid 2-methoxy-ethyl ester, 3,3-dimethyl-butan-1-one, 2,2-dimethyl-propan-1-one, carboxylic acid methyl ester, alkyl, halogen, CN, CO₂Rₐ, —C(O)Rₐ, —CORₐ, C(O)NRₐRₐ, aryl, and heterocycle, which may be optionally substituted with methoxy or 2-methoxy-ethoxy, wherein each Rₐ is independently selected from the group consisting of H, alkyl, aryl, and heterocycle;

R₃ is selected from the group consisting of -G₁, -G₁-G₂, —Y-G₂, and -G₁-Y-G₂;

G₁ and G₂ are independently selected from the group consisting of phenyl, cyclohexyl, cyclopentyl, 4-indanyl, pyrimidinyl, N-morpholino, furanyl, thiophenyl, pyrrolyl, N-1H-pyridin-2-onyl, bicyclo[4.2.0]octa-1,3,5-trien-3-yl, 1-indanyl, naphthalenyl, tetrahydro-naphthalenyl, pyrazine, [1,2,3]thiadiazolyl, 3-isoxazolyl, 5-indolyl, 2,3-dihydro-indol-6-yl, indazol-5-yl, benzo[2,1,3]thiadiazol-5-yl, cycloheptyl, isopropyl-[1,3,4]thiadiazolyl, benzothiazolyl, 3-methyl-butyl, 1H-pyrazolyl, oxazolyl, piperidinyl, 1H-imidazolyl, pyrrolidinyl, piperazinyl, 1H-[1,2,4]triazolyl, and pyridinyl, which may be optionally substituted with one or more of substituent moieties selected from the group consisting of CF₃, OCF₃, iodo, chloro, bromo, —C(O)NH₂, —O(CH₂)₅CH₃, carboxylic acid methyl ester, phenyl, p-methoxy phenyl, —NHC(O)NH₂, —C(O)O(CH₂)₂N(CH₂CH₃)₂, t-butyl, fluoro, methoxy, hydroxy, isopropyl, cyano, isopropenyl tetrahydropyran, benzyl, amino, —NHC(O)OC(CH₃)₃, —NHC(O)OC(CH₃)₃, —C(O)CH₃, —CH₂CO₂H, methyl, and —(CH₂)₂—OH;

Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂—, and any combination thereof, wherein each R_{y} is independently selected from the group consisting of H, alkyl, aryl, heterocycle, hydroxy, or alkoxy; and

R₄ is selected from the group consisting of H, phenyl, benzyl, isobutyl, cyclohexyl, cyclohexylmethyl, m-methoxy phenyl, alkyl, aryl, and heterocycle. In certain embodiments, Y is not —NH—.

### L. Compounds of Formula XII

In an additional embodiment, the compound of the invention is represented by Formula XII:

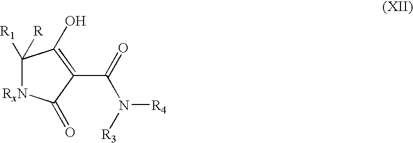

wherein

R is selected from the group consisting of H, alkyl, halogen, CN, CO₂Rₐ, and CONRₐRₐ, wherein each Rₐ is independently selected from the group consisting of H, alkyl, aryl, and heterocycle; or R and R₁, taken together, may form a substituted or unsubstituted spiro heterocyclic or carbocyclic ring, which may optionally be substituted with a benzyl group;

R₁ is selected from the group consisting of H, phenyl, benzyl, ethyl, methyl, isobutyl, pyridinyl, tetrahydro-pyranyl, methyl-1H-imidazolyl, cyclohexylmethyl, phenethyl, p-chlorobenzyl, carboxylic acid benzyl ester, propionic acid tert-butyl ester; or R and R₁, taken together, may form a substituted or unsubstituted spiro heterocyclic or carbocyclic ring, which may optionally be substituted with a benzyl group;

R₂ is selected from the group consisting of H, phenyl, benzyl, isobutyl, cyclohexyl, cyclohexylmethyl, m-methoxy phenyl;

R₃ is selected from the group consisting of -G₁, -G₁-G₂, —Y-G₂, and -G₁-Y-G₂;

G₁ and G₂ are independently selected from the group consisting of 4-indanyl, cyclohexyl, furanyl, pyrrolyl, N-1H-pyridin-2-onyl, and benzothiazolyl, thiophenyl, oxazolyl, pyridinyl, piperidinyl, piperazinyl, N-morpholino, 1H-Pyrazolyl, phenyl, 1H-[1,2,4]triazolyl, 1H-imidazolyl, and pyrimidinyl, which may be optionally substituted with one or more of substituent moieties selected from the group consisting of methoxy, ethyl, methyl, CF₃, cyano, benzyl, phenyl, p-methoxy phenyl, fluoro, tert-butyl, chloro, —(CH₂)₅CH₃, isopropyl, isopropenyl, carboxylic acid methyl ester, methyl-dimethyl-amine, —SCH₃, —C(O)NH, —NHC(O)OC(CH₃)₃, —(CH₂)₂—OH, and —S(O)₂CH₃;

Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂—, and any combination thereof, wherein each R_{y} is independently selected from the group consisting of H, alkyl, aryl, heterocycle, hydroxy, or alkoxy; and

Rₓ is selected from the group consisting of H, phenyl, benzyl, isobutyl, cyclohexyl, cyclohexylmethyl, m-methoxy phenyl, alkyl, aryl, and heterocycle. In certain embodiments, Y is not —NH—.

In certain embodiments of the invention, compounds or substituents that are not modified or altered in any way to enhance stability, and which would otherwise be understood as unstable by the ordinarily skilled artisan, are not included within the genus structures of the invention, i.e., Formulae I-XII. In one particular embodiment, such substituents may include substituents, or R groups, that are attached to the alpha carbon in the ring of the genus structures and wherein X is NRₓ; wherein such substituents are selected from the following general types of substituents: halogen, NO₂, CN, NRR (e.g., NRₐRₐ), NRC(O)R, NRC(O)NRR, and NRRC(O)O—. In another particular embodiment, such substituents may include substituents, or R groups, bonded to the nitrogen atoms of NR moieties of the formulae described herein (e.g., present in the genus structure as an NR type substituent or present in a markush group including an NR type substituent); wherein such substituents are selected from the following general types of substituents: halogen, NO₂, CN, NRR (e.g., NRₐRₐ), NRC(O)R, NRC(O)NRR, and NRRC(O)O—. For clarity, these embodiments comprise compounds of Formulae I-XII where the substituents listed above for the R groups are removed from the definitions/substituents indicated for the respective formulae (and where all other substituents/definitions are identical).

Moreover, it should be understood that the compounds of the present invention, comprise compounds that satisfy valency requirements known to the ordinarily skilled artisan. Additionally, compounds of the present invention comprise stable compounds (i.e., based upon empirical data or on the skilled artisan's understanding of stable bond formation) as well as those compounds that may be modified, e.g., chemically or through appropriate formulation, to become stable. In certain embodiments, such stability is guided by time periods that are sufficient to allow administration to and/or treatment of a subject.

Particular compounds of the invention include, but are not limited to, those set forth below in Tables 1 and 2 and salts thereof. Moreover, it should be understood that each of the compounds listed in Table 1 are separate embodiments of the invention, and are presented in tabular form only as a convenience, i.e., compounds 1-243 should be considered as separately listed and each compound could be the subject of a separate claim in this invention.

In addition, specific compounds of the invention further include derivatives of the compounds depicted below modified to adjust at least one chemical or physical property of depicted compound. In certain embodiments, the modification comprises substitution of a carbon atom with a heteroatom or addition of a heteroatom-containing substituent (e.g., substituted by a substituent selected from the group consisting of hydroxy, alkoxy, heterocycle and an acyl group), such that one or more of the chemical or physical properties of the depicted compound have been enhanced, e.g., with respect to potency or selectivity. In certain embodiments, the modification is made to adjust one or more of the following attributes: acidity, lypohilicity, solubility. Moreover, such adjustment may result from the substitution itself, i.e., a direct effect, or the adjustment may indirectly result from the affect on the compound as a whole, e.g., by conformation changes.

TABLE 1

| | |
|---|---|
| 1. | 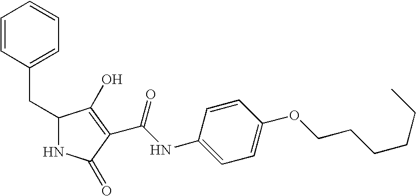 |
| 2. | 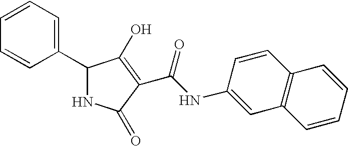 |
| 3. | 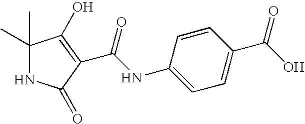 |
| 4. | 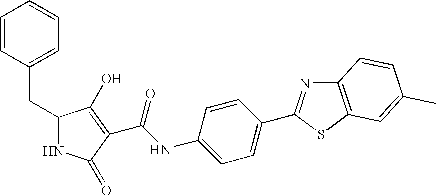 |
| 5. | 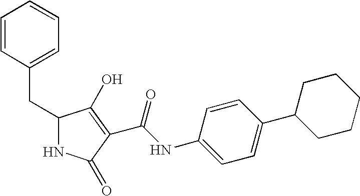 |
| 6. | 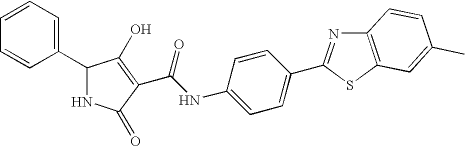 |
| 7. | 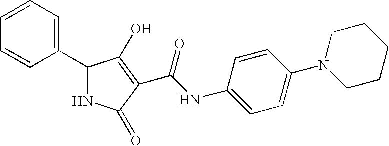 |
| 8. | 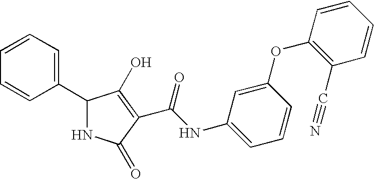 |
| 9. | 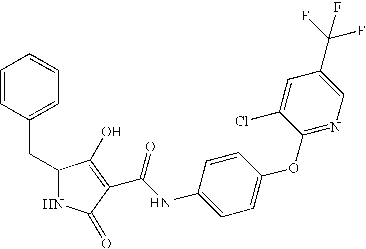 |
| 10. | 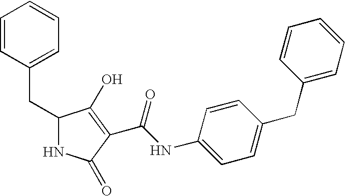 |
| 11. | 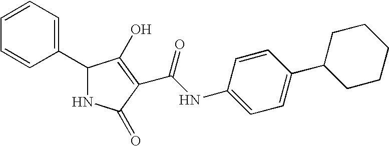 |
| 12. | 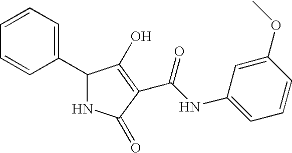 |
| 13. | 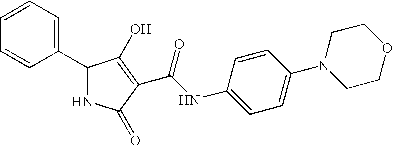 |
| 14. | 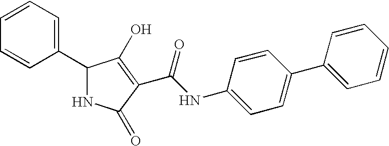 |
| 15. | 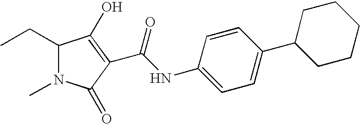 |
| 16. | 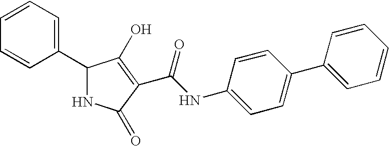 |
| 17. | 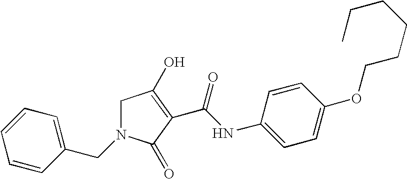 |
| 18. | 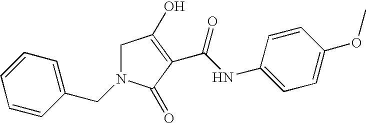 |
| 19. | 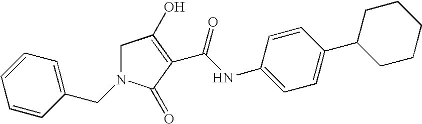 |
| 20. | 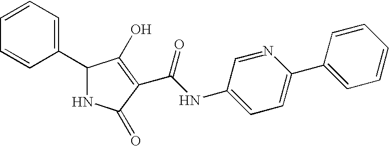 |
| 21. | 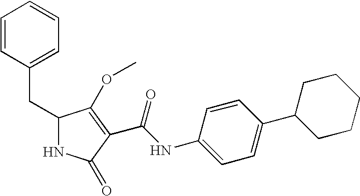 |
| 22. | 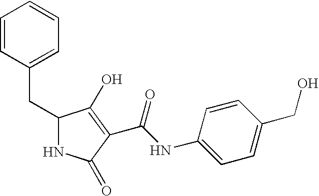 |
| 23. | 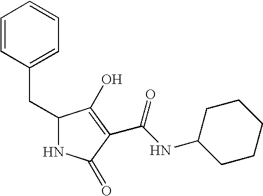 |
| 24. | 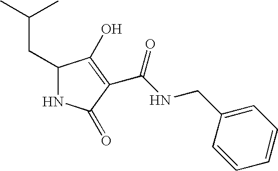 |
| 25. | 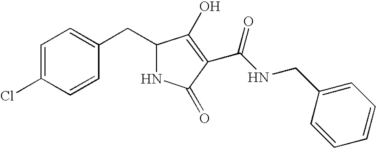 |
| 26. | 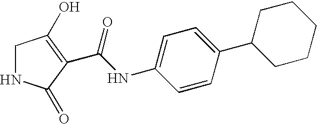 |
| 27. | 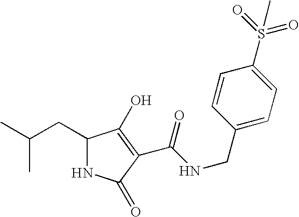 |
| 28. | 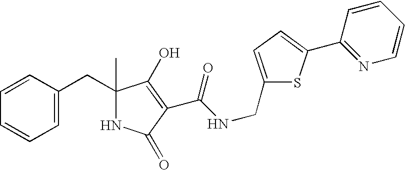 |
| 29. | 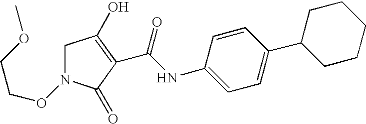 |
| 30. | 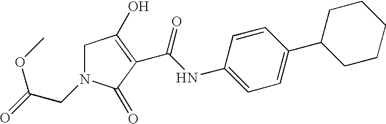 |
| 31. | 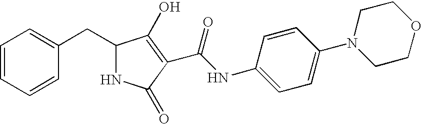 |
| 32. | 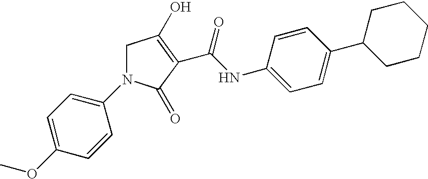 |
| 33. | 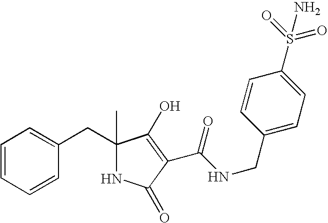 |
| 34. | 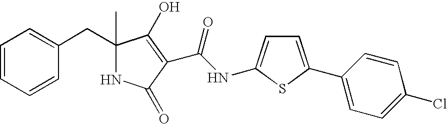 |
| 35. | 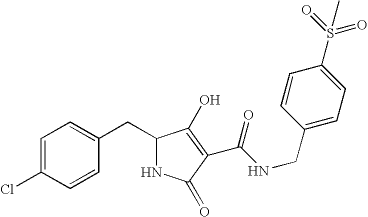 |
| 36. | 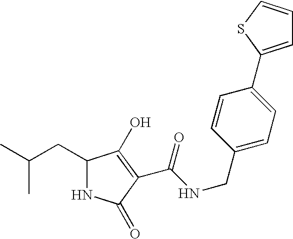 |
| 37. | 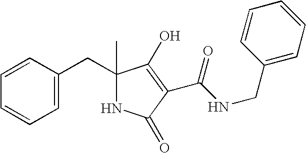 |
| 38. | 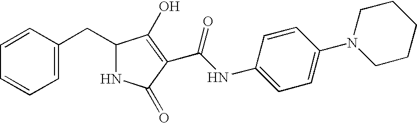 |
| 39. | 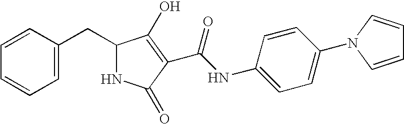 |
| 40. | 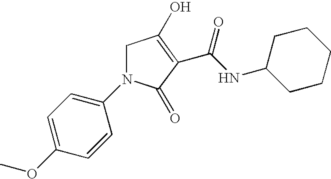 |
| 41. | 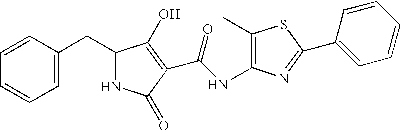 |
| 42. | 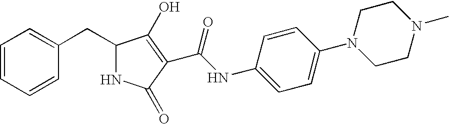 |
| 43. | 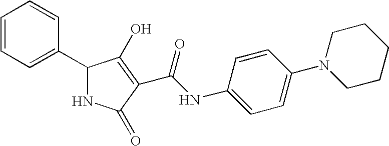 |
| 44. | 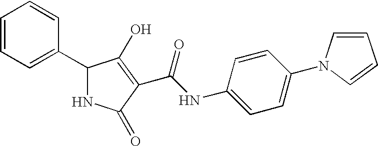 |
| 45. | 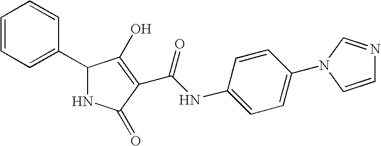 |
| 46. | 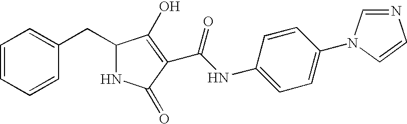 |
| 47. | 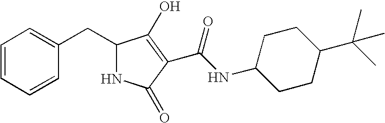 |
| 48. | 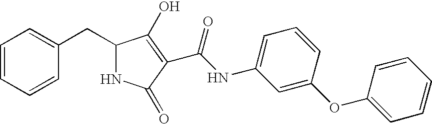 |
| 49. | 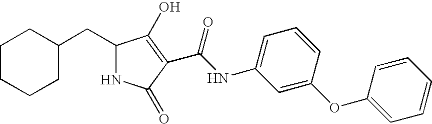 |
| 50. | 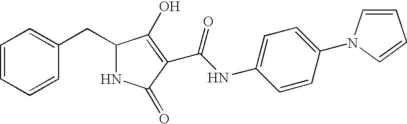 |
| 51. | 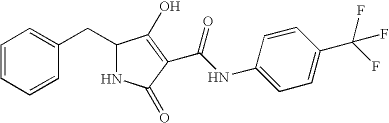 |
| 52. | 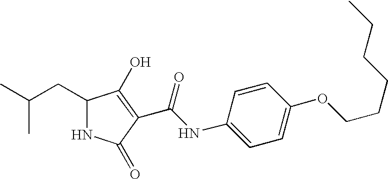 |
| 53. | 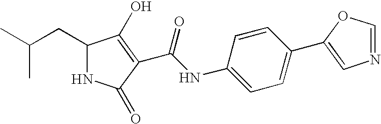 |
| 54. | 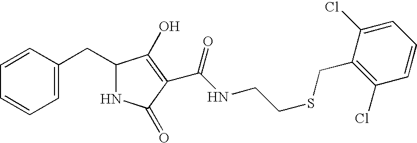 |
| 55. | 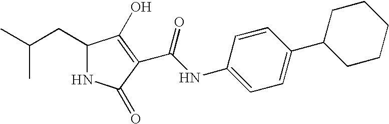 |
| 56. | 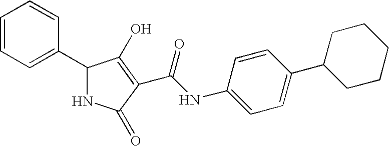 |
| 57. | 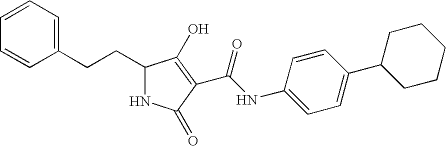 |
| 58. | 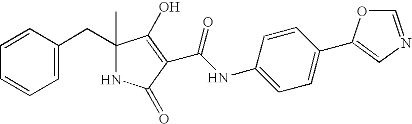 |
| 59. | 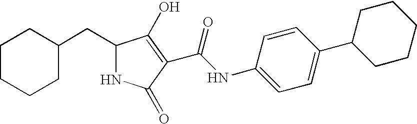 |
| 60. | 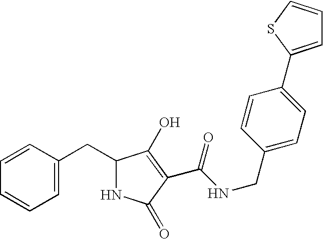 |
| 61. | 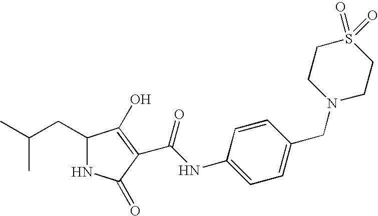 |
| 62. | 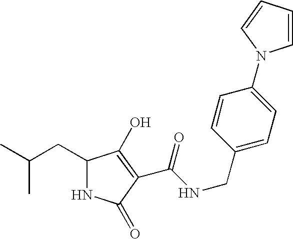 |
| 63. | 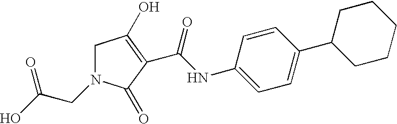 |
| 64. | 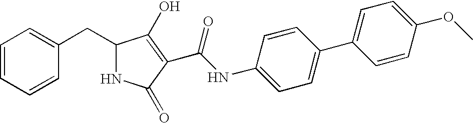 |
| 65. | 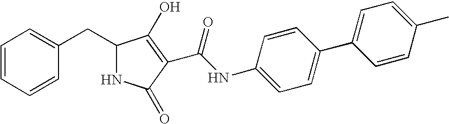 |
| 66. | 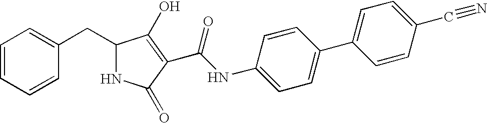 |
| 67. | 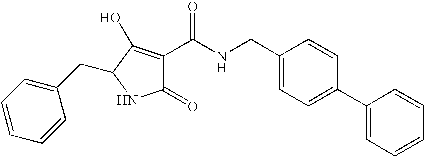 |
| 68. | 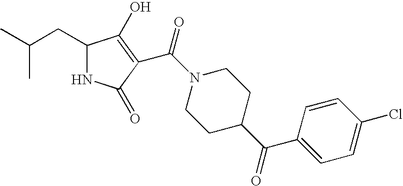 |
| 69. | 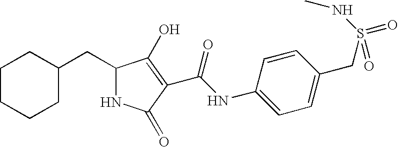 |
| 70. | 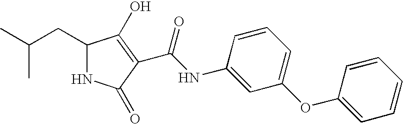 |
| 71. | 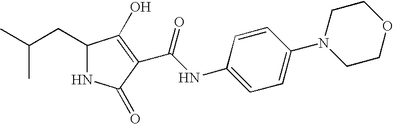 |
| 72. | 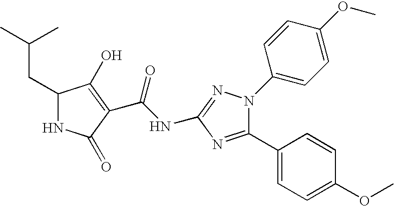 |
| 73. | 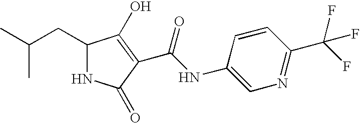 |
| 74. | 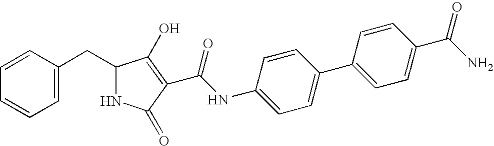 |
| 75. | 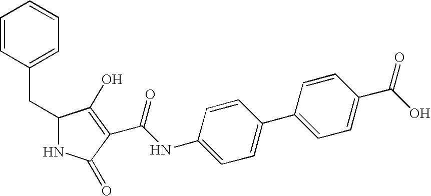 |
| 76. | 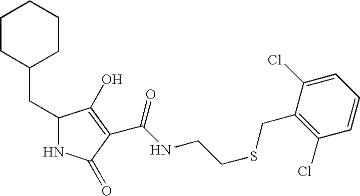 |
| 77. | 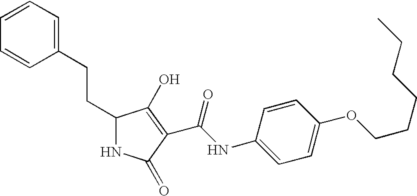 |
| 78. | 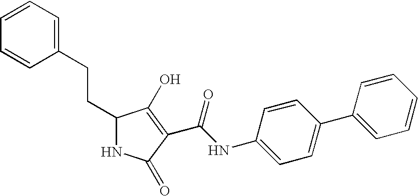 |
| 79. | 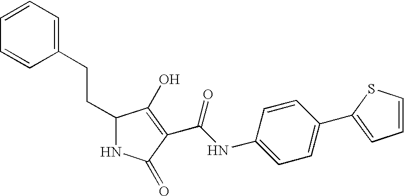 |
| 80. | 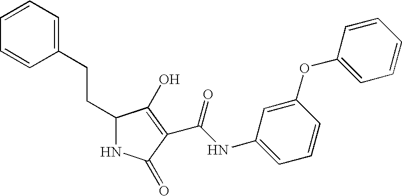 |
| 81. | 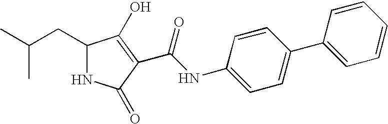 |
| 82. | 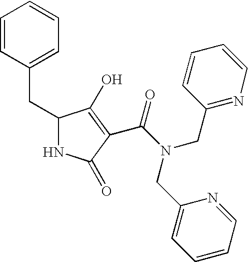 |
| 83. | 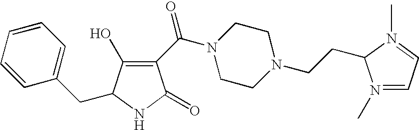 |
| 84. | 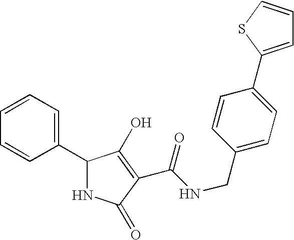 |
| 85. | 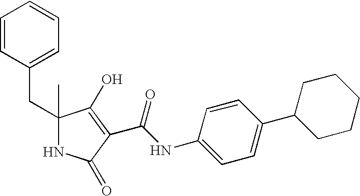 |
| 86. | 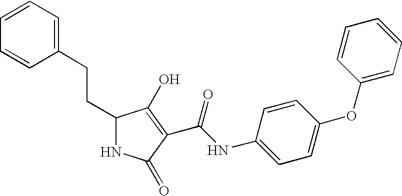 |
| 87. | 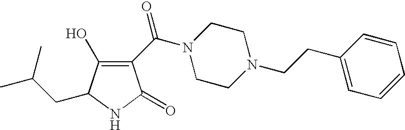 |
| 88. | 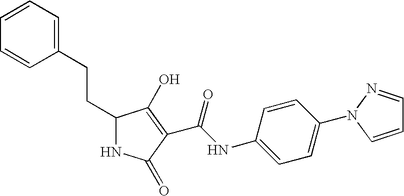 |
| 89. | 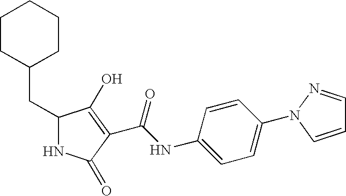 |
| 90. | 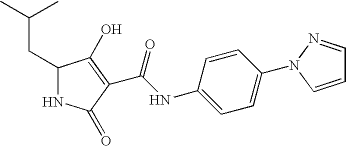 |
| 91. | 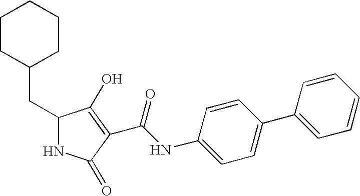 |
| 92. | 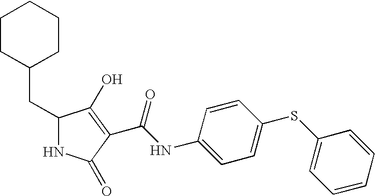 |
| 93. | 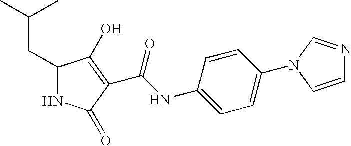 |
| 94. | 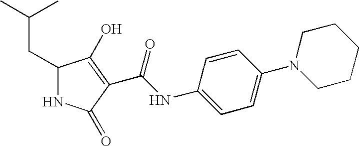 |
| 95. | 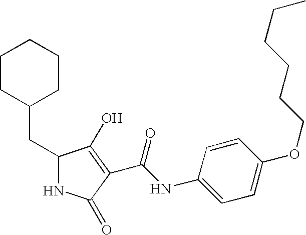 |
| 96. | 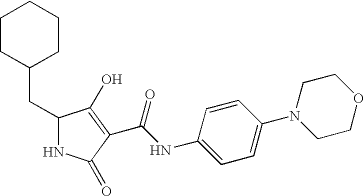 |
| 97. | 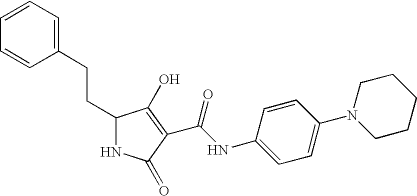 |
| 98. | 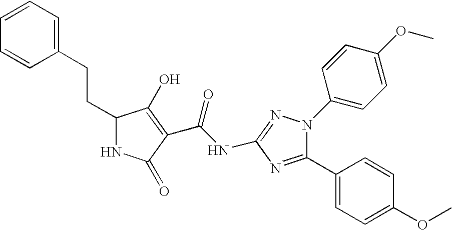 |
| 99. | 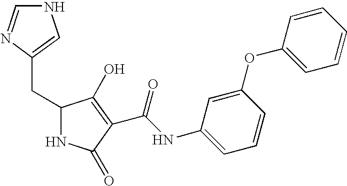 |
| 100. | 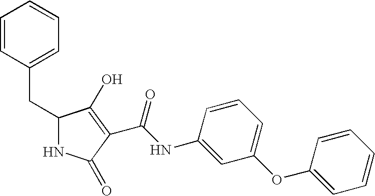 |
| 101. | 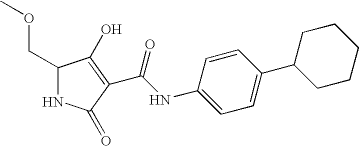 |
| 102. | 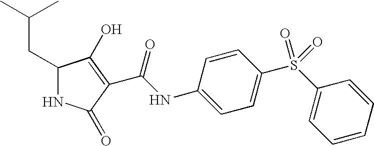 |
| 103. | 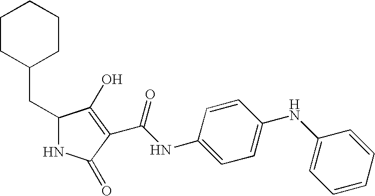 |
| 104. | 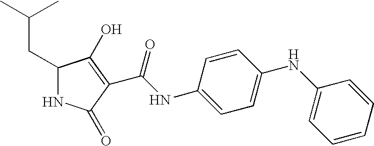 |
| 105. | 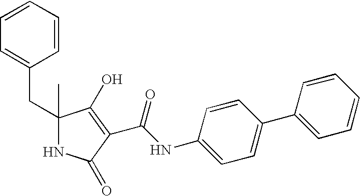 |
| 106. | 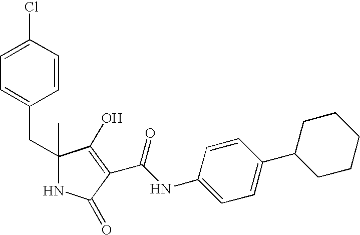 |
| 107. | 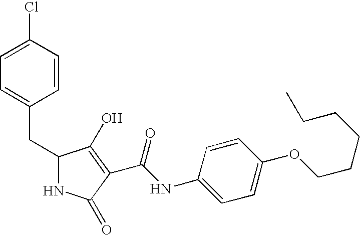 |
| 108. | 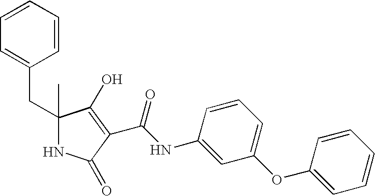 |
| 109. | 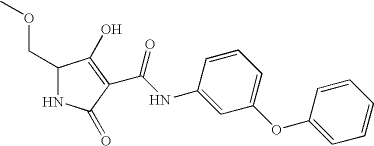 |
| 110. | 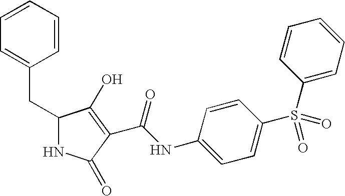 |
| 111. | 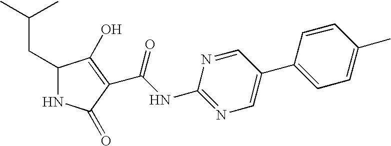 |
| 112. | 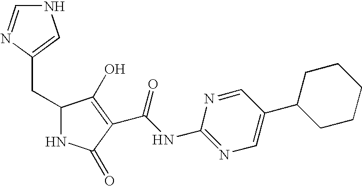 |
| 113. | 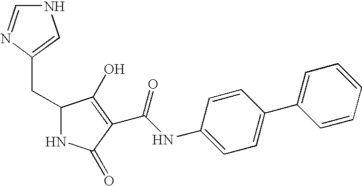 |
| 114. | 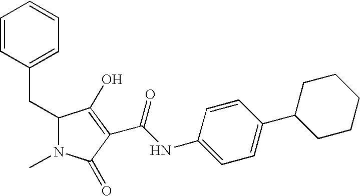 |
| 115. | 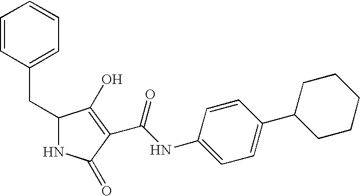 |
| 116. | 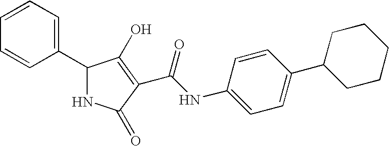 |
| 117. | 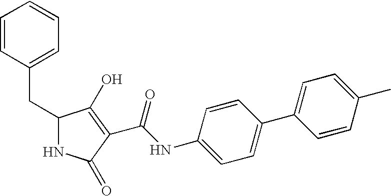 |
| 118. | 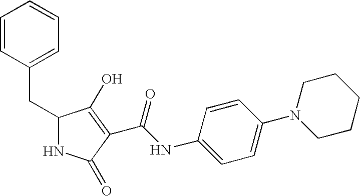 |
| 119. | 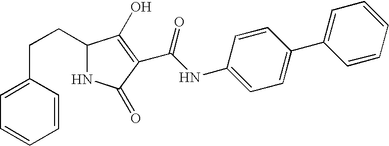 |
| 120. | 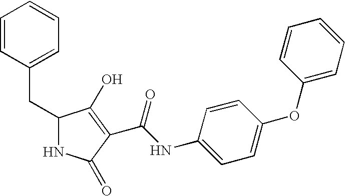 |
| 121. | 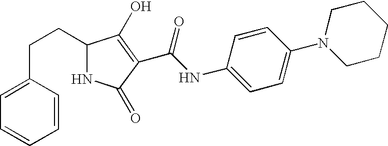 |
| 122. | 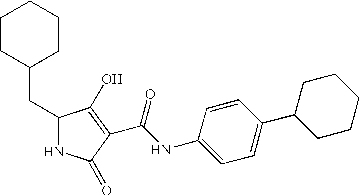 |
| 123. | 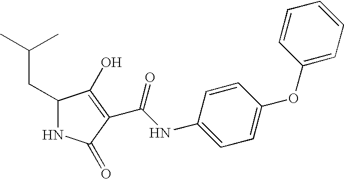 |
| 124. | 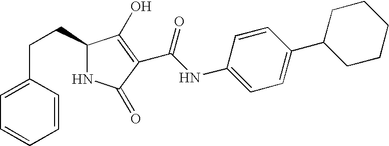 |
| 125. | 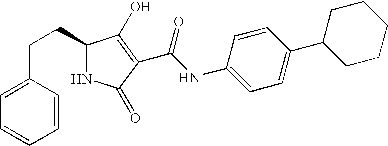 |
| 126. | 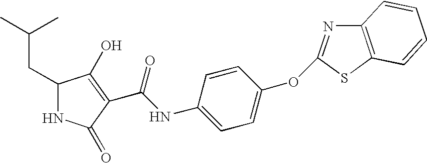 |
| 127. | 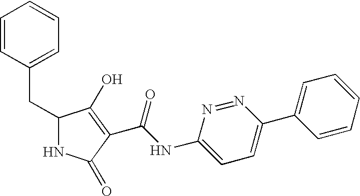 |
| 128. | 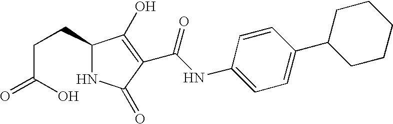 |
| 129. | 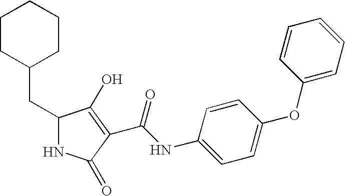 |
| 130. | 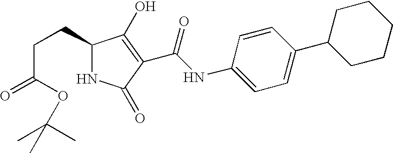 |
| 131. | 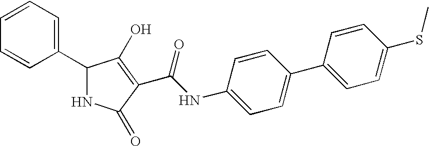 |
| 132. | 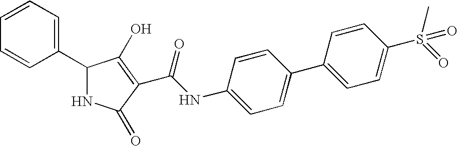 |
| 133. | 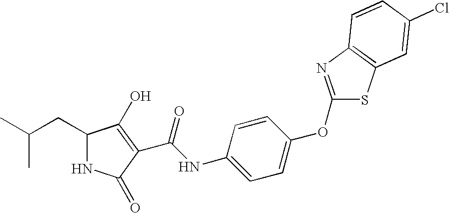 |
| 134. | 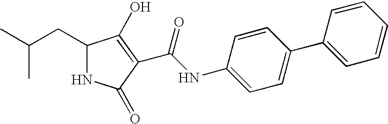 |
| 135. | 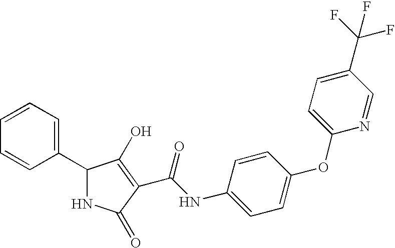 |
| 136. | 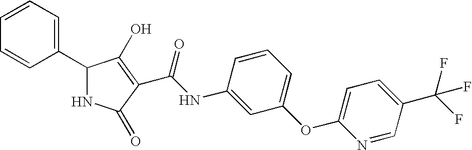 |
| 137. | 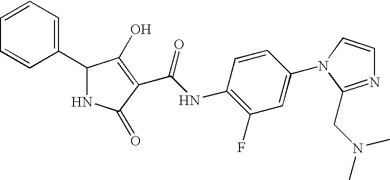 |
| 138. | 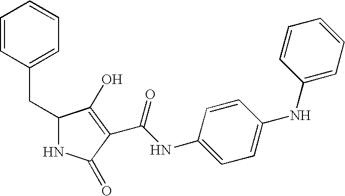 |
| 139. | 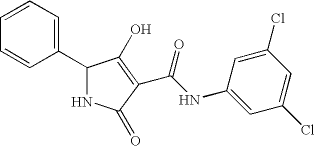 |
| 140. | 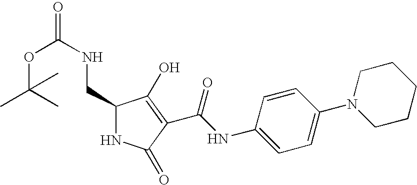 |
| 141. | 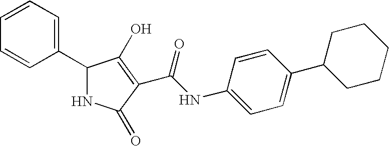 |
| 142. | 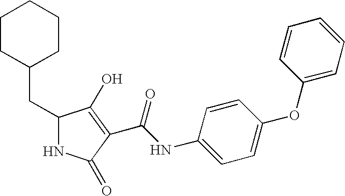 |
| 143. | 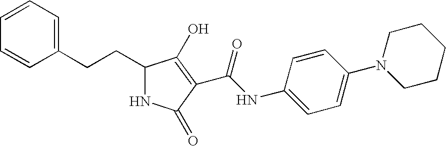 |
| 144. | 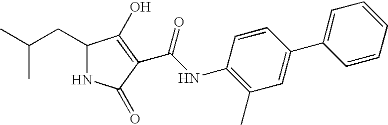 |
| 145. | 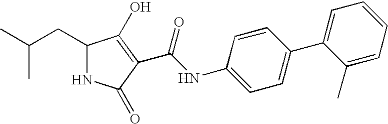 |
| 146. | 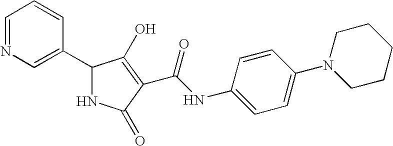 |
| 147. | 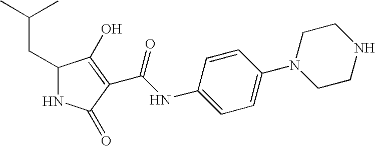 |
| 148. | 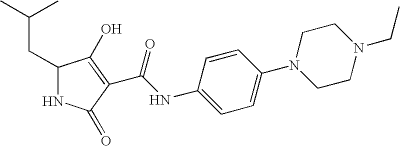 |
| 149. | 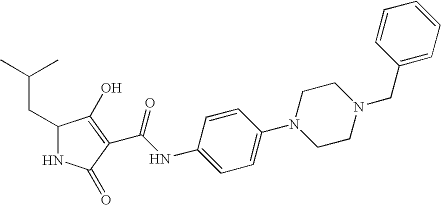 |
| 150. | 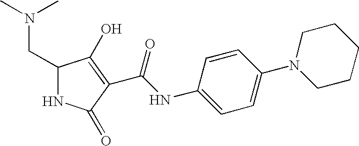 |
| 151. | 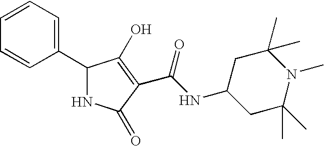 |
| 152. | 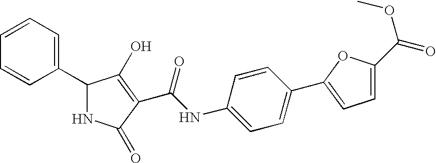 |
| 153. | 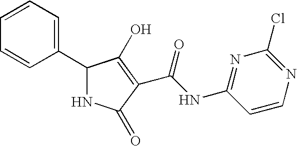 |
| 154. | 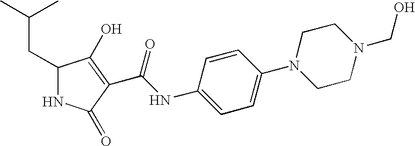 |
| 155. | 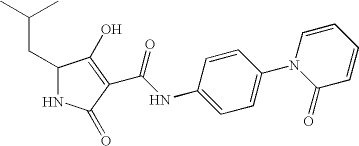 |
| 156. | 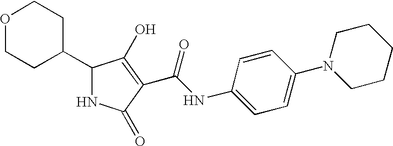 |
| 157. | 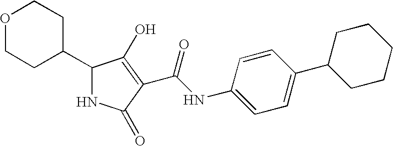 |
| 158. | 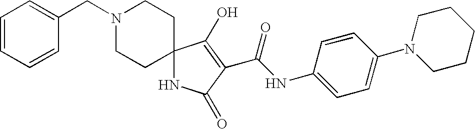 |
| 159. | 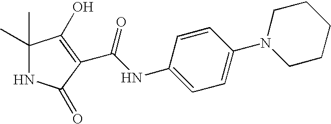 |
| 160. | 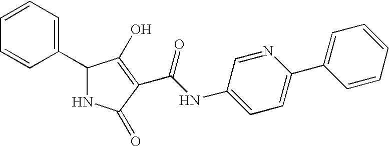 |
| 161. | 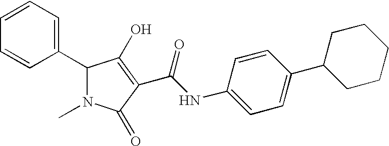 |
| 162. | 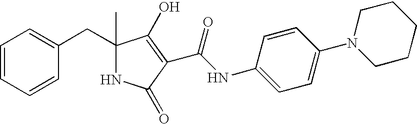 |
| 163. | 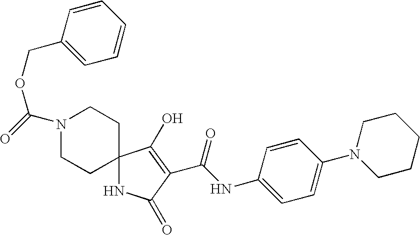 |
| 164. | 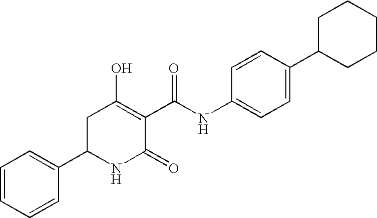 |
| 165. | 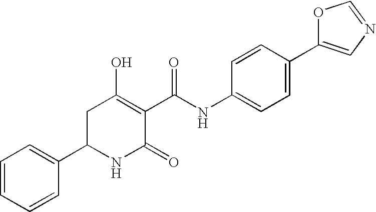 |
| 166. | 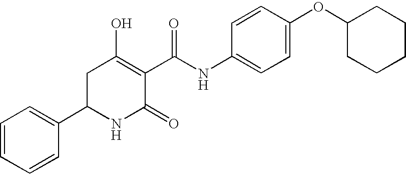 |
| 167. | 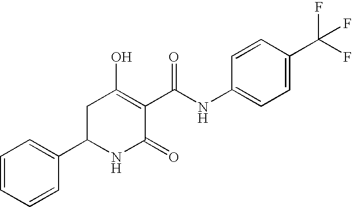 |
| 168. | 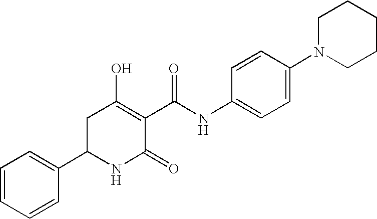 |
| 169. | 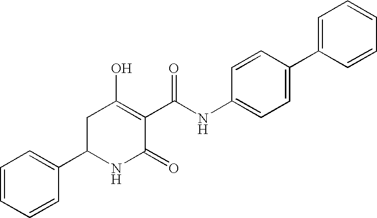 |
| 170. | 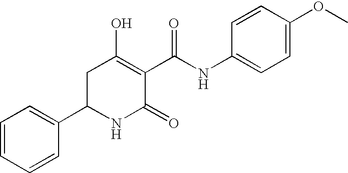 |
| 171. | 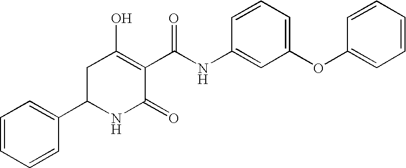 |
| 172. | 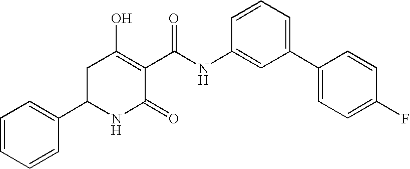 |
| 173. | 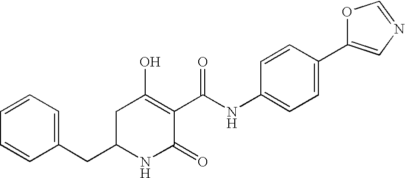 |
| 174. | 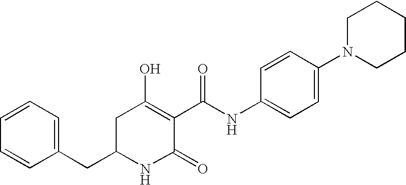 |
| 175. | 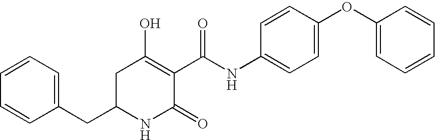 |
| 176. | 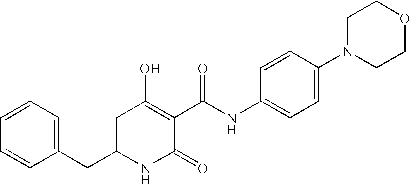 |
| 177. | 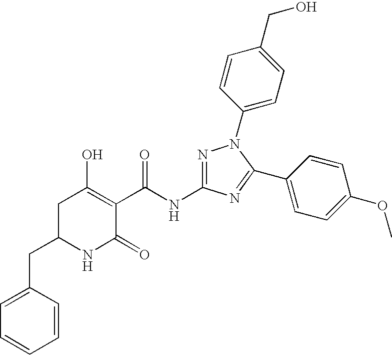 |
| 178. | 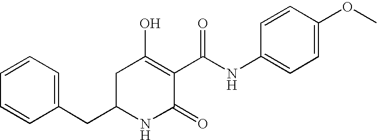 |
| 179. | 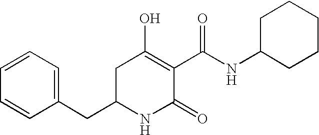 |
| 180. | 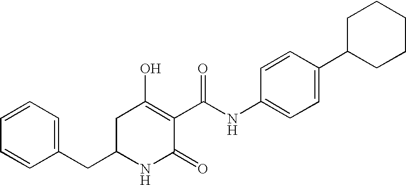 |
| 181. | 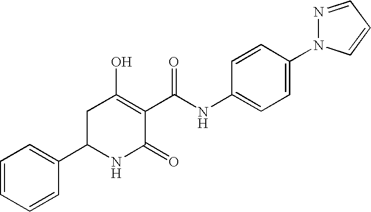 |
| 182. | 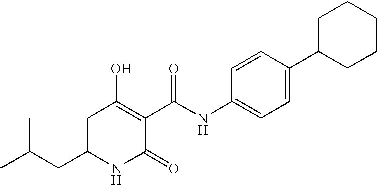 |
| 183. | 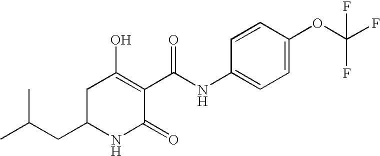 |
| 184. | 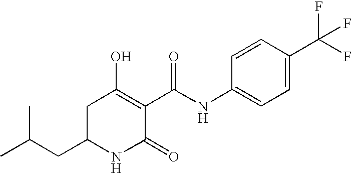 |
| 185. | 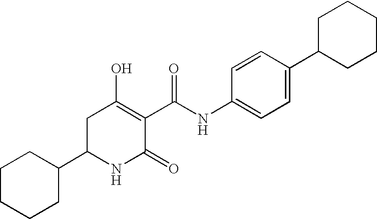 |
| 186. | 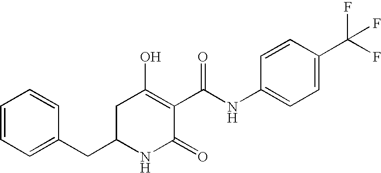 |
| 187. | 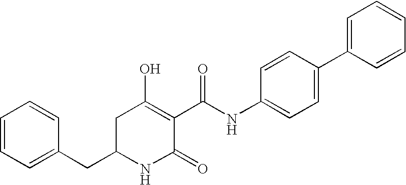 |
| 188. | 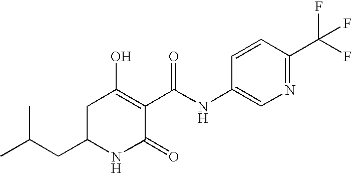 |
| 189. | 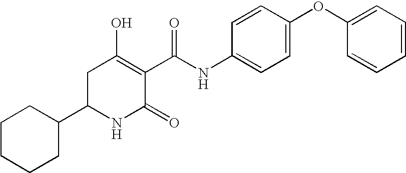 |
| 190. | 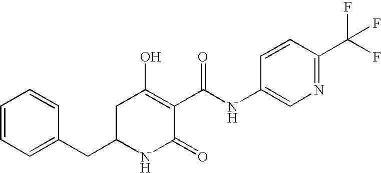 |
| 191. | 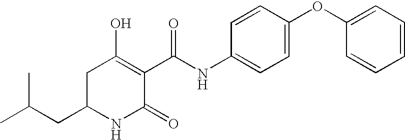 |
| 192. | 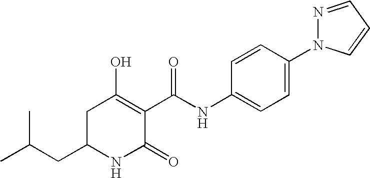 |
| 193. | 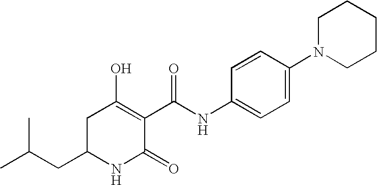 |
| 194. | 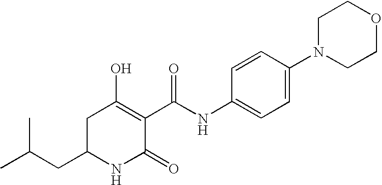 |
| 195. | 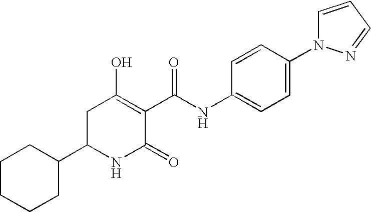 |
| 196. | 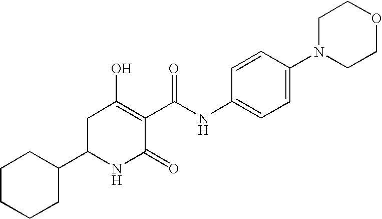 |
| 197. | 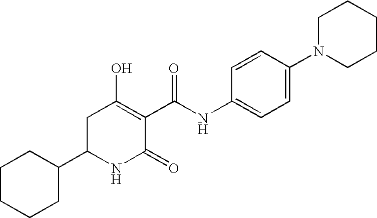 |
| 198. | 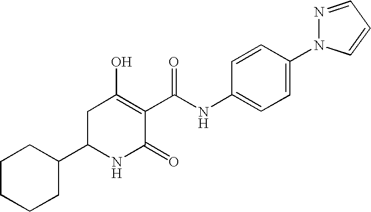 |
| 199. | 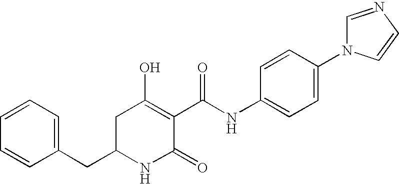 |
| 200. | 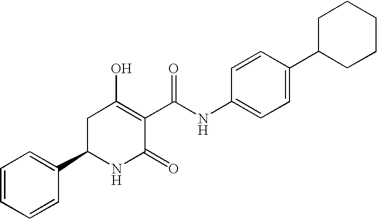 |
| 201. | 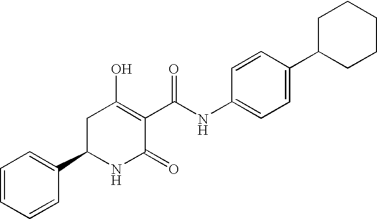 |
| 202. | 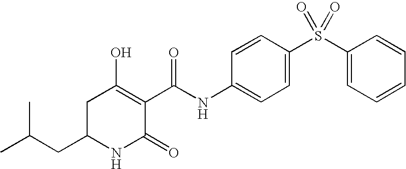 |
| 203. | 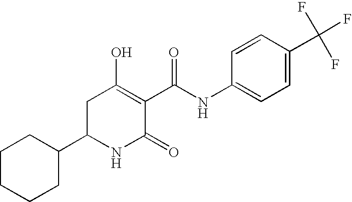 |
| 204. | 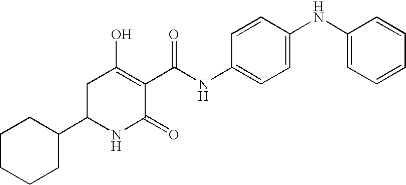 |
| 205. | 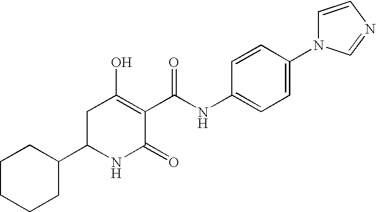 |
| 206. | 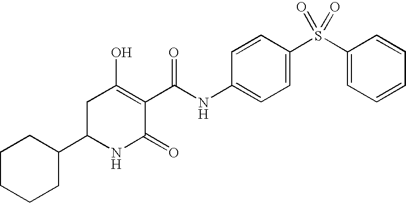 |
| 207. | 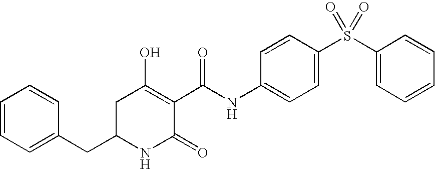 |
| 208. | 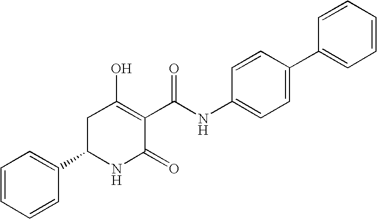 |
| 209. | 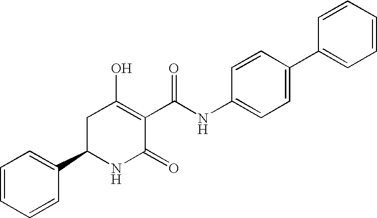 |
| 210. | 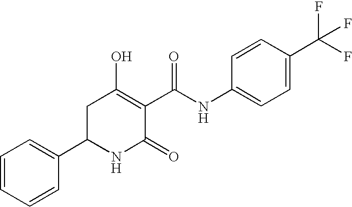 |
| 211. | 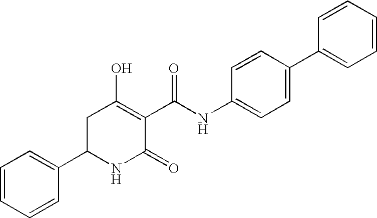 |
| 212. | 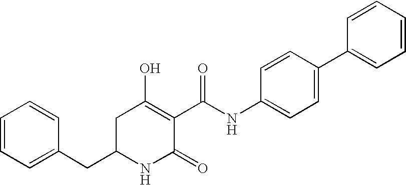 |
| 213. | 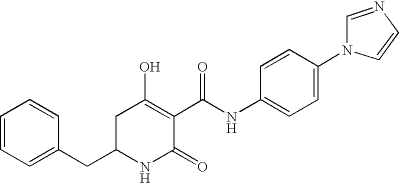 |
| 214. | 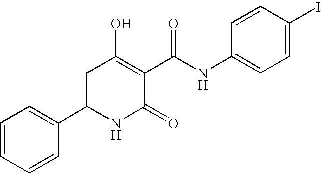 |
| 215. | 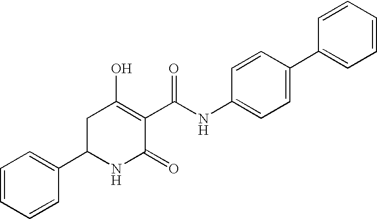 |
| 216. | 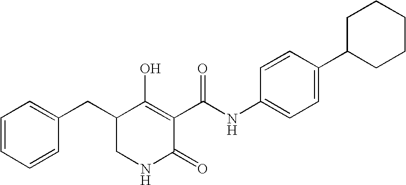 |
| 217. | 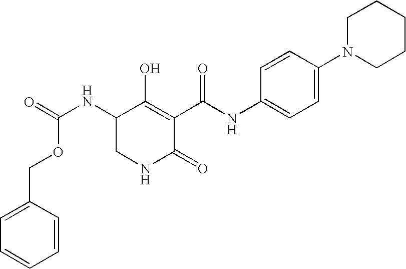 |
| 218. | 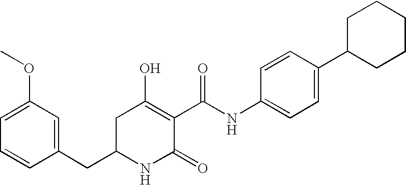 |
| 219. | 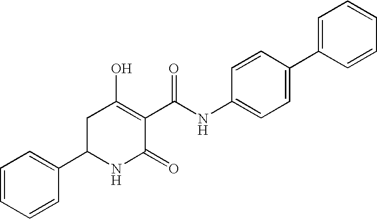 |
| 220. | 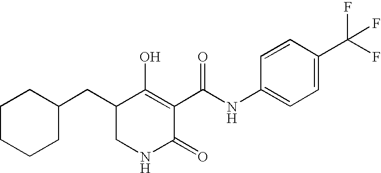 |
| 221. | 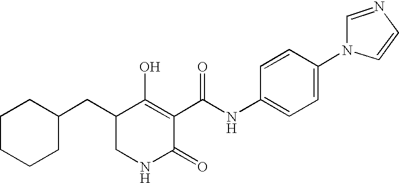 |
| 222. | 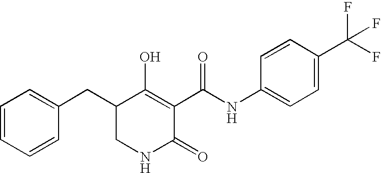 |
| 223. | 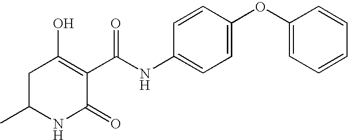 |
| 224. | 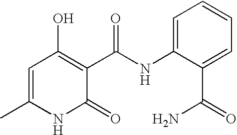 |
| 225. | 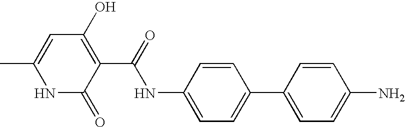 |
| 226. | 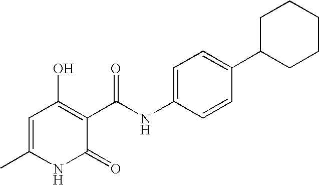 |
| 227. | 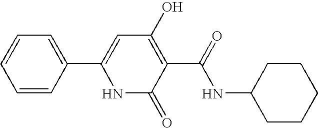 |
| 228. | 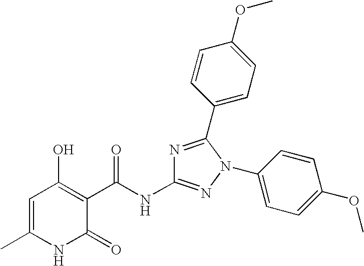 |
| 229. | 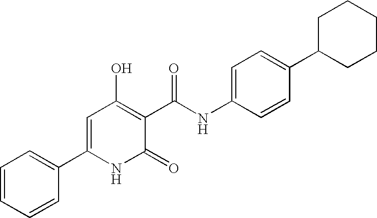 |
| 230. | 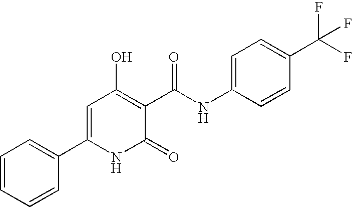 |
| 231. | 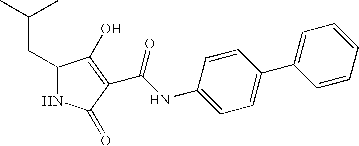 |
| 232. | 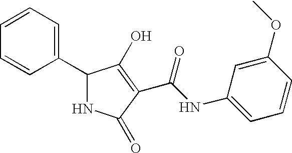 |
| 233. | 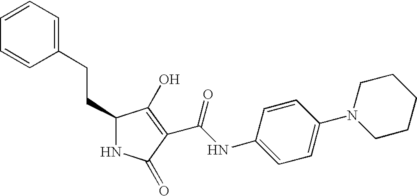 |
| 234. | 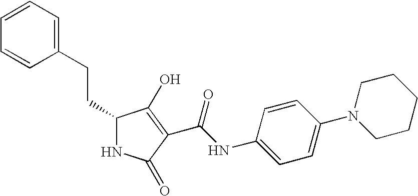 |
| 235. | 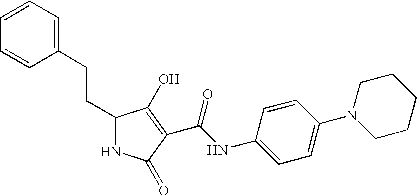 |
| 236. | 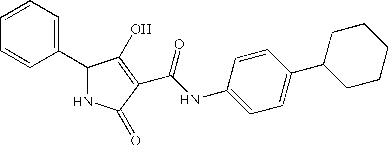 |
| 237. | 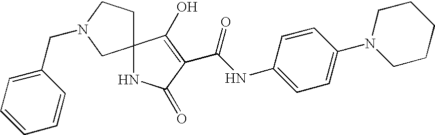 |
| 238. | 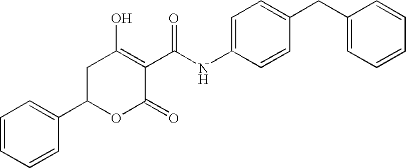 |
| 239. | 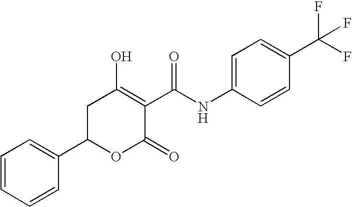 |
| 240. | 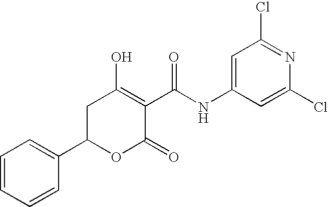 |
| 241. | 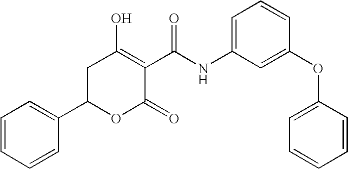 |
| 242. | 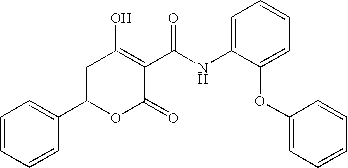 |
| 243. | 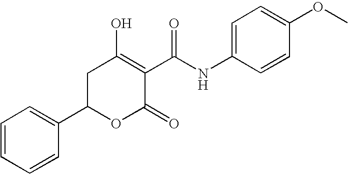 |

TABLE 2

| | |
|---|---|
| 244. | 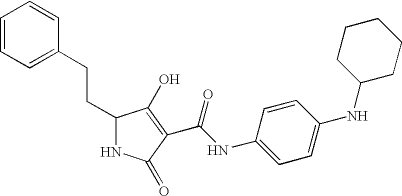 |
| 245. | 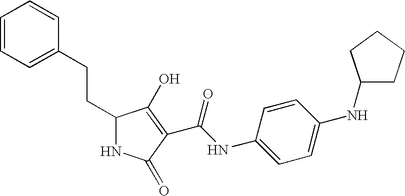 |
| 246. | 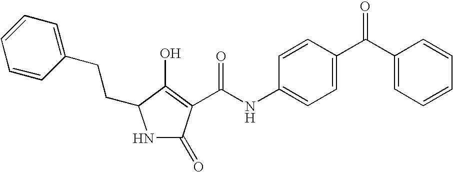 |
| 247. | 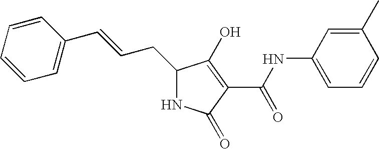 |
| 248. | 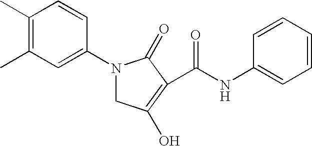 |
| 249. | 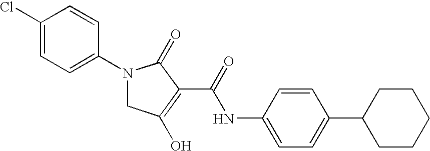 |
| 250. | 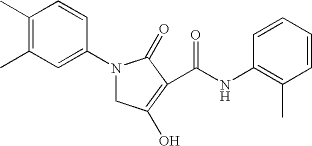 |
| 251. | 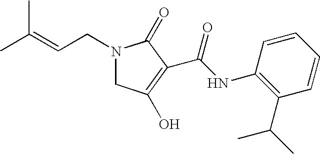 |
| 252. | 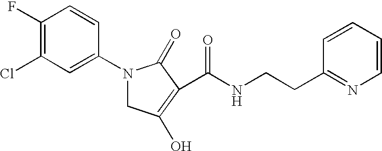 |
| 253. | 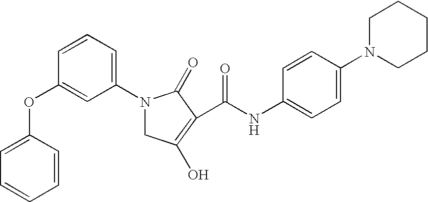 |
| 254. | 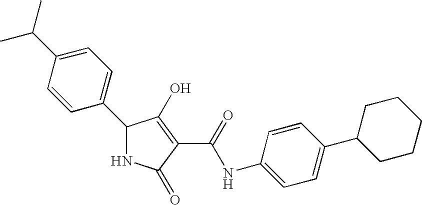 |
| 255. | 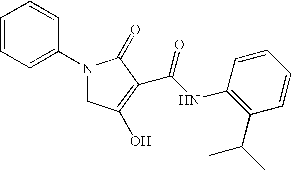 |
| 256. | 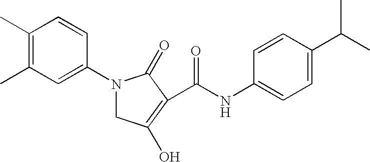 |
| 257. | 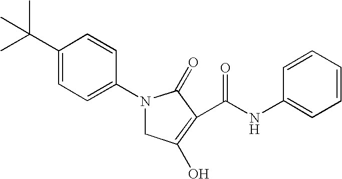 |
| 258. | 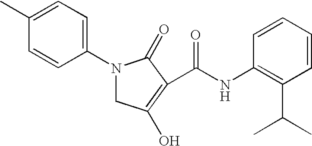 |
| 259. | 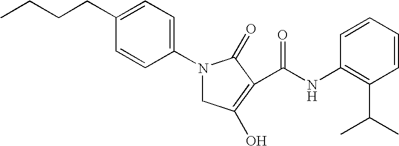 |
| 260. | 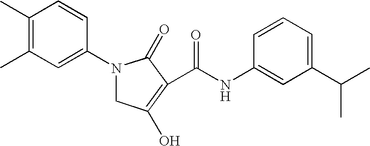 |
| 261. | 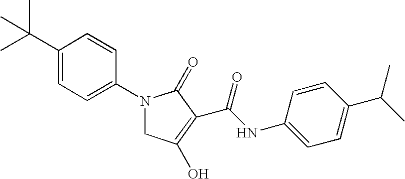 |
| 262. | 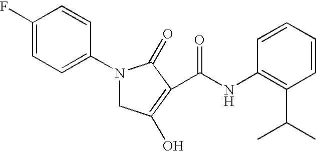 |
| 263. | 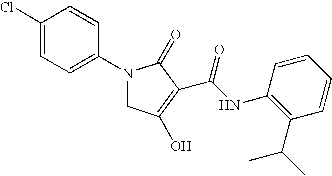 |
| 264. | 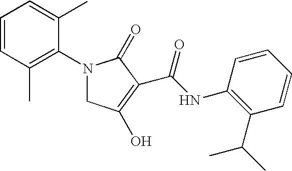 |
| 265. | 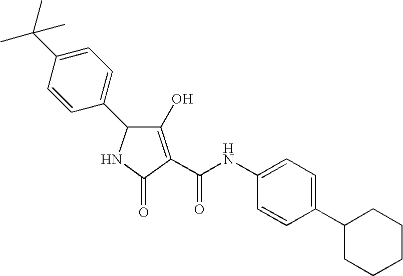 |
| 266. | 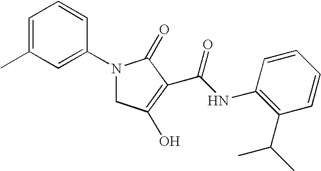 |
| 267. | 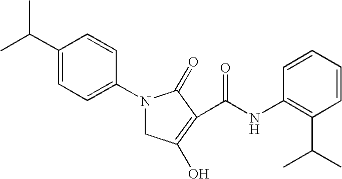 |
| 268. | 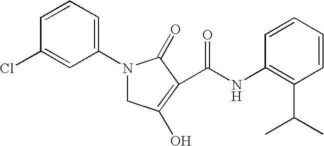 |
| 269. | 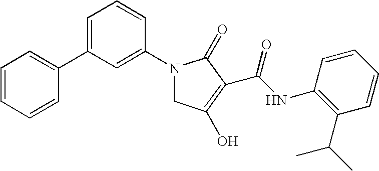 |
| 270. | 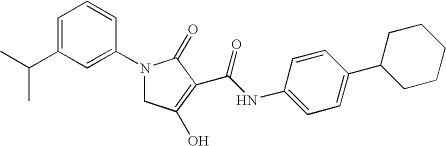 |
| 271. | 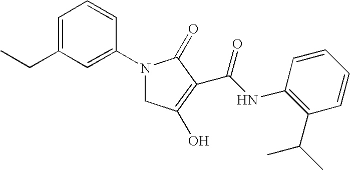 |
| 272. | 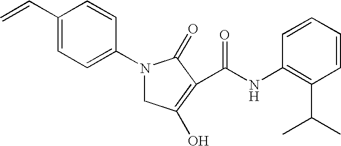 |
| 273. | 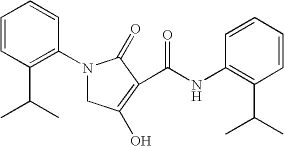 |
| 274. | 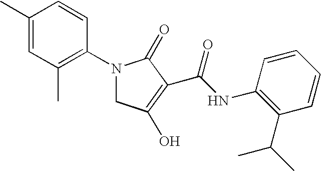 |
| 275. | 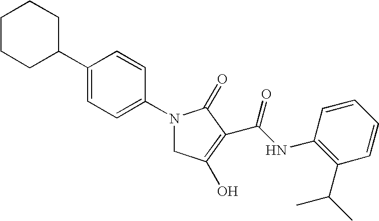 |
| 276. | 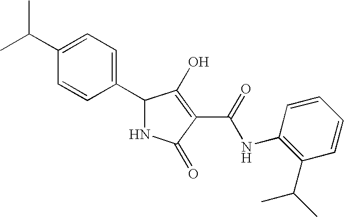 |
| 277. | 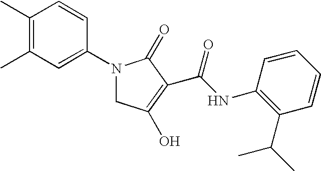 |
| 278. | 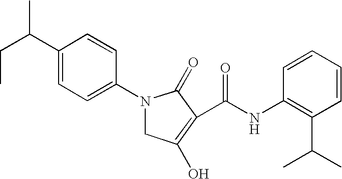 |
| 279. | 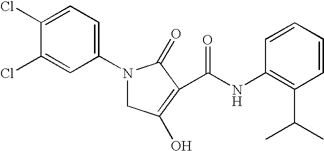 |
| 280. | 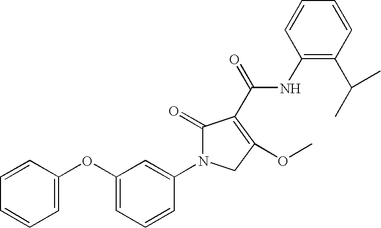 |
| 281. | 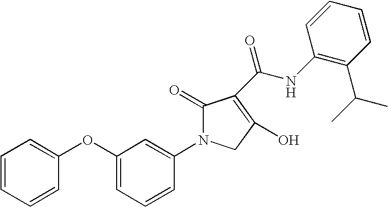 |
| 282. | 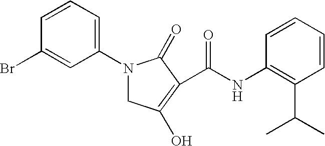 |
| 283. | 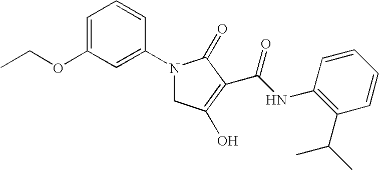 |
| 284. | 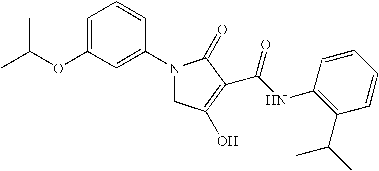 |
| 285. | 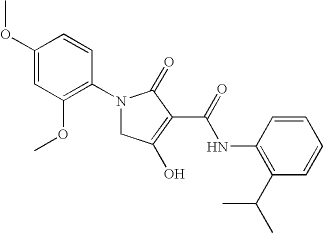 |
| 286. | 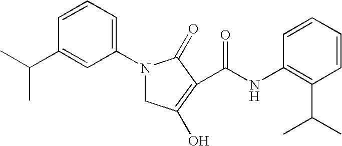 |
| 287. | 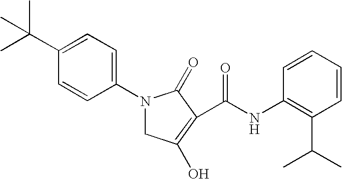 |
| 288. | 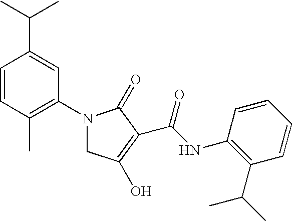 |
| 289. | 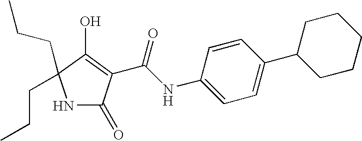 |
| 290. | 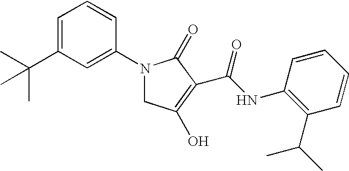 |
| 291. | 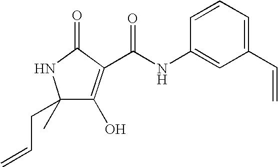 |
| 292. | 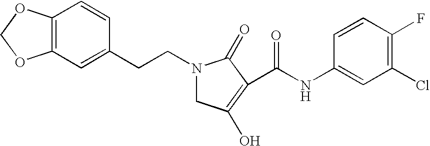 |
| 293. | 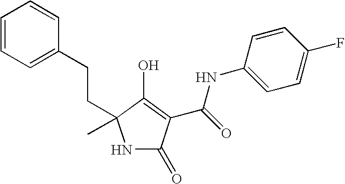 |
| 294. | 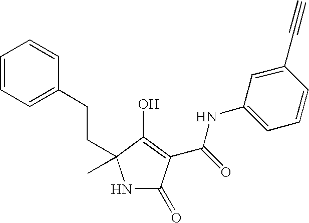 |
| 295. | 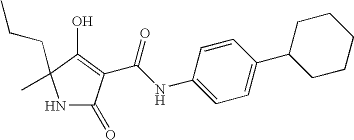 |
| 296. | 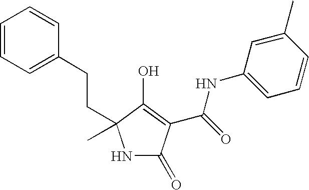 |
| 297. | 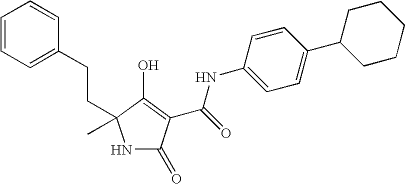 |
| 298. | 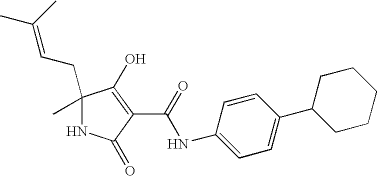 |
| 299. | 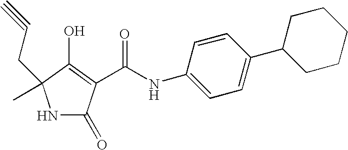 |
| 300. | 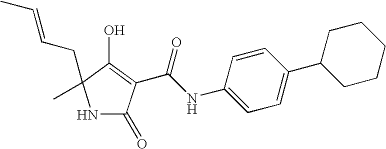 |
| 301. | 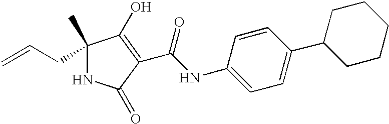 |
| 302. | 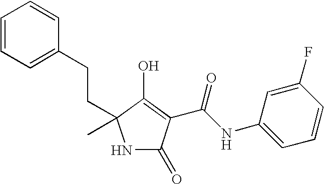 |
| 303. | 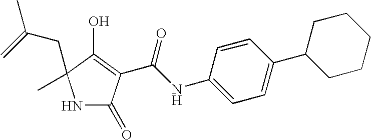 |
| 304. | 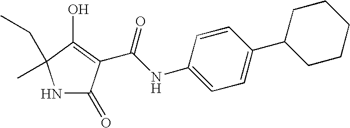 |
| 305. | 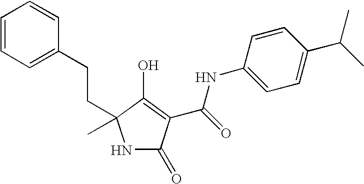 |
| 306. | 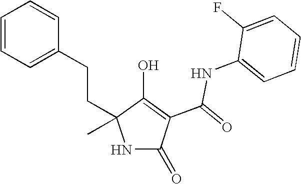 |
| 307. | 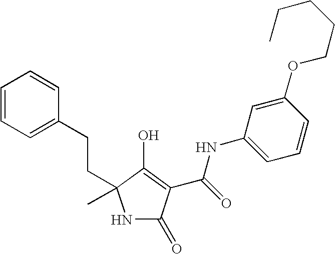 |
| 308. | 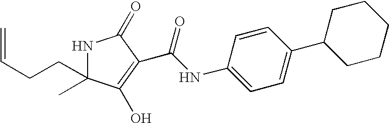 |
| 309. | 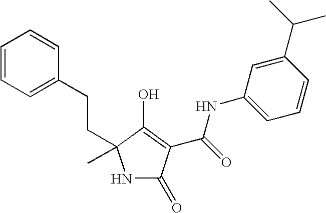 |
| 310. | 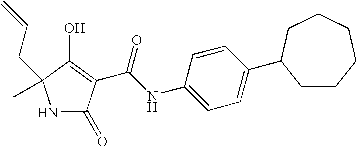 |
| 311. | 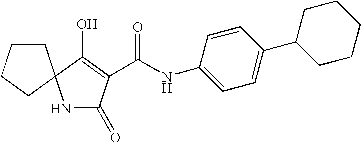 |
| 312. | 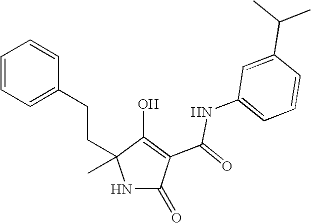 |
| 313. | 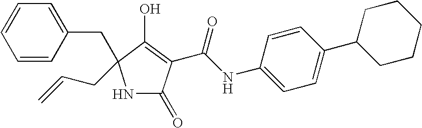 |
| 314. | 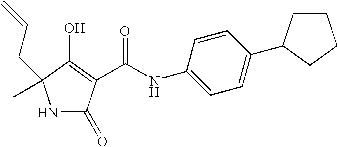 |
| 315. | 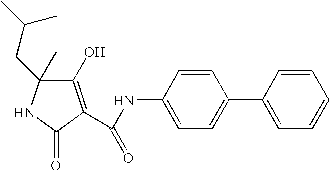 |
| 316. | 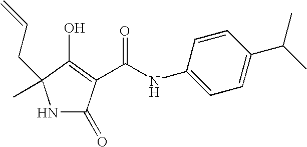 |
| 317. | 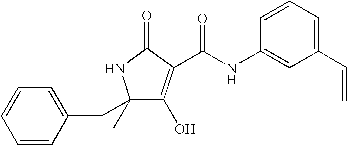 |
| 318. | 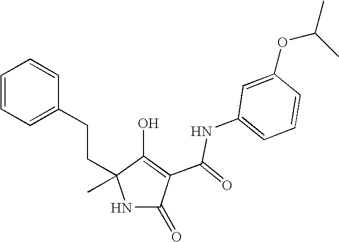 |
| 319. | 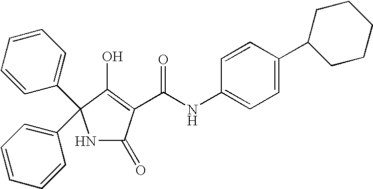 |
| 320. | 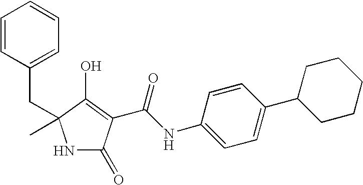 |
| 321. | 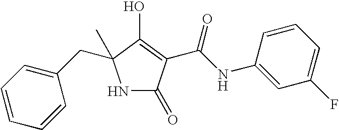 |
| 322. | 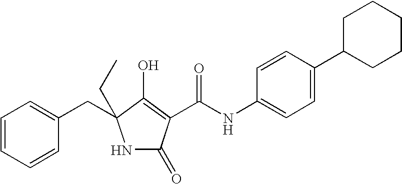 |
| 323. | 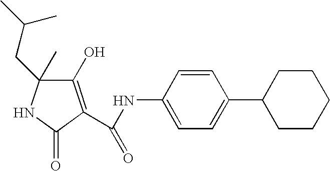 |
| 324. | 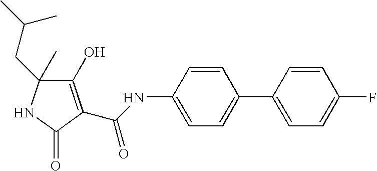 |
| 325. | 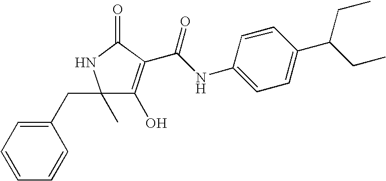 |
| 326. | 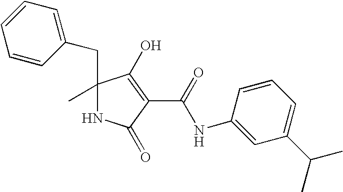 |
| 327. | 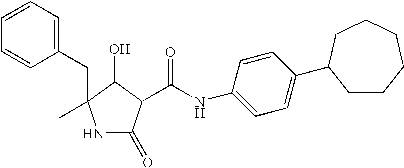 |
| 328. | 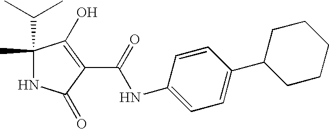 |
| 329. | 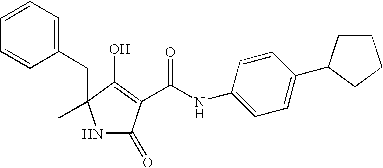 |
| 330. | 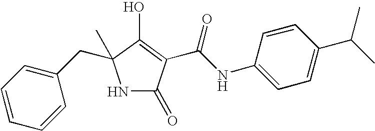 |
| 331. | 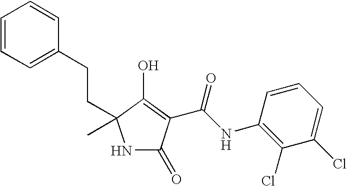 |
| 332. | 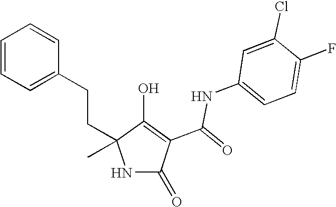 |
| 333. | 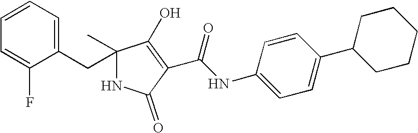 |
| 334. | 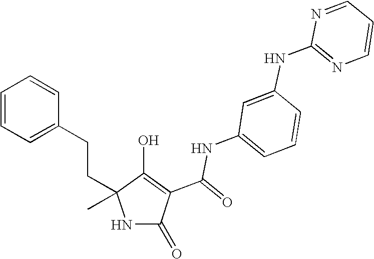 |
| 335. | 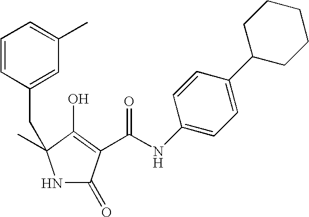 |
| 336. | 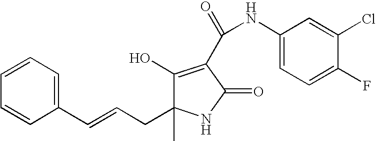 |
| 337. | 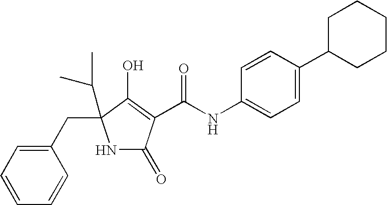 |
| 338. | 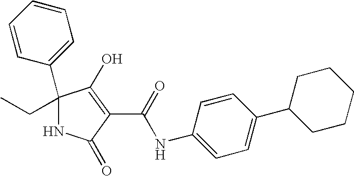 |
| 339. | 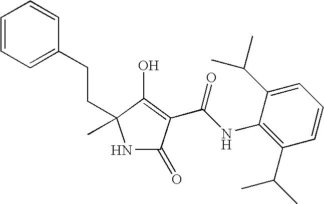 |
| 340. | 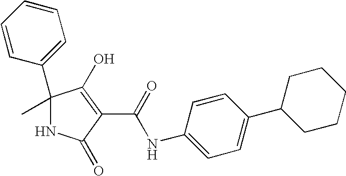 |
| 341. | 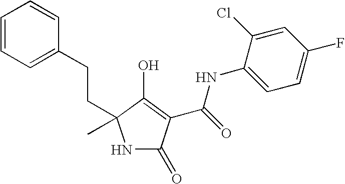 |
| 342. | 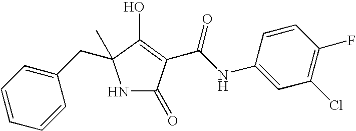 |
| 343. | 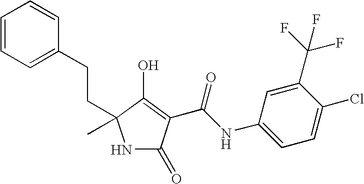 |
| 344. | 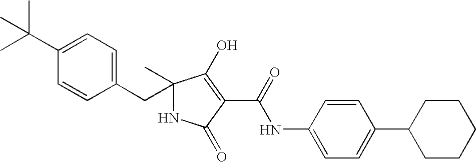 |
| 345. | 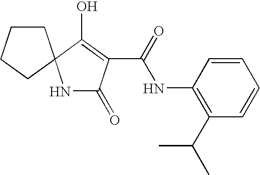 |
| 346. | 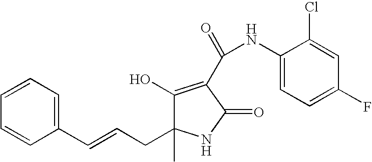 |
| 347. | 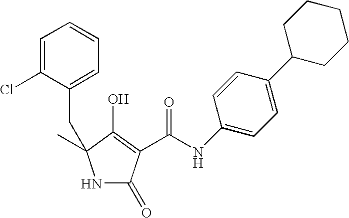 |
| 348. | 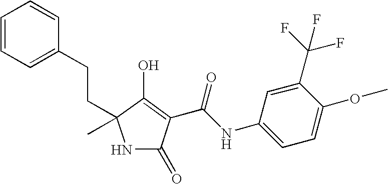 |
| 349. | 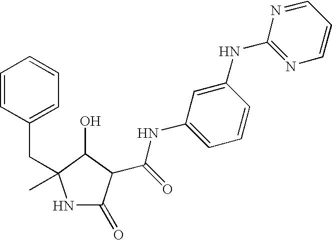 |
| 350. | 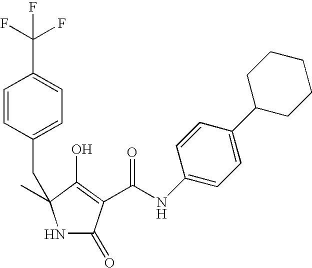 |
| 351. | 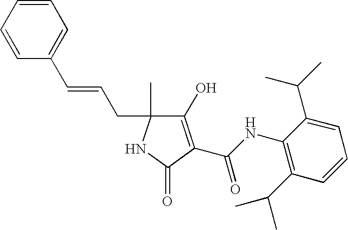 |
| 352. | 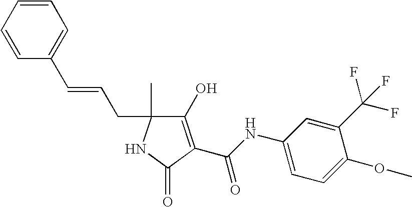 |
| 353. | 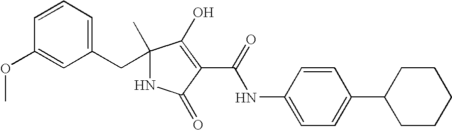 |
| 354. | 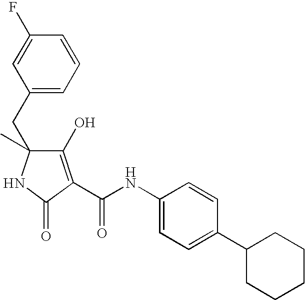 |
| 355. | 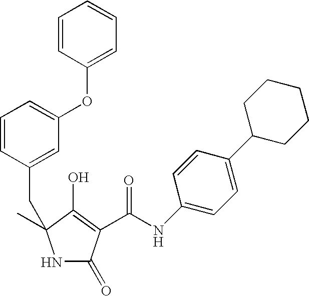 |
| 356. | 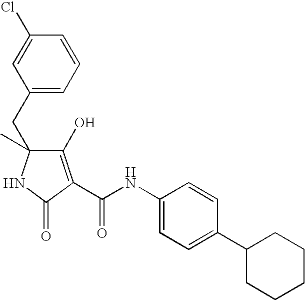 |
| 357. | 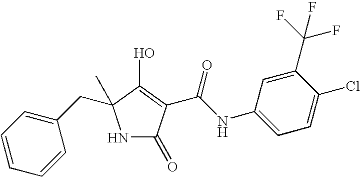 |
| 358. | 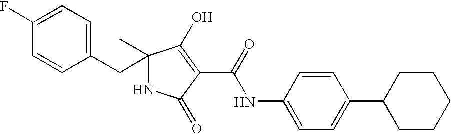 |
| 359. | 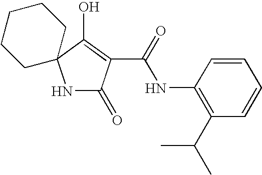 |
| 360. | 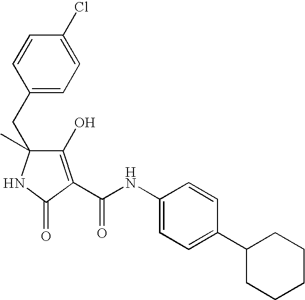 |
| 361. | 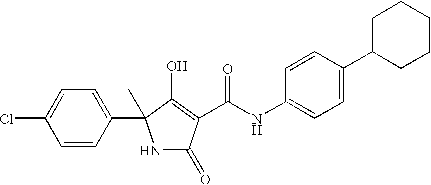 |
| 362. | 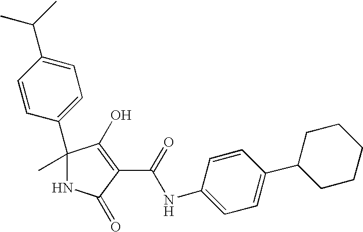 |
| 363. | 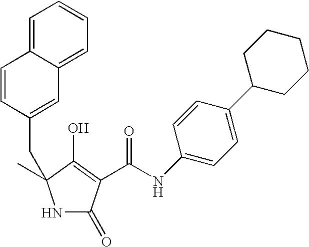 |
| 364. | 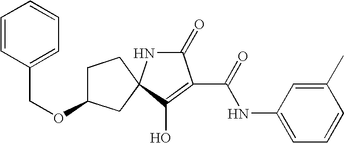 |
| 365. | 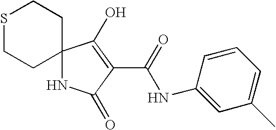 |
| 366. | 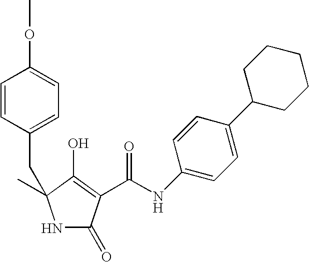 |
| 367. | 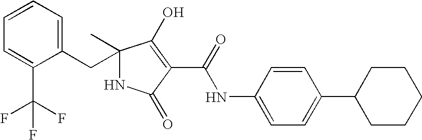 |
| 368. | 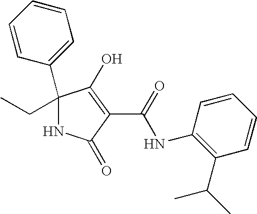 |
| 369. | 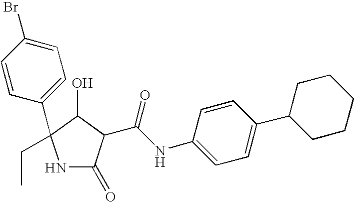 |
| 370. | 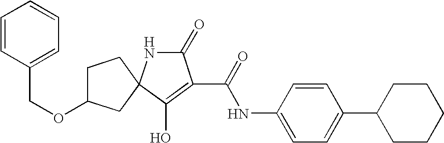 |
| 371. | 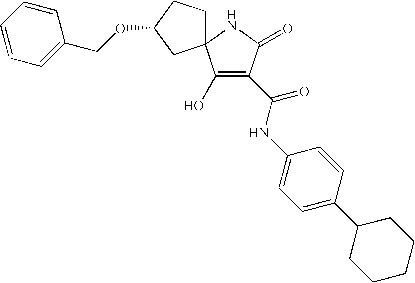 |
| 372. | 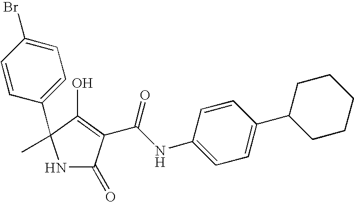 |
| 373. | 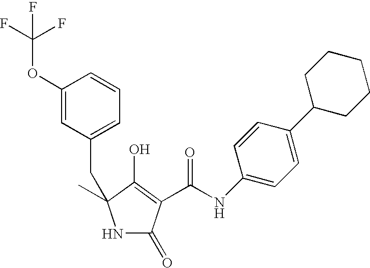 |
| 374. | 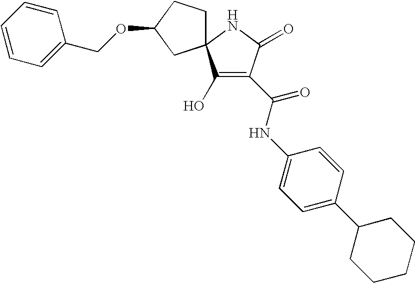 |
| 375. | 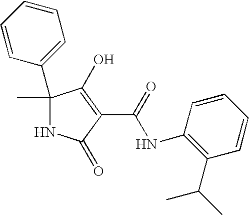 |
| 376. | 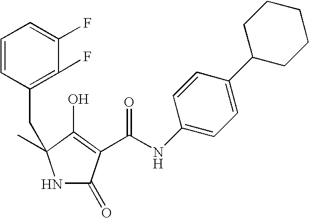 |
| 377. | 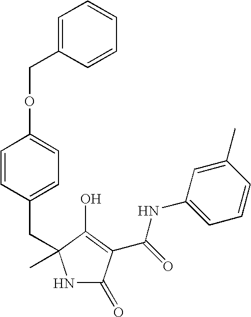 |
| 378. | 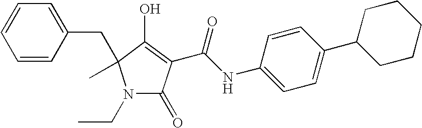 |
| 379. | 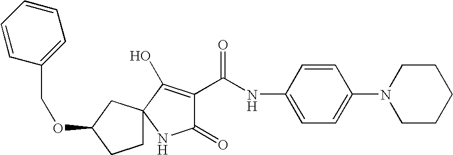 |
| 380. | 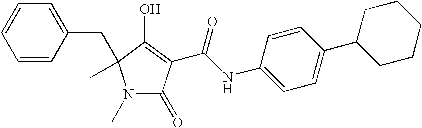 |
| 381. | 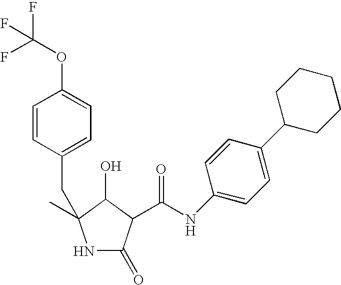 |
| 382. | 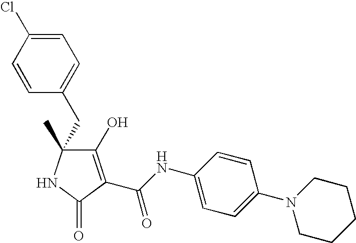 |
| 383. | 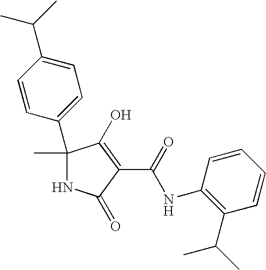 |
| 384. | 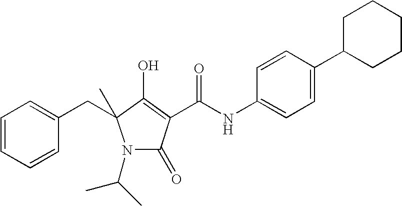 |
| 385. | 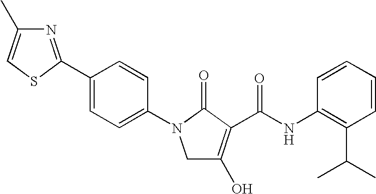 |
| 386. | 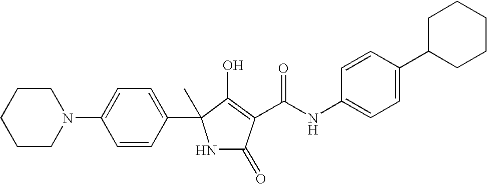 |
| 387. | 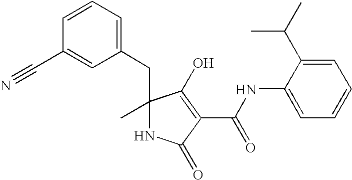 |
| 388. | 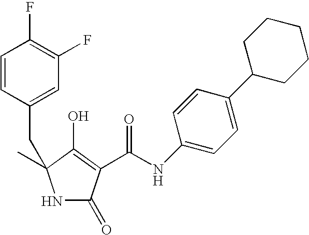 |
| 389. | 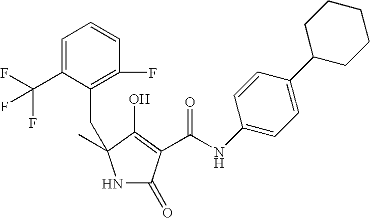 |
| 390. | 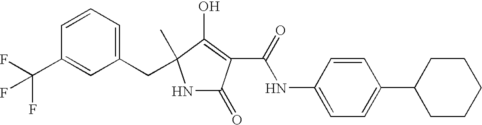 |
| 391. | 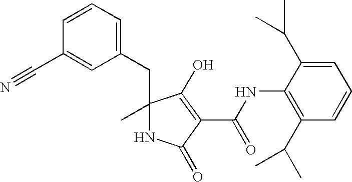 |
| 392. | 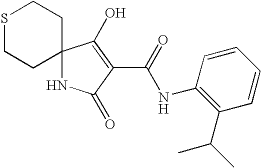 |
| 393. | 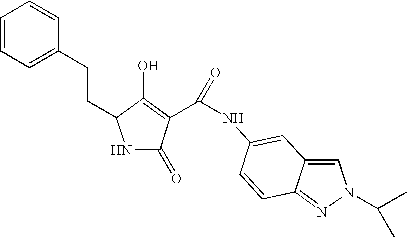 |
| 394. | 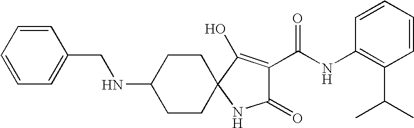 |
| 395. | 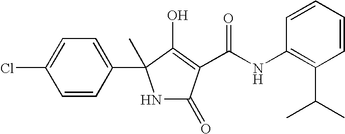 |
| 396. | 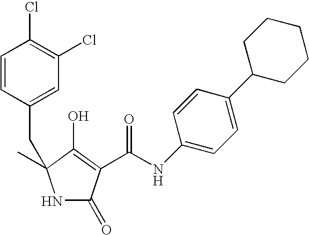 |
| 397. | 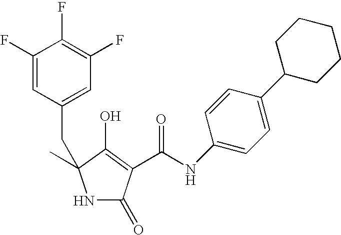 |
| 398. | 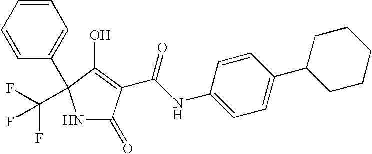 |
| 399. | 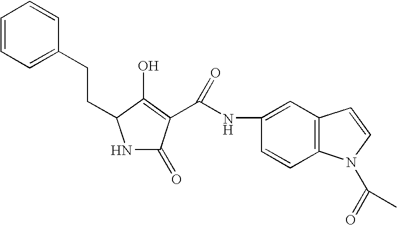 |
| 400. | 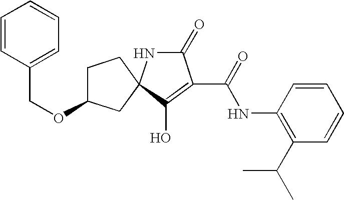 |
| 401. | 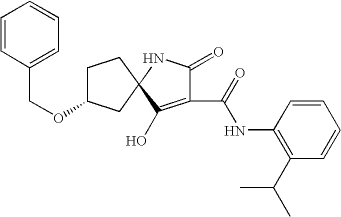 |
| 402. | 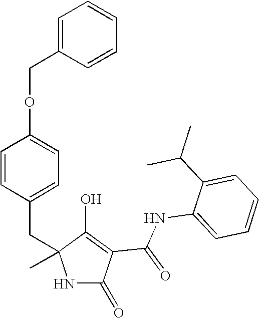 |
| 403. | 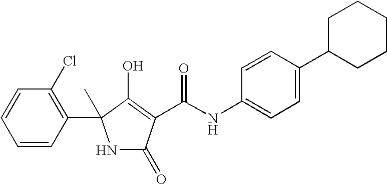 |
| 404. | 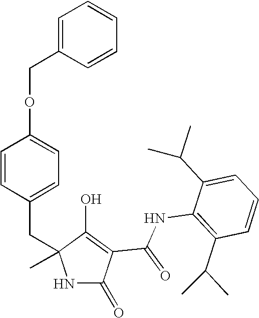 |
| 405. | 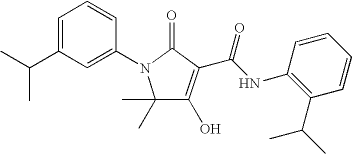 |
| 406. | 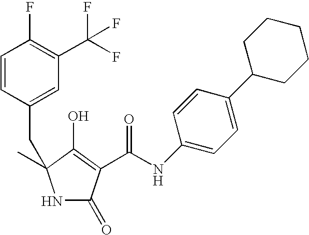 |
| 407. | 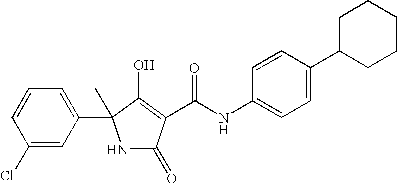 |
| 408. | 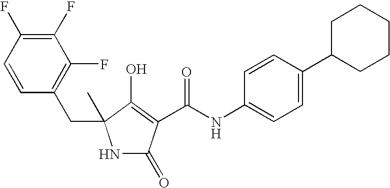 |
| 409. | 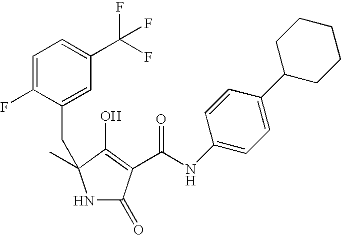 |
| 410. | 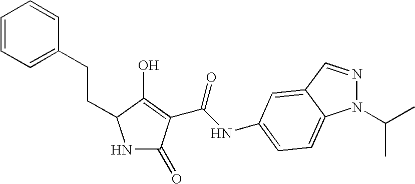 |
| 411. | 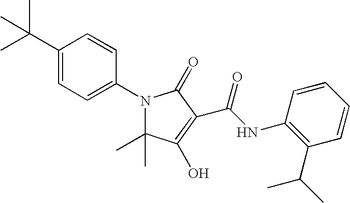 |
| 412. | 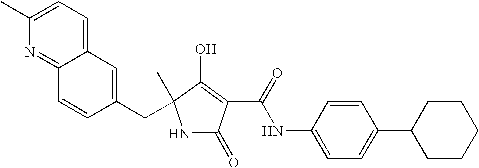 |
| 413. | 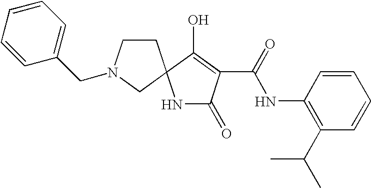 |
| 414. | 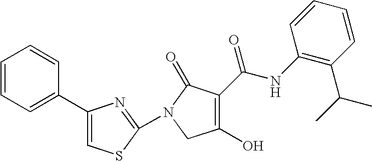 |
| 415. | 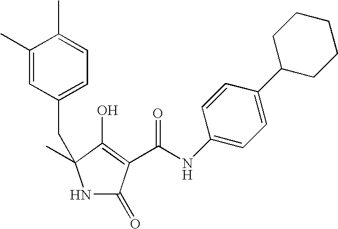 |
| 416. | 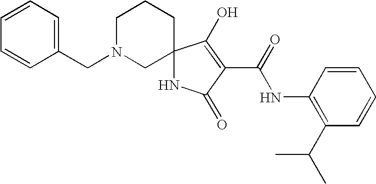 |
| 417. | 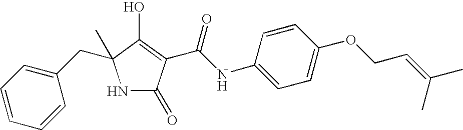 |
| 418. | 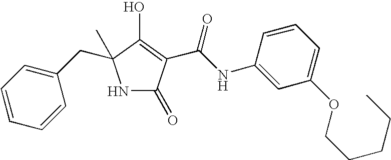 |
| 419. | 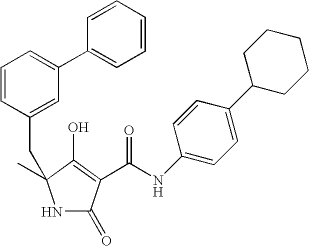 |
| 420. | 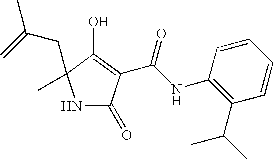 |
| 421. | 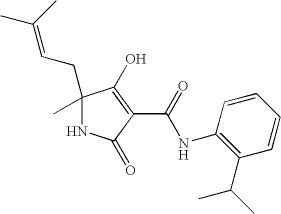 |
| 422. | 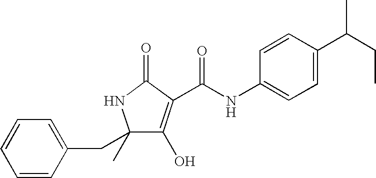 |
| 423. | 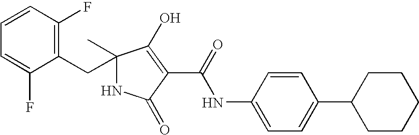 |
| 424. | 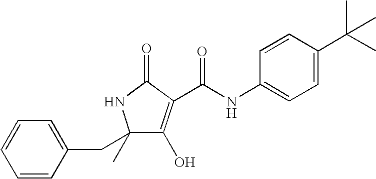 |
| 425. | 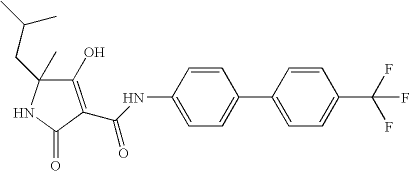 |
| 426. | 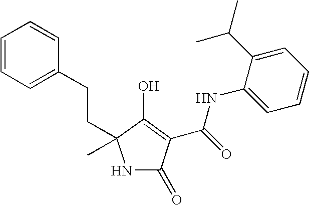 |
| 427. | 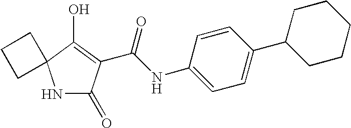 |
| 428. | 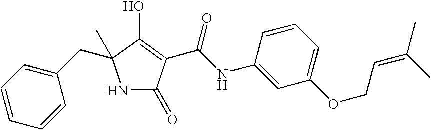 |
| 429. | 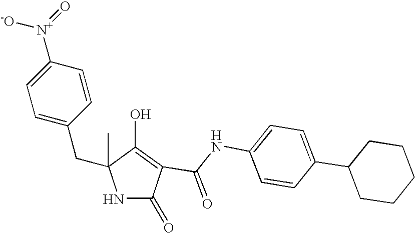 |
| 430. | 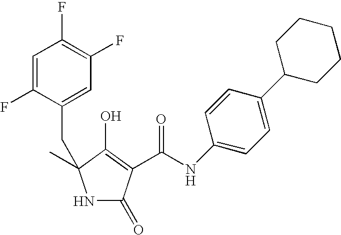 |
| 431. | 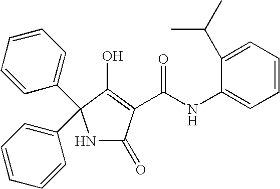 |
| 432. | 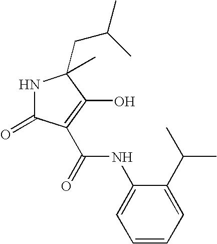 |
| 433. | 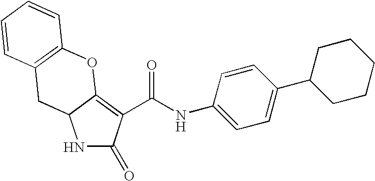 |
| 434. | 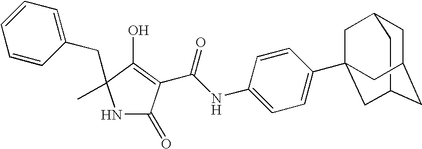 |
| 435. | 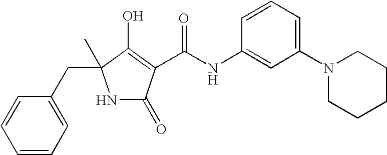 |
| 436. | 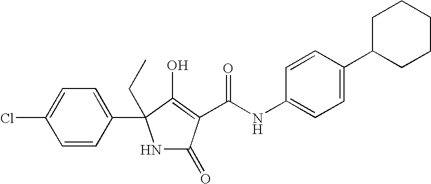 |
| 437. | 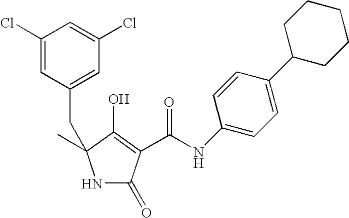 |
| 438. | 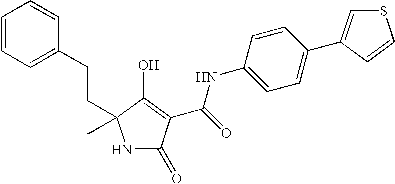 |
| 439. | 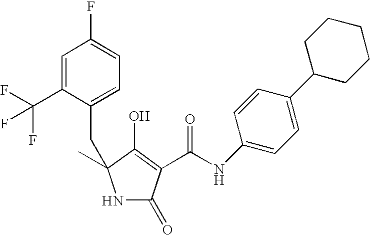 |
| 440. | 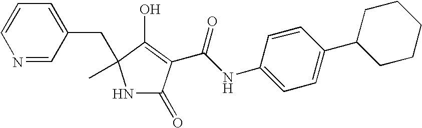 |
| 441. | 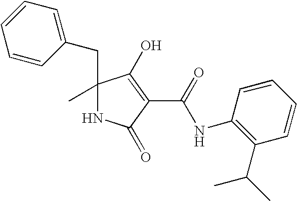 |
| 442. | 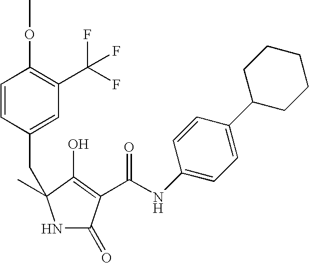 |
| 443. | 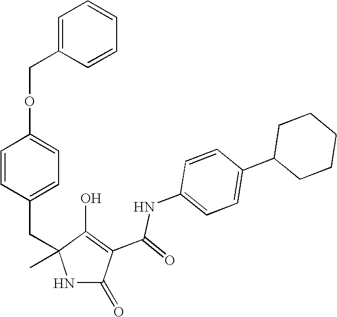 |
| 444. | 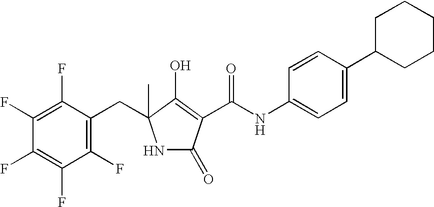 |
| 445. | 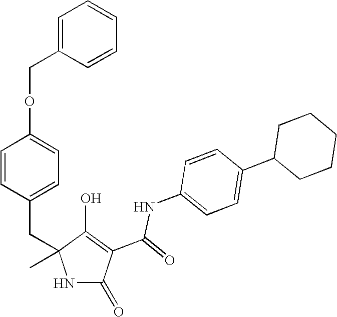 |
| 446. | 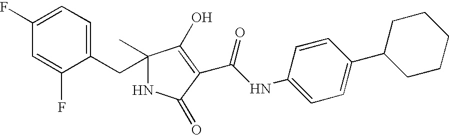 |
| 447. | 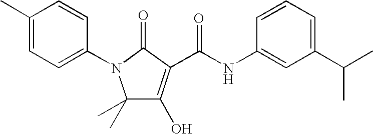 |
| 448. | 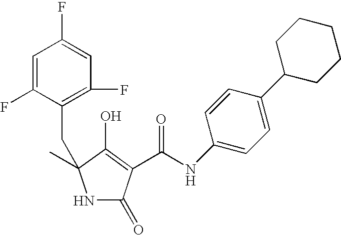 |
| 449. | 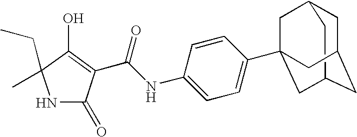 |
| 450. | 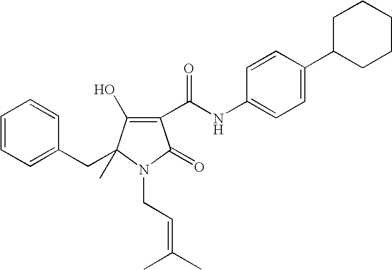 |
| 451. | 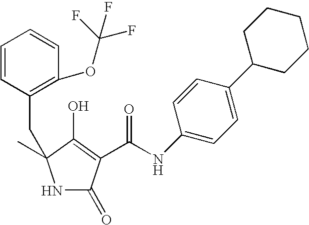 |
| 452. | 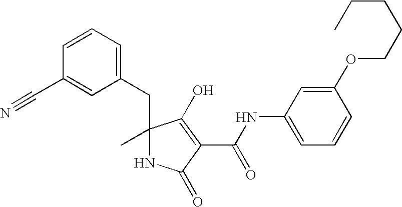 |
| 453. | 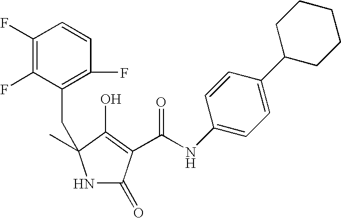 |
| 454. | 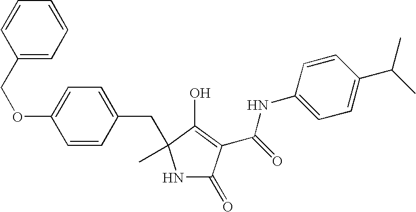 |
| 455. | 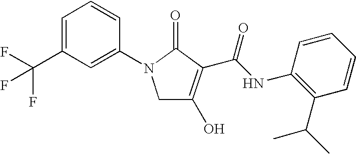 |
| 456. | 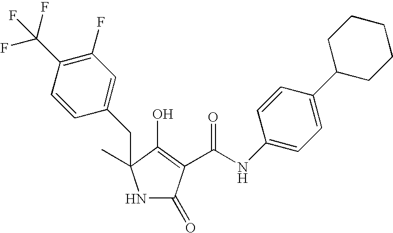 |
| 457. | 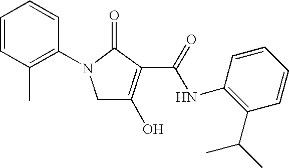 |
| 458. | 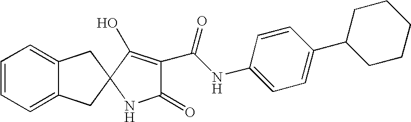 |
| 459. | 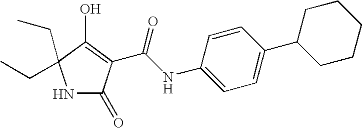 |
| 460. | 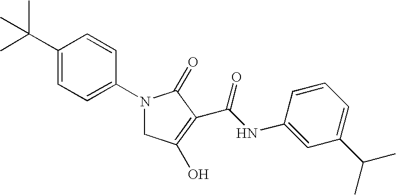 |
| 461. | 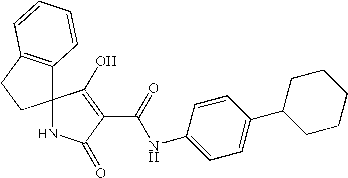 |
| 462. | 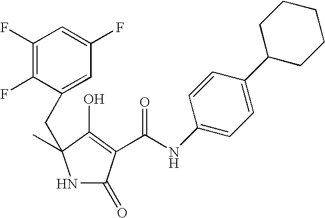 |
| 463. | 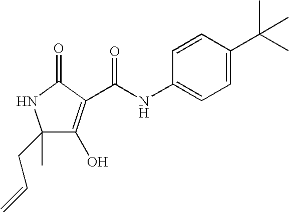 |
| 464. | 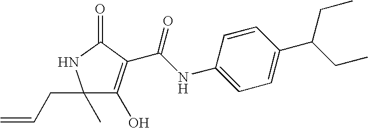 |
| 465. | 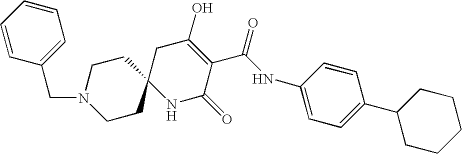 |
| 466. | 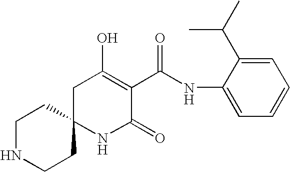 |
| 467. | 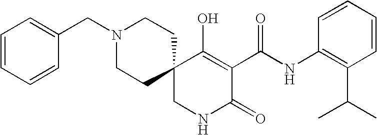 |
| 468. | 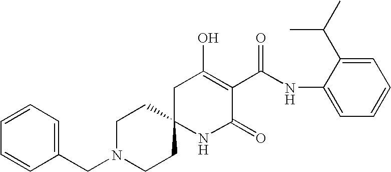 |
| 469. | 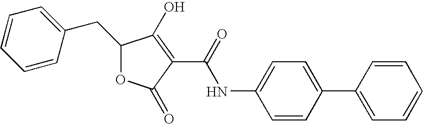 |
| 470. | 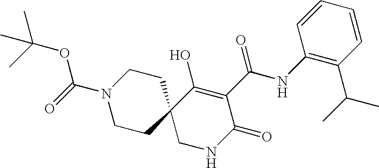 |
| 471. | 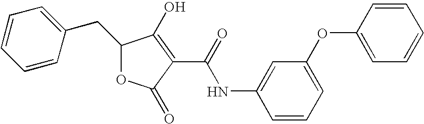 |
| 472. | 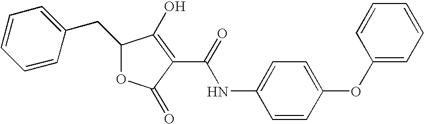 |
| 473. | 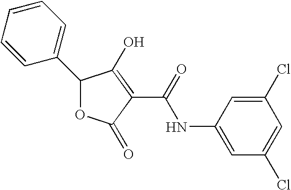 |
| 474. | 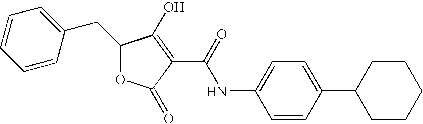 |
| 475. | 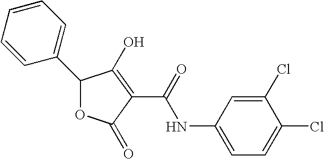 |
| 476. | 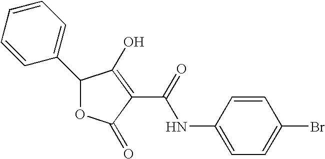 |
| 477. | 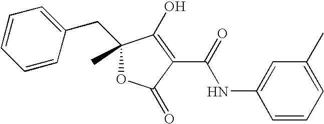 |
| 478. | 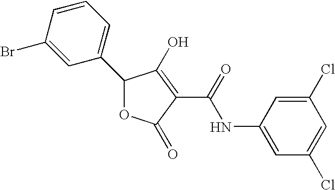 |
| 479. | 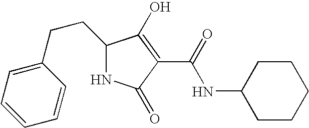 |
| 480. | 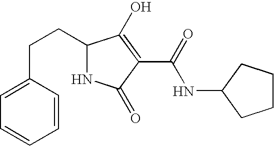 |
| 481. | 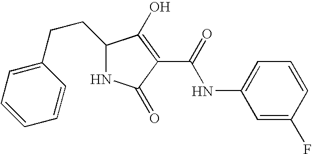 |
| 482. | 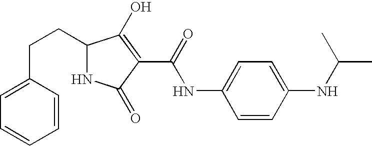 |
| 483. | 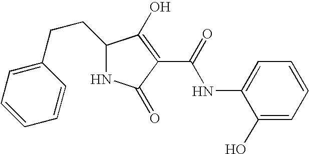 |
| 484. | 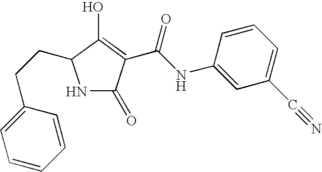 |
| 485. | 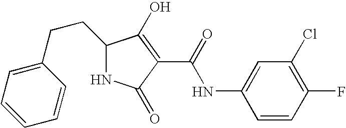 |
| 486. | 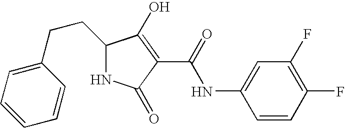 |
| 487. | 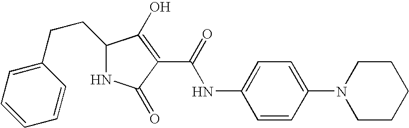 |
| 488. | 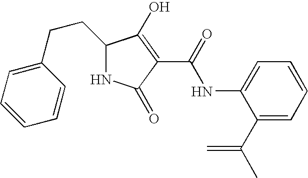 |
| 489. | 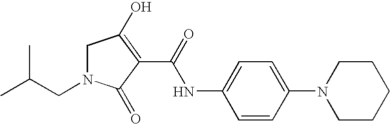 |
| 490. | 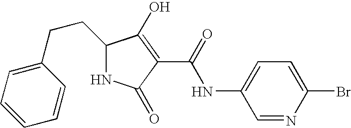 |
| 491. | 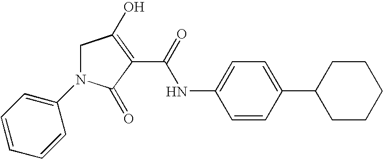 |
| 492. | 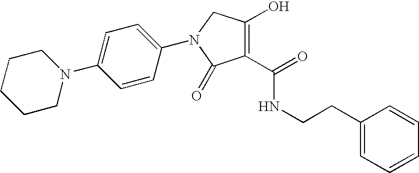 |
| 493. | 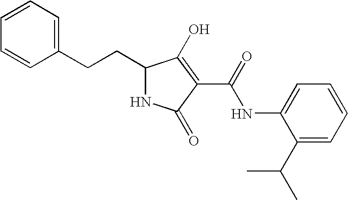 |
| 494. | 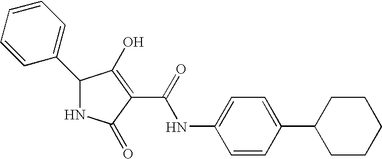 |
| 495. | 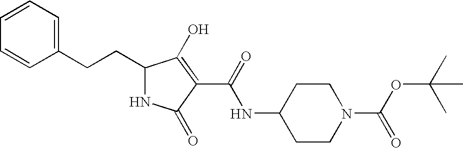 |
| 496. | 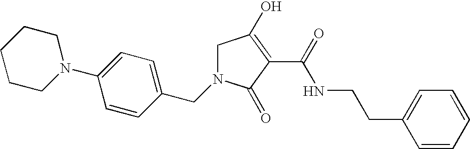 |
| 497. | 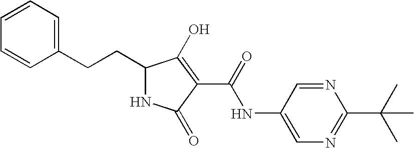 |
| 498. | 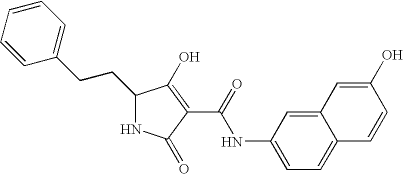 |
| 499. | 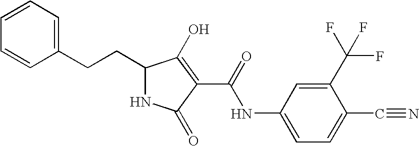 |
| 500. | 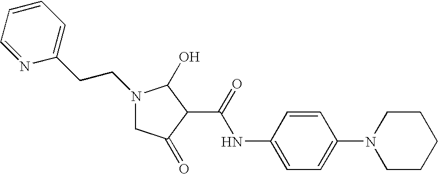 |
| 501. | 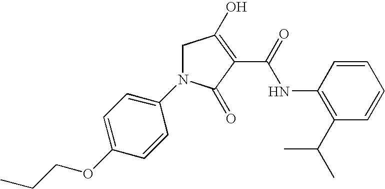 |
| 502. | 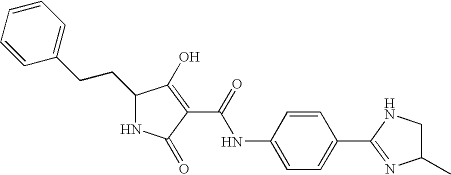 |
| 503. | 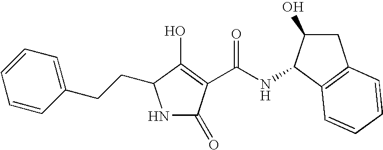 |
| 504. | 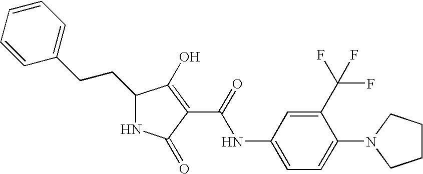 |
| 505. | 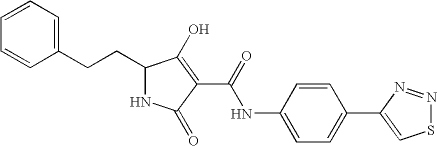 |
| 506. | 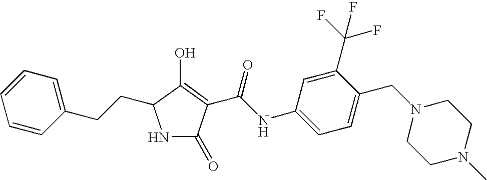 |
| 507. | 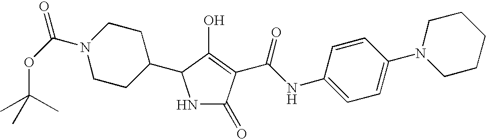 |
| 508. | 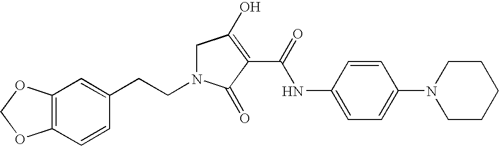 |
| 509. | 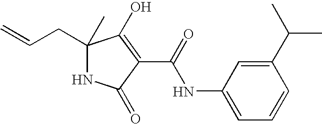 |
| 510. | 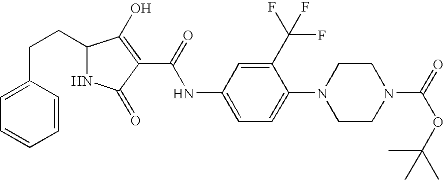 |
| 511. | 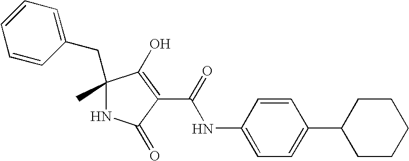 |
| 512. | 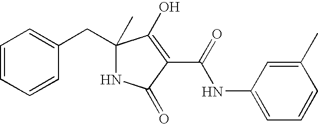 |
| 513. | 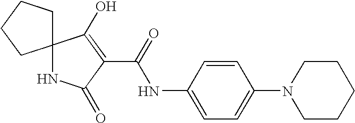 |
| 514. | 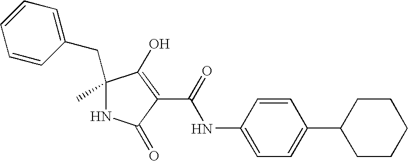 |
| 515. | 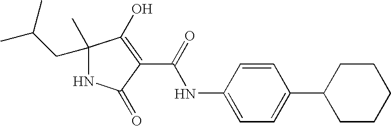 |
| 516. | 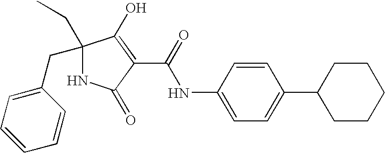 |
| 517. | 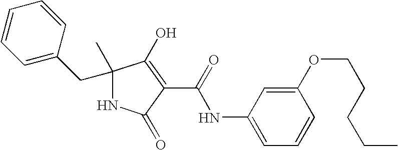 |
| 518. | 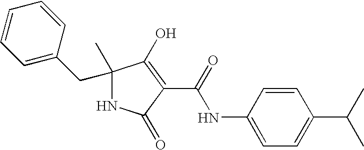 |
| 519. | 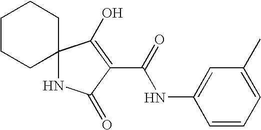 |
| 520. | 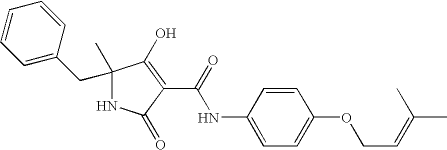 |
| 521. | 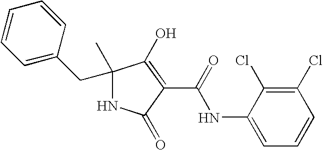 |
| 522. | 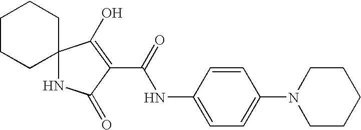 |
| 523. | 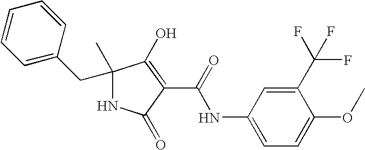 |
| 524. | 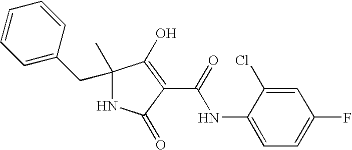 |
| 525. | 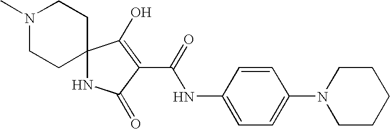 |
| 526. | 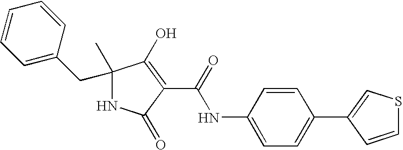 |
| 527. | 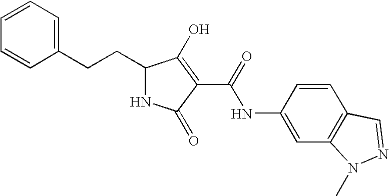 |
| 528. | 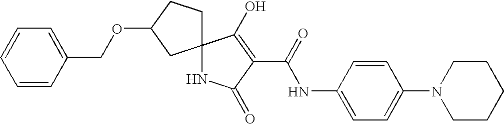 |
| 529. | 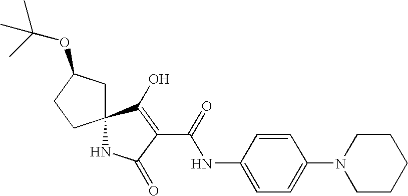 |
| 530. | 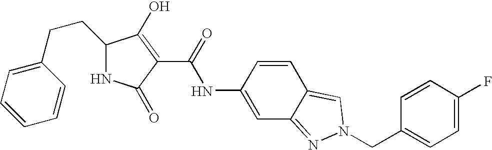 |
| 531. | 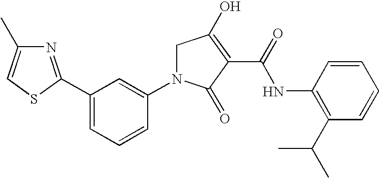 |
| 532. | 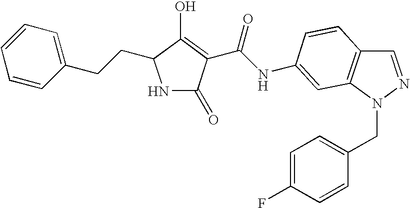 |
| 533. | 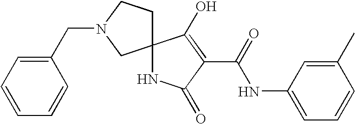 |
| 534. | 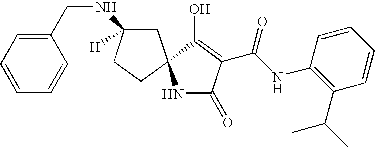 |
| 535. | 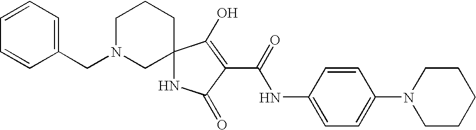 |
| 536. | 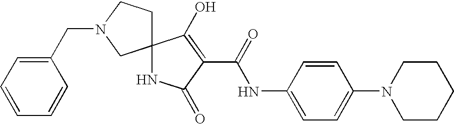 |
| 537. | 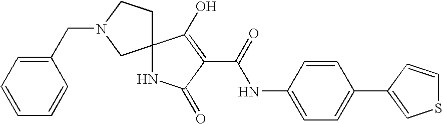 |
| 538. | 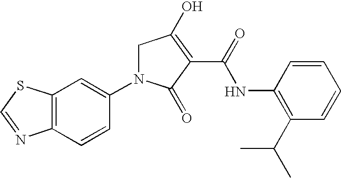 |
| 539. | 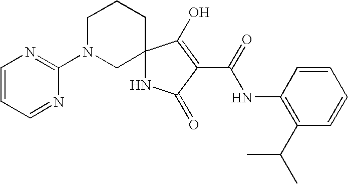 |
| 540. | 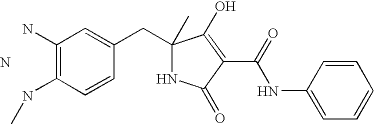 |
| 541. | 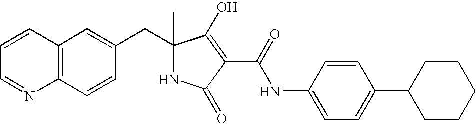 |
| 542. | 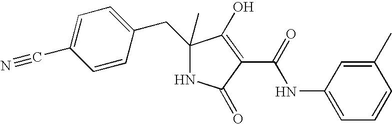 |
| 543. | 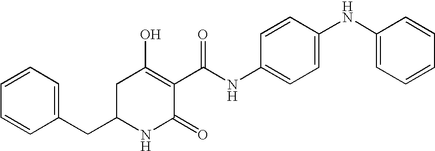 |
| 544. | 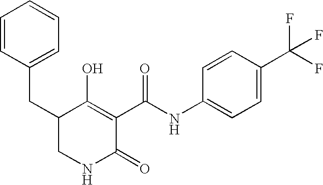 |
| 545. | 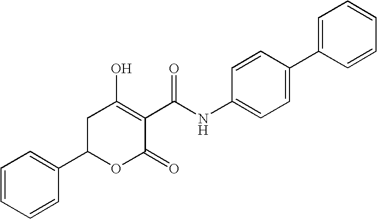 |
| 546. | 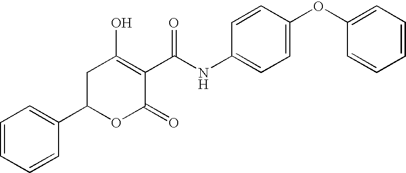 |
| 547. | 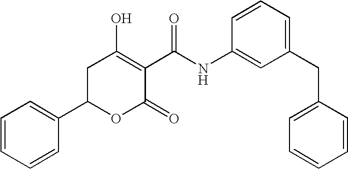 |
| 548. | 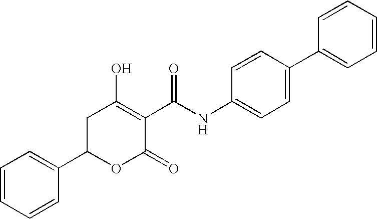 |
| 549. | 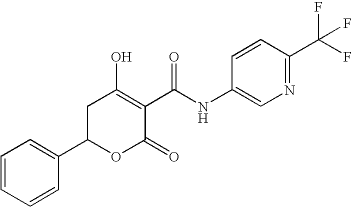 |
| 550. | 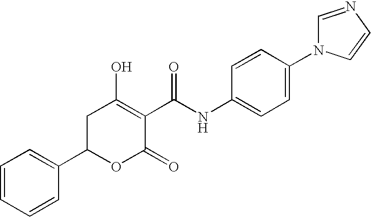 |
| 551. | 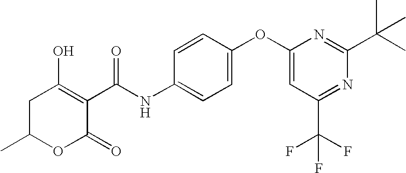 |
| 552. | 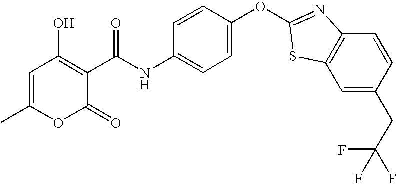 |
| 553. | 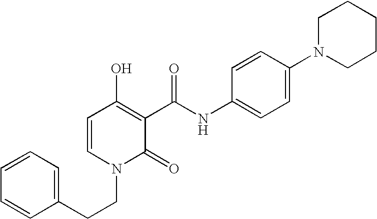 |

In a particular embodiment, the compound of the invention is

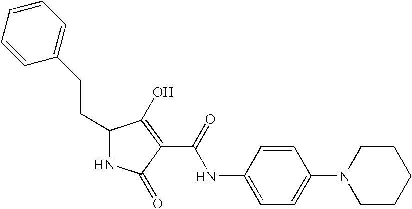

In another embodiment, the invention includes any novel compound or pharmaceutical compositions containing compounds of the invention described herein. For example, compounds and pharmaceutical compositions containing compounds set forth herein (e.g., Tables 1 and 2) are part of this invention, including salts thereof, e.g., a pharmaceutically acceptable salt.

In particular embodiments, the compounds in Tables 1 and 2 can be administered using all of the methods described herein, such as combining the compound with a carrier material suitable for oral, nasal, topical, transdermal, buccal, sublingual, rectal, vaginal and/or parenteral administration. For example, formulations of the invention suitable for oral administration may be in the form of capsules, cachets, pills, tablets and lozenges.

The invention also relates to salts of the compounds of the invention and, in particular, to pharmaceutically acceptable salts. A “pharmaceutically acceptable salt” includes a salt that retains the desired biological activity of the parent compound and does not impart any undesired toxicological effects. The salts can be, for example, salts with a suitable acid, such as hydrochloric acid, hydrobromic acid, sulfuric acid, phosphoric acid, nitric acid, and the like; acetic acid, oxalic acid, tartaric acid, succinic acid, malic acid, benzoic acid, pamoic acid, alginic acid, methanesulfonic acid, naphthalenesulfonic acid, and the like. Also included are salts of cations such as ammonium, sodium, potassium, lithium, zinc, copper, barium, bismuth, calcium, and the like; or organic cations such as tetraalkylammonium and trialkylammonium cations. Combinations of the above salts are also useful. Salts of other acids and/or cations are also included, such as salts with trifluoroacetic acid, chloroacetic acid, and trichloroacetic acid.

It will be noted that the structure of some of the compounds of this invention includes asymmetric carbon atoms. It is to be understood accordingly that the isomers arising from such asymmetry (e.g., all enantiomers and diastereomers) are included within the scope of this invention unless indicated otherwise. Such isomers can be obtained in substantially pure form by classical separation techniques and by stereochemically controlled synthesis. That is, unless otherwise stipulated, any chiral carbon center may be of either (R)- or (S)-stereochemistry. Furthermore, alkenes can include either the E- or Z-geometry, where appropriate. Additionally, one skilled in the art will appreciate that the chemical structures as drawn may represent a number of possible tautomers, and the present invention also includes those tautomers.

Accordingly, another embodiment of the invention is a substantially pure single stereoisomer or a mixture of stereoisomers, e.g., pre-determined to be within specific amounts.

It will further be noted that, depending upon, e.g., the methods for isolating and purifying the compounds of the present invention, there may exist a number of polymorphs of each individual compound. As used herein, the term “polymorph” refers to a solid crystalline phase of a compound of the invention, resulting from the possibility of at least two different arrangements of the molecules of the compound in the solid state. Crystalline forms of a particular compound of the invention, e.g., a compound of Table 1 or Table 2, are of particular importance because they may be formulated in various oral unit dosage forms as for example as tablets or capsules for the treatment of bacterial disease in patients. Variations in crystal structure of a pharmaceutical drug substance may affect the dissolution, manufacturability and stability of a pharmaceutical drug product, specifically in a solid oral dosage form formulation. Therefore it may be preferred to produce a compound of the invention in a pure form consisting of a single, thermodynamically stable crystal structure. It has been determined, for example, that the crystal structure of known compounds produced in accordance with commonly utilized synthesis may not be the most thermodynamically stable polymorphic form. Furthermore, it has been demonstrated that a polymorphic form may undergo conversion to a different polymorphic form when subjected to conventional manufacturing processes, such as grinding and milling. As such, certain polymorphic forms, which may not be the most thermodynamically stable form of the compound, could undergo polymorph conversion over time.

Polymorphs of a given compound will be different in crystal structure but identical in liquid or vapor states. Moreover, solubility, melting point, density, hardness, crystal shape, optical and electrical properties, vapor pressure, stability, etc., may all vary with the polymorphic form. Remington's Pharmaceutical Sciences, 18th Edition, Mack Publishing Co. (1990), Chapter 75, pages 1439-1443. Such polymorphs are also meant to be included in the scope of this invention. Varying polymorphs may be created, for example, by applying kinetic energy, e.g., by grinding, milling, or stirring, preferably at low temperature or by applying heat and subsequently cooling in a controlled manner. The compounds of the present invention may exist as a single polymorphic form or as a mixture of multiple polymorphic forms.

Furthermore, the compounds of the present invention may be suitable for silicon switching as described, e.g., in Drug Discovery Today 8 (12): 551-6 (2003) “Chemistry challenges in lead optimization: silicon isoteres in drug discovery.” Briefly, it has recently been discovered that certain carbon atoms in organic compounds, such as the compounds of the present invention, may be replaced by silicon atoms without noticeable loss in activity. Accordingly, in one embodiment, the present invention is directed to a compound of the invention as described herein, e.g., Table 1 or Table 2, wherein one or more of the carbons in the molecule has been replaced by a silicon. The skilled artisan can readily determine which compounds are eligible for silicon switching, which carbons of such compounds may be replaced, and how to effect the switch using no more than routine experimentation found, e.g., in Drug Discovery Today 8 (12): 551-6 (2003) “Chemistry challenges in lead optimization: silicon isoteres in drug discovery”, cited above.

In certain embodiments, the compounds of the present invention are characterized by a unique structure which imparts surprisingly improved properties to these compounds as compared to the prior art compounds, e.g., for use in inhibiting UPPS or treating bacterial disease. Specifically, the compounds of the present invention are characterized by the presence of a hydroxydicarbonyl moiety. This moiety, in combination with a functionalizing moiety and tail moiety, e.g., R-Q-T, within the core of the structure, enhances the selectivity of the compounds described herein for UPP synthase versus other synthases, such as FPPS. In fact, many of the compounds of the present invention are further characterized by their potent and/or selective binding to UPPS.

### Methods of Using the Compounds of the Invention

The compounds of the invention have been determined to useful at least in the treatment of bacterial disease, e.g., bacterial infection. Accordingly, in one embodiment, the invention relates to a method for treating bacterial disease comprising administering to a subject a compound of the invention, e.g., a compound of the following formula

R-Q-T

wherein R is a functionalizing moiety; Q is a hydroxydicarbonyl moiety, e.g., a monocyclic hydroxydicarbonyl moiety; and T is a tail moiety, such that a bacterial disease is treated in the subject.

The language “bacterial disease” describes disease states that are the result of the actions of one or more bacterium. For example, bacterial disease includes, but is not limited to bacterial infection or the symptomology and disease state in a subject associated with a bacterium, e.g., the actions of a bacterium. In certain embodiments, the symptomology and disease state associated with the bacterium is selected from the group consisting of inflammation, fever, and bacterial infection related pain. In certain embodiments, the bacterial disease is a bacterial infection, e.g., an acute bacterial infection or a chronic bacterial infection.

The language “bacterial infection” is art-recognized, and describes disease states resulting from the infection or attack of a host or subject by one or more bacterium types. Moreover, the bacterial infection may be associated with, for example, a gram negative bacterium; a gram positive bacterium, e.g., hospital gram positive infection; or in particular embodiments, a bacterium selected from the group consisting of S. aureus, Group A Streptococcus, E. faecalis, and Coagulase-negative Staphylococcus; with E. coli, S. aureus, E. faecalis, or S. pneumoniae.

In certain embodiments, the bacterial infection is an outpatient skin infection or a skin structure infection, e.g., wherein the bacterial infection is associated with a bacterium selected from the group consisting of S. aureus and Group A Streptococcus.

In certain embodiments, the bacterial infection is community-acquired methicillin-resistant Staphylococcus aureus (CA-MRSA), e.g., wherein the bacterial infection is associated with methicillin-resistant Staphylococcus aureus (MRSA).

In yet other embodiments, the bacterial infection is an antibiotic-associated colitis infection, e.g., wherein the bacterial infection is associated with C. difficile. In still yet another embodiment, the bacterial infection is nosocomial pneumonia, e.g., wherein the bacterial infection is associated with S. aureus or wherein the bacterial infection is associated with gram negative bacterium, e.g., P. aeruginosa, Klebsiella, Enterobacter, E. coli, or Acinetobacter.

In particular embodiments, the bacterial infection is selected from the group consisting of Actinomycosis; Anthrax; Aspergillosis; Bacteremia; Bacterial Infections and Mycoses; Bacterial Meningitis; Bartonella Infections; Botulism; Brucellosis; Bubonic plague; Burkholderia Infections; Campylobacter Infections; Candidiasis; Cat-Scratch Disease; Chlamydia Infections; Cholera; Clostridium Infections; Coccidioidomycosis; Cross Infection; Cryptococcosis; Dermatomycoses; Diphtheria; Ehrlichiosis; Epidemic Typhus; Escherichia coli Infections; Fasciitis, Necrotizing; Fusobacterium Infections; Gas Gangrene; Gonorrhea; Gram-Negative Bacterial Infections; Gram-Positive Bacterial Infections; Hansen's Disease; Histoplasmosis; Impetigo; Klebsiella Infections; Legionellosis; Leprosy; Leptospirosis; Listeria Infections; Lyme Disease; Maduromycosis; Melioidosis; MRSA infection; Mycobacterium Infections; Mycoplasma Infections; Nocardia Infections; Onychomycosis; Pertussis; Plague; Pneumococcal Infections; Pseudomonas Infections; Psittacosis; Q Fever; Rat-Bite Fever; Relapsing Fever; Rheumatic Fever; Rickettsia Infections; Rocky Mountain Spotted Fever; Salmonella Infections; Scarlet Fever; Scrub Typhus; Sepsis; Sexually Transmitted Diseases, Bacterial; Shigellosis; Shock, Septic; Skin Diseases, Bacterial; Staphylococcal Infections; Streptococcal Infections; Syphilis; Tetanus; Tick-Borne Diseases; Trachoma; Tuberculosis; Tularemia; Typhoid Fever; Typhus, Epidemic Louse-Borne; Whooping Cough; Vibrio Infections; Yaws; Yersinia Infections; Zoonoses; and Zygomycosis.

In another embodiment, the bacterial infection is a respiratory tract infection, e.g., wherein the bacterial infection is associated with S. pneumonia, H. influenza, Moraxella, L. pneumonia, Chlamydia, or mycoplasma.

In yet another embodiment, the bacterial infection is a sexually transmitted disease, e.g., wherein the bacterial infection is Chlamydia trachomatis or Neisseria gonorrheae.

In certain embodiments, the compounds of the invention are useful in treating bacterial infection wherein said bacterial infection is resistant to other antibiotics.

The term “subject,” includes living organisms in which a bacterial disease can occur, or which are susceptible bacterial disease. Examples include animals such as mammals, including, but not limited to, primates (e.g., humans), cows, sheep, goats, horses, pigs, dogs, cats, rabbits, guinea pigs, rats, mice or other bovine, ovine, equine, canine, feline, rodent, murine species, or transgenic species thereof. In particular embodiments, the subject is human, e.g., the compound of the invention is pre-selected for its use in treating bacterial disease in humans.

In certain embodiments of the invention, the subject is in need of treatment by the methods of the invention, e.g., by a UPPS inhibitor selected for its UPPS inhibition, and is selected for treatment based on this need. A subject in need of treatment is art-recognized, and includes subjects that have been identified as having a disease or disorder associated with UPPS or having a bacterial disease, having a symptom of such a disease or disorder, or at risk of such a disease or disorder, and would be expected, based on diagnosis, e.g., medical diagnosis, to benefit from treatment (e.g., curing, healing, preventing, alleviating, relieving, altering, remedying, ameliorating, improving, or affecting the disease or disorder, the symptom of the disease or disorder, or the risk of the disease or disorder). For example, the subject may be a “bacterium compromised subject,” wherein such subject is identified as being infected by at least one bacterium.

In particular embodiment, the subject is in need of treatment by the compounds of the invention, and is selected for treatment based on this need. In another particular embodiment, the subject is in need of treatment by the compounds of the invention and a pre-determined additional agent, and is selected for treatment based on this need.

As used herein, the term “administering” to a subject includes dispensing, delivering or applying a compound of the invention in a pharmaceutical formulation (as described herein), to a subject by any suitable route for delivery of the compound to the desired location in the subject, including delivery by either the parenteral or oral route, intramuscular injection, subcutaneous/intradermal injection, intravenous injection, buccal administration, topical delivery, transdermal delivery and administration by the rectal, colonic, vaginal, intranasal or respiratory tract route. In certain embodiments, the route for delivery of the compound is oral.

In certain embodiments, the compound of any of the formulae described herein, e.g., R-Q-T (and particular embodiments thereof, e.g., Tables 1 or Table 2) is an inhibitor of UPPS.

The terms “inhibitor” or “UPPS inhibitor,” as used herein, include compounds, e.g., compounds described herein, which bind to and/or inhibit the UPPS enzyme. In certain embodiments of the invention, the inhibitors described herein are activity enhanced with respect to known compounds which interact with UPPS. The language “activity enhanced” describes inhibitors of the invention that are at least one of either potent or selective. In particular embodiments, the compounds of the invention are pre-selected for their UPPS inhibition.

In one embodiment, the compound of the invention is “potent,” or possesses enhanced potency, against UPPS. A compound is “potent” against UPP synthase if the IC₅₀ value for binding to UPPS is less than or equal to about 2.0 μM, e.g., less than or equal to about 1.0 μM, e.g., less than or equal to about 0.5 μM, e.g., less than or equal to about 0.1 μM, e.g., less than or equal to about 0.05 μM, e.g., less than or equal to about 0.01 μM, e.g., less than or equal to about 0.005 μM. It should be understood that embodiments of the invention include compounds that fall within Formulae I-XII, having IC₅₀ value for binding to UPPS, for example, of less than or equal to about 2.0 μM, e.g., less than or equal to about 1.0 μM, e.g., less than or equal to about 0.5 μM, e.g., less than or equal to about 0.1 μM, e.g., less than or equal to about 0.05 μM, e.g., less than or equal to about 0.01 μM, e.g., less than or equal to about 0.005 μM. Furthermore, it should be understood that all values and ranges encompassed by these ranges, are meant to be encompassed within the scope of the present invention. Moreover, all values that fall within these ranges, as well as the upper or lower limits of a range of values, are also contemplated by the present application. For example, the range “less than or equal to about 1.0 μM” includes values such as, 0.75 μM, 0.69 μM, and 0.50-0.35 μM.

In another embodiments, the compound of the invention is “selective,” or possesses enhanced selectivity, for UPPS. For example, the present invention includes compounds that are selective, or possess enhanced selectivity, for UPPS relative to FPPS. A compound is “selective” for the UPP synthase relative to a second synthase, if the IC₅₀ of the compound for the second enzyme is at least 50-fold, e.g., at least 100-fold, e.g., at least 1,000-fold, e.g., at least 10,000-fold greater than the IC₅₀ for UPPS. Moreover, the IC₅₀ of a compound is determined as described in Example 15. It should be understood that embodiments of the invention include compounds that fall within Formulae I-XII, having a selectivity of at least 50-fold, e.g., at least 100-fold, e.g., at least 1,000-fold, e.g., at least 10,000-fold greater than the IC₅₀ for UPPS over a second enzyme. Furthermore, it should be understood that all values and ranges encompassed by these ranges, are meant to be encompassed within the scope of the present invention. Moreover, all values that fall within these ranges, as well as the upper or lower limits of a range of values, are also contemplated by the present application. For example, the range “at least 50-fold” includes values such as, 65 fold, 85 fold, and 100-200 fold.

Additionally, the selectivity may be quantified by means of a specificity ratio defined as

UPPS IC₅₀/FPPS IC₅₀.

In certain embodiments, the specificity ratio of a compound of the invention with enhanced selectivity is less than or equal to about 0.02, e.g., less than or equal to about 0.01, e.g., less than or equal to about 0.002, e.g., less than or equal to about 0.001, e.g., less than or equal to about 0.0002, e.g., less than or equal to about 0.0001. Furthermore, all values and ranges encompassed by these ranges are meant to be encompassed within the scope of the present invention. Moreover, all values that fall within these ranges, as well as the upper or lower limits of a range of values, are also contemplated by the present application. For example, the range “less than or equal to about 0.002” includes values such as, 0.002, 0.001, and 0.001-0.0001.

In another embodiment, the present invention is a method for treating bacterial disease comprising administering a potent and selective undecaprenyl pyrophosphate synthase (UPPS) inhibitor to a subject, such that a bacterial disease is treated in the subject.

In yet another embodiment of the invention pertains to a method for treating bacterial disease comprising administering a potent UPPS inhibitor to a subject, such that a bacterial disease is treated in the subject.

Another embodiment of the invention pertains to a method for treating bacterial disease comprising administering a selective UPPS inhibitor to a subject, such that a bacterial disease is treated in the subject.

An additional embodiment of the invention is directed to a method for inhibiting undecaprenyl pyrophosphate synthase (UPPS) comprising the step of contacting UPPS with an activity-enhanced UPPS inhibitor, such that UPPS is inhibited. In certain embodiments, the activity-enhanced UPPS inhibitor possesses enhanced selectivity for UPPS, e.g., enhanced selectivity for UPPS over farnesyl pyrophosphate synthetase (FPPS). In certain embodiments, the activity-enhanced UPPS inhibitor possesses enhanced potency in inhibiting UPPS. In particular embodiments, the activity-enhanced UPPS inhibitor is used as an antibacterial. In other particular embodiments, the activity-enhanced UPPS inhibitor is used as an antibiotic. As used herein, the term “antibacterial” is distinct from “antibiotic,” in that antibacterial is intended to describe an agent that is used directly on the bacteria, e.g., on a surface, while antibiotic is intended to describe an agent that is administered to a subject infected with the bacteria to inhibit/treat the bacteria.

Another embodiment of the invention is a method for inhibiting undecaprenyl pyrophosphate synthase (UPPS) comprising administering to a bacterium compromised subject an activity-enhanced UPPS inhibitor, such that UPPS is inhibited in the subject.

An additional embodiment of the invention relates to a method for selectively inhibiting undecaprenyl pyrophosphate synthase (UPPS) comprising the step of administering to a bacterium compromised subject an activity-enhanced UPPS inhibitor wherein the UPPS/FPPS specificity ratio is less than or equal to about 0.02, e.g., less than or equal to about 0.01, e.g., less than or equal to about 0.002, e.g., less than or equal to about 0.001, e.g., less than or equal to about 0.0002, e.g., less than or equal to about 0.0001, such that UPPS is selectively inhibited in the subject.

In another embodiment, the invention is directed to a method for treating a bacterium compromised subject comprising the step of administering to a bacterium compromised subject an activity-enhanced UPPS inhibitor effective to treat a disease or disorder associated with a UPPS enabled bacterium, such that the bacterium compromised subject is treated.

An additional embodiment of the invention pertains to a method for treating a subject suffering from a bacterial disorder, comprising administering to a subject a compound, such that the subject is treated for a bacterial disorder by a compound of the invention, e.g., compounds of Table 1 or Table 2.

Another embodiment of the invention pertains to a method for identifying an activity-enhanced UPPS inhibitor comprising

screening drug candidates for threshold activity;

confirming that the molecular structure of a selected drug candidate contains a hydroxydicarbonyl moiety;

analyzing said selected drug candidate to ensure enhanced selectivity or potency;

determining that said selected drug candidate possesses a UPPS/FPPS specificity ratio is less than or equal to about 0.02, e.g., less than or equal to about 0.01, e.g., less than or equal to about 0.002, e.g., less than or equal to about 0.001, e.g., less than or equal to about 0.0002, e.g., less than or equal to about 0.0001, or the selected IC₅₀ of the drug candidate against UPPS is less than or equal to about 2.0 μM, e.g., less than or equal to about 1.0 μM, e.g., less than or equal to about 0.5 μM, e.g., less than or equal to about 0.1 μM, e.g., less than or equal to about 0.05 μM, e.g., less than or equal to about 0.01 μM, e.g., less than or equal to about 0.005 μM; and identifying said selected drug candidate as an activity-enhanced UPPS inhibitor.

As used herein, the term “effective amount” includes an amount effective, at dosages and for periods of time necessary, to achieve the desired result, e.g., sufficient to treat the condition, i.e., bacterial disease, in a subject. An effective amount of a compound of the invention, as defined herein, may vary according to factors such as the disease state, age, and weight of the subject, and the ability of the compound to elicit a desired response in the subject. Dosage regimens may be adjusted to provide the optimum therapeutic response. An effective amount is also one in which any toxic or detrimental effects (e.g., side effects) of the compound are outweighed by the therapeutically beneficial effects.

A therapeutically effective amount of a compound of the invention (i.e., an effective dosage) may range from about 0.001 to 30 mg/kg body weight, for example, about 0.01 to 25 mg/kg body weight, for example, about 0.1 to 20 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound of the invention can include a single treatment or, for example, can include a series of treatments. It will also be appreciated that the effective dosage of the compound used for treatment may increase or decrease over the course of a particular treatment.

The methods of the invention further include administering to a subject a therapeutically effective amount of a compound of the invention in combination with another pharmaceutically active compound known to treat the disease or condition, e.g., an antibiotic. Pharmaceutically active compounds that may be used depend upon the condition to be treated, but include as examples Penicillin, Cephalosporin, Griseofulvin, Bacitracin, Polymyxin B, Amphotericin B, Erythromycin, Neomycin, Streptomycin, Tetracycline, Vancomycin, Gentamicin, and Rifamycin. The compound of the invention and the additional pharmaceutically active compound may be administered to the subject in the same pharmaceutical composition or in different pharmaceutical compositions (at the same time or at different times).

### Pharmaceutical Compositions of the Compounds of the Invention

The present invention also provides pharmaceutically acceptable formulations and compositions comprising one or more compounds of the invention. In certain embodiments, the compound of the invention is present in the formulation in a therapeutically effective amount, e.g., an amount effective to inhibit UPPS or treat a bacterial disease.

Accordingly, in one embodiment, the invention pertains to a pharmaceutical composition comprising a therapeutically effective amount of a compound of the invention, and a pharmaceutically acceptable carrier.

In another embodiment, the invention is directed to a packaged pharmaceutical composition comprising a container holding a therapeutically effective amount of a compound of the invention, e.g., a potent and/or selective UPPS inhibitor; and instructions for using the compound to treat a bacterial disease.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions can contain information pertaining to the compound's ability to perform its intended function, e.g., treating, preventing, or reducing a UPPS associated disorder in a subject.

Another embodiment of the invention relates to a packaged pharmaceutical composition comprising a container holding a therapeutically effective amount of a compound of the invention, and instructions for using the compound to selectively treat a bacterial disease in a subject.

Such pharmaceutically acceptable formulations typically include one or more compounds of the invention as well as one or more pharmaceutically acceptable carriers and/or excipients. As used herein, “pharmaceutically acceptable carrier” includes any and all solvents, dispersion media, coatings, antibacterial and antifungal agents, isotonic and absorption delaying agents, and the like that are physiologically compatible. The use of such media and agents for pharmaceutically active substances is well known in the art. Except insofar as any conventional media or agent is incompatible with the compounds of the invention, use thereof in the pharmaceutical compositions is contemplated.

Supplementary pharmaceutically active compounds known to treat bacterial disease, i.e., antibiotic agents, as described above, can also be incorporated into the compositions of the invention. Suitable pharmaceutically active compounds that may be used are art-recognized.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

### Formulations for Administration

The compounds for use in the invention can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

Oral Administration

For example, for oral administration the compounds can be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets can be coated using suitable methods and coating materials such as OPADRY™ film coating systems available from Colorcon, West Point, Pa. (e.g., OPADRY™ OY Type, OY-C Type, Organic Enteric OY-P Type, Aqueous Enteric OY-A Type, OY-PM Type and OPADRY™ White, 32K18400). Liquid preparation for oral administration can be in the form of solutions, syrups or suspensions. The liquid preparations can be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Tablets may be manufactured using standard tablet processing procedures and equipment. One method for forming tablets is by direct compression of a powdered, crystalline or granular composition containing the active agent(s), alone or in combination with one or more carriers, additives, or the like. As an alternative to direct compression, tablets can be prepared using wet-granulation or dry-granulation processes. Tablets may also be molded rather than compressed, starting with a moist or otherwise tractable material; however, compression and granulation techniques are preferred.

The dosage form may also be a capsule, in which case the active agent-containing composition may be encapsulated in the form of a liquid or solid (including particulates such as granules, beads, powders or pellets). Suitable capsules can be hard or soft, and are generally made of gelatin, starch, or a cellulosic material, with gelatin capsules preferred. Two-piece hard gelatin capsules are preferably sealed, such as with gelatin bands or the like. (See, for e.g., Remington: The Science and Practice of Pharmacy, supra), which describes materials and methods for preparing encapsulated pharmaceuticals. If the active agent-containing composition is present within the capsule in liquid form, a liquid carrier can be used to dissolve the active agent(s). The carrier should be compatible with the capsule material and all components of the pharmaceutical composition, and should be suitable for ingestion.

Parenteral Administration

For parenteral administration, the compounds for use in the method of the invention can be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents can be used.

Transmucosal Administration

Transmucosal administration is carried out using any type of formulation or dosage unit suitable for application to mucosal tissue. For example, the selected active agent can be administered to the buccal mucosa in an adhesive tablet or patch, sublingually administered by placing a solid dosage form under the tongue, lingually administered by placing a solid dosage form on the tongue, administered nasally as droplets or a nasal spray, administered by inhalation of an aerosol formulation, a non-aerosol liquid formulation, or a dry powder, placed within or near the rectum (“transrectal” formulations), or administered to the urethra as a suppository, ointment, or the like.

Preferred buccal dosage forms will typically comprise a therapeutically effective amount of an active agent and a bioerodible (hydrolyzable) polymeric carrier that may also serve to adhere the dosage form to the buccal mucosa. The buccal dosage unit can be fabricated so as to erode over a predetermined time period, wherein drug delivery is provided essentially throughout. The time period is typically in the range of from about 1 hour to about 72 hours. Preferred buccal delivery preferably occurs over a time period of from about 2 hours to about 24 hours. Buccal drug delivery for short term use should preferably occur over a time period of from about 2 hours to about 8 hours, more preferably over a time period of from about 3 hours to about 4 hours. As needed buccal drug delivery preferably will occur over a time period of from about 1 hour to about 12 hours, more preferably from about 2 hours to about 8 hours, most preferably from about 3 hours to about 6 hours. Sustained buccal drug delivery will preferably occur over a time period of from about 6 hours to about 72 hours, more preferably from about 12 hours to about 48 hours, most preferably from about 24 hours to about 48 hours. Buccal drug delivery, as will be appreciated by those skilled in the art, avoids the disadvantages encountered with oral drug administration, e.g., slow absorption, degradation of the active agent by fluids present in the gastrointestinal tract and/or first-pass inactivation in the liver.

The amount of the active agent in the buccal dosage unit will of course depend on the potency of the agent and the intended dosage, which, in turn, is dependent on the particular individual undergoing treatment, the specific indication, and the like. The buccal dosage unit will generally contain from about 1.0 wt. % to about 60 wt. % active agent, preferably on the order of from about 1 wt. % to about 30 wt. % active agent. With regard to the bioerodible (hydrolyzable) polymeric carrier, it will be appreciated that virtually any such carrier can be used, so long as the desired drug release profile is not compromised, and the carrier is compatible with the active agents to be administered and any other components of the buccal dosage unit. Generally, the polymeric carrier comprises a hydrophilic (water-soluble and water-swellable) polymer that adheres to the wet surface of the buccal mucosa. Examples of polymeric carriers useful herein include acrylic acid polymers and co, e.g., those known as “carbomers” (Carbopol™, which may be obtained from B. F. Goodrich, is one such polymer). Other suitable polymers include, but are not limited to: hydrolyzed polyvinylalcohol; polyethylene oxides (e.g., Sentry Polyox™ water soluble resins, available from Union Carbide); polyacrylates (e.g., Gantrez™, which may be obtained from GAF); vinyl polymers and copolymers; polyvinylpyrrolidone; dextran; guar gum; pectins; starches; and cellulosic polymers such as hydroxypropyl methylcellulose, (e.g., Methocel™, which may be obtained from the Dow Chemical Company), hydroxypropyl cellulose (e.g., Klucel™, which may also be obtained from Dow), hydroxypropyl cellulose ethers (see, e.g., U.S. Pat. No. 4,704,285 to Alderman), hydroxyethyl cellulose, carboxymethyl cellulose, sodium carboxymethyl cellulose, methyl cellulose, ethyl cellulose, cellulose acetate phthalate, cellulose acetate butyrate, and the like.

Other components can also be incorporated into the buccal dosage forms described herein. The additional components include, but are not limited to, disintegrants, diluents, binders, lubricants, flavoring, colorants, preservatives, and the like. Examples of disintegrants that may be used include, but are not limited to, cross-linked polyvinylpyrrolidones, such as crospovidone (e.g., Polyplasdone™ XL, which may be obtained from GAF), cross-linked carboxylic methylcelluloses, such as croscarmelose (e.g., Ac-di-sol™, which may be obtained from FMC), alginic acid, and sodium carboxymethyl starches (e.g., Explotab™, which can be obtained from Edward Medell Co., Inc.), methylcellulose, agar bentonite and alginic acid. Suitable diluents include those which are generally useful in pharmaceutical formulations prepared using compression techniques, e.g., dicalcium phosphate dihydrate (e.g., Di-Tab™, which may be obtained from Stauffer), sugars that have been processed by cocrystallization with dextrin (e.g., co-crystallized sucrose and dextrin such as Di-Pak™, which may be obtained from Amstar), calcium phosphate, cellulose, kaolin, mannitol, sodium chloride, dry starch, powdered sugar and the like. Binders, if used, include those that enhance adhesion. Examples of such binders include, but are not limited to, starch, gelatin and sugars such as sucrose, dextrose, molasses, and lactose. Particularly preferred lubricants are stearates and stearic acid, and an optimal lubricant is magnesium stearate.

Sublingual and lingual dosage forms include tablets, creams, ointments, lozenges, pastes, and any other suitable dosage form where the active ingredient is admixed into a disintegrate matrix. The tablet, cream, ointment or paste for sublingual or lingual delivery comprises a therapeutically effective amount of the selected active agent and one or more conventional nontoxic carriers suitable for sublingual or lingual drug administration. The sublingual and lingual dosage forms of the present invention can be manufactured using conventional processes. The sublingual and lingual dosage units can be fabricated to disintegrate rapidly. The time period for complete disintegration of the dosage unit is typically in the range of from about 10 seconds to about 30 minutes, and optimally is less than 5 minutes.

Other components can also be incorporated into the sublingual and lingual dosage forms described herein. The additional components include, but are not limited to binders, disintegrants, wetting agents, lubricants, and the like. Examples of binders that can be used include water, ethanol, polyvinylpyrrolidone; starch solution gelatin solution, and the like. Suitable disintegrants include dry starch, calcium carbonate, polyoxyethylene sorbitan fatty acid esters, sodium lauryl sulfate, stearic monoglyceride, lactose, and the like. Wetting agents, if used, include glycerin, starches, and the like. Particularly preferred lubricants are stearates and polyethylene glycol. Additional components that may be incorporated into sublingual and lingual dosage forms are known, or will be apparent, to those skilled in this art (See, e.g., Remington: The Science and Practice of Pharmacy, supra).

Transurethal Administration

With regard to transurethal administration, the formulation can comprise a urethral dosage form containing the active agent and one or more selected carriers or excipients, such as water, silicone, waxes, petroleum jelly, polyethylene glycol (“PEG”), propylene glycol (“PG”), liposomes, sugars such as mannitol and lactose, and/or a variety of other materials, with polyethylene glycol and derivatives thereof particularly preferred. A transurethral permeation enhancer can be included in the dosage from. Examples of suitable permeation enhancers include dimethylsulfoxide (“DMSO”), dimethyl formamide (“DMF”), N,N-dimethylacetamide (“DMA”), decylmethylsulfoxide (“C10 MSO”), polyethylene glycol monolaurate (“PEGML”), glycerol monolaurate, lecithin, the 1-substituted azacycloheptan-2-ones, particularly 1-n-dodecylcyclazacycloheptan-2-one (available under the trademark Azone™ from Nelson Research & Development Co., Irvine, Calif.), SEPA™ (available from Macrochem Co., Lexington, Mass.), surfactants as discussed above, including, for example, Tergitol™, Nonoxynol-9™ and TWEEN-80™, and lower alkanols such as ethanol.

Transurethral drug administration, as explained in U.S. Pat. Nos. 5,242,391, 5,474,535, 5,686,093 and 5,773,020, can be carried out in a number of different ways using a variety of urethral dosage forms. For example, the drug can be introduced into the urethra from a flexible tube, squeeze bottle, pump or aerosol spray. The drug can also be contained in coatings, pellets or suppositories that are absorbed, melted or bioeroded in the urethra. In certain embodiments, the drug is included in a coating on the exterior surface of a penile insert. It is preferred, although not essential, that the drug be delivered from at least about 3 cm into the urethra, and preferably from at least about 7 cm into the urethra. Generally, delivery from at least about 3 cm to about 8 cm into the urethra will provide effective results in conjunction with the present method.

Urethral suppository formulations containing PEG or a PEG derivative can be conveniently formulated using conventional techniques, e.g., compression molding, heat molding or the like, as will be appreciated by those skilled in the art and as described in the pertinent literature and pharmaceutical texts. (See, e.g., Remington: The Science and Practice of Pharmacy, supra), which discloses typical methods of preparing pharmaceutical compositions in the form of urethral suppositories. The PEG or PEG derivative preferably has a molecular weight in the range of from about 200 to about 2,500 g/mol, more preferably in the range of from about 1,000 to about 2,000 g/mol. Suitable polyethylene glycol derivatives include polyethylene glycol fatty acid esters, for example, polyethylene glycol monostearate, polyethylene glycol sorbitan esters, e.g., polysorbates, and the like. Depending on the particular active agent, urethral suppositories may contain one or more solubilizing agents effective to increase the solubility of the active agent in the PEG or other transurethral vehicle.

It may be desirable to deliver the active agent in a urethral dosage form that provides for controlled or sustained release of the agent. In such a case, the dosage form can comprise a biocompatible, biodegradable material, typically a biodegradable polymer. Examples of such polymers include polyesters, polyalkylcyanoacrylates, polyorthoesters, polyanhydrides, albumin, gelatin and starch. As explained, for example, in PCT Publication No. WO 96/40054, these and other polymers can be used to provide biodegradable microparticles that enable controlled and sustained drug release, in turn minimizing the required dosing frequency.

The urethral dosage form will preferably comprise a suppository that is from about 2 to about 20 mm in length, preferably from about 5 to about 10 mm in length, and less than about 5 mm in width, preferably less than about 2 mm in width. The weight of the suppository will typically be in the range of from about 1 mg to about 100 mg, preferably in the range of from about 1 mg to about 50 mg. However, it will be appreciated by those skilled in the art that the size of the suppository can and will vary, depending on the potency of the drug, the nature of the formulation, and other factors.

Transurethral drug delivery may involve an “active” delivery mechanism such as iontophoresis, electroporation or phonophoresis. Devices and methods for delivering drugs in this way are well known in the art. Iontophoretically assisted drug delivery is, for example, described in PCT Publication No. WO 96/40054, cited above. Briefly, the active agent is driven through the urethral wall by means of an electric current passed from an external electrode to a second electrode contained within or affixed to a urethral probe.

Transrectal Administration

Preferred transrectal dosage forms can include rectal suppositories, creams, ointments, and liquid formulations (enemas). The suppository, cream, ointment or liquid formulation for transrectal delivery comprises a therapeutically effective amount of the selected active agent and one or more conventional nontoxic carriers suitable for transrectal drug administration. The transrectal dosage forms of the present invention can be manufactured using conventional processes. The transrectal dosage unit can be fabricated to disintegrate rapidly or over a period of several hours. The time period for complete disintegration is preferably in the range of from about 10 minutes to about 6 hours, and optimally is less than about 3 hours.

Other components can also be incorporated into the transrectal dosage forms described herein. The additional components include, but are not limited to, stiffening agents, antioxidants, preservatives, and the like. Examples of stiffening agents that may be used include, for example, paraffin, white wax and yellow wax. Preferred antioxidants, if used, include sodium bisulfite and sodium metabisulfite.

Vaginal or Perivaginal Administration

Preferred vaginal or perivaginal dosage forms include vaginal suppositories, creams, ointments, liquid formulations, pessaries, tampons, gels, pastes, foams or sprays. The suppository, cream, ointment, liquid formulation, pessary, tampon, gel, paste, foam or spray for vaginal or perivaginal delivery comprises a therapeutically effective amount of the selected active agent and one or more conventional nontoxic carriers suitable for vaginal or perivaginal drug administration. The vaginal or perivaginal forms of the present invention can be manufactured using conventional processes as disclosed in Remington: The Science and Practice of Pharmacy, supra (see also drug formulations as adapted in U.S. Pat. Nos. 6,515,198; 6,500,822; 6,417,186; 6,416,779; 6,376,500; 6,355,641; 6,258,819; 6,172,062; and 6,086,909). The vaginal or perivaginal dosage unit can be fabricated to disintegrate rapidly or over a period of several hours. The time period for complete disintegration is preferably in the range of from about 10 minutes to about 6 hours, and optimally is less than about 3 hours.

Other components can also be incorporated into the vaginal or perivaginal dosage forms described herein. The additional components include, but are not limited to, stiffening agents, antioxidants, preservatives, and the like. Examples of stiffening agents that may be used include, for example, paraffin, white wax and yellow wax. Preferred antioxidants, if used, include sodium bisulfite and sodium metabisulfite.

Intranasal or Inhalation Administration

The active agents can also be administered intranasally or by inhalation. Compositions for intranasal administration are generally liquid formulations for administration as a spray or in the form of drops, although powder formulations for intranasal administration, e.g., insufflations, nasal gels, creams, pastes or ointments or other suitable formulators can be used. For liquid formulations, the active agent can be formulated into a solution, e.g., water or isotonic saline, buffered or unbuffered, or as a suspension. Preferably, such solutions or suspensions are isotonic relative to nasal secretions and of about the same pH, ranging e.g., from about pH 4.0 to about pH 7.4 or, from about pH 6.0 to about pH 7.0. Buffers should be physiologically compatible and include, for example, phosphate buffers. Furthermore, various devices are available in the art for the generation of drops, droplets and sprays, including droppers, squeeze bottles, and manually and electrically powered intranasal pump dispensers. Active agent containing intranasal carriers can also include nasal gels, creams, pastes or ointments with a viscosity of, e.g., from about 10 to about 6500 cps, or greater, depending on the desired sustained contact with the nasal mucosal surfaces. Such carrier viscous formulations can be based upon, for example, alkylcelluloses and/or other biocompatible carriers of high viscosity well known to the art (see e.g., Remington: The Science and Practice of Pharmacy, supra). Other ingredients, such as preservatives, colorants, lubricating or viscous mineral or vegetable oils, perfumes, natural or synthetic plant extracts such as aromatic oils, and humectants and viscosity enhancers such as, e.g., glycerol, can also be included to provide additional viscosity, moisture retention and a pleasant texture and odor for the formulation. Formulations for inhalation may be prepared as an aerosol, either a solution aerosol in which the active agent is solubilized in a carrier (e.g., propellant) or a dispersion aerosol in which the active agent is suspended or dispersed throughout a carrier and an optional solvent. Non-aerosol formulations for inhalation can take the form of a liquid, typically an aqueous suspension, although aqueous solutions may be used as well. In such a case, the carrier is typically a sodium chloride solution having a concentration such that the formulation is isotonic relative to normal body fluid. In addition to the carrier, the liquid formulations can contain water and/or excipients including an antimicrobial preservative (e.g., benzalkonium chloride, benzethonium chloride, chlorobutanol, phenylethyl alcohol, thimerosal and combinations thereof), a buffering agent (e.g., citric acid, potassium metaphosphate, potassium phosphate, sodium acetate, sodium citrate, and combinations thereof), a surfactant (e.g., polysorbate 80, sodium lauryl sulfate, sorbitan monopalmitate and combinations thereof), and/or a suspending agent (e.g., agar, bentonite, microcrystalline cellulose, sodium carboxymethylcellulose, hydroxypropyl methylcellulose, tragacanth, veegum and combinations thereof). Non-aerosol formulations for inhalation can also comprise dry powder formulations, particularly insufflations in which the powder has an average particle size of from about 0.1 μm to about 50 μm, preferably from about 1 μm to about 25 μm.

Topical Formulations

Topical formulations can be in any form suitable for application to the body surface, and may comprise, for example, an ointment, cream, gel, lotion, solution, paste or the like, and/or may be prepared so as to contain liposomes, micelles, and/or microspheres. Preferred topical formulations herein are ointments, creams and gels.

Ointments, as is well known in the art of pharmaceutical formulation, are semisolid preparations that are typically based on petrolatum or other petroleum derivatives. The specific ointment base to be used, preferably provides for optimum drug delivery, and, preferably, will provides for other desired characteristics as well, e.g., emolliency or the like. The ointment base is preferably inert, stable, nonirritating and nonsensitizing. As explained in Remington: The Science and Practice of Pharmacy, supra, ointment bases can be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight (See, e.g., Remington: The Science and Practice of Pharmacy, supra).

Creams, as also well known in the art, are viscous liquids or semisolid emulsions, either oil-in-water or water-in-oil. Cream bases are water-washable, and contain an oil phase, an emulsifier and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

As will be appreciated by those working in the field of pharmaceutical formulation, gels-are semisolid, suspension-type systems. Single-phase gels contain organic macromolecules distributed substantially uniformly throughout the carrier liquid, which is typically aqueous, but also, preferably, contain an alcohol and, optionally, an oil. Preferred “organic macromolecules,” i.e., gelling agents, are crosslinked acrylic acid polymers such as the “carbomer” family of polymers, e.g., carboxypolyalkylenes that may be obtained commercially under the Carbopol™ trademark. Also preferred are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methylcellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

Various additives, known to those skilled in the art, may be included in the topical formulations. For example, solubilizers may be used to solubilize certain active agents. For those drugs having an unusually low rate of permeation through the skin or mucosal tissue, it may be desirable to include a permeation enhancer in the formulation; suitable enhancers are as described elsewhere herein.

Transdermal Administration

The compounds of the invention may also be administered through the skin or mucosal tissue using conventional transdermal drug delivery systems, wherein the agent is contained within a laminated structure (typically referred to as a transdermal “patch”) that serves as a drug delivery device to be affixed to the skin. Transdermal drug delivery may involve passive diffusion or it may be facilitated using electrotransport, e.g., iontophoresis. In a typical transdermal “patch,” the drug composition is contained in a layer, or “reservoir,” underlying an upper backing layer. The laminated structure may contain a single reservoir, or it may contain multiple reservoirs. In one type of patch, referred to as a “monolithic” system, the reservoir is comprised of a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are separate and distinct layers, with the adhesive underlying the reservoir which, in this case, may be either a polymeric matrix as described above, or it may be a liquid or hydrogel reservoir, or may take some other form.

The backing layer in these laminates, which serves as the upper surface of the device, functions as the primary structural element of the laminated structure and provides the device with much of its flexibility. The material selected for the backing material should be selected so that it is substantially impermeable to the active agent and any other materials that are present, the backing is preferably made of a sheet or film of a flexible elastomeric material. Examples of polymers that are suitable for the backing layer include polyethylene, polypropylene, polyesters, and the like.

During storage and prior to use, the laminated structure includes a release liner. Immediately prior to use, this layer is removed from the device to expose the basal surface thereof, either the drug reservoir or a separate contact adhesive layer, so that the system may be affixed to the skin. The release liner should be made from a drug/vehicle impermeable material.

Transdermal drug delivery systems may in addition contain a skin permeation enhancer. That is, because the inherent permeability of the skin to some drugs may be too low to allow therapeutic levels of the drug to pass through a reasonably sized area of unbroken skin, it is necessary to coadminister a skin permeation enhancer with such drugs. Suitable enhancers are well known in the art and include, for example, those enhancers listed above in transmucosal compositions.

Intrathecal Administration

One common system utilized for intrathecal administration is the APT Intrathecal treatment system available from Medtronic, Inc. APT Intrathecal uses a small pump that is surgically placed under the skin of the abdomen to deliver medication directly into the intrathecal space. The medication is delivered through a small tube called a catheter that is also surgically placed. The medication can then be administered directly to cells in the spinal cord involved in conveying sensory and motor signals associated with lower urinary tract disorders.

Another system available from Medtronic that is commonly utilized for intrathecal administration is the fully implantable, programmable SynchroMed™ Infusion System. The SynchroMed™ Infusion System has two parts that are both placed in the body during a surgical procedure: the catheter and the pump. The catheter is a small, soft tube. One end is connected to the catheter port of the pump, and the other end is placed in the intrathecal space. The pump is a round metal device about one inch (2.5 cm) thick, three inches (8.5 cm) in diameter, and weighs about six ounces (205 g) that stores and releases prescribed amounts of medication directly into the intrathecal space. It can be made of titanium, a lightweight, medical-grade metal. The reservoir is the space inside the pump that holds the medication. The fill port is a raised center portion of the pump through which the pump is refilled. The doctor or a nurse inserts a needle through the patient's skin and through the fill port to fill the pump. Some pumps have a side catheter access port that allows the doctor to inject other medications or sterile solutions directly into the catheter, bypassing the pump.

The SynchroMed™ pump automatically delivers a controlled amount of medication through the catheter to the intrathecal space around the spinal cord, where it is most effective. The exact dosage, rate and timing prescribed by the doctor are entered in the pump using a programmer, an external computer-like device that controls the pump's memory. Information about the patient's prescription can be stored in the pump's memory. The doctor can easily review this information by using the programmer. The programmer communicates with the pump by radio signals that allow the doctor to tell how the pump is operating at any given time. The doctor also can use the programmer to change your medication dosage.

Methods of intrathecal administration can include those described above available from Medtronic, as well as other methods that are known to one of skill in the art.

Intravesical Administration

The term intravesical administration is used herein in its conventional sense to mean delivery of a drug directly into the bladder. Suitable methods for intravesical administration can be found in U.S. Pat. Nos. 6,207,180 and 6,039,967, for example.

Additional Administration Forms

Additional dosage forms of this invention include dosage forms as described in U.S. Pat. No. 6,340,475, U.S. Pat. No. 6,488,962, U.S. Pat. No. 6,451,808, U.S. Pat. No. 5,972,389, U.S. Pat. No. 5,582,837, and U.S. Pat. No. 5,007,790. Additional dosage forms of this invention also include dosage forms as described in U.S. patent application Ser. No. 20030147952, U.S. patent application Ser. No. 20030104062, U.S. patent application Ser. No. 20030104053, U.S. patent application Ser. No. 20030044466, U.S. patent Application Ser. No. 20030039688, and U.S. patent application Ser. No. 20020051820. Additional dosage forms of this invention also include dosage forms as described in PCT Patent Application WO 03/35041, PCT Patent Application WO 03/35040, PCT Patent Application WO 03/35029, PCT Patent Application WO 03/35177, PCT Patent Application WO 03/35039, PCT Patent Application WO 02/96404, PCT Patent Application WO 02/32416, PCT Patent Application WO 01/97783, PCT Patent Application WO 01/56544, PCT Patent Application WO 01/32217, PCT Patent Application WO 98/55107, PCT Patent Application WO 98/11879, PCT Patent Application WO 97/47285, PCT Patent Application WO 93/18755, and PCT Patent Application WO 90/11757.

For intrabronchial or intrapulmonary administration, conventional formulations can be employed.

Further, the compounds for use in the method of the invention can be formulated in a sustained release preparation, further described herein. For example, the compounds can be formulated with a suitable polymer or hydrophobic material which provides sustained and/or controlled release properties to the active agent compound. As such, the compounds for use the method of the invention can be administered in the form of microparticles for example, by injection or in the form of wafers or discs by implantation.

In one embodiment, the dosage forms of the present invention include pharmaceutical tablets for oral administration as described in U.S. patent application Ser. No. 20030104053. For example, suitable dosage forms of the present invention can combine both immediate-release and prolonged-release modes of drug delivery. The dosage forms of this invention include dosage forms in which the same drug is used in both the immediate-release and the prolonged-release portions as well as those in which one drug is formulated for immediate release and another drug, different from the first, is formulated for prolonged release. This invention encompasses dosage forms in which the immediate-release drug is at most sparingly soluble in water, i.e., either sparingly soluble or insoluble in water, while the prolonged-release drug can be of any level of solubility.

### EXAMPLES

This invention is further illustrated by the following examples, which should not be construed as limiting.

### Example 1

### Preparation of Tetramic Acid Compounds

The general synthetic preparation of tetramic acid compounds of the invention are described below.

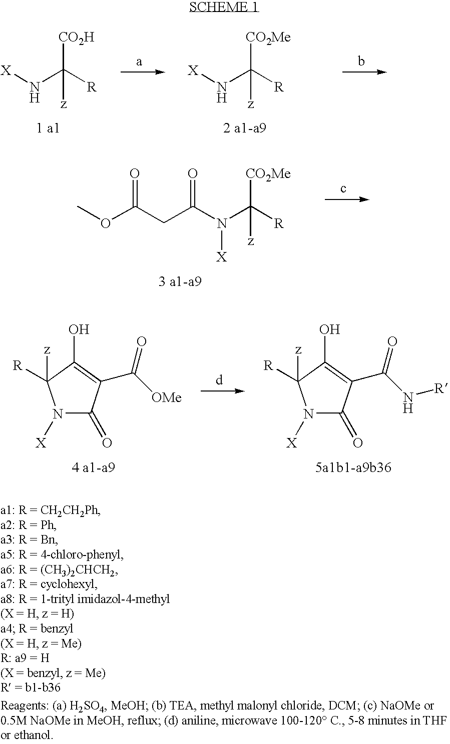

### I. Synthesis of Intermediates 4

### A. 2-Amino-4-phenyl-butyric acid Methyl Ester

### 2a1

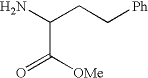

To a stirred solution of homo-phenyl alanine (3.5 g, 19.5 mmol) in 60 mL MeOH was added concentrated H₂SO₄ via syringe (1.03 mL, 19.5 mmol), the reaction mixture was stirred at rt for 5 minutes and heated up to 70° C. for 120 minutes. The reaction mixture was cooled down to rt. MeOH was evaporated and the mixture was diluted with 100 mL EtOAc, washed with NaHCO₃, water, brine, then dried over Na₂SO₄. The solvent was then removed to give the title compound as a light yellow solid (3.63 g, 96.3%). The material was used without further purification. MS (ES+): m/z=194 (M+1) 1H NMR (400 MHz, CHLOROFORM-D) δ=1.79; 1.89 (m, 1H) 2.01; 2.10 (m, 1H) 2.67; 2.77 (m, 2H) 3.43 (dd, J=7.83, 5.31 Hz, 1H) 3.68 (s, 3H) 7.15; 7.21 (m, 3H) 7.26; 7.29 (m, 2H)

### B. 2-(2-Methoxycarbonyl-acetylamino)-4-phenyl-butyric acid Methyl Ester

### 3a1

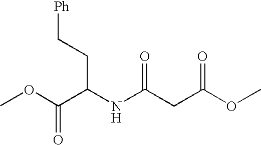

To a stirred solution of 2-amino-4-phenyl-butyric acid methyl ester, 2a1 (3.0 g, 15.5 mmol) and triethylamine (2.28 mL, 16.2 mmol) in dichloromethane (25 mL) was added methyl malonyl chloride (1.74 mL, 16.2 mmol) portionwise at 0° C. under nitrogen atmosphere. The reaction mixture was stirred for further 90 minutes, evaporated and then diluted with 45 mL EtOAc. The organic solution was washed with water and brine, dried over Na₂SO₄. The solvent was then removed to give the title compound as a yellow solid (3.12 g, 72.1%). The material was used without further purification in the next step. MS (ES+): m/z=294 (M+1)

### C. N-(Methoxycarbonyl-phenyl-methyl)-malonamic acid Methyl Ester

### 3a2

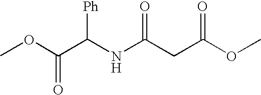

Analogous to 3a1 compound 3a2 was prepared from 3.0 g of 2a2 to yield 4.65 g (96.8% yield) of the title compound as a light yellow solid. MS (ES+): m/z=266 (M+1)

### D. 2-(2-Methoxycarbonyl-acetylamino)-3-phenyl-propionic acid Methyl Ester

### 3a3

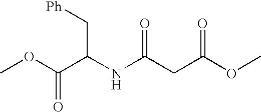

Analogous to 3a1 compound 3a3 was prepared from 5.0 g of 2a3 to yield 7.6 g (97.6% yield) of the title compound as a light yellow solid. MS (ES+): m/z= 280 (M+1)

### E. 2-(2-Methoxycarbonyl-acetylamino)-2-methyl-3-phenyl-propionic acid Methyl Ester

### 3a4

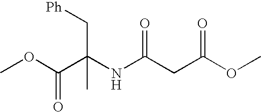

Analogous to 3a1 compound 3a4 was prepared from 5.0 g of 2a4 to yield 8.5 g (yield 96.5%) of the title compound as a yellow solid. MS (ES+): m/z=294 (M+1)

### F. 3-(4-Chloro-phenyl)-2-(2-methoxycarbonyl-acetylamino)-propionic acid Methyl

### 3a5

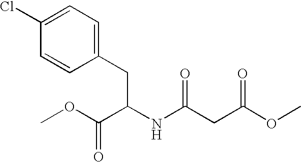

Analogous to 3a1 compound 3a5 was prepared from 5.0 g of 2a5 to yield 6.9 g (94.5% yield) of the title compound as a yellow powder. MS (ES+): m/z=314 (M+1)

### G. 2-(2-Methoxycarbonyl-acetylamino)-4-methyl-pentanoic acid Methyl Ester

### 3a6

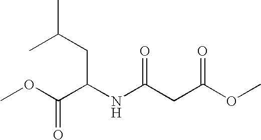

Analogous to 3a1 compound 3a6 was prepared from 5.0 g of 2a6 to yield 8.2 g (97.6% yield) of the title compound as a white powder, MS (ES+): m/z=246 (M+1)

### H. 3-Cyclohexyl-2-(2-methoxycarbonyl-acetylamino)-propionic acid Methyl Ester

### 3a7

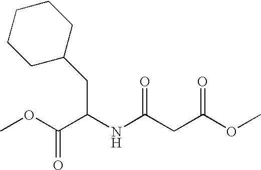

Analogous to 3a1 compound 3a7 was prepared from 5.0 g of 2a7 to yield 7.1 g (92.2% yield) of the title compound as a light yellow solid, MS (ES+): m/z=286 (M+1)

### I. 2-(2-Methoxycarbonyl-acetylamino)-3-(1-trityl-1H-imidazol-4-yl)-propionic acid Methyl Ester

### 3a8

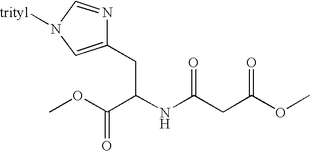

Analogous to 3a1 compound 3a8 was prepared from 1.0 g of 2a8 to yield 1 g (79.4% yield) of the title compound as a yellow powder. MS (ES+): m/z=480 (M+1)

### J. N-Benzyl-N-methoxycarbonylmethyl-malonamic acid Methyl Ester

### 3a9

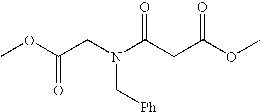

To a stirred solution of N-benzyl glycine methyl ester (179 mg, 0.84 mmol) and triethylamine (244.3 μL, 1.75 mmol) in dichloromethane (8 mL) was added methyl malonyl chloride (89.39 μL, 0.84 mmol) portionwise at 0° C. under N₂ atmosphere. The reaction mixture was stirred for further 90 minutes, evaporated and then diluted with 10 mL EtOAc. The organic solution was washed with water and brine, dried over Na₂SO₄. The solvent was then removed to give the title compound as a yellow solid (212 mg, 91%). The material was used without further purification in the next step. MS (ES+): m/z=280 (M+1)

### II. General Procedure for the Formation of Tetramic Acid Methyl Esters 4a from Compounds 3a

To a solution of the ester 3a (2 mmol, 1 eq.) in MeOH was added NaOMe or 0.5M NaOMe (4 mmol, 2.0 eq.) in MeOH and the mixture was heated at reflux for 2 hours. The solid was collected by filtration and washed with diethyl ether, the resulting cake was dissolved by adding iced water and 1N HCl and the separated solids were filtered, washed with water, brine and dried over Na₂SO₄ and concentrated under vacuum to afford the tetramic methyl esters 4a.

### A. 4-Hydroxy-2-oxo-5-phenethyl-2,5-dihydro-1H-pyrrole-3-carboxylic acid Methyl Ester

### 4a1

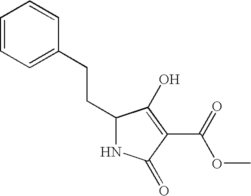

To a stirred solution of 2-(2-methoxycarbonyl-acetylamino)-4-phenyl-butyric acid methyl ester (3 g, 9.2 mmol) in 50 ml MeOH was added NaOMe (998 mg, 18.4 mmol), the reaction mixture was heated at reflux for 2 hours. The solid was collected by filtration and washed with diethyl ether, the resulting cake was dissolved by adding iced water with 10 ml 1N HCl and the separated solids were filtered, washed with water, brine and dried over NaSO₄ and concentrated under vacuum to afford white solid. MS (ES+): m/z=262 (M+1) 1H NMR (500 MHz, DMSO-D6) δ=1.70 (dd, J=8.83, 5.04 Hz, 1H) 2.02 (ddd, J=6.78, 3.31, 3.15 Hz, 1H) 2.57; 2.66 (m, 2H) 3.65 (s, 3H) 3.93 (d, J=4.41 Hz, 1H) 7.17; 7.23 (m, 3H) 7.29 (t, J=7.25 Hz, 2H)

### B. 5-Phenyl-4-hydroxy-2-oxo-2,5-dihydro-1H-pyrrole-3-carboxylic acid Methyl Ester

### 4a2

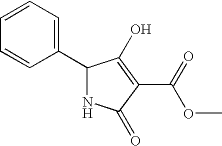

Analogous to 4a1 compound 4a2 was prepared from 4.65 g 3a2 to yield 3.2 g (78% yield) of the title compound as light yellow powder. MS (ES+): m/z=234 (M+1) 1H NMR (400 MHz, CHLOROFORM-D) δ=3.97 (s, 3H) 5.21 (s, 1H) 7.39 (m, 5H)

### C. 5-Benzyl-4-hydroxy-2-oxo-2,5-dihydro-1H-pyrrole-3-carboxylic acid Methyl Ester

### 4a3

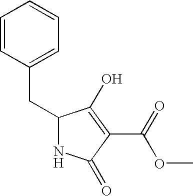

Analogous to 4a1 compound 4a3 was prepared from 5.0 g 3a3 to yield 4.39 g (99% yield) of the title compound as white powder. MS (ES+); m/z=248 (M+1) 1H NMR (400 MHz, DMSO-D6) δ=2.76; 2.86 (m, 1H) 2.89; 2.98 (m, 1H) 3.52 (s, 3H) 4.19 (t, J−4.55 Hz, 1H) 7.09; 7.20 (m, 5H)

### D. 5-Benzyl-4-hydroxy-5-methyl-2-oxo-2,5-dihydro-1H-pyrrole-3-carboxylic acid Methyl Ester

### 4a4

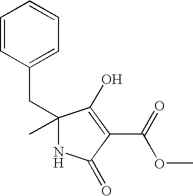

Analogous to 4a1 compound 4a3 was prepared from 5.5 g 3a3 to yield 4.0 g (81.6% yield) of the title compound as light yellow powder. MS (ES+): m/z=262 (M+1) 1H NMR (400 MHz, CHLOROFORM-D) δ=1.44 (s, 3H) 2.97 (s, 2H) 3.89 (s, 3H) 7.27 (m, 5H)

### E. 5-(4-Chloro-benzyl)-4-hydroxy-2-oxo-2,5-dihydro-1H-pyrrole-3-carboxylic acid Methyl Ester

### 4a5

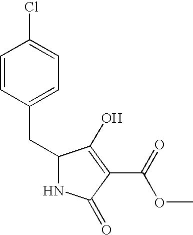

Analogous to 4a1 compound 4a5 was prepared from 5.0 g 3a5 to yield 3.89 g (86.4% yield) of the title compound as light yellow solid. MS (ES+): m/z=282 (M+1) 1H NMR (500 MHz, DMSO-D6) δ=2.87 (dd, J=13.87, 5.67 Hz, 1H) 2.99 (dd, J=13.87, 4.41 Hz, 1H) 3.58 (s, 3H) 4.20 (t, J=5.04 Hz, 1H) 7.18 (d, J=8.20 Hz, 2H) 7.30 (d, J=8.20 Hz, 2H)

### F. 4-Hydroxy-5-isobutyl-2-oxo-2,5-dihydro-1H-pyrrole-3-carboxylic acid Methyl Ester

### 4a6

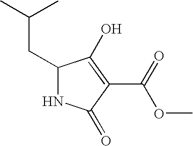

Analogous to 4a1 compounds 4a6 was prepared from 5.0 g 3a5 to yield 4.1 g (94% yield) of the title compound as white powder. MS (ES+): m/z=214 (M+1) 1H NMR (500 MHz, DMSO-D6) δ=0.88 (m, 6H) 1.27 (m, 1H) 1.54 (m, 1H) 1.77 (s, 1H) 3.65 (s, 3H) 3.95 (dd, J=9.77, 3.47 Hz, 1H)

### G. 5-Cyclohexylmethyl-4-hydroxy-2-oxo-2,5-dihydro-1H-pyrrole-3-carboxylic acid Methyl Ester

### 4a7

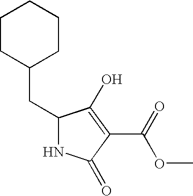

Analogous to 4a1 compound 4a7 was prepared from 5.0 g 3a7 to yield 4.31 g (96.8% yield) of the title compound as white powder. MS (ES+): m/z=254 (M+1) 1H NMR (400 MHz, DMSO-D6) δ=0.82; 0.93 (m, 2H) 1.13; 1.25 (m, 4H) 1.47 (dd, J=8.84, 3.28 Hz, 1H) 1.56; 1.68 (m, 5H) 1.77 (d, J=12.63 Hz, 1H) 3.65 (s, 3H) 3.98 (dd, J=9.35, 3.28 Hz, 1H)

### H. 4-Hydroxy-2-oxo-5-(1-trityl-1H-imidazol-4-ylmethyl)-2,5-dihydro-1H pyrrole-3-carboxylic acid Methyl Ester

### 4a8

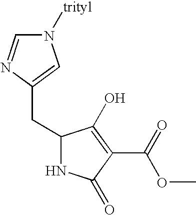

Analogous to 4a1 compound 4a8 was prepared from 21 mg 3a8 to yield 15 mg (76.1% yield) of the title compound as a light yellow solid MS (ES+): m/z=480 (M+1)

### I. 1-Benzyl-4-hydroxy-2-oxo-2,5-dihydro 1H-pyrrole-3-carboxylic acid Methyl Ester

### 4a9

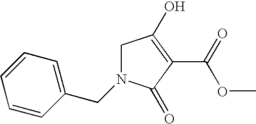

Analogous to 4a1 compound 4a9 was prepared from 613 mg 3a9 to yield 500 mg (92.5% yield) of the title compound as a yellow powder, MS (ES+): 248=(M+1) 1H NMR (400 MHz, MeOH-D4) δ=3.83 (s, 3H) 3.93 (s, 2H) 4.60 (s, 2H) 7.27; 7.38 (m, 5H)

### III. Synthesis of Examples

### A. General Procedure for the Amide Formation of Compounds 5a with Compounds 4a and Amines

To a solution of the ester 4a (2 mmol, 1 eq.) in THF or ethanol was added the amine (2.2 mmol, 1.1 eq.) and mixture was heated in the microwave synthesizer (Biotage at 100-120° C. for 5-8 min and concentrated in vacuum afterwards. The residue was suspended in ether, collected by filtration, and rinsed with ether and little methanol to afford the amides 5a. In some case purification by reversed phase HPLC was necessary thereafter to isolate the pure amides 5a.

### B. 4-Hydroxy-2-oxo-5-phenethyl-2,5-dihydro-1H-pyrrole-3-carboxylic acid(4-piperidin-1-yl-phenyl)-amide

### 5a1b14

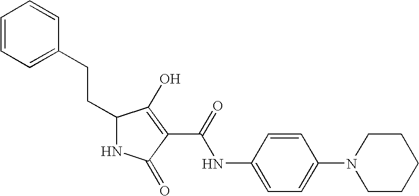

To a solution of 4a1 (65.25 mg, 0.25 mmol) in THF was added 4-piperidin-1-yl-phenylamine and the resulting mixture was heated in microwave at 100° C. for 5 min, then concentrated in vacuum. The residue was suspended in ether, collected by filtration, and rinsed with ether and methanol to afford the title compound as a white solid (52.4 mg, 52%). MS (ES+): m/z=406 (M+1) 1H NMR (400 MHz, DMSO-D6) δ=1.46 (d, J=5.05 Hz, 2H) 1.55 (d, J=4.55 Hz, 4H) 1.64; 1.74 (m, 1H) 1.92; 2.02 (m, 1H) 2.54; 2.65 (m, 2H) 2.99; 3.07 (m, 4H) 3.99 (d, J=3.54 Hz, 1H) 6.87 (d, J=8.59 Hz, 2H) 7.10; 7.18 (m, 3H) 7.23 (t, J=7.33 Hz, 2H) 7.38 (d, J=8.59 Hz, 2H) 8.36 (s, 1H) 9.95 (s, 1H)

The following compounds were prepared according to the general protocol from above;

| | | | MS |
|---|---|---|---|
| Compoud | | | m/z |
| # | Structure | Name | (M + 1) |
| 5a₁b₁ | 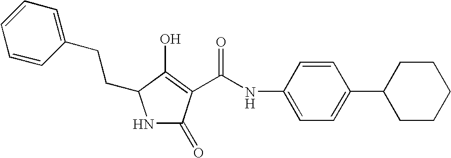 | 4-Hydroxy-2-oxo-5- phenyl-2,5-dihydro- 1H-pyrrole-3- carboxylic acid (4- cyclohexyl-phenyl)- amide | 406 |
| 5a₁b₂ | 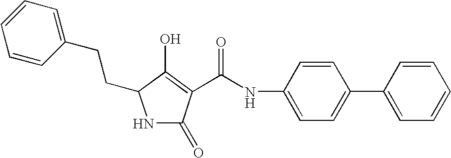 | 4-Hydroxy-2-oxo-5- phenethyl-2,5-dihydro- 1H-pyrrole-3- carboxylic acid biphenyl-4-ylamide | 399 |
| 5a₁b₃ | 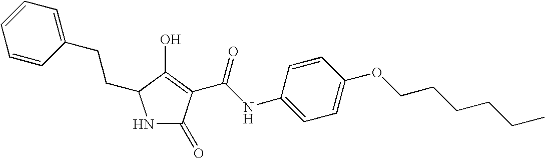 | 4-Hydroxy-2-oxo-5- phenethyl-2,5-dihydro- 1H-pyrrole-3- carboxylic acid (4- hexyloxy- phenyl)-amide | 423 |
| 5a₁b₆ | 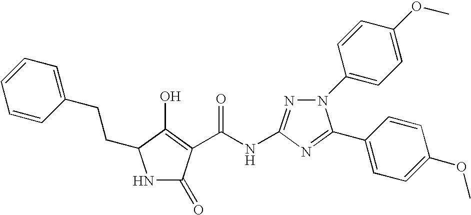 | 4-Hydroxy-2-oxo-5- phenethyl-2,5-dihydro- 1H-pyrrole-3- carboxylic acid [1,5- bis-(4-methoxy- phenyl-1H- [1,2,4]triazol-3-yl]- amide | 526 |
| 5a₁b₇ | 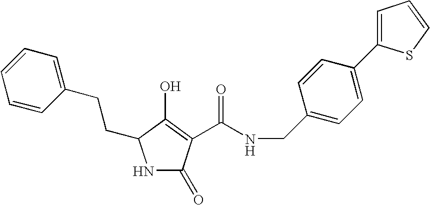 | 4-Hydroxy-2-oxo-5- phenethyl-2,5-dihydro- 1H-pyrrole-3- carboxylic acid 4- thiophen-2-yl- benzylamide | 419 |
| 5a₁b₁₄ | 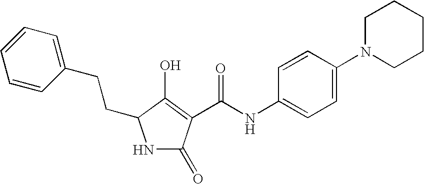 | 4-Hydroxy-2-oxo-5- phenethyl-2,5-dihydro- 1H-pyrrole-3- carboxylic acid(4- piperidin-1-yl-phenyl)- amide | 406 |
| 5a₁b₁₈ | 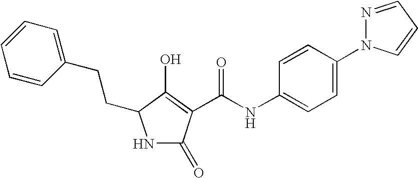 | 4-Hydroxy-2-oxo-5- phenethyl-2,5-dihydro- 1H-pyrrole-3- carboxylic acid(4- pyrazol-1-yl-phenyl)- amide | 389 |
| 5a₁b₂₂ | 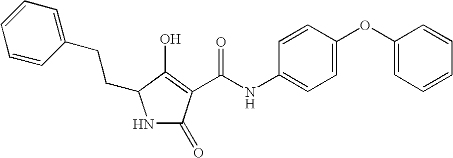 | 4-Hydroxy-2-oxo-5- phenethyl-2,5-dihydro- 1H-pyrrole-3- carboxylic acid (4- phenoxy-phenyl)- amide | 415 |
| 5a₁b₂₃ | 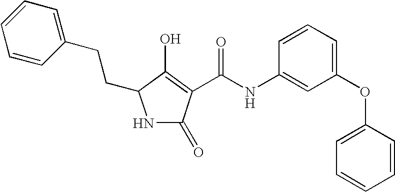 | 4-Hydroxy-2-oxo-5- phenethyl-2,5-dihydro- 1H-pyrrole-3- carboxylic acid (3- phenoxy-phenyl)- amide | 415 |
| 5a₂b1 | 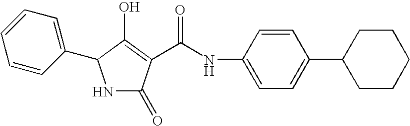 | 4-Hydroxy-2-oxo-5- phenyl-2,5-dihydro- 1H-pyrrole-3- carboxylic acid (4- cyclohexyl- phenyl) amide | 377 |
| 5a₂b₇ | 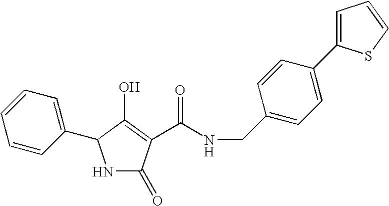 | 4-Hydroxy-2-oxo-5- phenyl-2,5-dihydro- 1H-pyrrole-3- carboxylic acid4- thiophen-2-yl benzyamide | 391 |
| 5a₃b₄ | 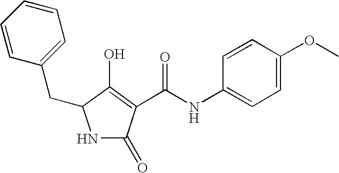 | 5-Benzyl-4-hydroxy-2- oxo-2,5-dihydro-1H- pyrrole-3-carboxylic acid (4-methoxy- phenyl-amide | 339 |
| 5a₃b₅ | 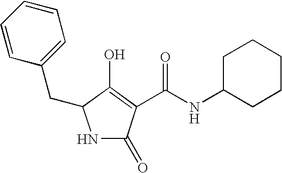 | 5-Benzyl-4-hydroxy-2- oxo-2,5-dihydro-1H- pyrrole-3-carboxylic acid cyclohexyl amide | 315 |
| 5a₃b₇ | 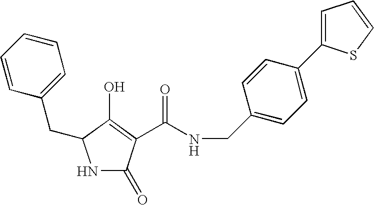 | 5-Benzyl-4-hydroxy-2- oxo-2,5-dihydro-1H- pyrrole-3-carboxylic acid4-thiophen-2-yl- benzylamide | 405 |
| 5a₃b₁₄ | 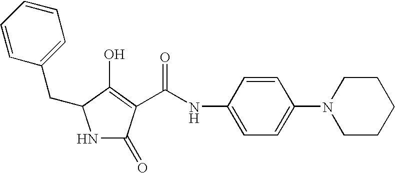 | 5-Benzyl-4-hydroxy-2- oxo-2,5-dihydro-1H- pyrrole-3-carboxylic acid (4-piperidin-1-yl- phenyl-amide | 392 |
| 5a₃b₂₁ | 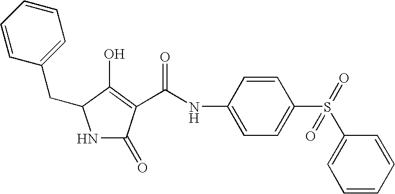 | 5-Benzyl-4-hydroxy-2- oxo-2,5-dihydro-1H- pyrrole-3-carboxylic acid(4- benzenesulfonyl- phenyl-amide | 449 |
| 5a₃b₂₃ | 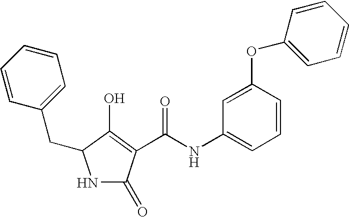 | 5-Benzyl-4-hydroxy-2- oxo-2,5-dihydro-1H- pyrrole-3-carboxylic acid (3-phenoxy- phenyl-amide | 401 |
| 5a₃b₂₅ | 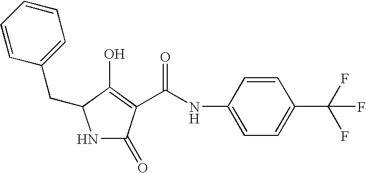 | 5-Benzyl-4-hydroxy-2- oxo-2,5-dihydro-1H- pyrrole-3-carboxylic acid (trifluoro methyl)- amide | 377 |
| 5a₃b₂₆ | 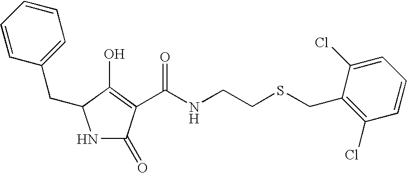 | 5-Benzyl-4-hydroxy-2- oxo-2,5-dihydro-1H- pyrrole-3-carboxylic acid [2-(2,6-dichloro- benzylsulfanyl)-ethyl]- amide | 452 |
| 5a₃b₃₁ | 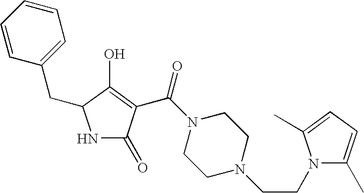 | 5-Benzyl-3-{4-[2-(2,5- dimethyl-pyrrol-1-yl)- ethyl]-piperazine-1- carbonyl}-4-hydroxy- 1,5-dihydro-pyrrol-2- one | 423 |
| 5a₃b₃₃ | 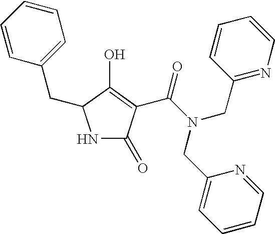 | 5-Benzyl-4-hydroxy-2- oxo-2,5-dihydro-1H- pyrrole-3-carboxylic acid bis-pyridin-2- ylmethyl-amide | 415 |
| 5a₄b₁ | 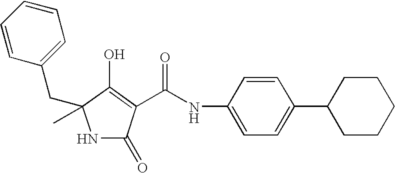 | 5-Benzyl-4-hydroxy-5- methyl-2-oxo-2,5- dihydro-1H-pyrrole-3- carboxylic acid(4- cyclohexyl-phenyl)- amide | 405 |
| 5a₄b₂ | 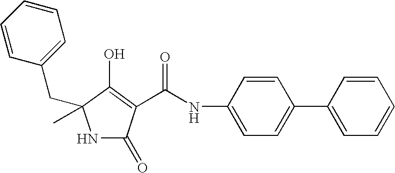 | 5-Benzyl-4-hydroxy-5- methyl-2-oxo-2,5- dihydro-1H-pyrrole-3- carboxylicacid biphenyl-4-ylamide | 399 |
| 5a₄b₇ | 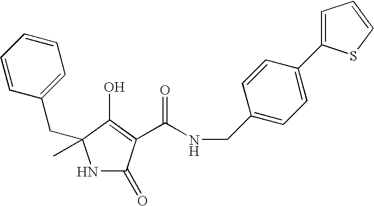 | 5-Benzyl-4-hydroxy-5- methyl-2-oxo-2,5- dihydro-1H-pyrrole-3- carboxylic acid 4- thiophen-2-yl- benzylamide | 419 |
| 5a₄b₈ | 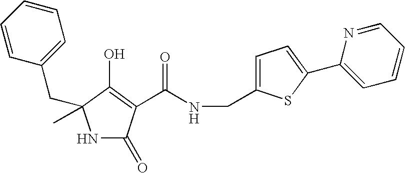 | 5-Benzyl-4-hydroxy-5- methyl-2-oxo-2,5- dihydro-1H-pyrrole-3- carboxylic acid (5- pyridin-2-yl-thiophen-2-ylmethyl)-amide | 420 |
| 5a₄b₉ | 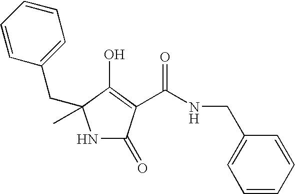 | 5-Benzyl-4-hydroxy-5- methyl-2-oxo-2,5- dihydro-1H-pyrrole-3- carboxylic acid benzylamide | 337 |
| 5a₄b₁₀ | 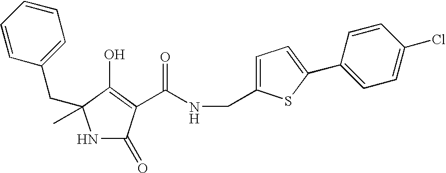 | 5-Benzyl-4-hydroxy-5- methyl-2-oxo-2,5- dihydro-1H-pyrrole-3- carboxylicacid[5-(4- chloro-phenyl)- thiophen-2-ylemthyl]- amide | 453 |
| 5a₄b₁₁ | 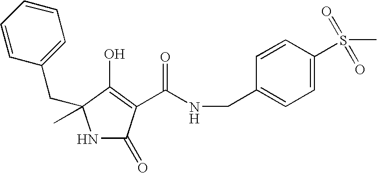 | 5-Benzyl-4-hydroxy-5- methyl-2-oxo-2,5- dihydro-1H-pyrrole-3- carboxylicacid 4- methanesulfonyl- benzylamide | 415 |
| 5a₄b₁₂ | 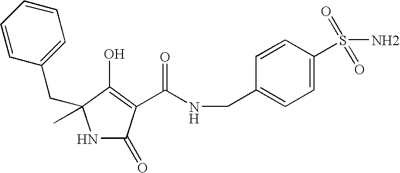 | 5-Benzyl-4-hydroxy-5- methyl-2-oxo-2,5- dihydro-1H-pyrrole-3- carboxylic acid 4- sulfamoyl-benzylamide | 416 |
| 5a₄b₁₉ | 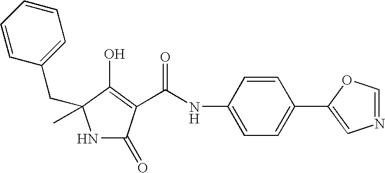 | 5-Benzyl-4-hydroxy-5- methyl-2-oxo-2,5- dihydro-1H-pyrrole-3- carboxylic acid (4- oxazol-5-yl-phenyl)- amide | 390 |
| 5a₄b₂₃ | 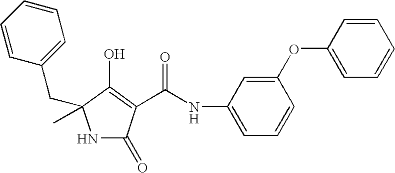 | 5-Benzyl-4-hydroxy-5- methyl-2-oxo-2,5- dihydro-1H-pyrrole-3- carboxylic acid (3- phenoxy-phenyl)- amide | 415 |
| 5a₄b₂₈ | 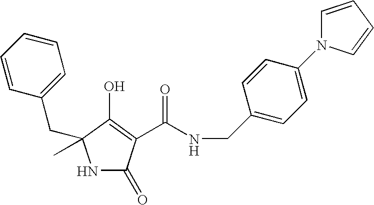 | 5-Benzyl-4-hydroxy-5- methyl-2-oxo-2,5- dihydro-1H-pyrrole-3- carboxylic acid 4- pyrrol-1-yl- benzylamide | 402 |
| 5a₄b₂₉ | 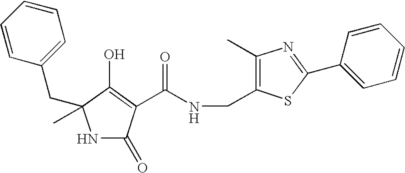 | 5-Benzyl-4-hydroxy-5- methyl-2-oxo-2,5- dihydro-1H-pyrrole-3- carboxylic acid (4- methyl-2-phenyl- thiazol-5-ylmethyl)- amide | 434 |
| 5a₅b₁ | 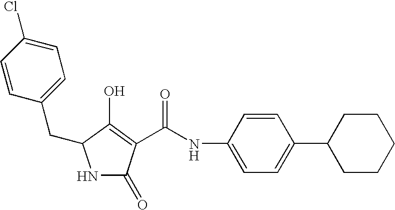 | 5-(4-Chloro-benzyl)-4- hydroxy-2-oxo-2,5- dihydro-1H-pyrrole-3- carboxylic acid (4- cyclohexyl-phenyl)- amide | 425 |
| 5a₅b₉ | 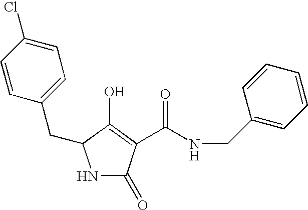 | 5-(4-Chloro-benzyl)-4- hydroxy-2-oxo-2,5- dihydro-1H-pyrrole-3- carboxylic acid benzylamide | 357 |
| 5a₅b₁₁ | 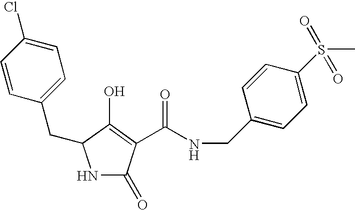 | 5-(4-Chloro-benzyl)-4- hydroxy-2-oxo-2,5- dihydro-1H-pyrrole-3- carboxylicacid 4- methanesulfonyl- benzylamide | 435 |
| 5a₆b₁ | 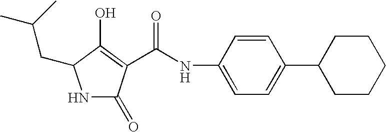 | 4-Hydroxy-5-isobutyl- 2-oxo-2,5-dihydro-1H- pyrrole-3-carboxylic acid (4-cyclohexyl- phenyl)-amide | 357 |
| 5a₆b₂ | 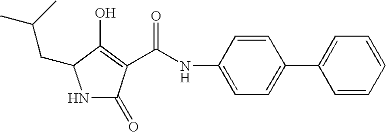 | 4-Hydroxy-5-isobutyl- 2-oxo-2,5-dihydro-1H- pyrrole-3-carboxylic acid biphenyl-4- ylamide | 351 |
| 5a₆b₃ | 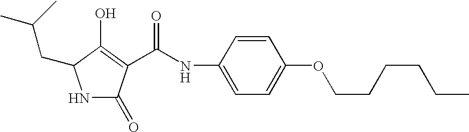 | 4-Hydroxy-5-isobutyl- 2-oxo-2,5-dihydro-1H- pyrrole-3-carboxylic acid (4-hexyloxy- phenyl)-amide | 375 |
| 5a₆b₆ | 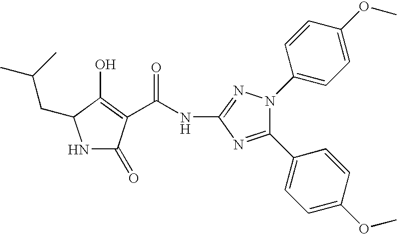 | 4-Hydroxy-5-isobutyl- 2-oxo-2,5-dihydro-1H- pyrrole-3-carboxylic acid [1,5-bis-(4- methoxy-phenyl)-1H- [1,2,4]triazol-3-yl]- amide | 478 |
| 5a₆b₇ | 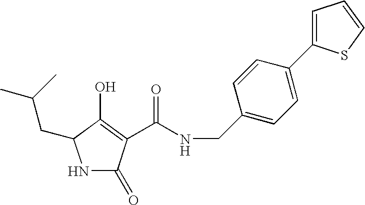 | 4-Hydroxy-5-isobutyl- 2-oxo-2,5-dihydro-1H- pyrrole-3-carboxylic acid 4-thiophen-2-yl- benzylamide | 371 |
| 5a₆b₉ | 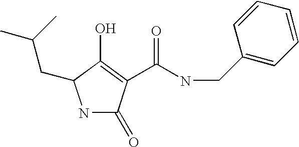 | 4-Hydroxy-5-isobutyl- 2-oxo-2,5-dihydro-1H- pyrrole-3-carboxylic acid benzylamide | 289 |
| 5a₆b₁₁ | 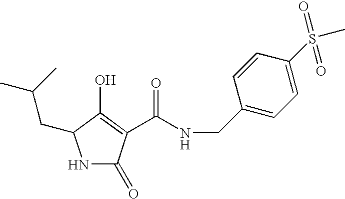 | 4-Hydroxy-5-isobutyl- 2-oxo-2,5-dihydro-1H- pyrrole-3-carboxylic acid4-methanesulfonyl- benzylamide | 367 |
| 5a₆b₁₄ | 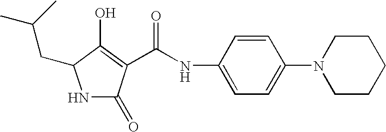 | 4-Hydroxy-5-isobutyl- 2-oxo-2,5-dihydro-1H- pyrrole-3-carboxylic acid(4-piperidin-1-yl- phenyl)-amide | 358 |
| 5a₆b₁₅ | 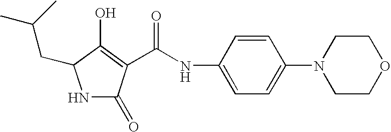 | 4-Hydroxy-5-isobutyl- 2-oxo-2,5-dihydro-1H- pyrrole-3-carboxylic acid (4-morpholin-4-yl- phenyl)-amide | 360 |
| 5a₆b₁₆ | 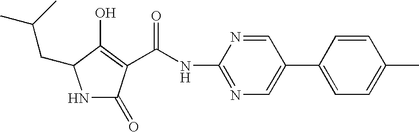 | 4-Hydroxy-5-isobutyl- 2-oxo-2,5-dihydro-1H- pyrrole-3-carboxylic acid(5-p-tolyl- pyrimidin-2-yl)-amide | 367 |
| 5a₆b₁₇ | 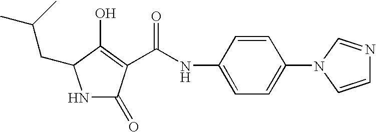 | 4-Hydroxy-5-isobutyl- 2-oxo-2,5-dihydro-1H- pyrrole-3-carboxylic acid(4-imidazol-1-yl phenyl)-amide | 341 |
| 5a₆b₁₈ | 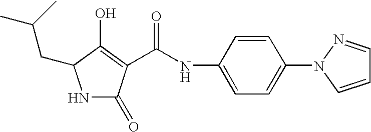 | 4-Hydroxy-5-isobutyl- 2-oxo-2,5-dihydro-1H- pyrrole-3-carboxylic acid(4-pyrazol-1-yl- phenyl)-amide | 341 |
| 5a₆b₁₉ | 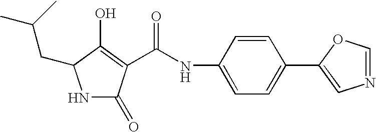 | 4-Hydroxy-5-isobutyl- 2-oxo-2,5-dihydro-1H- pyrrole-3-carboxylic acid (4-oxazol-5-yl- phenyl)-amide | 342 |
| 5a₆b₂₀ | 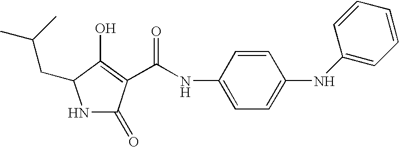 | 4-Hydroxy-5-isobutyl- 2-oxo-2,5-dihydro-1H- pyrrole-3-carboxylic acid(4-phenylamino- phenyl)-amide | 366 |
| 5a₆b₂₁ | 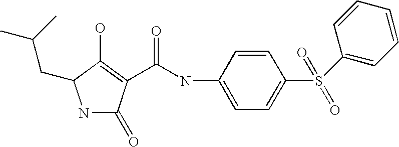 | 4-Hydroxy-5-isobutyl- 2-oxo-2,5-dihydro-1H- pyrrole-3-carboxylic acid (4- benzenesulfonyl- phenyl)-amide | 415 |
| 5a₆b₂₃ | 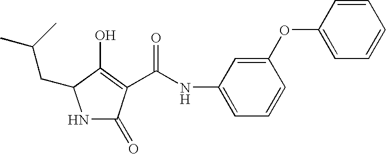 | 4-Hydroxy-5-isobutyl- 2-oxo-2,5-dihydro-1H- pyrrole-3-carboxylic acid (3-phenyoxy- phenyl)-amide | 367 |
| 5a₆b₂₄ | 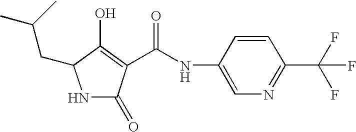 | 4-Hydroxy-5-isobutyl- 2-oxo-2,5-dihydro-1H- pyrrole-3-carboxylic acid(4-trifluromethyl- peridine)-amide | 344 |
| 5a₆b₂₈ | 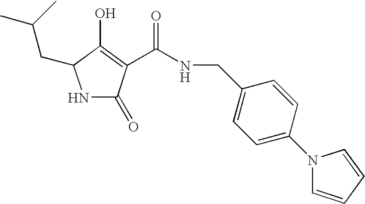 | 4-Hydroxy-5-isobutyl- 2-oxo-2,5-dihydro-1H- pyrrole-3-carboxylic acid 4-pyrrol-1-yl- benzylamide | 354 |
| 5a₆b₃₀ | 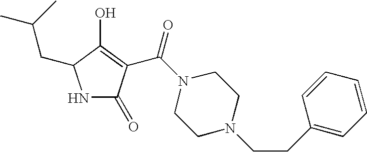 | 4-Hydroxy-5-isobutyl- 3-(4-phenethyl- piperazine-1-carbonyl)- 1,5-dihydro-pyrrol-2- one | 372 |
| 5a₆b₃₂ | 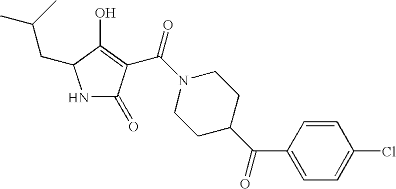 | 3-[4-(4-Chloro- benzoyl)-piperidine-1-carbonyl]-4-hydroxy-5- isobutyl-1,5-dihydro- pyrrol-2-one | 405 |
| 5a₆b₃₃ | 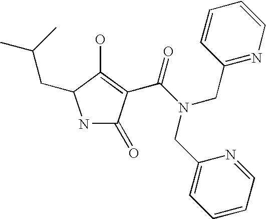 | 4-Hydroxy-5-isobutyl- 2-oxo-2,5-dihydro-1H- pyrrole-3-carboxylic acid bis-pyridin-2- ylmethyl-amide | 381 |
| 5a₆b₃₄ | 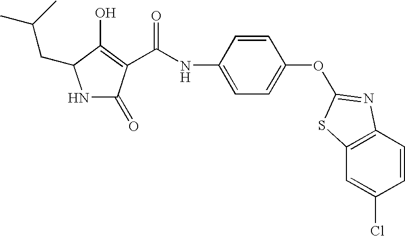 | 4-Hydroxy-5-isobutyl- 2-oxo-2,5-dihydro-1H- pyrrole-3-carboxylic acid [4-(6-chloro- benzothiazol-2-yloxy)- phenyl]-amide | 458 |
| 5a₆b₃₅ | 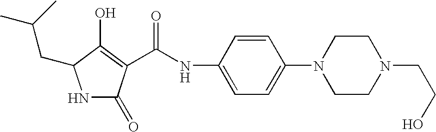 | 4-Hydroxy-5-isobutyl- 2-oxo-2,5-dihydro-1H- pyrrole-3-carboxylic acid{4-[4-(2-hydroxy- ethyl)-piperazin-1-yl]- phenyl}-amide | 403 |
| 5a₆b₃₆ | 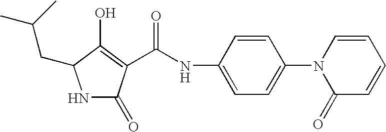 | 4-Hydroxy-5-isobutyl- 2-oxo-2,5-dihydro-1H- pyrrole-3-carboxylic acid [4-(2-oxo-2H- pyridin-1-yl)-phenyl]- amide | 368 |
| 5a₇b₁ | 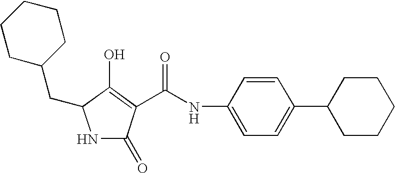 | 5-Cyclohexylmethyl-4- hydroxy-2-oxo-2,5- dihydro-1H-pyrrole-3- carboxylic acid (4- cyclohexyl-phenyl)- amide | 397 |
| 5a₇b₂ | 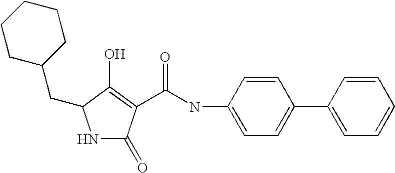 | 5-Cyclohexylmethyl-4- hydroxy-2-oxo-2,5- dihydro-1H-pyrrole-3- carboxylicacid biphenyl-4-ylamide | 391 |
| 5a₇b₃ | 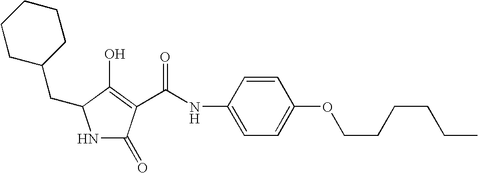 | 5-Cyclohexylmethyl-4- hydroxy-2-oxo-2,5- dihydro-1H-pyrrole-3- carboxylic acid (4- hexyloxy-phenyl)- amide | 415 |
| 5a₇b₁₃ | 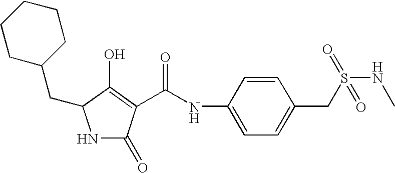 | 5-Cyclohexylmethyl-4- hydroxy-2-oxo-2,5- dihydro-1H-pyrrole-3- carboxylicacid(4methyl sulfamoylmethyl- phenyl)-amide | 422 |
| 5a₇b₁₅ | 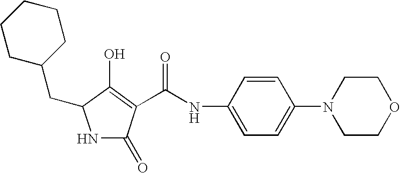 | 5-Cyclohexylmethyl-4- hydroxy-2-oxo-2,5- dihydro-1H-pyrrole-3- carboxylicacid (4- morpholin-4-yl- phenyl)-amide | 400 |
| 5a₇b₁₈ | 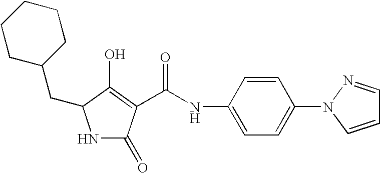 | 5-Cyclohexylmethyl-4- hydroxy-2-oxo-2,5- dihydro-1H-pyrrole-3- carboxylicacid (4- pyrazol-1-yl-phenyl)- amide | 381 |
| 5a₇b₂₀ | 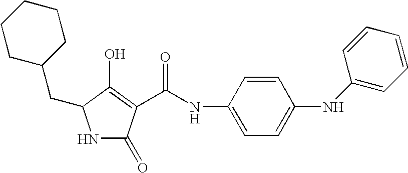 | 5-Cyclohexylmethyl-4- hydroxy-2-oxo-2,5- dihydro-1H-pyrrole-3- carboxylicacid (4- phenylamino-phenyl)- amide | 406 |
| 5a₇b₂₁ | 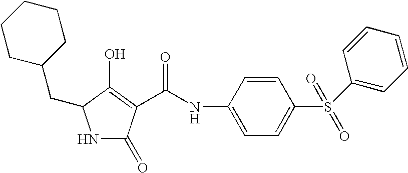 | 5-Cyclohexylmethyl-4- hydroxy-2-oxo-2,5- dihydro-1H-pyrrole-3- carboxylicacid(4benz- enesulfonyl-phenyl)- amide | 455 |
| 5a₇b₂₂ | 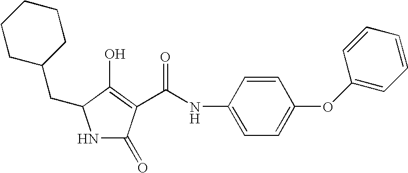 | 5-Cyclohexylmethyl-4- hydroxy-2-oxo-2,5- dihydro-1H-pyrrole-3- carboxylic acid (4- phenoxy-phenyl)- amide | 407 |
| 5a₇b₂₃ | 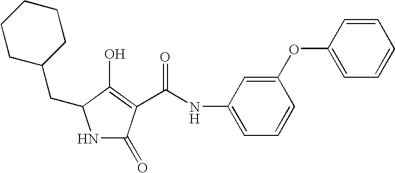 | 5-Cyclohexylmethyl-4- hydroxy-2-oxo-2,5- dihydro-1H-pyrrole-3- carboxylic acid (3- phenyoxy-phenyl)- amide | 407 |
| 5a₇b₂₆ | 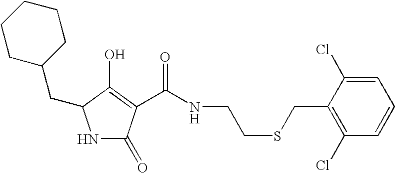 | 5-Cyclohexylmethyl-4- hydroxy-2-oxo-2,5- dihydro-1H-pyrrole-3- carboxylicacid [2-(2,6- dichloro- benzylsulfanyl)-ethyl]- amide | 458 |
| 5a₈b₁ | 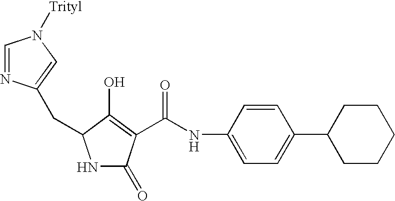 | 4-Hydroxy-2-oxo-5-(1- trityl-1H-imidazol-4- ylmethyl)-2,5 dihydro-1H-pyrrole-3- carboxylic acid(4- cyclohexyl-phenyl)- amide | 623 |
| 5a₈b₂ | 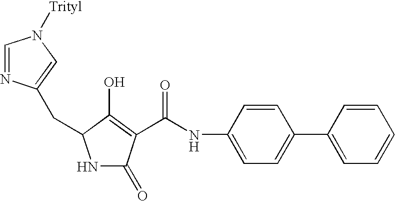 | 4-Hydroxy-2-oxo-5-(1- trityl-1H-imidazol-4- ylmethyl)-2,5 dihydro-1H-pyrrole-3- carboxylic acid biphenyl-4-yl-amide | 618 |
| 5a₉b₃ | 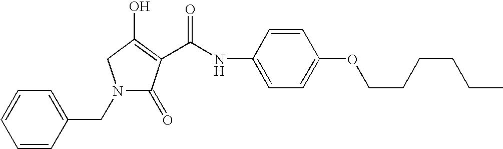 | 1-Benzyl-4-hydroxy-2- oxo-2,5-dihydro-1H- pyrrole-3-carboxylic acid (4-hexyloxy- phenyl)-amide | 409 |
| 5a₉b₄ | 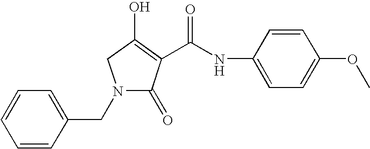 | 1-Benzyl-4-hydroxy-2- oxo-2,5-dihydro-1H- pyrrole-3-carboxylic acid (4-methoxy- phenyl)-amide | 339 |
| 5a₉b₁ | 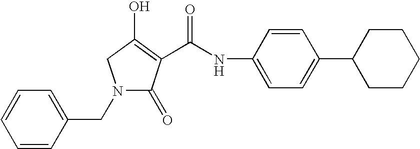 | 1-Benzyl-4-hydroxy-2- oxo-2,5-dihydro-1H- pyrrole-3-carboxylic acid (4-cyclohexyl- phenyl)-amide | 391 |
| 5a₁₀b₁ | 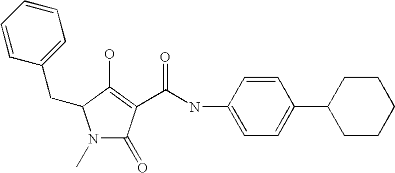 | 5-Benzyl-4-hydroxy-1- methyl-2-oxo-2,5- dihydro-1H-pyrrole-3- carboxylic acid (4- cyclohexyl-phenyl)- amide | 405 |

### MS and NMR Data for Selected Examples

### i. 4-Hydroxy-5-isobutyl-2-oxo-2,5-dihydro-1H-pyrrole-3-carboxylic acid (Phenylamino-phenyl)-amide

### 5a6b20, LCJ972

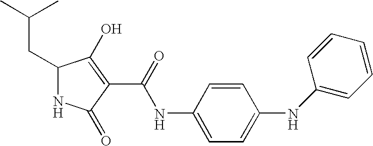

MS (ES+): m/z=366.44 (M+1) 1H NMR (400 MHz, DMSO-D6) δ=0.85 (dd, J=6.57, 3.03 Hz, 6H) 1.28 (ddd, J=13.64, 9.35, 4.80 Hz, 1H) 1.51 (ddd, J=13.52, 9.22, 4.04 Hz, 1H) 1.76 (dd, J=9.09, 5.05 Hz, 1H) 4.08 (d, J=7.07 Hz, 1H) 6.72 (t, J=7.33 Hz, 1H) 6.93; 7.01 (m, 4H) 7.14 (t, J=8.08 Hz, 2H) 7.40 (d, J=9.09 Hz, 2H) 8.01 (s, 1H)

### ii. 5-Benzyl-4-hydroxy-5-methyl-2-oxo-2,5-dihydro-1H-pyrrole-3-carboxylic acid (5-pyridin-2-yl-thiophen-2-ylmethyl)amide

### 5a4b10, LCH965

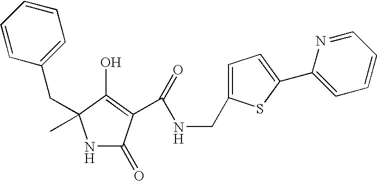

MS (ES+): m/z=420.51 (M+1) 1H NMR (400 MHz, CHLOROFORM-D) δ=1.47 (s, 3H) 3.01 (s, 2H) 4.66; 4.75 (m, 2H) 6.28 (s, 1H) 7.08 (d, J=4.04 Hz, 1H) 7.16 (s, 1H) 7.18 (d, J=2.02 Hz, 1H) 7.25; 7.33 (m, 3H) 7.44; 7.52 (m, 1H) 7.77 (d, J=8.08 Hz, 1H) 7.89 (d, J=3.54 Hz, 2H) 8.04 (t, J=7.83 Hz, 1H) 8.86 (d, J=4.55 Hz, 1H).

### iii. 5-Cyclohexylmethyl-4-hydroxy-2-oxo-2,5-dihydro-1H-pyrrole-3-carboxylic acid biphenyl-4-ylamide

### 5a7b2, LCJ440

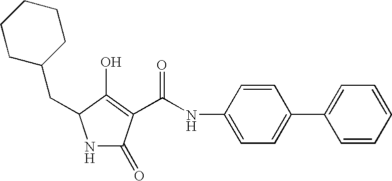

MS (ES+): m/z=391 (M+1) 1H NMR (400 MHz, CHLOROFORM-D) δ=0.99; 1.11 (m, 2H) 1.25; 1.34 (m, 3H) 1.46; 1.56 (m, 2H) 1.72; 1.83 (m, 6H) 1.86 (d, J=4.04 Hz, 1H) 4.29 (dd, J=8.84, 3.79 Hz, 1H) 5.60 (s, 1H) 7.35 (t, J=7.33 Hz, 1H) 7.46 (t, J=7.58 Hz, 2H) 7.61 (d, J=8.08 Hz, 4H) 7.71 (d, J=8.59 Hz, 2H)

### IV. General Procedure for Removal of the Trityl Group to Provide Examples 6a8 by Treatment of Compounds 5a8 with Trifluoro Acetic Acid

### Scheme 2

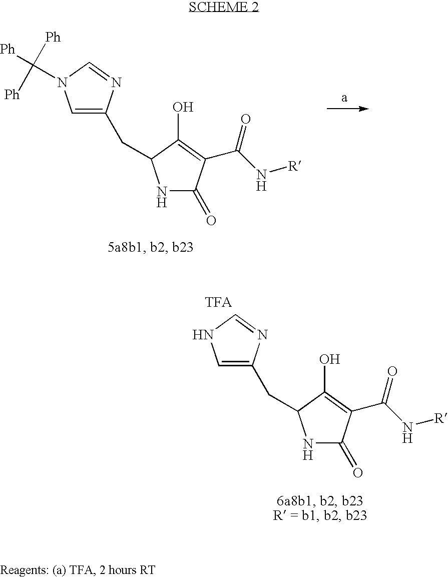

To amides 5a8 (0.2 mmol, 1 eq.) in THF was added TFA at rt, stirred for 120 minutes then evaporated to afford the crude product. The crude product was dissolved in THF for purification by reversed phase HPLC, 210 nm detection and 5%-95% elution with acetonitrile and water. The main fraction was collected and solvent was removed by lyophilization to afford a white powder.

### A. 4-Hydroxy-5 (3H-imidazol-4-ylmethyl)-2-oxo-2,5-dihydro-1H-pyrrole-3-carboxylic acid (4-cyclohexyl-phenyl)-amide

### 6a8b1

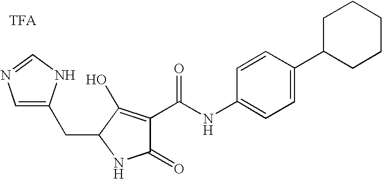

To a solution of 5a8b1 (12 mg, 0.02 mmol) in THF was added TFA and the mixture was stirred at rt for 2 hours, the resulting mixture was then concentrated in vacuum. The crude product was purified by HPLC to afford the title compound as a white solid (4.1 mg, 56%). MS (ES+): m/z=381.45 (M+1) 1H NMR (400 MHz, DMSO-D6) δ=1.27 (s, 1H) 1.35; 1.47 (m, 4H) 1.75 (d, J=11.62 Hz, 1H) 1.83 (dd, J=6.32, 2.78 Hz, 4H) 2.88 (d, J=6.57 Hz, 1H) 2.97 (d, J=6.06 Hz, 1H) 3.81 (t, J=6.06 Hz, 1H) 7.12 (d, J=8.08 Hz, 2H) 7.37 (s, 1H) 7.49 (d, J=8.59 Hz, 2H) 8.75 (s, 1H) 10.84 (s, 1H)

### B. 4-Hydroxy-5-(3H-imidazol-4-ylmethyl)-2-oxo-2,5-dihydro-1H-pyrrole-3-carboxylic acid biphenyl-4-ylamide

### 6a8b2

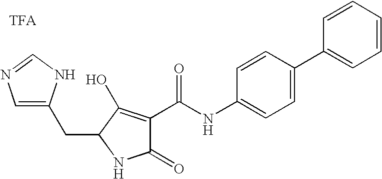

To as solution of 5a8b2 (15 mg, 0.024 mmol) in THF was added TFA and the resulting mixture was stirred at rt for 120 minutes then concentrated in vacuum. The residue was suspended in ether, collected by filtration, and rinsed with ether and methanol to afford the title compound as a white solid (7.3 mg, 61.3%). MS (ES+): m/z=489.42 (M+1) 1H NMR (400 MHz, MeOH-D4) δ=3.10 (dd, J=13.14, 5.05 Hz, 2H) 4.23 (s, 1H) 7.17 (dd, J=16.93, 7.83 Hz, 2H) 7.24; 7.35 (m, 3H) 7.44 (s, 4H) 7.58 (d, J=8.59 Hz, 2H) 8.65 (s, 1H)

### C. 4-Hydroxy-5-(3H-imidazol-4-ylmethyl)-2-oxo-2,5-dihydro-1H-pyrrole-3-carboxylic acid (3-phenoxy-phenyl)-amide

### 6a8b23

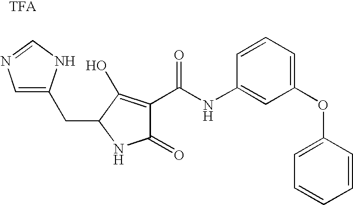

To a solution of 5a8b23 (15 mg, 0.024 mmol) was added TFA and the resulting mixture was stirred at RT for 120 minutes, then concentrated in vacuum. The residue was suspended in ether, collected by filtration, and rinsed with ether and methanol to afford the title compound as a white solid (1.3 mg, 13.8%). MS (ES+); m/z=391.38 (M+1)

### Example 2

### Preparation of Pyridine Compounds

The general synthetic preparation of pyridine compounds of the invention are described below.

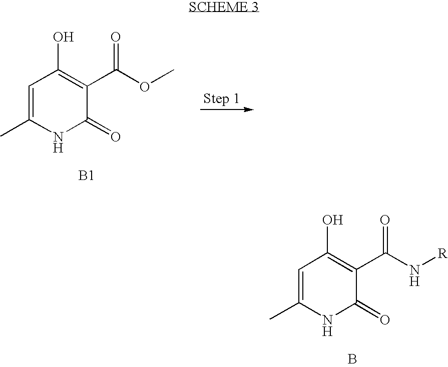

B2a. R=4-phenoxy-phenyl
B2b. R=4-cyclohexyl-phenyl
B2b. R=2-carbamoyl-phenyl
B2b. R=1,5-bis-(4-methoxy-phenyl)-1H-[1,2,4]triazol-3-yl

### I. Preparation of B2a

### Step 1

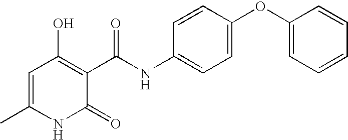

2,4-Dihydroxy-6-methyl-3-pyridinecarboxylic acid methyl ester B1 (100 mg, 0.50 mmol, Oakwood Chemical Company) and p-phenoxy aniline (188 mg, 1.00 mmol) were dissolved in tetrahydrofuran (6 mL). The resulting solution was heated using microwave irradiation at 180° C. for 10 min in a sealed tube. On cooling a precipitate formed. The solid was collected over a sintered glass funnel, washed with tetrahydrofuran and dried to provide 4-hydroxy-6-methyl-2-oxo-1,2-dihydro-pyridine-3-carboxylic acid (4-phenoxy-phenyl)-amide (B2a) as an off white amorphous powder (74 mg, 44%). ¹H-NMR (400 MHz, CHCl₃-d): δ ppm 2.23 (s, 3H), 6.00 (s, 1H), 7.03 (m, 4H), 7.13 (t, 1H, J=7 Hz), 7.39 (t, 2H, J=8 Hz), 7.62 (d, 2H, J=9 Hz), 11.96 (s, 1H), 12.47 (s, 1H) 15.06 (s, 1H). MS: m/z, (ES+)=337, (ES−)=335.

### II. Additional Compounds

The following compounds were prepared similarly:

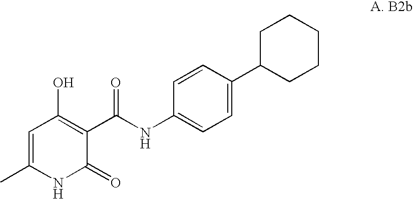

4-Hydroxy-6-methyl-2-oxo-1,2-dihydro-pyridine-3-carboxylic acid (4-cyclohexyl-phenyl)-amide. MS: m/z, (ES+)=327, (ES−)=325.

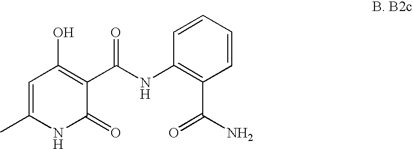

4-Hydroxy-6-methyl-2-oxo-1,2-dihydro-pyridine-3-carboxylic acid (2-carbamoyl-phenyl)-amide. MS: m/z, (ES+)=288, (ES−)=286.

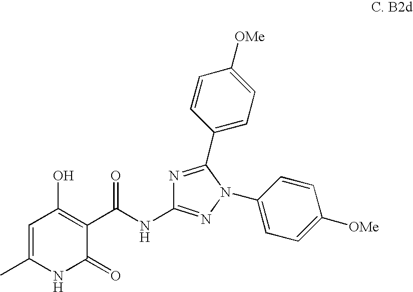

4-Hydroxy-6-methyl-2-oxo-1,2-dihydro-pyridine-3-carboxylic acid [1,5-bis-(4-methoxy-phenyl)-1H-[1,2,4]triazol-3-yl]-amide. MS: m/z, (ES+)=448, (ES−)=446

### Example 3

### Preparation of Spiro Piperidinyl Compounds

The general synthetic preparation of spiro piperidinyl compounds of the invention are described below.

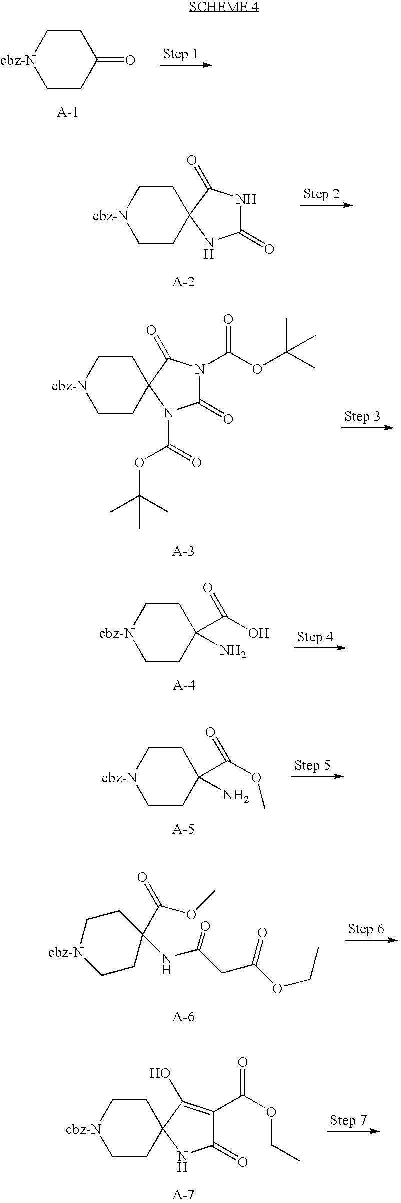

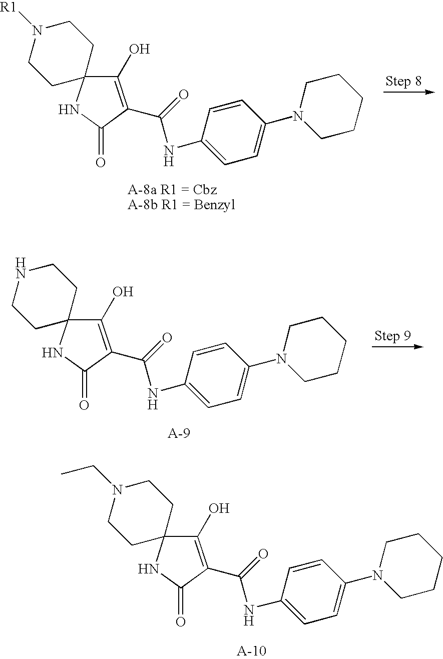

To a solution of benzyl-oxo-piperidone carboxylate (25 g, 107 mmol) in MeOH (110 mL) at room temperature in a pressure bottle, ammonium carbonate (20.5 g, 214 mmol) and 140 mL water were added. The mixture was stirred until all solid was dissolved. Potassium cyanide (13.9 g, 214 mmol) was added. The tube was sealed and stirred at room temperature for 90 hr. The resulting white solid was filtered and washed with water. Dried and collected white solid. Yield: 28.0 g (86%)

A mixture of A-2 (6.07 g, 20 mmol), di-butyl dicarbonate (17.4 g, 80 mmol), triethylamime (3.0 mL, 20 mmol) and DMAP (30 mg) in dry DME (200 ml) was stirred at room temperature over night. The solvent was removed. Solid was filtered and washed with diethyl ether to yield white solid. Yield: 8.5 g (84%)

To a solution of A-3 (8.2 g, 16.3 mmol) in THF (130 mL), 1.0N LiOH (130 mL 130 mmol) aqueous solution was added and the resulting mixture was stirred at room temperature over night. At this time, THF was removed, 130 mL (1.0N HCl) solution was added to the residue at 0 C. Removed some of water to 80 mL. Solid was filtered and dried to yield while solid. Yield: 3.6 g (79%)

To a solution of A-4 (2.0 g, 7.2 mmol) in MeOH (50 mL) at 0° C., thionyl chloride (1.7 g, 17.2 mmol) and 2 drops of DMF were added. The mixture was stirred at room temperature for 2 days. The solvent was removed to yield a light yellow oil. Yield: 2.1 g (89%)

To a solution of A-5 (0.7 g, 2.1 mmol), triethylamine (0.57 mL, 4.2 mmol) in dry THF (30 mL) and CH₂Cl₂ (30 mL), were added ethyl chloro oxo propionate (0.45 g, 3.0 mmol) at 0 C. The resulting mixture was stirred at room temperature over night. The crude was concentrated under reduced pressure and purified using silica-gel column chromatography to give the desired compounds A-6. Recrystallized to a white solid. Yield 0.65 g, (76%).

A mixture of A-6 (0.6 g, 1.5 mmol) in dry EtOH and 21% NaOEt in EtOH (1.9 g, 6.0 mmol) was stirred at room temperature over night. The crude was concentrated under reduced pressure. The residue was mixed with ice-water (10 mL) and 6.0 mL 1.0N HCl. The resulting solid was filtered and dried to yield light yellow solid. Yield: 440 mg (78%).

A mixture of A-7 (100 mg, 0.27 mmol) and 4-piperidinylaniline (48 mg, 0.27 mmol) in toluene (15 mL) was heated at reflux 62 hr. The crude was concentrated under reduced pressure and purified by HPLC to give the desired compounds A-8. Yield: 18 mg (10-20%).

To a solution of A-8 (250 mg, 0.5 mmol) in MeOH (40 mL) and CH₂Cl₂ (40 mL) in a 250 mL ROUND BOTTOM flask, were added 10% Pd/C (50 mg) and AcOH (0.5 mL). It was equipped with a Hydrogen filled balloon. The mixture was stirred at room temperature over night. The crude was filtered through a pad of celite and filtrate was concentrated under reduced pressure. The residue was crystallized to yield a white solid. Yield: 170 mg (90%).

To a solution of A-9 (74 mg, 0.20 mmol) in methanol (10 mL) were added acetaldehyde (20 mg, 0.44 mmol) and sodium cyanoborohydride (7 mg, 0.169 mmol) at room temperature. The resulting mixture was stirred overnight. The reaction mixture was concentrated under reduced pressure and the residue was crystallized from methanol to provide A-10. Yield: 66 mg.

A-8a ¹H-NMR (400 Mz, DMSO-d₆): δ 1.40 (m, 2H), 1.63 (m, 2H), 1.75 (m, 4H), 1.86 (m, 2H) 3.17 (m 4H), 4.07 (d, J=16 Hz, 2H), 5.21 (s, 2H), 7.01 (d, J=8 Hz, 2H), 7.47 (m, 5H), 7.50 (d, J=9.0 Hz, 2H), 4.48 (broad, 1H), 8.12 (broad, 1H), 10.57 (s, 1H); MS calcd for C₂₈H₃₂N₄O₅ 504, found ES⁺=505, ES⁻=503.

A-8b ¹H-NMR (400 Mz, DMSO-d₆): δ 1.07 (m, 2H), 1.43 (m, 2H), 1.55 (m, 4H), 1.76 (m, 2H) 2.20 (m, 2H), 2.65 (m, 2H), 2.93 (m, 4H), 3.40 (s, 2H), 6.72 (d, J=12, 2H), 6.90 (s, 1H), 7.16 (broad, 1H), 7.24 (m, 5H), 7.36 (d, J=9.0 Hz, 2H), 10.75 (s, 1H); MS calcd for C27H₃₂N₄O₃ 460, found ES⁺=461, ES⁻=459.

### Example 4

### Preparation of Spiro Pyrrolidinyl Compounds

The general synthetic preparation of spiro pyrrolidinyl compounds of the invention are described below.

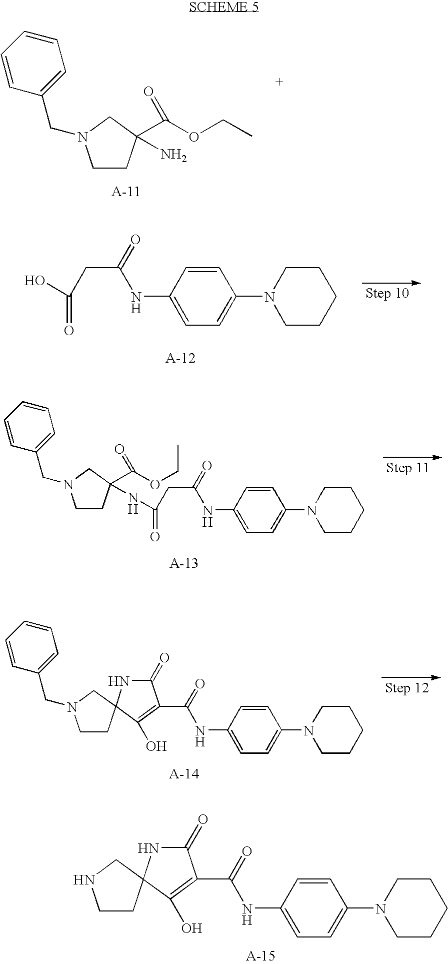

To a solution of A-11 (250 mg, 1.0 mmol), and A-12 (262 mg, 11.0 mmol) and diisopropylethylamine (0.35 mL, 2.0 mmol) in dry DMF (10 mL), was added EDCl (200 mg, 1.05 mmol). The mixture was stirred at room temperature over night. The crude was concentrated under reduced pressure and purified using silica-gel column chromatography to give the desired compounds A-13. Recrystallized to a white solid. Yield 0.300 mg, (65%).

A mixture of A-13 (350 mg, 0.71 mmol) in dry EtOH and 21% NaOEt in EtOH (920 mg, 2.84 mmol) was stirred at room temperature over night. The crude was concentrated under reduced pressure. The residue was mixed with ice-water (10 mL) and 2.84 mL 1.0N HCl. The resulting solid was filtered and dried to yield light yellow solid. Yield: 280 mg (90%).

To a solution of A-14 (250 mg, 0.56 mmol) in MeOH (50 mL) and CH₂Cl₂ (50 mL) in a 250 mL ROUND BOTTOM flask, were added 20% Pd(OH)2/C (50 mg) and AcOH (0.5 mL). It was equipped with a Hydrogen filled balloon. The mixture was stirred at room temperature over night. The crude was filtered through a pad of celite and filtrate was concentrated under reduced pressure. The residue was crystallized to yield a white solid. Yield: 160 mg (90%).

A-14 ¹H-NMR (400 Mz, DMSO-d₆): δ 0.75 (m, 2H), 0.81 (m, 1H), 0.98 (m, 2H), 1.24 (m, 2H), 1.36 (m, 4H), 1.45 (m, 2H), 1.84 (m, 1H), 2.45 (m, 2H) 2.65 (m, 1H), 3.43 (s, 1H), 5.31 (d, J=8.0 Hz, 1H), 6.54 (d, J=8.0, 2H), 6.62 (broad, 1H), 7.00 (broad, 1H), 7.07 (m, 1H), 7.11 (m, 5H), 10.49 (s, 1H); MS calcd for C₂₆H₃₀N₄O₃ 446, found ES⁺=447, ES⁻=445.

### Example 5

### Preparation of Additional Spiro Piperidinyl Compounds

The general synthetic preparation of additional spiro piperidinyl compounds of the invention are described below.

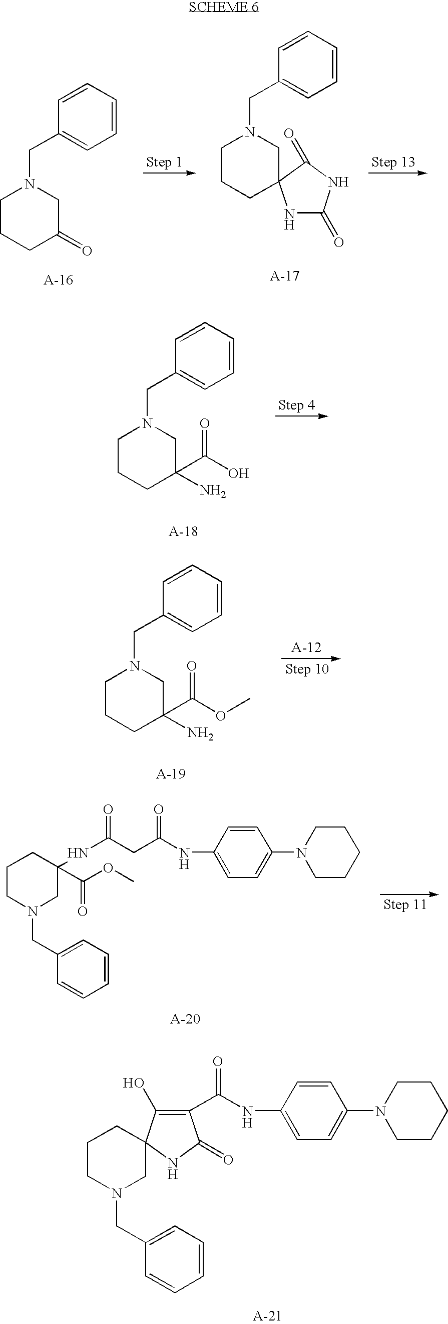

(Steps 1-12 are described above in Examples 3 and 4).

A mixture of A-17 (1.2 g, 4.63 mmol) in 10 mL concentrate HCl (12N) at room temperature in a sealed pressure bottle was heated at 160 C for 12 hr. The crude was concentrated and dried under reduced pressure to give the desired compounds A-18. Yield: 1.21 g, (90%).

### Example 6

### Preparation of Monocyclic Hydroxydicarbonyl Compounds

The general synthetic preparation of additional monocyclic hydroxydicarbonyl compounds of the invention are described below.

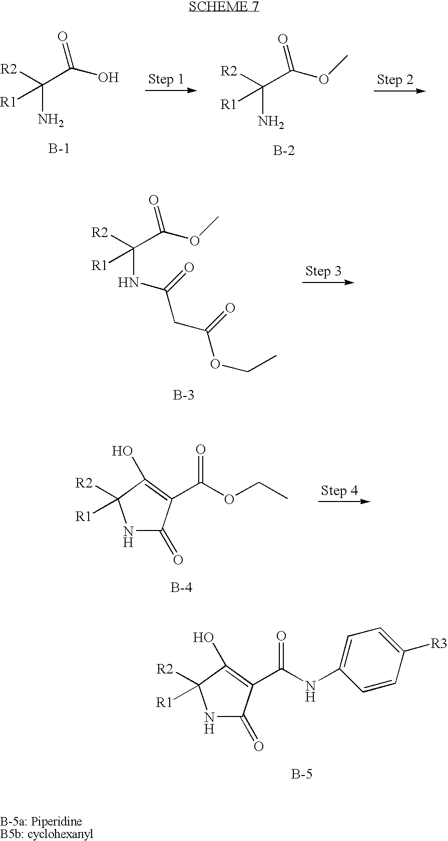

To a solution of B-1 (2.0 g, 7.2 mmol) in MeOH (50 mL), was added concentrate hydrochloric acid (12N, 1 mL). The mixture was heated at reflux overnight. The crude was concentrated and dried under reduced pressure to give the desired compound. Yield: 2.1 g (89%)

To a solution of B-2 (3.45 g, 15 mmol), triethylamine (4.2 mL, 30 mmol) in dry THF (80 mL) was added ethyl chloro oxo propionate (2.38 g, 15.8 mmol) at 0 C. The resulting mixture was stirred at room temperature over night. The crude was concentrated under reduced pressure and purified using silica-gel column chromatography to give the desired compounds B-3. Recrystallized to a white solid. Yield 4.0 g, (76%).

A mixture of B-3 (1.8 g, 5.8 mmol) in dry EtOH and 21% NaOEt in EtOH (7.5 g, 23.2 mmol) was stirred at room temperature over night. The crude was concentrated under reduced pressure. The residue was mixed with ice-water (10 mL) and 24 mL 1.0N HCl. The resulting solid was filtered and dried to yield light yellow solid. Yield: 1.2 (78%).

A mixture of B-4 (100 mg, 0.36 mmol) and N-(4-aminophenyl)piperidine (64 mg, 0.36 mmol) were dissolved in THF (4 mL). The resulting solution was heated (100-120 C) using microwave for 6-20 mins. The crude was concentrated under reduced pressure and purified using HPLC to give the desired compounds B-5

B5a ¹H-NMR (400 Mz, DMSO-d₆): δ 1.24 (s, 3H), 1.33 (m, 2H), 1.46 (m, 4H), 2.68 (d, J=16 Hz, 1H), 2.83 (d, J=12 Hz, 1H), 2.93 (m, 4H), 6.74 (d, J=8 Hz, 2H), 7.03 (m, 2H), 7.06 (m, 1H), 7.09 (m, 2H), 7.20 (d, J=9.0 Hz, 2H), 4.48 (broad, 1H), 8.18 (broad, 1H), 9.63 (s, 1H); MS: calcd for C₂₄H₂₇N₃O₃ 405, found ES⁺=406, ES⁻=404.

B-5b ¹H-NMR (400 Mz, DMSO-d₆): δ 1.20 (s, 3H), 1.22 (m, 1H), 1.34 (m, 4H), 1.67 (d, J=12 Hz, 2H), 1.75 (m, 3H) 2.40 (m, 1H), 2.65 (d, J=12 Hz, 1H), 2.87 (d, J=12 Hz, 1H), 7.04 (d, J=12 Hz, 2H), 7.14 (m, 5H), 7.42 (d, J=8.0 Hz, 2H), 4.48 (broad, 1H), 6.87 (broad, 3H), 10.74 (s, 1H); MS calcd for C₂₅H₂₈N₂O₃ 404, found ES⁺405, ES⁻=403.

### Example 7

### Preparation of Monocyclic Hydroxydicarbonyl Compounds

The general synthetic preparation of additional monocyclic hydroxydicarbonyl compounds of the invention are described below.

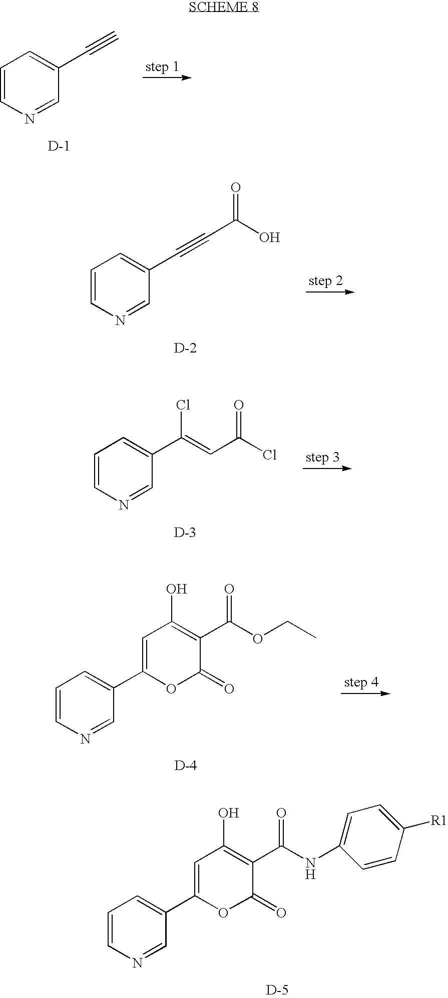

To a solution of D-1 3-ethyne pyridine (8.0 g, 77.6 mmol) in THF (150 mL) at −78° C., n-BuLi (1.6 M in hexanes, 54 mL, 85.3 mmol) was added dropwise (keeping the reaction temperature below −60° C.). It was stirred at this temperature for another 2 hrs and warmed up to 0° C. It was cooled to −30 C again and a fresh chopped dry ice was added. It was stirred and allowed to warm up to 0 C and 20 mL 4.0N NaOH was added. Organic layer was separated. Aqueous layer was acidified to ph<1. Solid was filtered to yield the final product. Yield: 6.0 g

To a solution of D-2 (0.5 g, 3.4 mmol) in CH2Cl2 (50 mL) at 0° C., Oxalyl chloride (0.86 g, 6.8 mmol) and 2 drops of DMF were added. The mixture was stirred at reflux over night. The solvent was removed to yield a light yellow oil.

To a solution of diethyl malonate (0.6 g, 3.74 mmol) in Xylene (40 mL), sodium metal (0.086 g, 3.74 mmol). The mixture was stirred at reflux until all solid was dissolved. The mixture was cooed and D-3 (3.4 mmol) was added. The mixture stirred at reflux over night. The crude was concentrated under reduced pressure and purified using silica-gel column chromatography to give the desired compounds. Yield 0.3 g.

A mixture of D-4 (140 mg, 0.57 mmol) and 4-piperidinylaniline (200 mg, 1.14 mmol) in toluene (15 mL) was heated at reflux over night. The crude was concentrated under reduced pressure and purified using silica-gel column chromatography and HPLC separation to give the desired compounds D5. Yield: 10 mg.

D-5a ¹H-NMR (400 Mz, DMSO-d₆): δ 1.30 (m, 4H), 1.22 (m, 2H), 2.60 (m, 2H), 2.66 (s, 1H), 2.82 (m, 1H), 6.25 (broad, 1H), 6.48 (t, J=8.0 Hz, 1H), 6.65 (broad, 2H), 7.22 (s, 1H), 7.36 (m, 2H), 8.11 (broad t, 1H), 8.52 (bs, 1H), 8.93 (broad d, 1H); MS calcd for C₂₂H₂₁N₃O₄, 391, found ES⁺=392, ES⁻=390.

### Example 8

### Preparation of Monocyclic Hydroxydicarbonyl Compounds

The general synthetic preparation of additional monocyclic hydroxydicarbonyl compounds of the invention are described below.

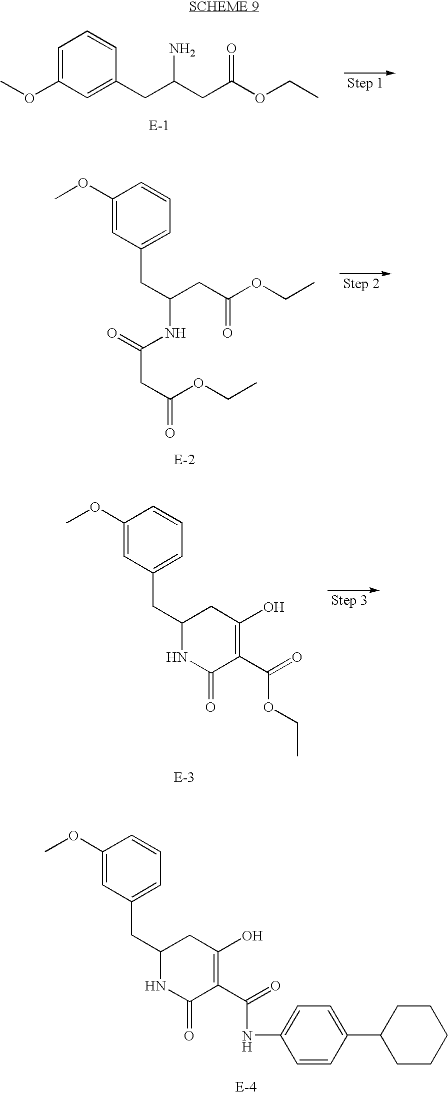

For specific methodology, see Example 6.

E-4 ¹H-NMR (400 Mz, CDCl₃-d₆): δ 1.18 (m, 1H), 1.29 (m, 4H), 1.65 (d, J=9.0 Hz, 1H), 1.78 (m, 5H), 2.40 (m, 1H), 2.53 (m, 1H), 2.62 (m, 1H), 2.66 (m, 1H), 2.76 (m, 1H), 3.75 (s, 3H), 3.78 (m, 1H), 5.16 (s, 1H), 6.66 (s, 1H), 6.73 (dd, J1=4.0 Hz, J2=20 Hz, 2), 7.12 (d, J=8.0 Hz, 2H), 7.30 (dd, J1=8.0 Hz, J2=48 Hz, 1H), 7.38 (d, J=8.0 Hz, 2H), 11.50 (s, 1H); MS calcd for C₂₆H₃₀N₂O₄, 434, found ES⁺=435, ES⁻=433.

### Example 9

### Preparation of Additional Monocyclic Hydroxydicarbonyl Compounds

The general synthetic preparation of additional monocyclic hydroxydicarbonyl compounds of the invention are described below.

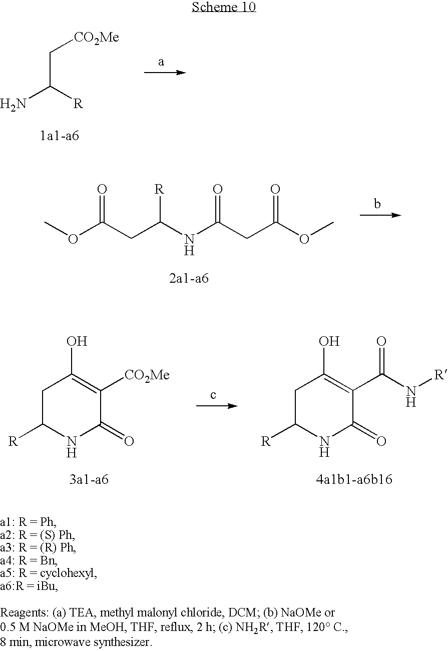

### I. Synthesis of Intermediates

### A. Methyl-3-[(3-methoxy-3-oxo-1-phenylpropyl)amino]-3-oxopropanoate

### 2a1

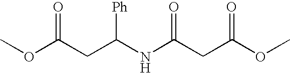

To a stirred solution of methyl 3-phenyl-amino-propionate (5.2 g, 24 mmol) and triethylamine (3.4 mL, 24 mmol) in dichloromethane (125 mL) was added methyl malonyl chloride (2.6 mL, 24 mmol) portionwise at 0° C. under N₂ atmosphere. The reaction mixture was stirred for further 16 h, then diluted with 250 mL dichloromethane. The organic solution was washed with water and brine, dried over Na₂SO₄. The solvent was then removed to give the title compound as a yellow solid (7.1 g, 100%). The material was used without further purification in the next step. MS (ES+): m/z=280 (M+1) 1H NMR (400 MHz, CHLOROFORM-D) δ=2.82-2.89 (m, 1H) 2.91-2.97 (m, 1H) 3.36 (d, J=2.53 Hz, 2H) 3.62 (s, 3H) 3.75-3.81 (m, 3H) 5.46 (d, J=8.08 Hz, 1H) 7.24-7.36 (m, 6H) 8.04 (d, J=7.58 Hz, 1H)

### B. Methyl 3-{[(1S)-3-ethoxy-3-oxo-1-phenylpropyl]amino}-3-oxopropanoate

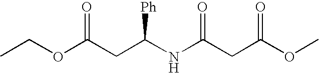

Analogous to 2a1 compound 2a2 was prepared from 2.3 g of 1a2 to yield 2.8 g (95% yield) of the title compound as a yellow solid. MS (ES+): m/z=294 (M+1)

### C. Methyl 3-{[(1R)-3-ethoxy-3-oxo-1-phenylpropyl]amino}-3-oxopropanoate

### 2a3

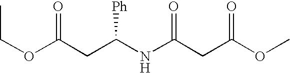

Analogous to 2a1 compound 2a3 was prepared from 3 g of 1a3 to yield 3.7 g (92% yield) of the title compound as a yellow solid, MS (ES+): m/z=294 (M+1)

### D. Methyl 3-[(3-ethoxy-3-oxopropanoyl)amino]-4-phenylbutanoate

### 2a4

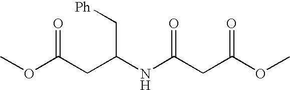

Analogous to 2a1 compound 2a4 was prepared from 5.2 g of 1a4 to yield 8.0 g (100% yield) of the title compound as a yellow solid, MS (ES+): m/z=294 (M+1)

### E. Methyl 3-cyclohexyl-3-[(3-ethoxy-3-oxopropanoyl)amino]propanoate

### 2a5

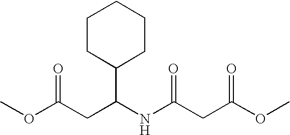

Analogous to 2a1 compound 2a5 was prepared from 4 g of 1a5 to yield 4.7 g (76% yield) of the title compound as a light yellow solid, MS (ES+): m/z=286 (M+1)

### F. Methyl 3-[(3-Methoxy-3-oxopropanoyl)amino]-5-methylhexanoate

### 2a6

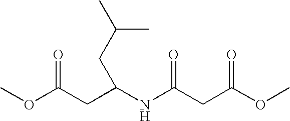

Analogous to 2a1 compound 2a6 was prepared from 3.6 g of 1a6 to yield 5.5 g (89% yield) of the title compound as a yellow oil. MS (ES+): m/z=260 (M+1)

### G. Methyl-4-hydroxy-2-oxo-6-phenyl-1,2,5,6-tetrahydro-pyridine-3-carboxylate

### 3a1

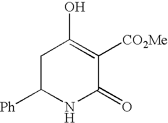

To a stirred solution of 2a1 (2 g, 7.2 mmol) in anhydrous THF (30 mL) was added sodium methoxide (0.4 g, 7.2 mmol) portionwise under N2 atmosphere. The resulting mixture was refluxed for 2 h, then concentrated in vacuo. The resulting residue was suspended in 50 mL ether and filtered, then the precipitate was dissolved in 50 mL water. The aqueous solution was adjusted to pH 2 with 1 N HCl and extracted with EtOAc (3 times). The organic phase was combined, dried over NaSO₄, and concentrated under the reduced pressure to afford the title compound as a yellow oil (1.7 g, 77%). MS (EST): m/z=248 (M+1) 1H NMR (400 MHz, CHLOROFORM-D) δ=2.92 (s, 2H) 3.92 (s, 3H) 4.70 (dd, J=10.61, 5.05 Hz, 1H) 5.80 (b, 1H) 7.28-7.41 (m, 5H) 14.13 (s, 1H)

### H. Methyl (6S)-4-hydroxy-2-oxo-6-phenyl-1,2,5,6-tetrahydropyridine-3-carboxylate (3a2)

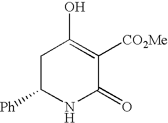

Analogous to 3a1 compound 3a2 was prepared from 2.8 g of 2a2 to yield 1.0 g (42% yield) of the title compound as a yellow solid. MS (ES+): m/z=248 (M+1)

### I. Methyl (6R)-4-hydroxy-2-oxo-6-phenyl-1,2,5,6-tetrahydropyridine-3-carboxylate

### 3a3

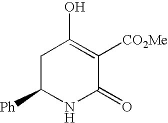

Analogous to 3a1 compound 3a3 was prepared from 1 g of 2a3 to yield 450 mg (48% yield) of the title compound as a yellow solid. MS (ES+): m/z=248 (M+1)

### J. Methyl 6-benzyl-4-hydroxy-2-oxo-1,2,5,6-tetrahydropyridine-3-carboxylate

### 3a4

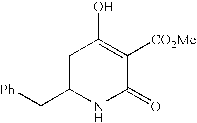

Analogous to 3a1 compound 3a4 was prepared from 180 mg of 2a4 to yield 154 mg (77% yield) of the title compound as a yellow solid. MS (ES+): m/z=262 (M+1)

### K. Methyl 6-cyclohexyl-4-hydroxy-2-oxo-1,2,5,6-tetrahydropyridine-3-carboxylate

### 3a5

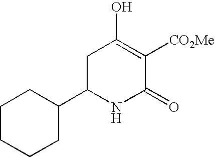

Analogous to 3a1 compound 3a5 was prepared from 1.1 g of 2a5 to yield 500 mg (46% yield) of the title compound as a yellow solid, MS (ES+): m/z=254 (M+1)

### L. Methyl 4-hydroxy-6-isobutyl-2-oxo-1,2,5,6-tetrahydropyridine-3-carboxylate

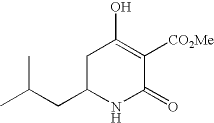

Analogous to 3a1 compound 3a6 was prepared from 2 g of 2a6 to yield 200 mg (12% yield) of the title compound as a yellow oil. MS (ES+): m/z=228 (M+1)

### II. Synthesis of Examples

### A. General Procedure for the Formation of Amides 4 with Amines and Esters 3a

To a solution of the ester 3a (0.2 mmol, 1 eq.) in THF was added the amine (0.2 mmol, 1 eq.) and the mixture was heated in the microwave synthesizer (Biotage Initiator) at 150°for 5 min and concentrated in vacuo afterwards. The residue was triturated with ether and methanol to afford amides 4. The crude material can also be diluted in DCM (40 mL) and washed with water and brine. The organic phase was dried over sodium sulfate and concentrated and the mixture was purified by automated flash chromatography (Biotage), eluting with 10-30% EtOAc and hexane to afford amides 4.

### i. N-biphenyl-4-yl-4-hydroxy-2-oxo-6-phenyl-1,2,5,6-tetrahydropyridine-3-carboxamide

### 4a1b2

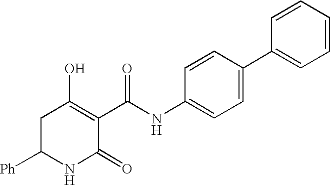

To a solution of 3a1 (52 mg, 0.2 mmol) in THF was added 4-aminobiphenyl (0.4 mL, 0.2 mmol, 0.5 M in DMF) and the resulting mixture was heated in the microwave synthesizer at 150° C. for 5 min, then concentrated in vacuo. The residue was suspended in ether, collected by filtration, and rinsed with ether and methanol to afford the title compound as a white solid (27.7 mg, 43%). MS (ES+): m/z=385 (M+1) 1H NMR (400 MHz, DMSO-D6) δ=2.69-2.77 (m, 0.5H) 2.83-2.91 (m, 1H) 3.13 (dd, J=17.18, 6.06 Hz, 0.5H) 4.77 (td, J=6.82, 2.53 Hz, 0.5H) 4.83-4.89 (m, 0.5H) 7.30-7.37 (m, 2H) 7.39 (t, J=3.79 Hz, 4H) 7.42-7.48 (m, 2H) 7.58-7.61 (m, 1H) 7.64-7.70 (m, 5H) 8.28 (d, J=2.02 Hz, 0.5H) 9.34 (s, 0.5H) 12.01 (s, 0.5H) 12.27 (s, 0.5H)

This compound series (4) exists as two tautomers (roughly 1:1) in DMSO-d6.

### B. General Procedure for the Sodium Salt Formation of Compounds 4

A solution of 4a (0.47-0.50 mmol, 1 eq) in EtOH was charged with NaOH 1 M aqueous solution (42-45 μL, 0.9 eq). The mixture was heated in the microwave synthesizer at 100° C. for 3 min. Solid precipitated out after sitting at room temperature for 16 h. The desired salt was collected by filtration and rinsed with cold EtOH.

### i. Sodium N-biphenyl-4-yl-4-hydroxy-2-oxo-6-phenyl-1,2,5,6-tetrahydropyridine-3-carboxamide

### 4a1b2-sodium salt

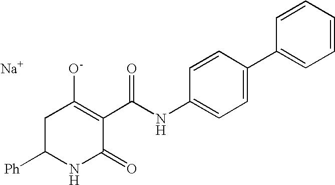

According the general protocol above the sodium salt of N-biphenyl-4-yl-4-hydroxy-2-oxo-6-phenyl-1,2,5,6-tetrahydropyridine-3-carboxamide was formed.

### ii. Magnesium N-biphenyl-4-yl-4-hydroxy-2-oxo-6-phenyl-1,2,5,6-tetrahydropyridine-3-carboxamide

### 4a1b2-magnesium salt

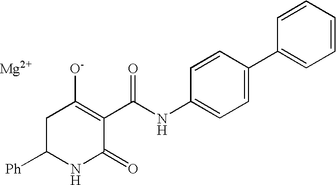

To a solution of 4a1b1 (46 mg, 0.18 mmol, 1 eq) in 2 ml EtOH was added Mg(OH)₂ (3.5 mg, 0.06 mmol, 0.5 eq) and 0.25 mL water. The mixture was heated in the microwave synthesizer at 100° C. for 15 min. Solid precipitated out after sitting at room temperature for 1 h. The desired salt was collected by filtration and rinsed with cold EtOH.

The compounds in the following fable were prepared according to the general procedure described above:

| | | | MS: |
|---|---|---|---|
| Compound | | | m/z |
| # | Structure | Name | (M + 1) |
| 4a₁b₁ | 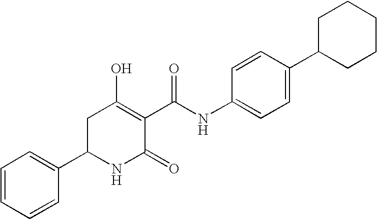 | N-(4-cyclohexylphenyl)- 4-hydroxy-2-oxo- 6-phenyl-1,2,5,6- tetrahydropyridine- 3-carboxamide | 391 |
| 4a₁b₃ | 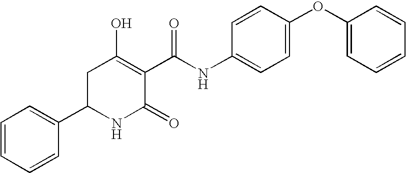 | 4-hydroxy-2-oxo- N-(4- phenoxyphenyl)- 6-phenyl-1,2,5,6- tetrahydropyridine- 3-carboxamide | 401 |
| 4a₁b₆ | 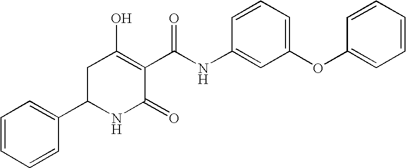 | 4-hydroxy-2-oxo- N-(3- phenoxyphenyl)- 6-phenyl-1,2,5,6- tetrahydropyridine- 3-carboxamide | 401 |
| 4a₁b₇ | 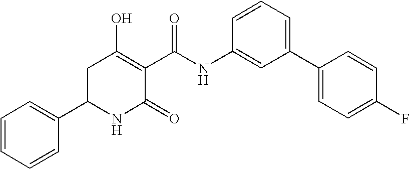 | N-(4′- fluorobiphenyl)-3- yl)-4-hydroxy-2- oxo-6-phenyl- 1,2,5,6- tetrahydropyridine- 3-carboxamide | 403 |
| 4a₁b₉ | 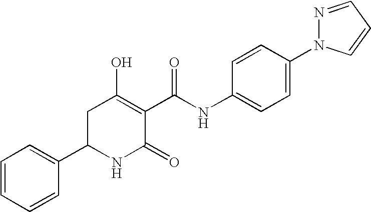 | 4-hydroxy-2-oxo- 6-phenyl-n-[4- (1H-pyrazol-1- yl)phenyl]-1,2,5,6- tetrahydropyridine- 3-carboxamide | 375 |
| 4a₁b₁₀ | 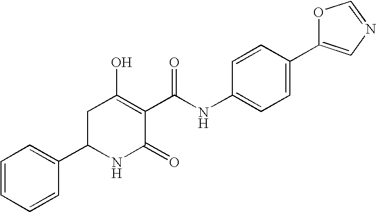 | 4-hydroxy-N-[4- (1,3-oxazol-5- yl)phenyl]-2-oxo- 6-phenyl-1,2,5,6- tetrahydropyridine- 3-carboxamide | 376 |
| 4a₁b₁₁ | 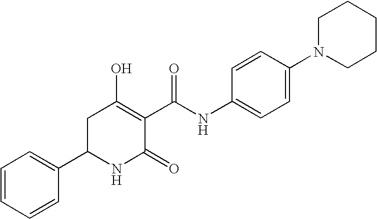 | 4-hydroxy-2-oxo- 6-phenyl-N-(4- piperidin-1- ylphenyl)-1,2,5,6- tetrahydropyridine- 3-carboxamide | 392 |
| 4a₁b₁₄ | 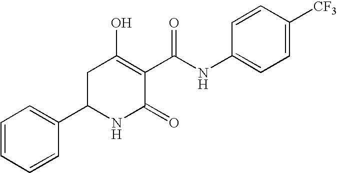 | 4-hydroxy-2-oxo- 6-phenyl-N-(4- trifluo methylphenyl)- 1,2,5,6- tetrahydropyridine- 3-carboxamide | 377 |
| 4a₁b₁₄- sodium salt | 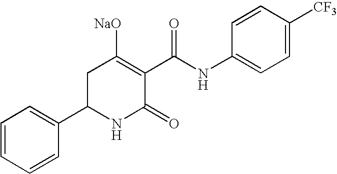 | Sodium 4- hydroxy-2-oxo-6- phenyl-N-(4- trifluoro methylphenyl)- 1,2,5,6- tetrahydropyridine- 3-carboxamide | 377 |
| 4a₁b₁₅ | 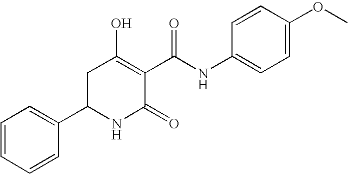 | 4-hydroxy-N-(4- methoxy-phenyl)- 2-oxo-6-phenyl- 1,2,5,6- tetrahydropyridine- 3-carboxamide | 339 |
| 4a₁b₁₉ | 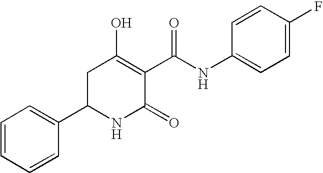 | N-(4- fluorophenyl-4- hydroxy-2-oxo-6- phenyl-1,2,5,6- tretrahydro pyridine- 3-carboxamide | 327 |
| 4a₂b₁ | 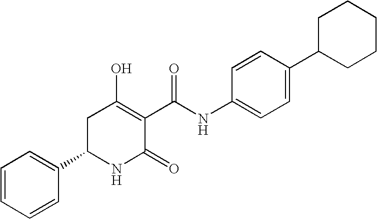 | (6S)-N-(4- cyclohexylphenyl)- 4-hydroxy-2-oxo- 6-phenyl-1,2,5,6- tetrahydropyridine- 3-carboxamide | 391 |
| 4a₂b₂ | 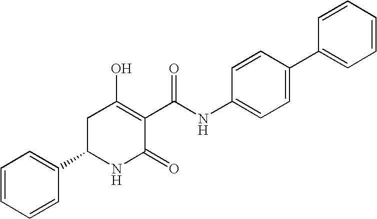 | (6S)-N-biphenyl)- 4-yl-4-hydroxy-2- oxo-6-phenyl- 1,2,5,6- tetrahydropyridine- 3-carboxamide | 385 |
| 4a₃b₁ | 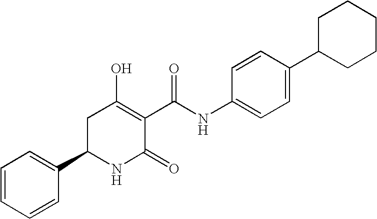 | (6R)-N-(4- cyclohexylphenyl)- 4-hydroxy-2-oxo- 6-phenyl-1,2,5,6- tetrahydropyridine- 3-carboxamide | 391 |
| 4a₃b₂ | 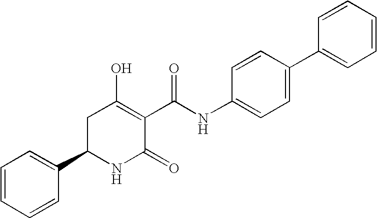 | (6R)-N-biphenyl)- 4-yl-4-hydroxy-2- oxo-6-phenyl- 1,2,5,6- tetrahydropyridine- 3-carboxamide | 385 |
| 4a₄b₁ | 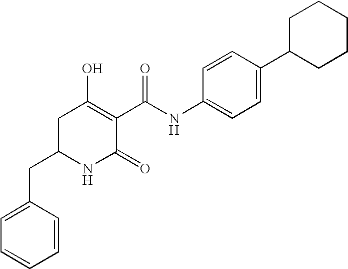 | N-(4- cyclohexylphenyl)- 6-benzyl-4- hydroxy-2-oxo- 1,2,5,6- tetrahydropyridine- 3-carboxamide | 405 |
| 4a₄b₂ | 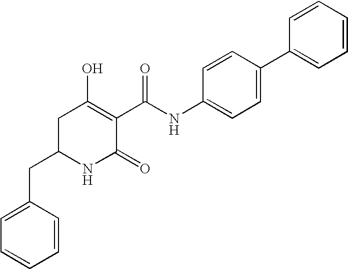 | N-biphenyl-4-yl- 6-benzyl-4- hydroxy-2-oxo- 1,2,5,6- tetrahydropyridine- 3-carboxamide | 399 |
| 4a₄b₂- sodium salt | 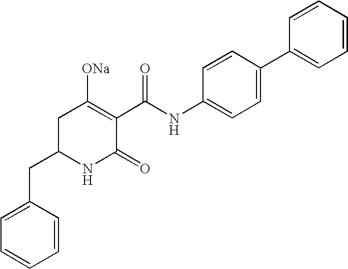 | Sodium N- biphenyl-4-yl-6- benzyl-4-hydroxy- 2-oxo-1,2,5,6- tetrahydro pyridine-3 carboxamide | 399 |
| 4a₄b₃ | 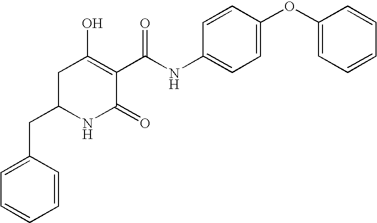 | 6-benzyl-4- hydroxy-2-oxo-N- (4-phenoxy phenyl)-1,2,5,6- tetrahydropyridine- 3-carboxamide | 415 |
| 4a₄b₄ | 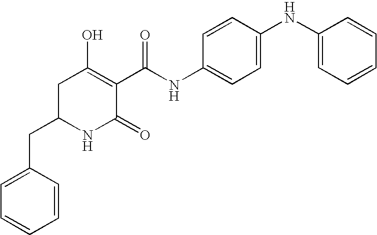 | N-(4- anilinophenyl)-6- benzyl-4-hydroxy- 2-oxo-1,2,5,6- tetrahydropyridine- 3-carboxamide | 414 |
| 4a₄b₅ | 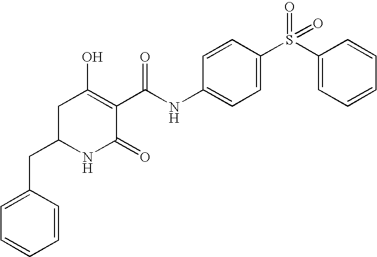 | 6-benzyl-4- hydroxy-2-oxo-N- [4- (phenylsulfonyl) phenyl]-1,2,5,6- tetrahydropyridine- 3-carboxamide | 463 |
| 4a₄b₈ | 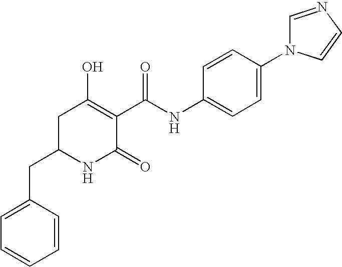 | 6-benzyl-4- hydroxy-N-[4- (1H-imidazol-1- yl)phenyl]-2-oxo- 1,2,5,6- tetrahydropyridine- 3-carboxamide | 389 |
| 4a₄b₈- sodium salt | 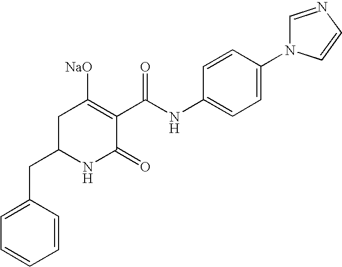 | Sodium 6-benzyl- 4-hydroxy-N-[4- (1H-imidazol-1- yl)phenyl]-2-oxo- 1,2,5,6-tetrahydro pyridine-3- carboxamide | 389 |
| 4a₄b₁₀ | 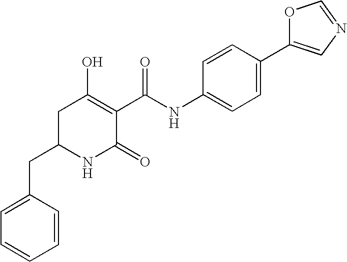 | 6-benzyl-4- hydroxy-N-[4- (1,3-oxazol-5- yl)phenyl]-2-oxo- 1,2,5,6- tetrahydropyridine- 3-carboxamide | 390 |
| 4a₄b₁₁ | 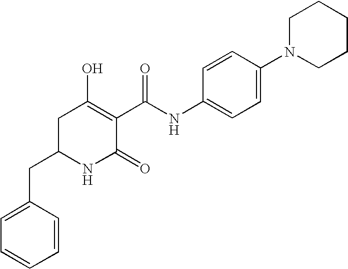 | 6-benzyl-4- hydroxy-2-oxo-N- (4-piperidin-1- ylphenyl)-1,2,5,6- tetrahydropyridine- 3-carboxamide | 406 |
| 4a₄b₁₂ | 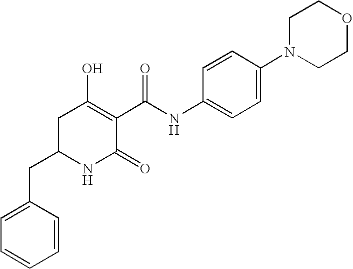 | 6-benzyl-4- hydroxy-2-oxo-N- (4-morpholin-4- ylphenyl)-1,2,5,6- tetrahydropyridine- 3-carboxamide | 408 |
| 4a₄b₁₃ | 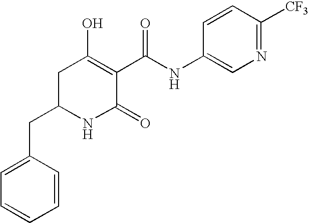 | 6-benzyl-4-hydroxy- 2-oxo-N-(6- trifluoromethyl-pyridin-3-yl)-1,2,5,6- tetrahydropyridine- 3-carboxamide | 392 |
| 4a₄b₁₄ | 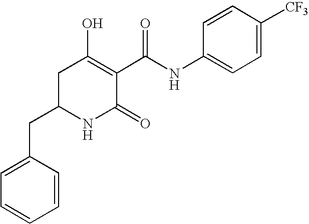 | 6-benzyl-4- hydroxy-2-oxo-N- (4- trifluoromethyl- phenyl)-1,2,5,6- tetrahydropyridine- 3-carboxamide | 391 |
| 4a₄b₁₅ | 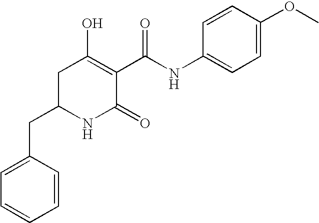 | 6-benzyl-4- hydroxy-N-(4- (methoxy-phenyl)- 2-oxo-1,2,5,6- tetrahydropyridine- 3-carboxamide | 353 |
| 4a₄b₁₇ | 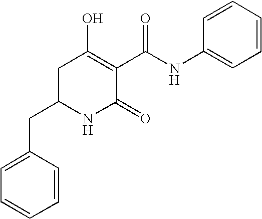 | 6-benzyl-N- cyclohexyl-4- hydroxy-2-oxo- 1,2,5,6- tetrahydropyridine- 3-carboxamide | 329 |
| 4a₄b₁₈ | 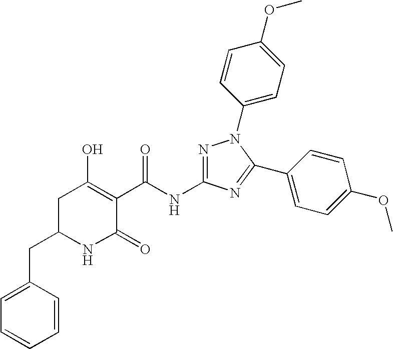 | 6-benzyl-N-[1,5- bis(4- methoxyphenyl)- 1H-1,2,4-triazol- 3-yl]-4-hydroxy- 2-oxo- -1,2,5,6- tetrahydropyridine- 3-carboxamide | 526 |
| 4a₅b₁ | 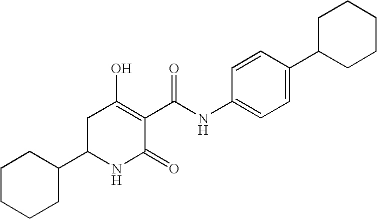 | 6-cyclohexyl-N-(4- cyclohexylphenyl)- 4-hydroxy-2- oxo-1,2,5,6- tetrahydropyridine- 3-carboxamide | 397 |
| 4a₅b₃ | 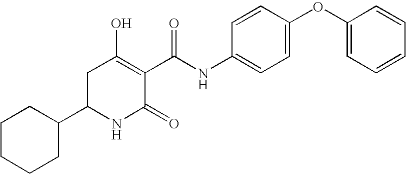 | 6-cyclohexyl-4- hydroxy-N-(4- phenoxyphenyl)- 2-oxo-1,2,5,6- tetrahydropyridine- 3-carboxamide | 407 |
| 4a₅b₄ | 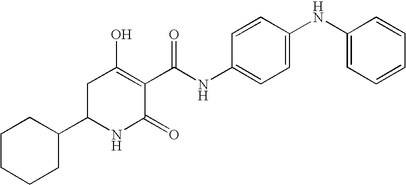 | N-(4-anilinophenyl)- 6-cyclohexyl-4- hydroxy-2-oxo- 1,2,5,6- tetrahydropyridine- 3-carboxamide | 406 |
| 4a₅b₅ | 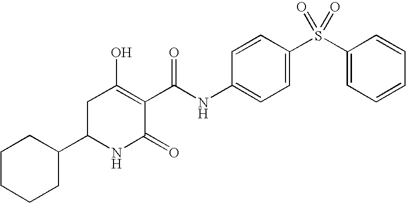 | 6-cyclohexyl-4- hydroxy-2-oxo-N- [4-(phenyl sulfonyl)phenyl]- 1,2,5,6- tetrahydropyridine- 3-carboxamide | 455 |
| 4a₅b₈ | 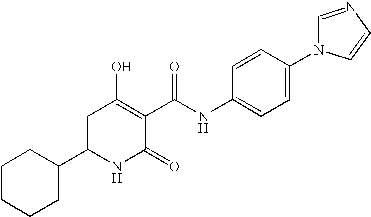 | 6-cyclohexyl-4- hydroxy-N-[4- (1H-imidazol-1- yl)phenyl]-2-oxo- 1,2,5,6- tetrahydropyridine- 3-carboxamide | 381 |
| 4a₅b₉ | 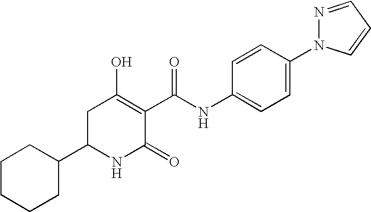 | 6-cyclohexyl-4- hydroxy-2-oxo-N- [4-(1H-pyrazol-1- yl)phenyl]- 1,2,5,6- tetrahydropyridine- 3-carboxamide | 381 |
| 4a₅b₁₁ | 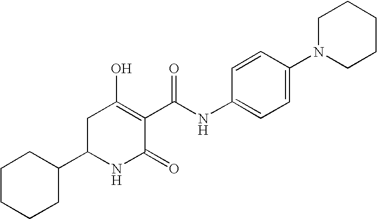 | 6-cyclohexyl-4- hydroxy-2-oxo-N- (4-piperidin-1- ylphenyl)-1,2,5,6- tetrahydropyridine- 3-carboxamide | 398 |
| 4a₅b₁₂ | 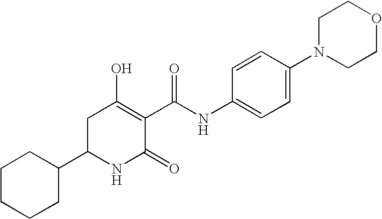 | 6-cyclohexyl-4- hydroxy-2-oxo-N- (4-morpholin-4- ylphenyl)-1,2,5,6- tetrahydropyridine- 3-carboxamide | 400 |
| 4a₅b₁₄ | 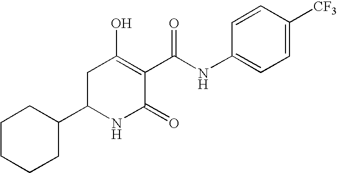 | 6-cyclohexyl-4- hydroxy-2-oxo-N- (4-trifluoro methylphenyl)- 1,2,5,6- tetrahydropyridine- 3-carboxamide | 383 |
| 4a₆b₁ | 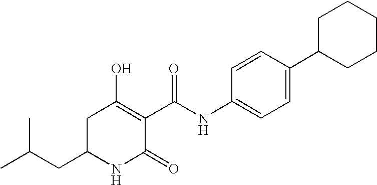 | N-(4- cyclohexylphenyl- 4-hydroxy-6- isobutyl-2-oxo- - 1,2,5,6- tetrahydropyridine- 3-carboxamide | 371 |
| 4a₆b₃ | 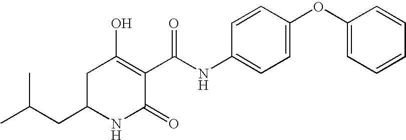 | 4-hydroxy-6- isobutyl-2-oxo-N- (4- phenoxyphenyl)- 1,2,5,6- tetrahydropyridine- 3-carboxamide | 381 |
| 4a₆b₅ | 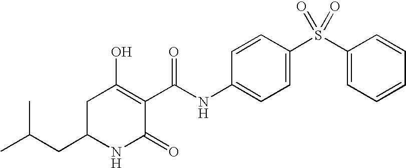 | 4-hydroxy-6- isobutyl-2-oxo-N- [4- phenylsulfonyl) phenyl]-1,2,5,6- tetrahydropyridine- 3-carboxamide | 429 |
| 4a₆b₉ | 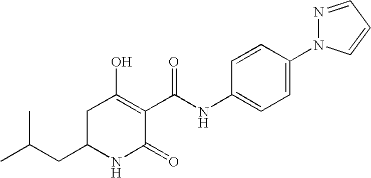 | 4-hydroxy-6- isobutyl-2-oxo-N- [4-(1H-pyrazol-1- ylphenyl]- 1,2,5,6- tetrahydropyridine- 3-carboxamide | 355 |
| 4a₆b₁₁ | 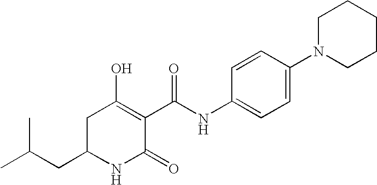 | 4-hydroxy-6- isobutyl-2-oxo-N- (4-piperidin-1- ylphenyl)-1,2,5,6- tetrahydropyridine- 3-carboxamide | 372 |
| 4a6₅b₁₂ | 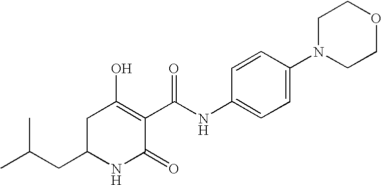 | 4-hydroxy-6- isobutyl-N-(4- morpholin-4- ylphenyl)-2-oxo- 1,2,5,6- tetrahydropyridine- 3-carboxamide | 373 |
| 4a₆b₁₃ | 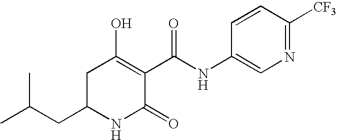 | 4-hydroxy-6- isobutyl-N-(6- trifluoromethyl- pyridin-3-yl)-2-oxo- 1,2,5,6-tetrahydro pyridine-3- carboxamide | 358 |
| 4a₅b₁₄ | 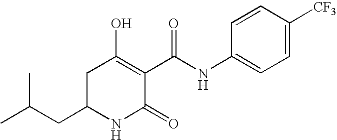 | 4-hydroxy-6- isobutyl-2-oxo-N- (4- trifluoromethyl- phenyl)-1,2,5,6- tetrahydropyridine- 3-carboxamide | 357 |
| 4a₅b₁₆ | 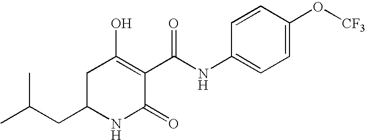 | 4-hydroxy-6- isobutyl-2-oxo-N- (4-trifluoro methoxyphenyl)- 1,2,5,6- tetrahydropyridine- 3-carboxamide | 373 |

### Example 10

### Preparation of Additional Monocyclic Hydroxydicarbonyl Compounds

The general synthetic preparation of additional monocyclic hydroxydicarbonyl compounds of the invention are described below.

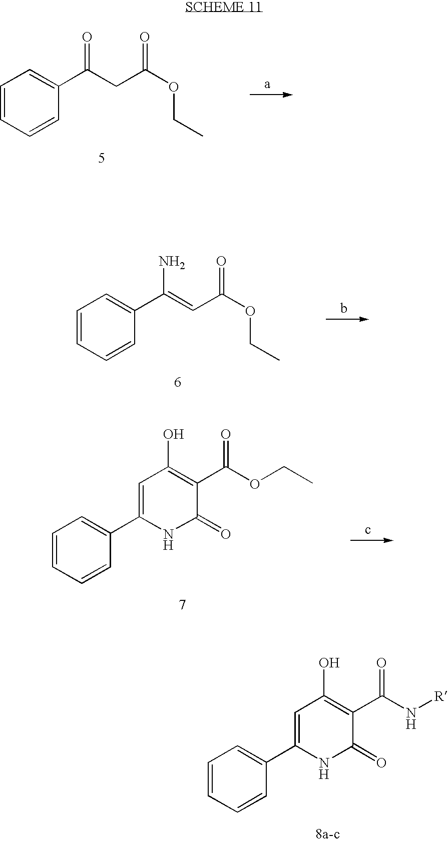

Reagents: (a) ammonium formate, anhydrous MeOH, reflux, 14 h; (b) NaOEt, diethyl malonate, EtOH, 180° C., 2 h, microwave synthesizer; (c) amine, DMF, 180° C., 10 min, microwave synthesizer.

### I Synthesis of Intermediates

### A. Ethyl (2Z)-3-amino-3-phenylacrylate

### 6

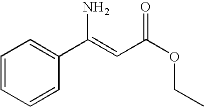

To a solution of ethyl benzoylacetate (5) (1.8 mL, 9.4 mmol) in anhydrous methanol (30 mL) was added ammonium formate (3 g, 47 mmol). The reaction mixture was refluxed for 14 h and concentrated in vacuo. The resulting residue was suspended in 100 mL EtOAc and 80 mL water and stirred for 30 min. The aqueous layer was extracted with EtOAc (2×100 mL), and the combined organic layers were dried (NaSO₄) and evaporated to give a dark color oil. The crude material was purified by distillation (180° C./80 pa) in a Kugelrohr apparatus to give the title compound as a colorless oil (1.5 g, 83%). MS (ES+): m/z=192 (M+1)

1H NMR (400 MHz, CHLOROFORM-D) δ=1.23-1.32 (m, 3H) 4.17 (q, J=7.41 Hz, 2H) 4.96 (s, 1H) 7.38-7.45 (m, 3H) 7.53 (dd, J=2.02 Hz, 8.00 Hz, 2H)

### B. 4-Hydroxy-2-oxo-6-phenyl-1,2-dihydro-pyridine-3-carboxylic acid Ethyl Ester

### 7

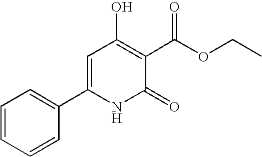

To a solution of 6 (500 mg, 2.35 mmol) in ethanol was added sodium ethoxide (384 mg, 5.7 mmol) and diethyl malonate (2.35 mL, 2.35 mmol). The reaction mixture was heated in a microwave synthesizer at 180° C. for 1.5 h. The resulting mixture was dissolved in water and adjusted to pH 1. The aqueous layer was extracted with DCM and the organic layer was combined, dried (Na₂SO₄) and concentrated to afford a grey solid. The crude material was purified by flash chromatography on silica gel, eluting with 2% MeOH in DCM to provide the title product as a white solid (60 mg, 10%). MS (ES+): m/z=260 (M+1) 1H NMR (400 MHz, CHLOROFORM-D) δ=1.40 (t, J=7.07 Hz, 3H) 4.45 (q, J=7.07 Hz, 2H) 6.25 (s, 1H) 7.48-7.54 (m, 3H) 7.67 (d, J=7.58 Hz, 2H) 13.49 (s, 1H)

### II. Synthesis of Examples

### A. General Procedure for the Formation of Amides 8 with Ester 7 and Amines

To a solution of the ester 7 (0.12-0.18 mmol, 1 eq.) in THF was added the amine (0.12-0.18 mmol, 1-1.4 eq.) and the mixture was heated in the microwave synthesizer at 180° C. for 10 min. After the mixture cooled down, the reaction mixture turned into a suspension and was filtered and rinsed with MeOH to afford amides 8.

### B. N-(4-Cyclohexylphenyl)-4-hydroxy-2-oxo-6-phenyl-1,2-dihydropyridine-3-carboxamide

### 8a

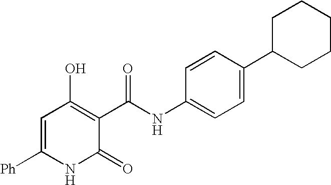

To a solution of 7 (30 mg, 0.12 mmol) in DMF was added 4-cyclohexylaniline (30 mg, 0.17 mmol) and the resulting mixture was heated in a microwave synthesizer at 180° C. for 10 min. After sitting at room temperature for 10 min, the reaction mixture turned into a suspension and was filtered and rinsed with MeOH to afford the title compound as a white crystalline solid (10 mg, 22%). MS (ES+): m/z=389 (M+1) 1H NMR (400 MHz, DMSO-D6) δ=1.24 (m, 1H) 1.31-1.42 (m, 4H) 1.70 (d, J=12.63 Hz, 1H) 1.78 (d, J=9.09 Hz, 4H) 6.45 (s, 1H) 7.24 (d, J=8.59 Hz, 2H) 7.50-7.58 (m, 5H) 7.77-7.84 (m, 2H) 12.17 (s, 3H) 12.46 (s, 1H) 15.29 (s, 3H).

According to the general method described above the following examples were prepared:

| Compound | | | MS m/z |
|---|---|---|---|
| # | Structure | Name | (M + 1) |
| 8b | 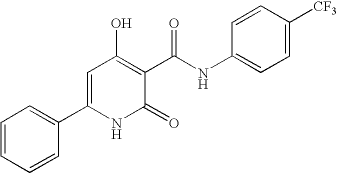 | 4-hydroxy-2-oxo-6 phenyl-N-[4- (trifluoromethyl)phenyl]- 1,2-dihydropyridine-3- carboxamide | 375 |
| 8c | 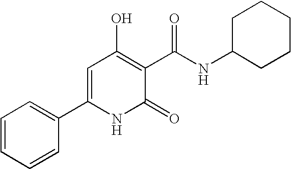 | N-cyc1ohexyl-4-hydroxy- 2-oxo-6-phenyl-1,2- dihydropyridine-3- carboxamide | 313 |

### Example 11

### Preparation of Additional Monocyclic Hydroxydicarbonyl Compounds

The general synthetic preparation of additional monocyclic hydroxydicarbonyl compounds of the invention are described below.

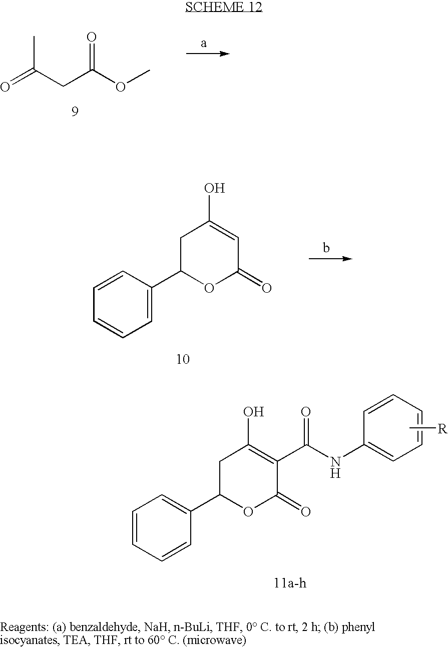

### I. Synthesis of Intermediates

### A. 4-Hydroxy-6-phenyl-5,6-dihydro-2H-pyran-2-one

### 10

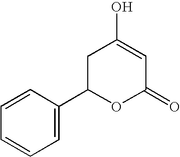

To a slurry of sodium hydride (44 mmol, 1.1 g) in anhydrous THF (100 mL) was added methyl acetoacetate (9) (37 mmol, 4 mL) dropwise at 0° C. under N₂ atmosphere and the reaction stirred at 0° C. for 15 mm. The reaction mixture was charged with n-butyllithium (40 mmol, 25 mL, 1.6 M in hexane) at 0° C. and stirred at 0° C. for 15 min. Benzaldehyde (4.08 mL, 40.4 mmol) was added to the dianion and the reaction was stirred at 0° C. for 1 h and stirred at room temperature for another 1 h. The mixture was poured into 0.1N NaOH aq. solution (30 mL) and stirred at room temperature for 15 min. The aqueous solution was washed with ether and acidified to pH 1-2 by using 2 N HCl at 0° C. The aqueous layer was extracted with DCM, and the organic layer was combined, dried (Na₂SO₄) and concentrated to give a light yellow solid. The crude material was recrystallized from DCM and hexane to afford the title product as an off white solid (5.0 g, 71%). MS (ES+): m/z=389 (M+1) 1H NMR (400 MHz, DMSO-D6) δ=2.57 (dd, J=17.18, 4.04 Hz, 1H) 2.81 (dd, J=16.93, 11.87 Hz, 1H) 5.05 (s, 1H) 5.44 (dd, J=13.62, 3.54 Hz, 1H) 7.35-7.46 (m, 5H) 11.53 (s, 1H)

### II Synthesis of Examples

### A. General Procedure for the Formation of Amides 11 with Isocyanates

To a solution of 10 (0.15-0.25 mmol, 1 eq) in THF (1.5 mL) was added the isocyanate (0.15-0.25 mmol) at 0° C. and triethylamine (0.22-0.37 mmol, 1.5 eq). The reaction mixture was stirred at room temperature for 30 min or heated at 60-80° C. in a microwave synthesizer for 5 min. The mixture was concentrated and purified by running through a silica gel column, eluting with 1-2% MeOH in DCM. Fractions containing the desired product were combined, concentrated and triturated with MeOH to afford amides 11.

### B. 4-Hydroxy-2-oxo-6-phenyl-N-(4-trifluoromethylphenyl)-5,6-dihydro-2H-pyran-3-carboxamide

### 11a

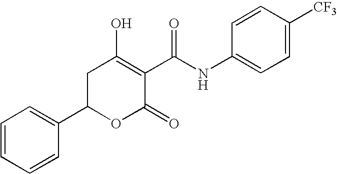

To a solution of 10 (30 mg, 0.15 mmol) in THF (1.5 mL) was added 4-(trifluoromethyl)phenyl isocyanate (21.4 μL, 0.15 mmol) and triethylamine (31.3 μL, 0.22 mmol) at 0° C. The reaction mixture was heated at 60° C. in a microwave for 5 min. The mixture was concentrated and purified by running through a silica gel column, eluting with 1-2% MeOH in DCM. Fractions containing the desired product were combined, concentrated and triturated with MeOH to afford the title compound as a gray solid (15 mg, 27%). MS (ES+): m/z=378 (M+1) 1H NMR (400 MHz, DMSO-D6) δ=2.99 (dd, J=17.68, 3.54 Hz, 1H) 3.32-3.42 (m, 2H) 5.72 (dd, J=12.63, 3.54 Hz, 1H) 7.40-7.49 (m, 3H) 7.50-7.55 (m, 2H) 7.76 (d, J=12 Hz, 2H) 7.85 (J=12 Hz, 2H) 11.20 (s, 1H)

According to the general method described above the following examples were prepared:

| Compound | | | MS: |
|---|---|---|---|
| # | Structure | Name | m/z |
| 11b | 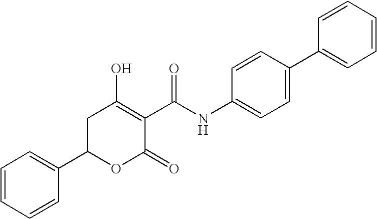 | N-biphenyl-4-yl-4-hydroxy-2-oxo-6- phenyl-5,6- dihydro-2H-pyran- 3-carboxamide | 386 |
| 11c | 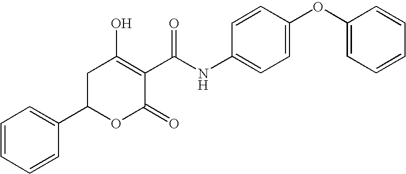 | 4-hydroxy-2-oxo- N-(4- phenoxyphenyl)-6- phenyl-1,2,5,6- tetrahydropyridine- 3-carboxamide | 402 |
| 11d | 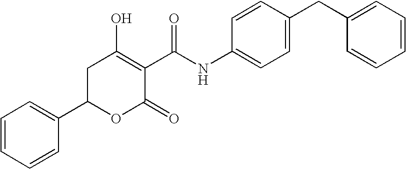 | N-(4- benzylphenyl-4- hydroxy-2-oxo-6- phenyl-5,6- dihydro-2H-pyran- 3-carboxamide | 400 |
| 11e | 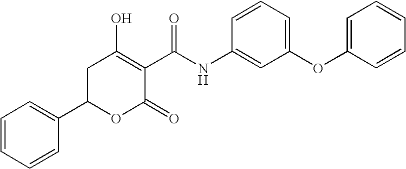 | 4-hydroxy-2-oxo- N-(3- phenoxylphenyl)-6 phenyl-5,6- dihydro-2H-pyran- 3-carboxamide | 402 |
| 11f | 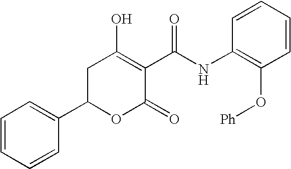 | 4-hydroxy-2-oxo- N-(2- phenoxyphenyl)-6 phenyl-5,6- dihydro-2H-pyran- 3-carboxamide | 402 |
| 11g | 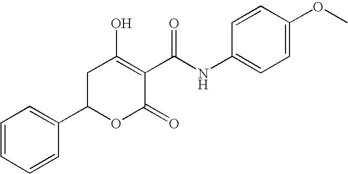 | 4-hydroxy-N-(4- methoxyphenyl)-2- oxo-6-phenyl-5,6- dihydro-2H-pyran- 3-carboxamide | 340 |
| 11h | 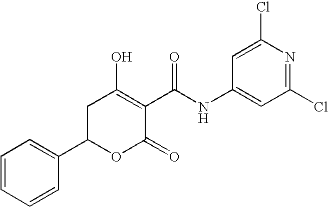 | N-(2,6- dichloropyridin-4- yl)-4-hydroxy-2- oxo-6-phenyl-5,6- dihydro-2H-pyran- 3-carboxamide | 379 |

### Example 12

### Preparation of Additional Monocyclic Hydroxydicarbonyl Compounds

The general synthetic preparation of additional monocyclic hydroxydicarbonyl compounds of the invention are described below.

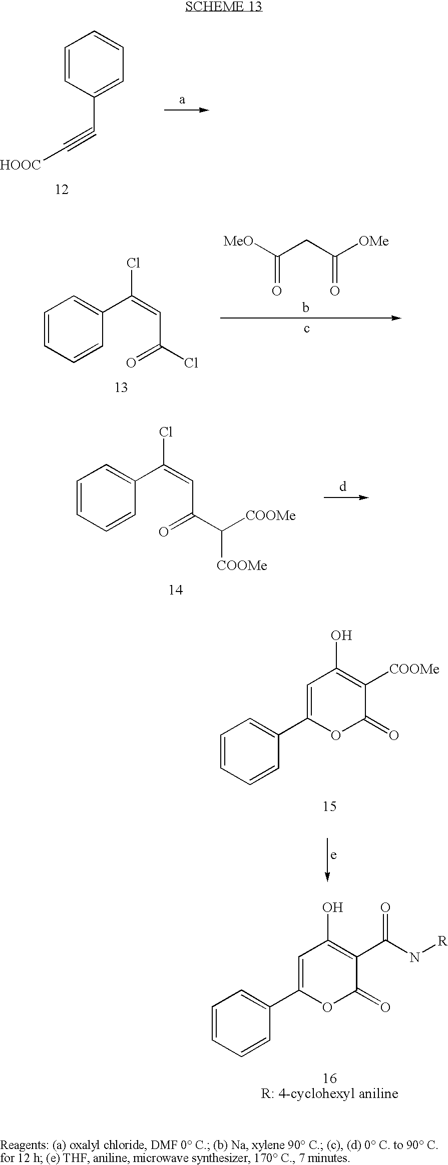

### I. Synthesis of Intermediate 15

### A. (E)-3-chloro-3-phenyl-acryloyl Chloride

### 13

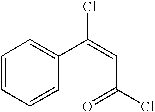

To the solution of phenylpropynoic acid (7.3 g, 50 mmol) in DMF (25 mL) at 0° C. was added oxalyl chloride. The mixture was stirred for 45 minutes and used without further purification in the next step. MS (ES+): m/z=201 (M+1)

### B. 2-((E)-3-Chloro-3-phenyl-acryloyl)-malonic acid Dimethyl Ester

### 14

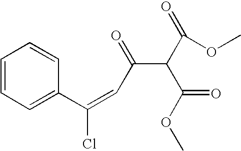

1.38 g (60 mmol) of sodium in 150 ml of xylene was heated at reflux, then (5.37 ml, 50 mmol) of dimethyl malonate was slowly added after the reaction mixture cooled to RT, then refluxed for 120 minutes. The color of the reaction mixture changed to yellow and the reaction mixture became cloudy. The dianion of malonic acid dimethyl ester was slowly added to (E)-3-Chloro-3-phenyl-acryloyl chloride in xylene solution at 0° C., then warmed up to rt for another 2 hours. The resultant mixture was poured into ice water with citric acid (50 mg), extracted with EtOAc, then washed with aqueous sodium carbonate solution (3×), with water, brine and dried over sodium sulfate to afford the title compound as a light yellow oil (7.6 g, 63.3%. MS (ES+): m/z=296 (M+1)

### C. 4-Hydroxy-2-oxo-6-phenyl-2H-pyran-3-carboxylic acid Methyl Ester

### 15

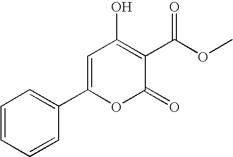

A solution of 14 (4.5 g, 15.17 mmol) in xylene was heated at 170° C. for 4 hours. Xylene was evaporated and the reaction mixture was diluted with EtOAc and washed with water, then dried over sodium sulfate to afford 15 as a white solid (3.23 g, 74.5%). MS (ES+): m/z=248 (M+1) 1H NMR (400 MHz, DMSO-D6) δ=3.66 (s, 3H) 6.51 (s, 1H) 7.49 (s, 1H) 7.50 (d, J=3.03 Hz, 3H) 7.82 (dd, J=6.57, 3.03 Hz, 2H)

### II Synthesis of Example

### A. 4-Hydroxy-2-oxo-6-phenyl-2H-pyran-3-carboxylic acid (4-cyclohexyl-phenyl)-amide

### 16ab1

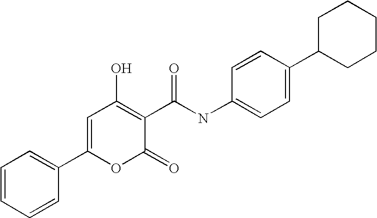

To the solution of 15 (49 mg, 0.2 mmol) in THF was added 4-cyclohexyl aniline (35 mg, 0.2 mmol) and heated up to 170° C. in a microwave synthesizer for 7 minutes. The reaction mixture was filtered, washed with diethyl ether to afford the desired product as a white powder (15.7 mg 20%) MS (ES+): m/z=390 1H NMR (400 MHz, DMSO-D6) δ=1.25 (s, 1H) 1.35-1.43 (m, 4H) 1.73 (s, 1H) 1.80 (d, J=9.09 Hz, 4H) 7.22-7.30 (m, 3H) 7.53-7.64 (m, 5H) 8.02 (d, J=6.57 Hz, 2H)

### Example 13

### Preparation of Additional Monocyclic Hydroxydicarbonyl Compounds

The general synthetic preparation of additional monocyclic hydroxydicarbonyl compounds of the invention are described below.

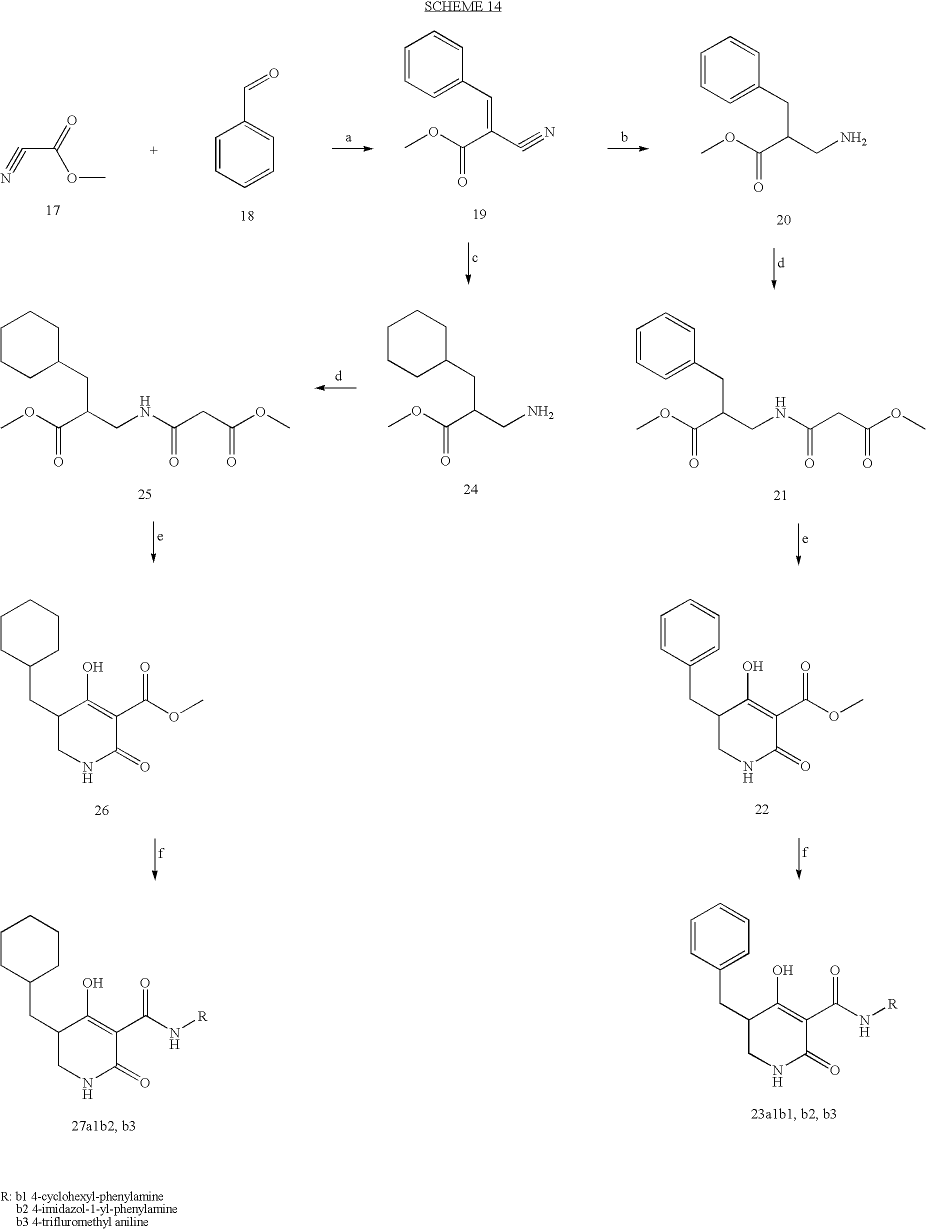

Reagents: (a) piperidine, toluene; (b) H₂, Pd(OH)₂, ethanol; (c) H₂, PtO₂, ethanol, 3 days; (d) TEA, methyl malonyl chloride, DCM; (e) NaOMe or 0.5M NaOMe in MeOH reflux; (f) aniline, microwave synthesizer 100-120° C., 5-8 minutes in THF or ethanol.

### I. Synthesis of Intermediate 22

### A. Z-2-Cyano-3-phenyl-acrylic acid Methyl Ester

### 19

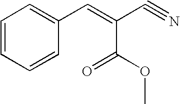

275 μL (2.72 mmol) of benzaldehyde and 240 μL (2.72 mmol) of methyl cyanoacetate and 1 ml of piperidine was dissolved in 4 ml of toluene, the solution was slowly heated to reflux with a Dean-Stark trap for 4 hours. The mixture was concentrated to about half of the volume and cooled downed to rt. The resulting precipitate was filtered off with suction to give a white crystalline solid 19 (376 mg, 74%). MS (ES+): m/z=188 (M+1) 1H NMR (400 MHz, DMSO-D6) δ=3.87 (s, 3H) 7.56-7.66 (m, 3H) 8.05 (d, J=7.58 Hz, 2H) 8.40 (s, 1H)

### B. 2-Aminomethyl-3-phenyl-propionic acid Methyl Ester

### 20

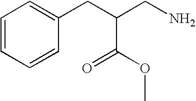

A solution of 19 (376 mg, 2 mmol) and 6 mL of concentrated HCl aq. in 40 mL ethanol was hydrogenated over Pearlman's catalyst under 50 psi H₂ atmosphere at rt for three days. After filtration of the catalyst, the filtrate was concentrated under reduced pressure, the residue was dissolved in water and washed with EtOAc. The aqueous solution was then extracted with DCM for three times. The organic phase was concentrated under reduced pressure to obtain the methyl ester hydrochloride as white crystals (350 mg, 90.6%). MS (ES+): m/z=193 (M+1)

### C. 2-[(2-Methoxycarbonyl-acetylamino)-methyl]-3-phenyl-propionic acid Methyl Ester

### 21

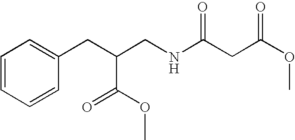

To a solution of 20 (3.58 g, 18.52 mmol) and 4.28 mL (30.7 mmol) of TEA in 25 mL DCM was added methyl malonyl chloride (1.79 mL, 16.8 mmol) via syringe. The reaction mixture was allowed to warm up to rt and stirred for 90 minutes. Subsequently it was poured into 20 mL iced 1 N HCl, the organic layer was separated and washed successively with cold water, 2% NaHCO₃, water and brine. The solution was dried over Na₂SO₄ and concentrated under vacuum to afford 11 (2.59 g, 63.3%). MS (ES+): m/z=294 (M+1)

### D. 5-Benzyl-4-hydroxy-2-oxo-1,2,5,6-tetrahydro-pyridine-3-carboxylic acid Methyl Ester

### 22

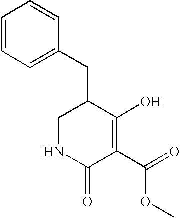

To a solution of 21 (270 mg, 1 mmol) in 25 mL MeOH was added sodium methoxide (100 mg, 1.24 mmol). The reaction mixture was heated to reflux at 80° C. for 12 hours. The solid was collected by filtration and washed with diethyl ether. The resulting cake was dissolved by adding iced water with stirring. 20 mL iced 1 N HCl was added, the separated solid was filtered, washed with cold water and dried one hour under suction to afford the white powder 22 (225 mg, 85.5%). MS (ES+): m/z=263 (M+1)

### II. Synthesis of Intermediates 26

### A. 2-Aminomethyl-3-cyclohexyl-propionic acid Methyl Ester

### 24

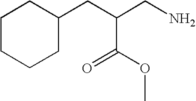

The solution of 19 (376 mg, 2 mmol) and 6 mL of concentrated HCl in 40 mL ethanol was hydrogenated over platinum oxide (0.2 eq.) at 50 psi H₂ atmosphere at rt for three days. After filtration of the catalyst, the filtrate was concentrated under reduced pressure, the residue was dissolved in water, washed with EtOAc, the aqueous solution was then extracted with DCM three times. The organic phase was concentrated under reduced pressure to obtain the methyl ester hydrochloride as white crystals (305 mg, 77.4%). MS (ES+): m/z=198 (M+1) 1H NMR (400 MHz, DMSO-D6) δ=0.78-0.89 (m, 2H) 1.10-1.21 (m, 4H) 1.40 (q, J=6.74 Hz, 2H) 1.56-1.67 (m, 4H) 1.70 (s, 1H) 2.84 (ddd, J=10.36, 5.05, 4.80 Hz, 2H) 2.91-3.01 (m, 1H) 3.64 (s, 3H)

### B. 3-Cyclohexyl-2-[(2-methoxycarbonyl-acetylamino)-methyl]-propionic acid Methyl Ester

### 25

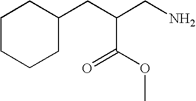

To a solution of 24 (2.0 g 10 mmol) and 3.07 mL of TEA in 25 mL DCM was added methyl malonyl chloride (1.18 mL) via syringe. The reaction mixture was allowed to warm up to it and stirred for 90 minutes. Subsequently, it was poured into 40 mL iced 1 N HCl, the organic layer was separated and washed successively with cold water, 2% NaHCO₃, water and brine. The solution was dried over Na₂SO₄ and concentrated under vacuum to afford crude product 25 without further purification. MS (ES+): m/z=298 (M+1)

### C. 5-Cyclohexylmethyl-4-hydroxy-2-oxo-1,2,5,6-tetrahydro-pyridine-3-carboxylic acid Methyl Ester

### 26

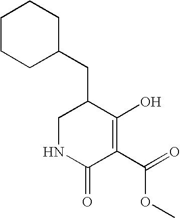

To solution of 25 (2.8 g, 936 mmol) in 25 mL MeOH was added sodium methoxide (758 mg, 14 mmol). The reaction mixture was heated to reflux at 80° C. for 12 hours. The solid was collected by filtration and washed with diethyl ether. The resulting cake was dissolved by adding iced water with stirring. 20 mL iced 1 N HCL was added, the separated solid was filtered, washed with cold water and dried one hour under suction to afford 26 as a white powder (300 mg, 12%). MS (ES+): m/z=268 (M+1) 1H NMR (400 MHz, CHLOROFORM-D) δ=0.84 (ddd, J=10.23, 5.31, 5.18 Hz, 2H) 0.89 (s, 1H) 1.09-1.21 (m, 4H) 1.21-1.31 (m, 2H) 1.59 (d, J=6.57 Hz, 3H) 1.65 (d, J=9.09 Hz, 6H) 2.59 (s, 1H) 3.04 (d, J=12.13 Hz, 1H) 3.42 (dd, 12.63, 2.53 Hz, 1H) 3.83 (s, 3H) 5.64 (s, 1H)

### III. Synthesis of Examples

### A. General Procedure for the Formation of Amides 23 and 27 from Methyl Ester 22 and 26

To a solution of 22 (0.2 mmol, 1 eq.) in THF was added aniline (0.24 mmol, 1.2 eq.). The reaction mixture was heated at 120° C. in the microwave synthesizer for 5 min. The mixture was concentrated and triturated with ether to afford the amides 23 as white solids.

### B. 5-Benzyl-4-hydroxy-2-oxo-1,2,5,6-tetrahydro-pyridine-3-carboxylic acid (4-cyclohexyl-phenyl)-amide

### 23a₁b₁

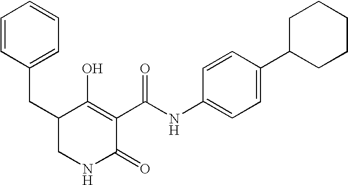

To a solution of 22 (52.2 mg, 0.2 mmol) in THF (1.5 mL) was added 4-cyclohexyl aniline (35 mg, 0.24 mmol). The reaction mixture was heated at 120° C. in a microwave synthesizer for 5 min. The mixture was concentrated and triturated with ether to afford the title compound as a white solid (6.6 mg, 8.25%), MS (ES+): m/z=405 (M+1) 1H NMR (400 MHz, DMSO-D6) δ=1.24 (m, 1H) 1.37 (t, J=10.61 Hz, 5H) 1.70 (d, J=12.63 Hz, 1H) 1.78 (d, J=9.09 Hz, 5H) 2.73 (m, 2H) 2.95 (s, 1H) 3.06 (s, 1H) 3.14-3.23 (m, 1H) 7.19-7.27 (m, 5H) 7.31 (d, J=7.07 Hz, 2H) 7.42 (d, J=8.08 Hz, 2H)

### C. 5-Benzyl-4-hydroxy-2-oxo-1,2,5,6-tetrahydro-pyridine-3-carboxylic acid (4-imidazol-1-yl-phenyl)-amide

### 23a₁b₁₇

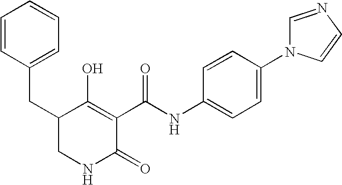

Analogous to 23a₁b was prepared 23a₁b₁₇ (10.9 mg, 11.2%) from 65 mg of 22. MS (ES+): m/z=388 (M+1)

### D. 5-Benzyl-4-hydroxy-2-oxo-1,2,5,6-tetrahydro-pyridine-3-carboxylic acid (4-trifluromethyl-1-yl-phenyl)-amide

### 23a₁b₂₅

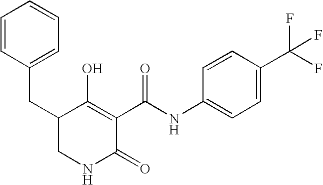

Analogous to 23a₁b was prepared 23a₁b₂₅ (8.1 mg, 8.3%) from 65 mg of 22. MS (ES+): m/z=391 (M+1)

### E. 5-Cyclohexylmethyl-4-hydroxy-2-oxo-1,2,5,6-tetrahydro-pyridine-3-carboxylic acid (4-imidazol-1-yl-phenyl)-amide

### 27a₁b₁₇

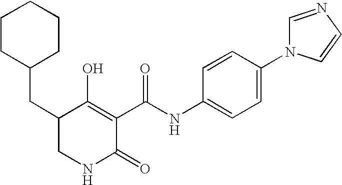

Analogous to 23a₁b₁ was prepared 27a₁b₁₇ (18.8 mg, 19.1%) from 67 mg of 26. MS (ES+): m/z=395 (M+1)

### F. 5-Cyclohexylmethyl-4-hydroxy-2-oxo-1,2,5,6-tetrahydro-pyridine-3-carboxylic acid (4-trifluromethyl-yl-phenyl)-amide

### 27a₁b₂₅

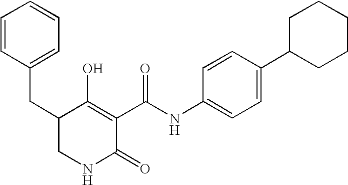

Analogous to 23a₁b was prepared 27a₁b₂₅ (11 mg, 11%) from 67 mg of 26, MS (ES+): m/z=397 (M+1)

### Example 14

### Preparation of Additional Monocyclic Hydroxydicarbonyl Compounds

The general synthetic preparation of additional monocyclic hydroxydicarbonyl compounds of the invention are described below.

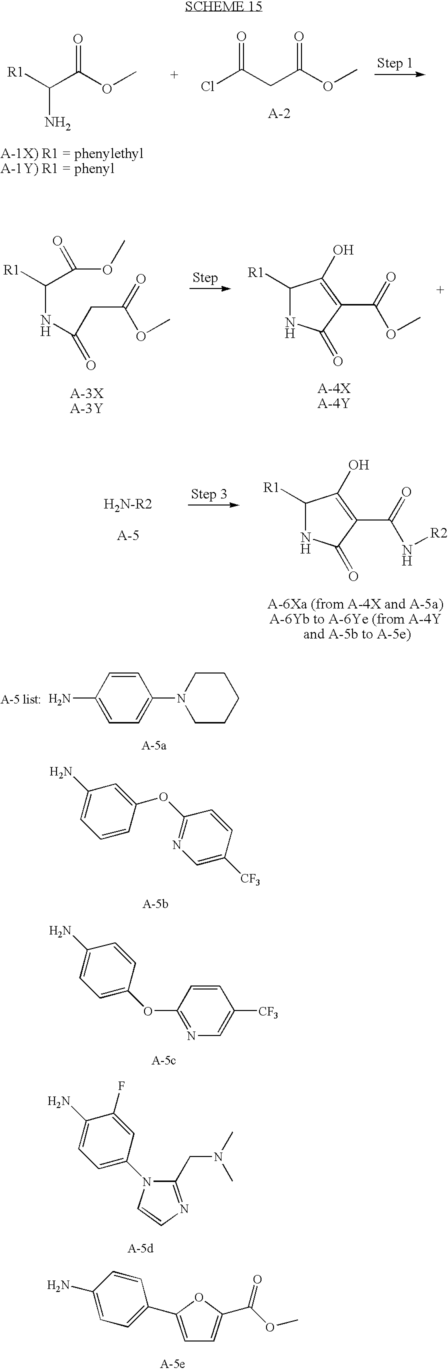

### I. Step 1

### A. A-3X

To a solution of A-1X (prepared from its corresponding amino acid by refluxing with concentrated HCl in MeOH, 1.00 g, ˜4.35 mmol), CH₂Cl₂ (10 mL), THF (10 mL) and Et₃N (1.36 mL, 987 mg, 9.78 mmol) was added A-2 (650 mg, 4.76 mmol) slowly at 0° C. The mixture was stirred at room temperature overnight. Volatiles were removed under vacuum and the residue was washed with saturated NaHCO₃/H₂O solution, extracted with CH₂Cl₂, concentrated and purified with silica gel chromatography to give yellow oil as desired product. Yield 70%.

### B. A-3Y

Similar procedure was used except CH₂Cl₂ was used as solvent. Yield 83%.

### II. Step 2

### A. A-4X

To a solution of compound A-3X (580 mg, 1.98 mmol) in MeOH (10 mL) was added NaOMe/MeOH (0.5 M, 9.90 mL, 4.95 mmol) at rt under the protection of N₂. The solution was stirred at rt for 4 h, then at 50° C. for 1 h. Volatiles were removed under vacuum to give yellow oil. Addition of HCl/H₂O (3 N, 3 mL) to the yellow oil gave yellow solid immediately. Filtration of the solid followed by washing with water gave off-white solid as product. Yield 81%.

### B. A-4Y

Similar procedure was used. Yield 85%.

### III. Step 3

### A. A-6Xa

A mixture of compound A-4X (200 mg, 0.765 mmol) and A-5a (148 mg, 0.840 mmol) in THF (15 mL) was microwaved at 100° C. for 5 min. The solution was cooled to rt, wherein a solid was precipitated from the solution. Filtration of the solid followed by washing with THF twice gave white solid as the desired product. Yield 49%.

### B. A-6Yb

A-4Y (100 mg, 0.429 mmol), A-5b (130 mg, 0.511 mmol), toluene (5 mL) and MeOH (1 mL) were mixed heated with 110° C. oil bath for 2 h. Filtration of the precipitated solid followed by washing with MeOH and CH₂Cl₂ gave the desired product. Yield 83%.

### C. A-6Yc to A-6Ye

Similar procedures as A-6Yb were used. Yield 9%-81%. Compound A-6Yd was purified with HPLC. (In this step, starting materials A-5a, A-5c and A-5e are commercially available. A-5d was synthesized as reported [J. Med. Chem. 2005, 48, 1729-1744]. A-5b was synthesized as this: 3-aminophenol (300 mg, 1.65 mmol), 2-Chloro-5-trifluoromethylpyridine (300 mg, 1.65 mmol), K₂CO₃ (342 mg, 2.48 mmol) were mixed in DMF (15 mL) and heated at 100° C. for 2 h. Volatiles were removed under reduced pressure followed by adding water and extracting with EtOAc. Purification with silica-gel chromatography gave A-5b in 57% yield.)

A-6Xa MS m/z (C₂₄H₂₈N₃O₃, Calcd. 406) found 407 (ES⁺), 405 (ES⁻); ¹H NMR (400 MHz, d⁶-DMSO) δ ppm 10.03 (s, 1H), 8.45 (s, 1H), 7.45 (d, J=8.0 Hz, 2H), 7.28-7.31 (m, 2H), 7.17-7.24 (m, 3H), 6.94 (d, J=8.0 Hz, 2H), 4.05 (ws, 1H), 3.11 (t, J=6.0 Hz, 4H), 2.64-2.69 (m, 2H); 2.02-2.06 (m, 1H), 1.75-1.78 (m, 1H), 1.60-1.66 (m, 4H), 1.51-1.55 (m, 2H).

A-6Yb MS m/z (C₂₃H₁₆F₃N₃O₃, Calcd. 455) found 456 (ES⁺) 454 (ES⁻); ¹H NMR (400 MHz, d⁶-DMSO) δ ppm 10.49 (s, 1H), 8.83 (s, 1H), 8.62-8.63 (m, 1H), 8.28 (dd, J=8.0, 4.0 Hz, 1H), 7.69-7.70 (m, 1H), 7.27-7.47 (m, 9H), 6.95-6.98 9 m, 1H), 5.27 (s, 1H).

A-6Yc MS m/z (C₂₃H₁₆F₃N₃O₃, Calcd. 455) found 456 (ES⁺); ¹H NMR (400 MHz, d⁶-DMSO) δ ppm 10.35 (s, 1H), 8.80 (s, 1H), 8.57 (s, 1H), 8.20-8.23 (m, 1H), 7.66-7.70 (m, 2H), 7.14-7.43 (m, 9H), 5.25 (s, 1H).

A-6Yd MS m/z (C₂₃H₂₂FN₃O₃, Calcd. 435) found 436 (ES⁺); ¹H NMR (400 MHz, d⁶-DMSO) δ ppm 11.62 (s, 1H), 8.59 (t, J=10.0 Hz, 1H), 8.21 (ws, 1H), 7.55 (s, 1H), 7.53 (s, 1H) 7.22-7.39 (m, 8H), 4.96 (s, 1H), 4.42 (s, 4H), 2.72 (s, 6H).

A-6Ye MS m/z (C₂₃H₁₈N₂O₆, Calcd. 418) found 419 (ES+), 417 (ES⁻); ¹H NMR (400 MHz, d⁶-DMSO) δ ppm 10.55 (s, 1H), 8.79 (s, 1H), 7.81 (d, J=8.0 Hz, 2H), 7.75 (d, J=8.0 Hz, 2H), 7.31-7.43 (m, 6H), 7.11 (d, J=4.0 Hz, 1H), 5.34 (s, 1H), 3.84 (s, 3H).

### Example 15

### Determination of Binding to UPPS

The ability of several of the compounds described herein to bind to UPPS was also tested as follows.

Streptococcus pneumonia UPPS was cloned into pET-15b, expressed and purified as an N-terminal His-tag fusion using affinity chromatography. The working stock of UPPS was prepared by mixing the purified enzyme with liposome made from E. coli total lipids extract (Avanti Polar Lipis, Inc., Alabaster, Ala.). The substrates FPP and IPP and inorganic pyrophosphatase were purchased from Sigma. Biomol Green reagent was from Biomol International (Plymouth Meeting, Pa.). All other chemicals were from Sigma at the highest grade.

For testing a compound, UPPS was first incubated with the compound at desired concentrations for 20 minutes in the UPPS reaction buffer that contained 100 mM Tris-HCl, pH 7.3, 50 mM KCl, 1 mM MgCl₂, 0.01% Triton X-100, and 20 μg/mL BSA. The reaction was then initiated by the addition of a mixture of FPP, IPP, and E. coli inorganic phosphatase made in the same UPPS reaction buffer. The final concentrations for FPP and IPP were 3 μM and 16 μM, respectively. The inorganic phosphate generated in the reactions was then quantified with Biomol Green reagent, which was then used to determine the rate of the reaction and the inhibitory activity of the compound.

For example, the results of the binding assay for several compounds are shown the table below:

TABLE 3
IC₅₀ Values for Binding to UPPS

| COMPOUND NO. | UPPS IC₅₀ (MM) |
|---|---|
| 4 | ** |
| 5 | *** |
| 6 | *** |
| 7 | *** |
| 8 | *** |
| 9 | *** |
| 10 | *** |
| 11 | *** |
| 12 | *** |
| 13 | ** |
| 14 | *** |
| 15 | ** |
| 16 | *** |
| 19 | *** |
| 22 | * |
| 23 | * |
| 36 | *** |
| 49 | *** |
| 52 | *** |
| 54 | ** |
| 56 | *** |
| 66 | *** |
| 70 | *** |
| 77 | *** |
| 80 | *** |
| 81 | *** |
| 86 | *** |
| 97 | *** |
| 103 | *** |
| 104 | *** |
| 113 | *** |
| 116 | *** |
| 123 | *** |
| 126 | *** |
| 148 | *** |
| 143 | *** |
| 164 | *** |
| 166 | *** |
| 167 | *** |
| 168 | *** |
| 169 | *** |
| 170 | *** |
| 174 | *** |
| 177 | *** |
| 181 | *** |
| 182 | *** |
| 183 | *** |
| 185 | *** |
| 187 | *** |
| 189 | *** |
| 192 | *** |
| 198 | *** |
| 202 | ** |
| 206 | *** |
| 221 | *** |
| 223 | * |

Key
IC₅₀
* limited enzyme interaction (IC₅₀ > 50 μM)
** some enzyme interaction (50 μM ≧ IC₅₀ > 10 μM)
*** good enzyme interaction (10 μM ≧ IC₅₀ > 0.01 μM)

Many of the compounds in Tables 1, 2, and 3, have also been tested to determine their minimum inhibitory concentration (MIC) for a variety of bacteria. The MIC values ranged from 0.125 μg/mL to greater than about 128 μg/mL. In particular embodiments, the MIC value was less than 64 μg/mL, e.g., less than 32 μg/mL.

### EQUIVALENTS

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including reaction times, reaction size/volume, and experimental reagents, such as solvents, catalysts, pressures, atmospheric conditions, e.g., nitrogen atmosphere, and reducing/oxidizing agents, etc., with art-recognized alternatives and using no more than routine experimentation, are within the scope of the present application.

It is to be understood that wherever values and ranges are provided herein, e.g., in ages of subject populations, dosages, blood levels, IC₅₀, and specificity ratios, all values and ranges encompassed by these values and ranges, are meant to be encompassed within the scope of the present invention. Moreover, all values that fall within these ranges, as well as the upper or lower limits of a range of values, are also contemplated by the present application.

### INCORPORATION BY REFERENCE

The contents of all references, issued patents, and published patent applications cited throughout this application are hereby expressly incorporated herein in their entireties by reference.

1. A method for treating bacterial disease comprising administering a potent and selective undecaprenyl pyrophosphate synthase (UPPS) inhibitor to a subject, such that a bacterial disease is treated in the subject wherein the UPPS inhibitor is represented by Formula I: 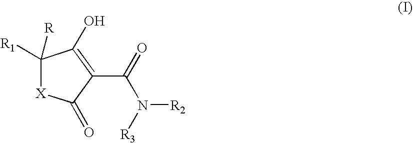
wherein
X is selected from the group consisting of NRₓCRₓRₓ and O;
R is selected from the group consisting of H, an aliphatic group, a carbocyclic group, a heterocyclic group, halogen, NO₂, CN, ORₐ, NRₐRₐ, CO₂Rₐ, —C(O)Rₐ, —CORₐ, NRₐC(O)Rₐ, NRₐC(O)NRₐRₐ, NRₐRₐC(O)O—, C(O)NRₐRₐ, which may be optionally substituted, wherein each Rₐ is independently selected from the group consisting of H, an aliphatic group, a carbocyclic group, and a heterocyclic group; or R and R₁, taken together, may form a substituted or unsubstituted spiro heterocyclic or carbocyclic ring, which may optionally be substituted;
R₁ and Rₓ are independently selected from the group consisting of H, -M₁, -M₁-M₂, -Z-M₂, and -M₁-Z-M₂; or R and R₁, taken together, may form a substituted or unsubstituted spiro heterocyclic or carbocyclic ring, which may optionally be substituted;
M₁ and M₂ are independently selected from the group consisting of H, an aliphatic group, a carbocyclic group, a heterocyclic group, which may be optionally substituted;
Z is selected from the group consisting of —O—, —NH—, —CR_{z}R_{z}—, —S—, —S(O)—, —C(O)—, —NHC(O)—, —C(O)NH—, —NHC(O)CH₂O—, —S(O)₂—, —CH(OH)—, —CH(OR_{z}), —C(O)CH₂—, —CH₂C(O)—, —CH₂CH(OH)—, —CH₂CH(OR_{z})—, —CH(OH)CH₂—, —CH(OR_{z})CH₂—, and any combination thereof, wherein each R_{z} is independently selected from the group consisting of H, an aliphatic group, a carbocyclic group, a heterocyclic group, hydroxy, and alkoxy;
R₂ is selected from the group consisting of H, an aliphatic group, a carbocyclic group, and a heterocyclic group;
R₃ is selected from the group consisting of -G₁, -G₁-G₂, —Y-G₂, and -G₁-Y-G₂;
G₁ and G₂ are independently selected from H, an aliphatic group, a carbocyclic group, and a heterocyclic group, which may be optionally substituted with one or more of substituents; and
Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂—, —CH(OH)—, —CH(OR_{y}), —C(O)CH₂—, —CH₂C(O)—, —CH₂CH(OH)—, —CH₂CH(OR_{y})—, —CH(OH)CH₂—, —CH(OR_{y})CH₂—, and any combination thereof, wherein each R_{y} is independently selected from the group consisting of H, an aliphatic group, a carbocyclic group, a heterocyclic group, hydroxy, and alkoxy.

2-36. (canceled)

37. The method of claim 1, wherein the UPPS inhibitor is represented by Formula II: 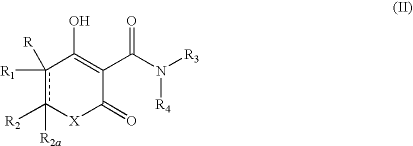
wherein [symbol]
represents a single or a double bond;
X is selected from the group consisting of NRₓCRₓRₓ and O;
R and R₂ₐ are absent or independently selected from the group consisting of H, an aliphatic group, a carbocyclic group, a heterocyclic group, halogen, NO₂, CN, ORₐ, NRₐRₐ, CO₂Rₐ, —C(O)Rₐ, —CORₐ, NRₐC(O)Rₐ, NRₐC(O)NRₐRₐ, NRₐRₐC(O)O—, C(O)NRₐRₐ, which may be optionally substituted, wherein each Rₐ is independently selected from the group consisting of H, an aliphatic group, a carbocyclic group, and a heterocyclic group; or R and R₁, taken together, may form a substituted or unsubstituted spiro heterocyclic or carbocyclic ring, which may optionally be substituted; or R₂ and R₂ₐ, taken together, may form a substituted or unsubstituted spiro heterocyclic or carbocyclic ring, which may optionally be substituted;
R₁, R₂, and Rₓ are independently selected from the group consisting of H, -M₁, -M₁-M₂, -Z-M₂, and -M₁-Z-M₂; or R and R₁, taken together, may form a substituted or unsubstituted spiro heterocyclic or carbocyclic ring, which may optionally be substituted; or R₂ and R₂ₐ, taken together, may form a substituted or unsubstituted spiro heterocyclic or carbocyclic ring, which may optionally be substituted;
M₁ and M₂ are independently selected from the group consisting of H, an aliphatic group, a carbocyclic group, a heterocyclic group, which may be optionally substituted;
Z is selected from the group consisting of —O—, —NH—, —CR_{z}R_{z}—, —S—, —S(O)—, —C(O)—, —NHC(O)—, —C(O)NH—, —NHC(O)CH₂O—, —S(O)₂—, —CH(OH)—, —CH(OR_{z}), —C(O)CH₂—, —CH₂C(O)—, —CH₂CH(OH)—, —CH₂CH(OR_{z})—, —CH(OH)CH₂—, —CH(OR_{z})CH₂—, and any combination thereof, wherein each R_{z} is independently selected from the group consisting of H, an aliphatic group, a carbocyclic group, a heterocyclic group, hydroxy, and alkoxy;
R₃ is selected from the group consisting of -G₁, -G₁-G₂, —Y-G₂, and -G₁-Y-G₂;
G₁ and G₂ are independently selected from H, an aliphatic group, a carbocyclic group, and a heterocyclic group, which may be optionally substituted with one or more of substituents;
Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂—, —CH(OH)—, —CH(OR_{y}), —C(O)CH₂—, —CH₂C(O)—, —CH₂CH(OH)—, —CH₂CH(OR_{y})—, —CH(OH)CH₂—, —CH(OR_{y})CH₂—, and any combination thereof, wherein each R_{y} is independently selected from the group consisting of H, an aliphatic group, a carbocyclic group, a heterocyclic group, hydroxy, and alkoxy; and
R₄ is selected from the group consisting of H, an aliphatic group, a carbocyclic group, and a heterocyclic group.

38. The method of claim 1, wherein the UPPS inhibitor is represented by Formula III: 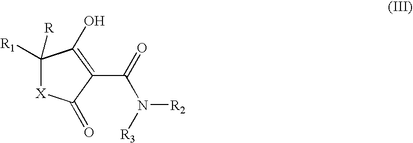
wherein
X is selected from the group consisting of NRₓCRₓRₓ and O;
R is selected from the group consisting of H, benzyl, pyridinyl, tetrahydro-pyranyl, methyl-1H-imidazolyl, cyclohexylmethyl, phenethyl, p-chlorobenzyl, carboxylic acid benzyl ester, propionic acid tert-butyl ester, tert-butyl ester, ethanone, hydroxy, methoxy, ethoxy, propoxy, butoxy, t-butoxy, phenyl, isobutyl, methyl, ethyl, propyl, butyl, cyclohexyl, cyclohexylmethyl, m-methoxy phenyl, carboxylic acid 2-methoxy-ethyl ester, 3,3-dimethyl-butan-1-one, 2,2-dimethyl-propan-1-one, carboxylic acid methyl ester, alkyl, halogen, NO₂, CN, ORₐ, NRₐRₐ, CO₂Rₐ, —C(O)Rₐ, —CORₐ, NRₐC(O)Rₐ, NRₐC(O)NRₐRₐ, NRₐRₐC(O)O—, C(O)NRₐRₐ, aryl, and heterocycle, which may be optionally substituted with methoxy or 2-methoxy-ethoxy, wherein each Rₐ is independently selected from the group consisting of H, alkyl, aryl, and heterocycle; or R and R₁, taken together, may form a substituted or unsubstituted spiro heterocyclic or carbocyclic ring, which may optionally be substituted with a benzyl group;
R₁ and Rₓ are independently selected from the group consisting of H, benzyl, pyridinyl, tetrahydro-pyranyl, methyl-1H-imidazolyl, cyclohexylmethyl, phenethyl, p-chlorobenzyl, carboxylic acid benzyl ester, propionic acid tert-butyl ester, tert-butyl ester, ethanone, hydroxy, methoxy, ethoxy, propoxy, butoxy, t-butoxy, phenyl, isobutyl, methyl, ethyl, propyl, butyl, cyclohexyl, cyclohexylmethyl, m-methoxy phenyl, carboxylic acid 2-methoxy-ethyl ester, 3,3-dimethyl-butan-1-one, 2,2-dimethyl-propan-1-one, carboxylic acid methyl ester, alkyl, halogen, NO₂, CN, OR_{b}, NR_{b}R_{b}, CO₂R_{b}, —C(O)R_{b}, —COR_{b}, NR_{b}C(O)R_{b}, NR_{b}C(O)NR_{b}R_{b}, NR_{b}R_{b}C(O)O—, C(O)NR_{b}R_{b}, aryl, and heterocycle, which may be optionally substituted with methoxy or 2-methoxy-ethoxy, wherein each R_{b} is independently selected from the group consisting of H, alkyl, aryl, and heterocycle; or R and R₁, taken together, may form a substituted or unsubstituted spiro heterocyclic or carbocyclic ring, which may optionally be substituted with a benzyl group;
R₂ is selected from the group consisting of H, phenyl, benzyl, isobutyl, cyclohexyl, cyclohexylmethyl, m-methoxy phenyl, alkyl, aryl, and heterocycle;
R₃ is selected from the group consisting of -G₁, -G₁-G₂, —Y-G₂, and -G₁-Y-G₂;
G₁ and G₂ are independently, selected from the group consisting of phenyl, cyclohexyl, cyclopentyl, 4-indanyl, pyrimidinyl, N-morpholino, furanyl, thiophenyl, pyrrolyl, N-1H-pyridin-2-onyl, bicyclo[4.2.0]octa-1,3,5-trien-3-yl, 1-indanyl, naphthalenyl, tetrahydro-naphthalenyl, pyrazine, [1,2,3]thiadiazolyl, 3-isoxazolyl, 5-indolyl, 2,3-dihydro-indol-6-yl, indazol-5-yl, benzo[2,1,3]thiadiazol-5-yl, cycloheptyl, isopropyl-[1,3,4]thiadiazolyl, benzothiazolyl, 3-methyl-butyl, 1H-pyrazolyl, oxazolyl, piperidinyl, 1H-imidazolyl, pyrrolidinyl, piperazinyl, 1H-[1,2,4]triazolyl, and pyridinyl, which may be optionally substituted with one or more of substituent moieties selected from the group consisting of CF₃, OCF₃, iodo, chloro, bromo, —C(O)NH₂, —O(CH₂)₅CH₃, carboxylic acid methyl ester, phenyl, p-methoxy phenyl, —NHC(O)NH₂, —C(O)O(CH₂)₂N(CH₂CH₃)₂, t-butyl, fluoro, methoxy, hydroxy, isopropyl, cyano, isopropenyl tetrahydropyran, benzyl, amino, —NHC(O)OC(CH₃)₃, —C(O)OH, —C(O)CH₃, —CH₂CO₂H, methyl, and —(CH₂)₂—OH; and
Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂—, and any combination thereof, wherein each R_{y} is independently selected from the group consisting of H, alkyl, aryl, heterocycle, hydroxy, or alkoxy.

39-40. (canceled)

41. The method of claim 1, wherein the UPPS inhibitor is represented by Formula IV: 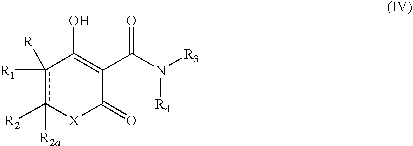
wherein [symbol]
represents a single or a double bond;
X is selected from the group consisting of NRₓCRₓRₓ and O;
R and R₂ₐ are absent or independently selected from the group consisting of H, benzyl, pyridinyl, tetrahydro-pyranyl, methyl-1H-imidazolyl, cyclohexylmethyl, phenethyl, p-chlorobenzyl, carboxylic acid benzyl ester, propionic acid tert-butyl ester, tert-butyl ester, ethanone, hydroxy, methoxy, ethoxy, propoxy, butoxy, t-butoxy, phenyl, isobutyl, methyl, ethyl, propyl, butyl, cyclohexyl, cyclohexylmethyl, m-methoxy phenyl, carboxylic acid 2-methoxy-ethyl ester, 3,3-dimethyl-butan-1-one, 2,2-dimethyl-propan-1-one, carboxylic acid methyl ester, alkyl, halogen, NO₂, CN, ORₐ, NRₐRₐ, CO₂Rₐ, —C(O)Rₐ, CORₐ, NRₐC(O)Rₐ, NRₐC(O)NRₐRₐ, NRₐRₐC(O)O—, C(O)NRₐRₐ, aryl, and heterocycle, which may be optionally substituted with methoxy or 2-methoxy-ethoxy, wherein each Rₐ is independently selected from the group consisting of H, alkyl, aryl, and heterocycle;
R₁, R₂, each Rₓ are independently selected from the group consisting of H, benzyl, pyridinyl, tetrahydro-pyranyl, methyl-1H-imidazolyl, cyclohexylmethyl, phenethyl, p-chlorobenzyl, carboxylic acid benzyl ester, propionic acid tert-butyl ester, tert-butyl ester, ethanone, hydroxy, methoxy, ethoxy, propoxy, butoxy, t-butoxy, phenyl, isobutyl, methyl, ethyl, propyl, butyl, cyclohexyl, cyclohexylmethyl, m-methoxy phenyl, carboxylic acid 2-methoxy-ethyl ester, 3,3-dimethyl-butan-1-one, 2,2-dimethyl-propan-1-one, carboxylic acid methyl ester, alkyl, halogen, NO₂, CN, OR_{b}, NR_{b}R_{b}, CO₂R_{b}, —C(O)R_{b}, —COR_{b}, NR_{b}C(O)R_{b}, NR_{b}C(O)NR_{b}R_{b}, NR_{b}R_{b}C(O)O—, C(O)NR_{b}R_{b}, aryl, and heterocycle, which may be optionally substituted with methoxy or 2-methoxy-ethoxy, wherein each R_{b} is independently selected from the group consisting of H, alkyl, aryl, and heterocycle;
R₃ is selected from the group consisting of -G₁, -G₁-G₂, —Y-G₂, and -G₁-Y-G₂;
G₁ and G₂ are independently selected from the group consisting of phenyl, cyclohexyl, cyclopentyl, 4-indanyl, pyrimidinyl, N-morpholino, furanyl, thiophenyl, pyrrolyl, N-1H-pyridin-2-onyl, bicyclo[4.2.0]octa-1,3,5-trien-3-yl, 1-indanyl, naphthalenyl, tetrahydro-naphthalenyl, pyrazine, [1,2,3]thiadiazolyl, 3-isoxazolyl, 5-indolyl, 2,3-dihydro-indol-6-yl, indazol-5-yl, benzo[2,1,3]thiadiazol-5-yl, cycloheptyl, isopropyl-[1,3,4]thiadiazolyl, benzothiazolyl, 3-methyl-butyl, 1H-pyrazolyl, oxazolyl, piperidinyl, 1H-imidazolyl, pyrrolidinyl, piperazinyl, 1H-[1,2,4]triazolyl, and pyridinyl, which may be optionally substituted with one or more of substituent moieties selected from the group consisting of CF₃, OCF₃, iodo, chloro, bromo, —C(O)NH₂, —O(CH₂)₅CH₃, carboxylic acid methyl ester, phenyl, p-methoxy phenyl, —NHC(O)NH₂, —C(O)O(CH₂)₂N(CH₂CH₃)₂, t-butyl, fluoro, methoxy, hydroxy, isopropyl, cyano, isopropenyl tetrahydropyran, benzyl, amino, —NHC(O)OC(CH₃)₃, —C(O)OH, —C(O)CH₃, —CH₂CO₂H, methyl, and —(CH₂)₂—OH;
Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂—, and any combination thereof, wherein each R_{y} is independently selected from the group consisting of H, alkyl, aryl, heterocycle, hydroxy, or alkoxy; and
R₄ is selected from the group consisting of H, phenyl, benzyl, isobutyl, cyclohexyl, cyclohexylmethyl, m-methoxy phenyl, alkyl, aryl, and heterocycle.

42-45. (canceled)

46. The method of claim 1, wherein the UPPS inhibitor is represented by Formula V: 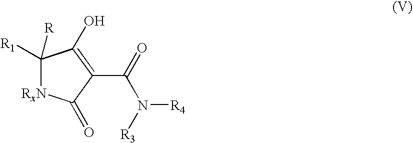
wherein
R₁, R, and Rₓ are independently selected from the group consisting of H, benzyl, pyridinyl, tetrahydro-pyranyl, methyl-1H-imidazolyl, cyclohexylmethyl, phenethyl, p-chlorobenzyl, carboxylic acid benzyl ester, propionic acid tert-butyl ester, tert-butyl ester, ethanone, hydroxy, methoxy, ethoxy, propoxy, butoxy, t-butoxy, phenyl, isobutyl, methyl, ethyl, propyl, butyl, cyclohexyl, cyclohexylmethyl, m-methoxy phenyl, carboxylic acid 2-methoxy-ethyl ester, 3,3-dimethyl-butan-1-one, 2,2-dimethyl-propan-1-one, carboxylic acid methyl ester, alkyl, halogen, NO₂, CN, ORₐ, NRₐRₐ, CO₂Rₐ, —C(O)Rₐ, —CORₐ, NRₐC(O)Rₐ, NRₐC(O)NRₐRₐ, NRₐRₐC(O)O—, C(O)NRₐRₐ, aryl, and heterocycle, which may be optionally substituted with methoxy or 2-methoxy-ethoxy, wherein each Rₐ is independently selected from the group consisting of H, alkyl, aryl, and heterocycle;
R₃ is selected from the group consisting of -G₁, -G₁-G₂, —Y-G₂, and -G₁-Y-G₂;
G₁ and G₂ are independently selected from the group consisting of phenyl, cyclohexyl, cyclopentyl, 4-indanyl, pyrimidinyl, N-morpholino, furanyl, thiophenyl, pyrrolyl, N-1H-pyridin-2-onyl, bicyclo[4.2.0]octa-1,3,5-trien-3-yl, 1-indanyl, naphthalenyl, tetrahydro-naphthalenyl, pyrazine, [1,2,3]thiadiazolyl, 3-isoxazolyl, 5-indolyl, 2,3-dihydro-indol-6-yl, indazol-5-yl, benzo[2,1,3]thiadiazol-5-yl, cycloheptyl, isopropyl-[1,3,4]thiadiazolyl, benzothiazolyl, 3-methyl-butyl, 1H-pyrazolyl, oxazolyl, piperidinyl, 1H-imidazolyl, pyrrolidinyl, piperazinyl, 1H-[1,2,4]triazolyl, and pyridinyl, which may be optionally substituted with one or more of substituent moieties selected from the group consisting of CF₃, OCF₃, iodo, chloro, bromo, —C(O)NH₂, —O(CH₂)₅CH₃, carboxylic acid methyl ester, phenyl, p-methoxy phenyl, —NHC(O)NH₂, —C(O)O(CH₂)₂N(CH₂CH₃)₂, t-butyl, fluoro, methoxy, hydroxy, isopropyl, cyano, isopropenyl tetrahydropyran, benzyl, amino, —NHC(O)OC(CH₃)₃, —C(O)OH, —C(O)CH₃, —CH₂CO₂H, methyl, and —(CH₂)₂—OH;
Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂—, and any combination thereof, wherein each R_{y} is independently selected from the group consisting of H, alkyl, aryl, heterocycle, hydroxy, or alkoxy; and
R₄ is selected from the group consisting of H, phenyl, benzyl, isobutyl, cyclohexyl, cyclohexylmethyl, m-methoxy phenyl, alkyl, aryl, and heterocycle.

47. The method of claim 1, wherein the UPPS inhibitor is represented by Formula VI: 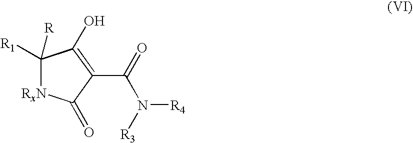
wherein
R is selected from the group consisting of H, alkyl, halogen, NO₂, CN, ORₐ, NRₐRₐ, CO₂Rₐ, and CONRₐRₐ, wherein each Rₐ is independently selected from the group consisting of H, alkyl, aryl, and heterocycle; or R and R₁, taken together, may form a substituted or unsubstituted spiro heterocyclic or carbocyclic ring, which may optionally be substituted with a benzyl group;
R₁ is selected from the group consisting of H, phenyl, benzyl, ethyl, methyl, isobutyl, pyridinyl, tetrahydro-pyranyl, methyl-1H-imidazolyl, cyclohexylmethyl, phenethyl, p-chlorobenzyl, carboxylic acid benzyl ester, propionic acid tert-butyl ester; or R and R₁, taken together, may form a substituted or unsubstituted spiro heterocyclic or carbocyclic ring, which may optionally be substituted with a benzyl group;
R₂ is selected from the group consisting of H, phenyl, benzyl, isobutyl, cyclohexyl, cyclohexylmethyl, m-methoxy phenyl;
R₃ is selected from the group consisting of -G₁, -G₁-G₂, —Y-G₂, and -G₁-Y-G₂;
G₁ and G₂ are independently selected from the group consisting of 4-indanyl, cyclohexyl, furanyl, pyrrolyl, N-1H-pyridin-2-onyl, and benzothiazolyl, thiophenyl, oxazolyl, pyridinyl, piperidinyl, piperazinyl, N-morpholino, 1H-Pyrazolyl, phenyl, 1H-[1,2,4]triazolyl, 1H-imidazolyl, and pyrimidinyl, which may be optionally substituted with one or more of substituent moieties selected from the group consisting of methoxy, ethyl, methyl, CF₃, cyano, benzyl, phenyl, p-methoxy phenyl, fluoro, tert-butyl, chloro, —(CH₂)₅CH₃, isopropyl, isopropenyl, carboxylic acid methyl ester, methyl-dimethyl-amine, —SCH₃, —C(O)NH, —NHC(O)OC(CH₃)₃, —(CH₂)₂—OH, and —S(O)₂CH₃;
Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂—, and any combination thereof, wherein each R_{y} is independently selected from the group consisting of H, alkyl, aryl, heterocycle, hydroxy, or alkoxy; and
Rₓ is selected from the group consisting of H, phenyl, benzyl, isobutyl, cyclohexyl, cyclohexylmethyl, m-methoxy phenyl, alkyl, aryl, and heterocycle.

48. A method for treating bacterial disease comprising administering a potent UPPS inhibitor to a subject, such that a bacterial disease is treated in the subject, wherein the UPPS inhibitor is represented by Formula I: 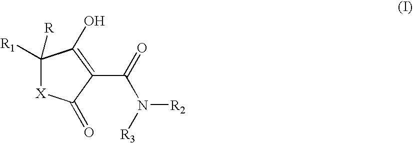
wherein
X is selected from the group consisting of NRₓCRₓRₓ and O;
R is selected from the group consisting of H, an aliphatic group, a carbocyclic group, a heterocyclic group, halogen, NO₂, CN, ORₐ, NRₐRₐ, CO₂Rₐ, —C(O)Rₐ, —CORₐ, NRₐC(O)Rₐ, NRₐC(O)NRₐRₐ, NRₐRₐC(O)O—, C(O)NRₐRₐ, which may be optionally substituted, wherein each Rₐ is independently selected from the group consisting of H, an aliphatic group, a carbocyclic group, and a heterocyclic group; or R and R₁, taken together, may form a substituted or unsubstituted spiro heterocyclic or carbocyclic ring, which may optionally be substituted;
R₁ and Rₓ are independently selected from the group consisting of H, -M₁, -M₁-M₂, -Z-M₂, and -M₁-Z-M₂; or R and R₁, taken together, may form a substituted or unsubstituted spiro heterocyclic or carbocyclic ring, which may optionally be substituted;
M₁ and M₂ are independently selected from the group consisting of H, an aliphatic group, a carbocyclic group, a heterocyclic group, which may be optionally substituted;
Z is selected from the group consisting of —O—, —NH—, —CR_{z}R_{z}, —S—, —S(O)—, —C(O)—, —NHC(O)—, —C(O)NH—, —NHC(O)CH₂O—, —S(O)₂, —CH(OH)—, —CH(OR_{z}), —C(O)CH₂, —CH₂C(O)—, —CH₂CH(OH)—, —CH₂CH(OR_{z})—, —CH(OH)CH₂—, —CH(OR_{z})CH₂—, and any combination thereof, wherein each R_{z} is independently selected from the group consisting of H, an aliphatic group, a carbocyclic group, a heterocyclic group, hydroxy, and alkoxy;
R₂ is selected from the group consisting of H, an aliphatic group, a carbocyclic group, and a heterocyclic group;
R₃ is selected from the group consisting of -G₁, -G₁-G₂, —Y-G₂, and -G₁-Y-G₂;
G₁ and G₂ are independently selected from H, an aliphatic group, a carbocyclic group, and a heterocyclic group, which may be optionally substituted with one or more of substituents; and
Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂, —CH(OH)—, —CH(OR_{y}), —C(O)CH₂—, —CH₂C(O)—, —CH₂CH(OH)—, —CH₂CH(OR_{y})—, —CH(OH)CH₂—, —CH(OR_{y})CH₁—, and any combination thereof, wherein each R_{y} is independently selected from the group consisting of H, an aliphatic group, a carbocyclic group, a heterocyclic group, hydroxy, and alkoxy.

49-66. (canceled)

67. The method for treating bacterial disease comprising administering a potent UPPS inhibitor to a subject, such that a bacterial disease is treated in the subject, wherein the UPPS inhibitor is represented by Formula II: 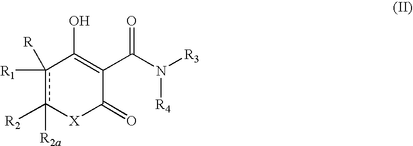
wherein [symbol]
represents a single or a double bond;
X is selected from the group consisting of NRₓCRₓRₓ and O;
R and R₂ₐ are absent or independently selected from the group consisting of H, an aliphatic group, a carbocyclic group, a heterocyclic group, halogen, NO₂, CN, ORₐ, NRₐRₐ, CO₂Rₐ, —C(O)Rₐ, —CORₐ, NRₐC(O)Rₐ, NRₐC(O)NRₐRₐ, NRₐRₐC(O)O—, C(O)NRₐRₐ, which may be optionally substituted, wherein each Rₐ is independently selected from the group consisting of H, an aliphatic group, a carbocyclic group, and a heterocyclic group; or R and R₁, taken together, may form a substituted or unsubstituted spiro heterocyclic or carbocyclic ring, which may optionally be substituted; or R₂ and R₂ₐ, taken together, may form a substituted or unsubstituted spiro heterocyclic or carbocyclic ring, which may optionally be substituted;
R₁, R₂, and Rₓ are independently selected from the group consisting of H, -M₁, -M₁-M₂, -Z-M₂, and -M₁-Z-M₂; or R and R₁, taken together, may form a substituted or unsubstituted spiro heterocyclic or carbocyclic ring, which may optionally be substituted; or R₂ and R₂ₐ, taken together, may form a substituted or unsubstituted spiro heterocyclic or carbocyclic ring, which may optionally be substituted;
M₁ and M₂ are independently selected from the group consisting of H, an aliphatic group, a carbocyclic group, a heterocyclic group, which may be optionally substituted;
Z is selected from the group consisting of —O—, —NH—, —CR_{z}R_{z}—, —S—, —S(O)—, —C(O)—, —NHC(O)—, —C(O)NH—, —NHC(O)CH₂O—, —S(O)₂—, —CH(OH)—, —CH(OR_{z}), —C(O)CH₂—, —CH₂C(O)—, —CH₂CH(OH)—, —CH₂CH(OR_{z})—, —CH(OH)CH₂—, —CH(OR₂)CH₂—, and any combination thereof, wherein each R_{z} is independently selected from the group consisting of H, an aliphatic group, a carbocyclic group, a heterocyclic group, hydroxy, and alkoxy;
R₃ is selected from the group consisting of -G₁, -G₁-G₂, —Y-G₂, and -G₁-Y-G₂;
G₁ and G₂ are independently selected from H, an aliphatic group, a carbocyclic group, and a heterocyclic group, which may be optionally substituted with one or more of substituents;
Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂—, —CH(OH)—, —CH(OR_{y}), —C(O)CH₂—, —CH₂C(O)—, —CH₂CH(OH)—, —CH₂CH(OR_{y})—, —CH(OH)CH₂—, —CH(OR_{y})CH₂—, and any combination thereof, wherein each R_{y} is independently selected from the group consisting of H, ah aliphatic group, a carbocyclic group, a heterocyclic group, hydroxy, and alkoxy; and
R₄ is selected from the group consisting of H, an aliphatic group, a carbocyclic group, and a heterocyclic group.

68-107. (canceled)

108. A compound of Formula VII: 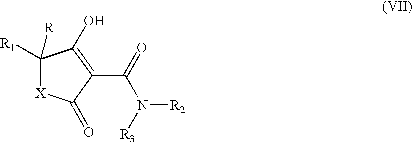
wherein
X is selected from the group consisting of NRₓCRₓRₓ and O;
R is selected from the group consisting of H, an aliphatic group, a carbocyclic group, a heterocyclic group, halogen, CN, CO₂Rₐ, —C(O)Rₐ, —CORₐ, C(O)NRₐRₐ, which may be optionally substituted, wherein each Rₐ is independently selected from the group consisting of H, an aliphatic group, a carbocyclic group, and a heterocyclic group; or R and R₁, taken together, may form a substituted or unsubstituted spiro heterocyclic or carbocyclic ring, which may optionally be substituted;
R₁ and Rₓ are independently selected from the group consisting of H, -M₁, -M₁-M₂, -Z-M₂, and -M₁-Z-M₂; or R and R₁, taken together, may form a substituted or unsubstituted spiro heterocyclic or carbocyclic ring, which may optionally be substituted;
M₁ and M₂ are independently selected from the group consisting of H, an aliphatic group, a carbocyclic group, a heterocyclic group, which may be optionally substituted;
Z is selected from the group consisting of —O—, —NH—, —CR_{z}R_{z}—, —S—, —S(O)—, —C(O)—, —NHC(O)—, —C(O)NH—, —NHC(O)CH₂O—, —S(O)₂—, —CH(OH)—, —CH(OR_{z}), —C(O)CH₂—, —CH₂C(O)—, —CH₂CH(OH)—, —CH₂CH(OR_{z})—, —CH(OH)CH₂—, —CH(OR_{z})CH₂—, and any combination thereof, wherein each R_{z} is independently selected from the group consisting of H, an aliphatic group, a carbocyclic group, a heterocyclic group, hydroxy, and alkoxy;
R₂ is selected from the group consisting of H, an aliphatic group, a carbocyclic group, and a heterocyclic group;
R₃ is selected from the group consisting of -G₁, -G₁-G₂, —Y-G₂, and -G₁-Y-G₂;
G₁ and G₂ are independently selected from H, an aliphatic group, a carbocyclic group, and a heterocyclic group, which may be optionally substituted with one or more of substituents; and
Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂—, —CH(OH)—, —CH(OR_{y}), —C(O)CH₂—, —CH₂C(O)—, —CH₂CH(OH)—, —CH₂CH(OR_{y})—, —CH(OH)CH₂—, —CH(OR_{y})CH₂—, and any combination thereof, wherein each R_{y} is independently selected from the group consisting of H, an aliphatic group, a carbocyclic group, a heterocyclic group, hydroxy, and alkoxy.

109-110. (canceled)

111. A compound of Formula VII: 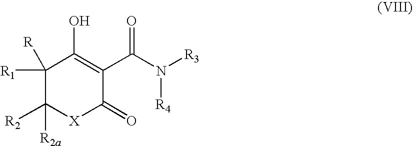
wherein
X is selected from the group consisting of NRₓ and O;
R is absent or selected from the group consisting of H, an aliphatic group, a carbocyclic group, a heterocyclic group, halogen, NO₂, CN, ORₐ, NRₐRₐ, CO₂Rₐ, —C(O)Rₐ, —CORₐ, NRₐC(O)Rₐ, NRₐC(O)NRₐRₐ, NRₐRₐC(O)O—, C(O)NRₐRₐ, which may be optionally substituted, wherein each Rₐ is independently selected from the group consisting of H, an aliphatic group, a carbocyclic group, and a heterocyclic group; or R and R₁, taken together, may form a substituted or unsubstituted spiro heterocyclic or carbocyclic ring, which may optionally be substituted; or R₂ and R₂ₐ, taken together, may form a substituted or unsubstituted spiro heterocyclic or carbocyclic ring, which may optionally be substituted;
R₂ₐ is absent or selected from the group consisting of H, an aliphatic group, a carbocyclic group, a heterocyclic group, halogen, CN, CO₂Rₐ, —C(O)Rₐ, —CORₐ, C(O)NRₐRₐ, which may be optionally substituted, wherein each Rₐ is independently selected from the group consisting of H, an aliphatic group, a carbocyclic group, and a heterocyclic group; of R and R₁, taken together, may form a substituted or unsubstituted spiro heterocyclic or carbocyclic ring, which may optionally be substituted; or R₂ and R₂ₐ, taken together, may form a substituted or unsubstituted spiro heterocyclic or carbocyclic ring, which may optionally be substituted;
R₁, R₂, and Rₓ are independently selected from the group consisting of H, -M₁, -M₁-M₂, -Z-M₂, and -M₁-Z-M₂; or R and R₁, taken together, may form a substituted or unsubstituted spiro heterocyclic or carbocyclic ring, which may optionally be substituted; or R₂ and R₂ₐ, taken together, may form a substituted or unsubstituted spiro heterocyclic or carbocyclic ring, which may optionally be substituted;
M₁ and M₂ are independently selected from the group consisting of H, an aliphatic group, a carbocyclic group, a heterocyclic group, which may be optionally substituted;
Z is selected from the group consisting of —O—, —NH—, —CR_{z}R_{z}—, —S—, —S(O)—, —C(O)—, —NHC(O)—, —C(O)NH—, —NHC(O)CH₂O—, —S(O)₂—, —CH(OH)—, —CH(OR_{z}), —C(O)CH₂—, —CH₂C(O)—, —CH₂CH(OH)—, —CH₂CH(OR_{z})—, —CH(OH)CH₂—, —CH(OR₂)CH₂—, and any combination thereof, wherein each R_{z} is independently selected from the group consisting of H, an aliphatic group, a carbocyclic group, a heterocyclic group, hydroxy, and alkoxy;
R₃ is selected from the group consisting of -G₁, -G₁-G₂, —Y-G₂, and -G₁-Y-G₂;
G₁ and G₂ are independently selected from H, an aliphatic group, a carbocyclic group, and a heterocyclic group, which may be optionally substituted with one or more of substituents;
Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂—, —CH(OH)—, —CH(OR_{y}), —C(O)CH₂—, —CH₂C(O)—, —CH₂CH(OH)—, —CH₂CH(OR_{y})—, —CH(OH)CH₂—, —CH(OR_{y})CH₂—, and any combination thereof, wherein each R_{y} is independently selected from the group consisting of H, an aliphatic group, a carbocyclic group, a heterocyclic group, hydroxy, and alkoxy; and
R₄ is selected from the group consisting of H, an aliphatic group, a carbocyclic group, and a heterocyclic group.

112-113. (canceled)

114. A compound of Formula IX: 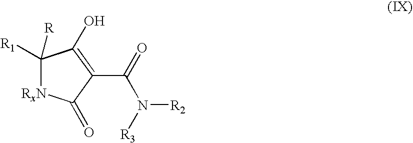
wherein
R is selected from the group consisting of H, benzyl, pyridinyl, tetrahydro-pyranyl, methyl-1H-imidazolyl, cyclohexylmethyl, phenethyl, p-chlorobenzyl, carboxylic acid benzyl ester, propionic acid tert-butyl ester, tert-butyl ester, ethanone, hydroxy, methoxy, ethoxy, propoxy, butoxy, t-butoxy, phenyl, isobutyl, methyl, ethyl, propyl, butyl, cyclohexyl, cyclohexylmethyl, m-methoxy phenyl, carboxylic acid 2-methoxy-ethyl ester, 3,3-dimethyl-butan-1-one, 2,2-dimethyl-propan-1-one, carboxylic acid methyl ester; alkyl, halogen, CN, CO₂Rₐ, —C(O)Rₐ, —CORₐ, C(O)NRₐRₐ, aryl, and heterocycle, which may be optionally substituted with methoxy or 2-methoxy-ethoxy, wherein each Rₐ is independently selected from the group consisting of H, alkyl, aryl, and heterocycle; or R and R₁, taken together, may form a substituted or unsubstituted spiro heterocyclic or carbocyclic ring, which may optionally be substituted with a benzyl group;
R₁ and Rₓ are independently selected from the group consisting of H, benzyl, pyridinyl, tetrahydro-pyranyl, methyl-1H-imidazolyl, cyclohexylmethyl, phenethyl, p-chlorobenzyl, carboxylic acid benzyl ester, propionic acid tert-butyl ester, tert-butyl ester, ethanone, propoxy, t-butoxy, phenyl, isobutyl, methyl, ethyl, propyl, butyl, cyclohexyl, cyclohexylmethyl, m-methoxy phenyl, carboxylic acid 2-methoxy-ethyl ester, 3,3-dimethyl-butan-1-one, 2,2-dimethyl-propan-1-one, carboxylic acid methyl ester, alkyl, halogen, CN, CO₂R_{b}, —C(O)R_{b}, —COR_{b}, C(O)NR_{b}R_{b}, aryl, and heterocycle, which may be optionally substituted with methoxy or 2-methoxy-ethoxy, wherein each R_{b} is independently selected from the group consisting of H, alkyl, aryl, and heterocycle; or R and R₁, taken together, may form a substituted or unsubstituted spiro heterocyclic or carbocyclic ring, which may optionally be substituted with a benzyl group;
R₂ is selected from the group consisting of H, phenyl, benzyl, isobutyl, cyclohexyl, cyclohexylmethyl, m-methoxy phenyl, alkyl, aryl, and heterocycle;
R₃ is selected from the group consisting of -G₁, -G₁-G₂, —Y-G₂, and -G₁-Y-G₂;
G₁ and G₂ are independently selected from the group consisting of phenyl, cyclohexyl, cyclopentyl, 4-indanyl, pyrimidinyl, N-morpholino, furanyl, thiophenyl, pyrrolyl, N-1H-pyridin-2-onyl, bicyclo[4.2.0]octa-1,3,5-trien-3-yl, 1-indanyl, naphthalenyl, tetrahydro-naphthalenyl, pyrazine, [1,2,3]thiadiazolyl, 3-isoxazolyl, 5-indolyl, 2,3-dihydro-indol-6-yl, indazol-5-yl, benzo[2,1,3]thiadiazol-5-yl, cycloheptyl, isopropyl-[1,3,4]thiadiazolyl, benzothiazolyl, 3-methyl-butyl, 1H-pyrazolyl, oxazolyl, piperidinyl, 1H-imidazolyl, pyrrolidinyl, piperazinyl, 1H-[1,2,4]triazolyl, and pyridinyl, which may be optionally substituted with one or more of substituent moieties selected from the group consisting of CF₃, OCF₃, iodo, chloro, bromo, —C(O)NH₂, —O(CH₂)₅CH₃, carboxylic acid methyl ester, phenyl, p-methoxy phenyl, —NHC(O)NH₂, —C(O)O(CH₂)₂N(CH₂CH₃)₂, t-butyl, fluoro, methoxy, hydroxy, isopropyl, cyano, isopropenyl tetrahydropyran, benzyl, amino, —NHC(O)OC(CH₃)₃, —C(O)OH, —C(O)CH₃, —CH₂CO₂H, methyl, and —(CH₂)₂—OH; and
Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂—, and any combination thereof, wherein each R_{y} is independently selected from the group consisting of H, alkyl, aryl, heterocycle, hydroxy, or alkoxy.

115-116. (canceled)

117. A compound of Formula X: 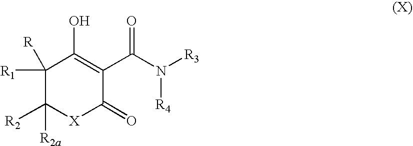
wherein
X is selected from the group consisting of NRₓCRₓRₓ and O;
R₂ and R₂ₐ are absent or independently selected from the group consisting of H, benzyl, pyridinyl, tetrahydro-pyranyl, methyl-1H-imidazolyl, cyclohexylmethyl, phenethyl, p-chlorobenzyl, carboxylic acid benzyl ester, propionic acid tert-butyl ester, tert-butyl ester, ethanone, hydroxy, methoxy, ethoxy, propoxy, butoxy, t-butoxy, phenyl, isobutyl, methyl, ethyl, propyl, butyl, cyclohexyl, cyclohexylmethyl, m-methoxy phenyl, carboxylic acid 2-methoxy-ethyl ester, 3,3-dimethyl-butan-1-one, 2,2-dimethyl-propan-1-one, carboxylic acid methyl ester, alkyl, halogen, CN, CO₂Rₐ, —C(O)Rₐ, —CORₐ, C(O)NRₐRₐ, aryl, and heterocycle, which may be optionally substituted with methoxy or 2-methoxy-ethoxy, wherein each Rₐ is independently selected from the group consisting of H, alkyl, aryl, and heterocycle; or R₂ and R₂ₐ, taken together, may form a substituted or unsubstituted spiro heterocyclic or carbocyclic ring, which may optionally be substituted; or R and R₂ are absent;
R₁, R, and each Rₓ are independently selected from the group consisting of H, benzyl, pyridinyl, tetrahydro-pyranyl, methyl-1H-imidazolyl, cyclohexylmethyl, phenethyl, p-chlorobenzyl, carboxylic acid benzyl ester, propionic acid tert-butyl ester, tert-butyl ester, ethanone, hydroxy, methoxy, ethoxy, propoxy, butoxy, t-butoxy, phenyl, isobutyl, methyl, ethyl, propyl, butyl, cyclohexyl, cyclohexylmethyl, m-methoxy phenyl, carboxylic acid 2-methoxy-ethyl ester, 3,3-dimethyl-butan-1-one, 2,2-dimethyl-propan-1-one, carboxylic acid methyl ester, alkyl, halogen, NO₂, CN, OR_{b}, NR_{b}R_{b}, CO₂R_{b}, —C(O)R_{b}, —COR_{b}, NR_{b}C(O)R_{b}, NR_{b}C(O)NR_{b}R_{b}, NR_{b}R_{b}C(O)O—, C(O)NR_{b}R_{b}, aryl, and heterocycle, which may be optionally substituted with methoxy or 2-methoxy-ethoxy, wherein each R_{b} is independently selected from the group consisting of H, alkyl, aryl, and heterocycle; or R and R₁, taken together, may form a substituted or unsubstituted spiro heterocyclic or carbocyclic ring, which may optionally be substituted; or R and R₂ are absent;
R₃ is selected from the group consisting of -G₁, -G₁-G₂, —Y-G₂, and -G₁-Y-G₂;
G₁ and G₂ are independently selected from the group consisting of phenyl, cyclohexyl, cyclopentyl, 4-indanyl, pyrimidinyl, N-morpholino, furanyl, thiophenyl, pyrrolyl, N-1H-pyridin-2-onyl, bicyclo[4.2.0]octa-1,3,5-trien-3-yl, 1-indanyl, naphthalenyl, tetrahydro-naphthalenyl, pyrazine, [1,2,3]thiadiazolyl, 3-isoxazolyl, 5-indolyl, 2,3-dihydro-indol-6-yl, indazol-5-yl, benzo[2,1,3]thiadiazol-5-yl, cycloheptyl, isopropyl-[1,3,4]thiadiazolyl, benzothiazolyl, 3-methyl-butyl, 1H-pyrazolyl, oxazolyl, piperidinyl, 1H-imidazolyl, pyrrolidinyl, piperazinyl, 1H-[1,2,4]triazolyl, and pyridinyl, which may be optionally substituted with one or more of substituent moieties selected from the group consisting of CF₃, OCF₃, iodo, chloro, bromo, —C(O)NH₂, —O(CH₂)₅CH₃, carboxylic acid methyl ester, phenyl, p-methoxy phenyl, —NHC(O)NH₂, —C(O)O(CH₂)₂N(CH₂CH₃)₂, t-butyl, fluoro, methoxy, hydroxy, isopropyl, cyano, isopropenyl tetrahydropyran, benzyl, amino, —NHC(O)OC(CH₃)₃, —C(O)OH, —C(O)CH₃, —CH₂CO₂H, methyl, and —(CH₂)₂—OH;
Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂—, and any combination thereof, wherein each R_{y} is independently selected from the group consisting of H, alkyl, aryl, heterocycle, hydroxy, or alkoxy; and
R₄ is selected from the group consisting of H, phenyl, benzyl, isobutyl, cyclohexyl, cyclohexylmethyl, m-methoxy phenyl, alkyl, aryl, and heterocycle.

118. (canceled)

119. A compound of Formula XI: 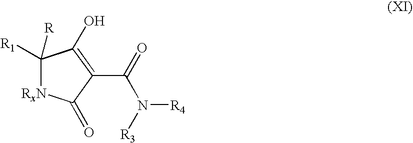
wherein
R₁, R, and Rₓ are independently selected from the group consisting of H, benzyl, pyridinyl, tetrahydro-pyranyl, methyl-1H-imidazolyl, cyclohexylmethyl, phenethyl, p-chlorobenzyl, carboxylic acid benzyl ester, propionic acid tert-butyl ester, tert-butyl ester, ethanone, hydroxy, methoxy, ethoxy, propoxy, butoxy, t-butoxy, phenyl, isobutyl, methyl, ethyl, propyl, butyl, cyclohexyl, cyclohexylmethyl, m-methoxy phenyl, carboxylic acid 2-methoxy-ethyl ester, 3,3-dimethyl-butan-1-one, 2,2-dimethyl-propan-1-one, carboxylic acid methyl ester, alkyl, halogen, CN, CO₂Rₐ, —C(O)Rₐ, —CORₐ, C(O)NRₐRₐ, aryl, and heterocycle, which may be optionally substituted with methoxy or 2-methoxy-ethoxy, wherein each Rₐ is independently selected from the group consisting of H, alkyl, aryl, and heterocycle;
R₃ is selected from the group consisting of -G₁, -G₁-G₂, —Y-G₂, and -G₁-Y-G₂;
G₁ and G₂ are independently selected from the group consisting of phenyl, cyclohexyl, cyclopentyl, 4-indanyl, pyrimidinyl, N-morpholino, furanyl, thiophenyl, pyrrolyl, N-1H-pyridin-2-onyl, bicyclo[4.2.0]octa-1,3,5-trien-3-yl, 1-indanyl, naphthalenyl, tetrahydro-naphthalenyl, pyrazine, [1,2,3]thiadiazolyl, 3-isoxazolyl, 5-indolyl, 2,3-dihydro-indol-6-yl, indazol-5-yl, benzo[2,1,3]thiadiazol-5-yl, cycloheptyl, isopropyl-[1,3,4]thiadiazolyl, benzothiazolyl, 3-methyl-butyl, 1H-pyrazolyl, oxazolyl, piperidinyl, 1H-imidazolyl, pyrrolidinyl, piperazinyl, 1H-[1,2,4]triazolyl, and pyridinyl, which may be optionally substituted with one or more of substituent moieties selected from the group consisting of CF₃, OCF₃, iodo, chloro, bromo, —C(O)NH₂, —O(CH₂)₅CH₃, carboxylic acid methyl ester, phenyl, p-methoxy phenyl, —NHC(O)NH₂, —C(O)O(CH₂)₂N(CH₂CH₃)₂, t-butyl, fluoro, methoxy, hydroxy, isopropyl, cyano, isopropenyl tetrahydropyran, benzyl, amino, —NHC(O)OC(CH₃)₃, —NHC(O)OC(CH₃)₃, —C(O)CH₃, —CH₂CO₂H, methyl, and —(CH₂)₂—OH;
Y is selected from the group consisting of —O—, —NH—, —CR_{y}R_{y}—, —S—, —S(O)—, —C(O)—, —NHC(O)—, —C(O)NH—, —NHC(O)CH₂O—, —S(O)₂—, and any combination thereof, wherein each R_{y} is independently selected from the group consisting of H, alkyl, aryl, heterocycle, hydroxy, or alkoxy; and
R₄ is selected from the group consisting of H, phenyl, benzyl, isobutyl, cyclohexyl, cyclohexylmethyl, m-methoxy phenyl, alkyl, aryl, and heterocycle.

120-122. (canceled)

123. A pharmaceutical composition comprising a therapeutically effective amount of a compound of claim 110, and a pharmaceutically acceptable carrier.

124-147. (canceled)
